(19) United States
(12) Patent Application Publication
(10) Pub. No.: US 2022/0290156 A1
(43) Pub. Date: Sep. 15, 2022

(54) COMPOSITIONS AND METHODS FOR INHIBITING PCSK9

(71) Applicant: SANOFI, Paris (FR)

(72) Inventors: Bodo BRUNNER, Frankfurt am Main (DE); Kerstin JAHN-HOFMANN, Frankfurt am Main (DE); Sabine SCHEIDLER, Frankfurt am Main (DE); Pierrick RIVAL, Paris (FR)

(73) Assignee: SANOFI, Paris (FR)

(21) Appl. No.: 17/638,339

(22) PCT Filed: Aug. 27, 2020
(86) PCT No.: PCT/EP2020/073961

§ 371 Date: Feb. 25, 2022

(30) Foreign Application Priority Data
Aug. 27, 2019 (EP) 19306036.5

(51) Int. Cl. C12N 15/113; A61K 31/713

(52) U.S. Cl. CPC C12N 15/1137; A61K 31/713; C12N 2310/14; C12N 2310/343; C12N 2320/51; C12N 2310/351; C12N 2320/11

(57) ABSTRACT

Provided herein, inter alia, are dsRNA compositions targeting PCSK9, methods of inhibiting PCSK9 gene expression, and methods of treating one or more diseases associated with PCSK9 gene expression.

### FIELD OF THE INVENTION

The present disclosure relates to dsRNA compositions targeting proprotein convertase subtilisin kexin 9 (PCSK9), methods of inhibiting PCSK9 gene expression, and methods of treating one or more diseases associated with PCSK9 gene expression.

### SUBMISSION OF SEQUENCE LISTING

Nucleic acid sequences are disclosed in the present specification that serve as references. The same sequences are also presented in a sequence listing formatted according to standard requirements for the purpose of patent matters. In case of any sequence discrepancy with the standard sequence listing, the sequences described in the present specification shall be the reference.

### BACKGROUND

PCSK9 is a member of the subtilisin serine protease family. The other eight mammalian subtilisin proteases, PCSK1-8, are proprotein convertases that process a wide variety of proteins in the secretory pathway and play roles in diverse biological processes. PCSK9 has been proposed to play a role in cholesterol metabolism. PCSK9 messenger RNA (mRNA) expression is down-regulated by dietary cholesterol feeding in mice (Maxwell, K. N. (2003) J. Lipid Res. 44, 2109-2119), up-regulated by statins in HepG2 cells (Duboc, G. (2004) Arterioscler. Thromb. Vasc. Biol. 24, 1454-1459), and up-regulated in sterol regulatory element binding protein (SREBP) transgenic mice (Horton, J. D. (2003) PNAS 100 12027-12032), similar to the cholesterol biosynthetic enzymes and low-density lipoprotein receptor (LDLR). Furthermore, PCSK9 missense mutations have been found to be associated with a form of autosomal dominant hypercholesterolemia (Abifadel, M. (2003) Nat. Genet. 34, 154-156; Timms, K. M. (2004) Hum. Genet. 114, 349-353; Leren, T. P. (2004) Clin. Genet. 65, 419-422). PCSK9 may also play a role in determining low-density lipoprotein (LDL) cholesterol levels in the general population, as single-nucleotide polymorphisms (SNPs) have been associated with cholesterol levels in a Japanese population (Shioji, K. (2004) J. Hum. Genet. 49, 109-114).

Autosomal dominant hypercholesterolemias (ADHs) are monogenic diseases in which patients exhibit elevated total and LDL cholesterol levels, tendon xanthomas, and premature atherosclerosis (Rader, D. J. (2003) J. Clin. Invest. 111, 1795-1803). The pathogenesis of ADHs and a recessive form, autosomal recessive hypercholesterolemia (ARH) (Cohen, J. C. (2003) Curr. Opin. Lipidol. 14, 121-127), is due to defects in LDL uptake by the liver. ADH may be caused by LDLR mutations, which prevent LDL uptake, or by mutations in the protein on LDL, apolipoprotein B, which binds to the LDLR. ARH is caused by mutations in the low density lipoprotein receptor adapter protein 1 (LDLRAP1) protein that is necessary for endocytosis of the LDLR-LDL complex via its interactions with clathrin.

Overexpression studies point to a role for PCSK9 in controlling LDLR levels and, hence, LDL uptake by the liver (Maxwell, K. N. (2004) PNAS 101, 7100-7105; Benjannet, S. et al. (2004) J. Biol. Chem. 279, 48865-48875; Park, S. W. (2004) J. Biol. Chem. 279, 50630-50638). Adenoviral-mediated overexpression of mouse or human PCSK9 in mice results in elevated total and LDL cholesterol levels; this effect is not seen in LDLR knockout animals (Maxwell, K. N. (2004) PNAS 101, 7100-7105; Benjannet, S. et al. (2004) J. Biol. Chem. 279, 48865-48875; Park, S. W. (2004) J. Biol. Chem. 279, 50630-50638). In addition, PCSK9 overexpression results in a severe reduction in hepatic LDLR protein, without affecting LDLR mRNA levels, SREBP protein levels, or SREBP protein nuclear to cytoplasmic ratio.

Loss of function mutations in PCSK9 have been designed in mouse models (Rashid et al. (2005) PNAS, 102, 5374-5379), and identified in human individuals (Cohen et al. (2005) Nature Genetics 37: 161-165). In both cases, loss of PCSK9 function leads to lowering of total LDL cholesterol (LDL-C). The effect of lifelong reductions in plasma LDL-C associated with sequence variations in PCSK9 gene was studied, and the data indicated that moderate lifelong reduction in the plasma level of LDL-C was associated with a substantial reduction in the incidence of coronary events and conferred protection against coronary heart disease. (Cohen et al. (2006) N. Engl. J. Med. 354: 1264-1272).

Double-stranded RNA molecules (dsRNA) have been shown to block gene expression in a highly conserved regulatory mechanism known as RNA interference (RNAi). WO 99/32619 disclosed the use of a dsRNA of at least 25 nucleotides in length to inhibit the expression of genes in C. elegans. dsRNA has also been shown to degrade target RNA in other organisms, including plants (See e.g., WO 99/53050; WO 99/61631), Drosophila (See e.g., Yang, D. et al. (2000) Curr. Biol. 10: 1191-1200), and mammals (See e.g., WO 00/44895). This natural mechanism has now become the focus for the development of a new class of pharmaceutical agents for treating disorders that are cause by the aberrant or unwanted regulation of a gene.

Due to the importance of PCSK9 in regulating LDL cholesterol and the prevalence of cardiovascular diseases such as hypercholesterolemias, there is a continuing need to identify inhibitors of PCSK9 expression such as dsRNAs and to test such inhibitors for efficacy and unwanted side effects, such as cytotoxicity.

All references cited herein, including patent applications, patent publications, non-patent literature, and UniProtKB/Swiss-Prot Accession numbers are herein incorporated by reference in their entirety, as if each individual reference were specifically and individually indicated to be incorporated by reference.

### BRIEF SUMMARY

To meet these and other needs, provided herein are double-stranded ribonucleic acids (dsRNAs) useful for inhibiting expression of a Proprotein Convertase Subtilisin Kexin 9 (PCKS9) gene.

Accordingly, in one aspect, provided herein is a double-stranded ribonucleic acid (dsRNA), wherein the dsRNA comprises a sense strand comprising a first sequence and an antisense strand comprising a second sequence, wherein the first sequence and the second sequence are complementary, and wherein the first sequence comprises a sequence selected from the group consisting of SEQ ID NOS: 6-11 and 310-321.

According to another aspect, the present disclosure provides a double-stranded ribonucleic acid (dsRNA), wherein the dsRNA comprises a sense strand comprising a first sequence and an antisense strand comprising a second sequence, wherein the first sequence and the second sequence are complementary, wherein the first sequence comprises a sequence selected from the group consisting of SEQ ID NOS: 6-11 and 310-321, wherein the dsRNA is optionally a small interfering RNA (siRNA) or short hairpin RNA (shRNA), and wherein the dsRNA optionally inhibits expression of a Proprotein Convertase Subtilisin Kexin 9 (PCSK9) gene.

In another embodiment, the disclosure provides a double-stranded ribonucleic acid (dsRNA), wherein the dsRNA comprises a sense strand comprising a first sequence and an antisense strand comprising a second sequence, wherein the first sequence and the second sequence are complementary, wherein the first sequence comprises a sequence selected from the group consisting of SEQ ID NOS: 6-11 and 310-321, wherein said selected sequence comprises less than 30% GC, wherein the dsRNA is optionally a small interfering RNA (siRNA) or short hairpin RNA (shRNA), and wherein the dsRNA optionally inhibits expression of a Proprotein Convertase Subtilisin Kexin 9 (PCSK9) gene.

In some embodiments, the dsRNA comprises (1) UUUUAUUAAUAUGGUGACU (SEQ ID NO:6) in the sense strand and AGUCACCAUAUUAAUAAAA (SEQ ID NO:373) in the antisense strand; (2) UAUUAAUAUGGUGACUUUU (SEQ ID NO:7) in the sense strand and AAAAGUCACCAUAUUAAUA (SEQ ID NO:374) in the antisense strand; (3) AUUAAUAUGGUGACUUUUU (SEQ ID NO:8) in the sense strand and AAAAAGUCACCAUAUUAAU (SEQ ID NO:375) in the antisense strand; (4) UUAAUAUGGUGACUUUUUA (SEQ ID NO:9) in the sense strand and UAAAAAGUCACCAUAUUAA (SEQ ID NO:376) in the antisense strand; (5) UAAUAUGGUGACUUUUUAA (SEQ ID NO:10) in the sense strand and UUAAAAAGUCACCAUAUUA (SEQ ID NO:377) in the antisense strand; (6) UAUGGUGACUUUUUAAAAU (SEQ ID NO:11) in the sense strand and AUUUUAAAAAGUCACCAUA (SEQ ID NO:378) in the antisense strand; (7) UUAUUAAUAUGGUGACUUU (SEQ ID NO:310) in the sense strand and AAAGUCACCAUAUUAAUAA (SEQ ID NO:380) in the antisense strand; (8) AUAUGGUGACUUUUUAAAA (SEQ ID NO:311) in the sense strand and UUUUAAAAAGUCACCAUAU (SEQ ID NO:381) in the antisense strand; (9) AUUUUUAUUAAUAUGGUGACU (SEQ ID NO:312) in the sense strand and AGUCACCAUAUUAAUAAAAAU (SEQ ID NO:382) in the antisense strand; (10) UUUUAUUAAUAUGGUGACUUU (SEQ ID NO:313) in the sense strand and AAAGUCACCAUAUUAAUAAAA (SEQ ID NO:383) in the antisense strand; (11) UUUAUUAAUAUGGUGACUUUU (SEQ ID NO:314) in the sense strand and AAAAGUCACCAUAUUAAUAAA (SEQ ID NO:384) in the antisense strand; (12) UAUUAAUAUGGUGACUUUUUA (SEQ ID NO:315) in the sense strand and UAAAAAGUCACCAUAUUAAUA (SEQ ID NO:385) in the antisense strand; (13) AAUAUGGUGACUUUUUAAAAU (SEQ ID NO:316) in the sense strand and AUUUUAAAAAGUCACCAUAUU (SEQ ID NO:386) in the antisense strand; (14) GCAUUUUUAUUAAUAUGGUGACU (SEQ ID NO:317) in the sense strand and AGUCACCAUAUUAAUAAAAAUGC (SEQ ID NO:387) in the antisense strand; (15) AUUUUUAUUAAUAUGGUGACUUU (SEQ ID NO:318) in the sense strand and AAAGUCACCAUAUUAAUAAAAAU (SEQ ID NO:388) in the antisense strand; (16) UUUUUAUUAAUAUGGUGACUUUU (SEQ ID NO:319) in the sense strand and AAAAGUCACCAUAUUAAUAAAAA (SEQ ID NO:389) in the antisense strand; (17) UUUAUUAAUAUGGUGACUUUUUA (SEQ ID NO:320) in the sense strand and UAAAAAGUCACCAUAUUAAUAAA (SEQ ID NO:390) in the antisense strand; or (18) UUAUUAAUAUGGUGACUUUUUAA (SEQ ID NO:321) in the sense strand and UUAAAAAGUCACCAUAUUAAUAA (SEQ ID NO:391) in the antisense strand. In some embodiments, the dsRNA comprises (1) CCAUUUUAUUAAUAUGGUGACUinvdT (SEQ ID NO:176) in the sense strand and AGUCACCAUAUUAAUAAAAdTdT (SEQ ID NO:177) in the antisense strand; (2) CCAUAUUAAUAUGGUGACUUUUinvdT (SEQ ID NO:180) in the sense strand and AAAAGUCACCAUAUUAAUAdTdT (SEQ ID NO:181) in the antisense strand; (3) CCAAUUAAUAUGGUGACUUUUUinvdT (SEQ ID NO:182) in the sense strand and AAAAAGUCACCAUAUUAAUdTdT (SEQ ID NO:183) in the antisense strand; (4) CCAUUAAUAUGGUGACUUUUUAinvdT (SEQ ID NO:184) in the sense strand and UAAAAAGUCACCAUAUUAAdTdT (SEQ ID NO:185) in the antisense strand; (5) CCAUAAUAUGGUGACUUUUUAAinvdT (SEQ ID NO:186) in the sense strand and UUAAAAAGUCACCAUAUUAdTdT (SEQ ID NO:187) in the antisense strand; (6) CCAUAUGGUGACUUUUUAAAAUinvdT (SEQ ID NO:188) in the sense strand and AUUUUAAAAAGUCACCAUAdTdT (SEQ ID NO:189) in the antisense strand; (7) CCAUUAUUAAUAUGGUGACUUUinvdT (SEQ ID NO:322) in the sense strand and AAAGUCACCAUAUUAAUAAdTdT (SEQ ID NO:323) in the antisense strand; (8) CCAAUAUGGUGACUUUUUAAAAinvdT (SEQ ID NO:324) in the sense strand and UUUUAAAAAGUCACCAUAUdtdt (SEQ ID NO:325) in the antisense strand; (9) CCAAUUUUUAUUAAUAUGGUGACUinvdT (SEQ ID NO:326) in the sense strand and AGUCACCAUAUUAAUAAAAAUdTdT (SEQ ID NO:327) in the antisense strand; (10) CCAUUUUAUUAAUAUGGUGACUUUinvdT (SEQ ID NO:328) in the sense strand and AAAGUCACCAUAUUAAUAAAAdTdT (SEQ ID NO:329) in the antisense strand; (11) CCAUUUAUUAAUAUGGUGACUUUUinvdT (SEQ ID NO:330) in the sense strand and AAAAGUCACCAUAUUAAUAAAdTdT (SEQ ID NO:331) in the antisense strand; (12) CCAUAUUAAUAUGGUGACUUUUUAinvdT (SEQ ID NO:332) in the sense strand and UAAAAAGUCACCAUAUUAAUAdTdT (SEQ ID NO:333) in the antisense strand; (13) CCAAAUAUGGUGACUUUUUAAAAUinvdT (SEQ ID NO:334) in the sense strand and AUUUUAAAAAGUCACCAUAUUdTdT (SEQ ID NO:335) in the antisense strand; (14) CCAGCAUUUUUAUUAAUAUGGUGACUinvdT (SEQ ID NO:336) in the sense strand and AGUCACCAUAUUAAUAAAAAUGCdTdT (SEQ ID NO:337) in the antisense strand; (15) CCAAUUUUUAUUAAUAUGGUGACUUUinvdT (SEQ ID NO:338) in the sense strand and AAAGUCACCAUAUUAAUAAAAAUdTdT (SEQ ID NO:339) in the antisense strand; (16) CCAUUUUUAUUAAUAUGGUGACUUUUinvdT (SEQ ID NO:340) in the sense strand and AAAAGUCACCAUAUUAAUAAAAAdTdT (SEQ ID NO:341) in the antisense strand; (17) CCAUUUAUUAAUAUGGUGACUUUUUAinvdT (SEQ ID NO:342) in the sense strand and UAAAAAGUCACCAUAUUAAUAAAdTdT (SEQ ID NO:343) in the antisense strand; or (18) CCAUUAUUAAUAUGGUGACUUUUUAAinvdT (SEQ ID NO:344) in the sense strand and UUAAAAAGUCACCAUAUUAAUAAdTdT (SEQ ID NO:345) in the antisense strand. In some embodiments, the first sequence is identical to at least 15 contiguous nucleotides of UUGUAGCAUUUUUAUUAAUAUGGUGACUUUUUAAAAUAAAAACAAACA (SEQ ID NO:2) and is not one of GCAUUUUUAUUAAUAUGGU (SEQ ID NO: 5), UUUGUAGCAUUUUUAUUAAUAUGGU (SEQ ID NO: 576), or AUUUUUAUUAAUAUGGUGA (SEQ ID NO: 577).

In another aspect, the present disclosure relates to a double-stranded ribonucleic acid (dsRNA), wherein the dsRNA comprises a sense strand comprising a first sequence and an antisense strand comprising a second sequence, wherein only the first sequence and the second sequence are complementary, and wherein the first sequence is one of SEQ ID NOS: 3, 4, and 13. In another aspect, the present disclosure relates to a double-stranded ribonucleic acid (dsRNA), wherein the dsRNA comprises a sense strand comprising a first sequence and an antisense strand comprising a second sequence, wherein only the first sequence and the second sequence are complementary, wherein the first sequence is one of SEQ ID NOS: 3, 4, and 13, wherein the dsRNA is optionally a small interfering RNA (siRNA) or short hairpin RNA (shRNA), and wherein the dsRNA optionally inhibits expression of a Proprotein Convertase Subtilisin Kexin 9 (PCSK9) gene. In some embodiments, the disclosure provides a dsRNA comprises a sense strand comprising a first sequence and an antisense strand comprising a second sequence, wherein only the first sequence and the second sequence are complementary, wherein the first sequence is one of SEQ ID NOS: 3, 4, and 13, wherein the first sequence comprises less than 30% GC wherein the dsRNA is optionally a small interfering RNA (siRNA) or short hairpin RNA (shRNA), and wherein the dsRNA optionally inhibits expression of a Proprotein Convertase Subtilisin Kexin 9 (PCSK9) gene. In some embodiments, the dsRNA comprises: (19) UUGUAGCAUUUUUAUUAAU (SEQ ID NO:3) in the sense strand and AUUAAUAAAAAUGCUACAA (SEQ ID NO:370) in the antisense strand; (20) GUAGCAUUUUUAUUAAUAU (SEQ ID NO:4) in the sense strand and AUAUUAAUAAAAAUGCUAC (SEQ ID NO:371) in the antisense strand; or (21) GAGUGUGAAAGGUGCUGAU (SEQ ID NO:13) in the sense strand and AUCAGCACCUUUCACACUC (SEQ ID NO:379) in the antisense strand. In some embodiments, the dsRNA comprises: (19) CCAUUGUAGCAUUUUUAUUAAUinvdT (SEQ ID NO:162) in the sense strand and AUUAAUAAAAAUGCUACAAdTdT (SEQ ID NO:163) in the antisense strand; (20) CCAGUAGCAUUUUUAUUAAUAUinvdT (SEQ ID NO:166) in the sense strand and AUAUUAAUAAAAAUGCUACdTdT (SEQ ID NO:167) in the antisense strand; or (21) CCAGAGUGUGAAAGGUGCUGAUinvdT (SEQ ID NO:290) in the sense strand and AUCAGCACCUUUCACACUCdTdT (SEQ ID NO:291) in the antisense strand.

In some embodiments that may be combined with any of the preceding embodiments, the first and second sequences are each less than or equal to 30 nucleotides in length. In some embodiments that may be combined with any of the preceding embodiments, the first and second sequences are each at least 19 and/or less than or equal to 23 nucleotides in length. In some embodiments that may be combined with any of the preceding embodiments, the dsRNA is a small interfering RNA (siRNA) or short hairpin RNA (shRNA).

In some embodiments that may be combined with any of the preceding embodiments, the dsRNA comprises one or more modified nucleotides. In some embodiments, at least one of the one or more modified nucleotides is a 2′-O-methyl nucleotide, 5′-phosphorothioate nucleotide, or a terminal nucleotide linked to a cholesterol derivative or lipophilic moiety. In some embodiments, at least one of the one or more modified nucleotides is a 2′-fluoro, 2′-deoxy, 2′-O-methoxyethyl, constrained ethyl (cEt), deoxy, inverted deoxy, inverted dideoxy, locked nucleic acid, abasic, 2′-amino, 2′-alkyl, morpholino, phosphoramidate, or a non-natural base-containing nucleotide. In some embodiments, the dsRNA comprises one or more 2′-O-methyl nucleotides and one or more 2′-fluoro nucleotides. In some embodiments, the dsRNA comprises two or more 2′-O-methyl nucleotides and two or more 2′-fluoro nucleotides in the pattern OMe-F-OMe-F or F-OMe-F-OMe, wherein OMe represents a 2′-O-methyl nucleotide, and wherein F represents a 2′-fluoro nucleotide. In some embodiments, the dsRNA comprises up to 10 contiguous nucleotides that are each a 2′-O-methyl nucleotide or up to 10 contiguous nucleotides that are each a 2′-fluoro nucleotide.

In some embodiments that may be combined with any of the preceding embodiments, the dsRNA comprises one or more phosphorothioate groups. In some embodiments that may be combined with any of the preceding embodiments, the dsRNA does not comprise a phosphorothioate group. In some embodiments that may be combined with any of the preceding embodiments, the dsRNA comprises one or more phosphotriester groups. In some embodiments that may be combined with any of the preceding embodiments, the dsRNA does not comprise a phosphotriester group.

In some embodiments that may be combined with any of the preceding embodiments, the dsRNA is attached to one or more GalNAc derivatives via a linker. In some embodiments, the dsRNA is attached to three GalNAc derivatives via a trivalent branched linker. In some embodiments, at least one of the one or more GalNAc derivatives is attached to the 3′ end of the sense strand, the 3′ end of the antisense strand, or the 5′ end of the sense strand of the dsRNA.

In some embodiments that may be combined with any of the preceding embodiments, one or both of the sense strand and the antisense strand further comprises a 5′ overhang comprising one or more nucleotides. In some embodiments that may be combined with any of the preceding embodiments, one or both of the sense strand and the antisense strand further comprises a 3′ overhang comprising one or more nucleotides. In some embodiments, the 3′ overhang comprises two nucleotides. In some embodiments, the overhang comprises one or more thymines.

In some embodiments, one or both of strands of the dsRNA comprise one or more compounds having the structure of formula (I):

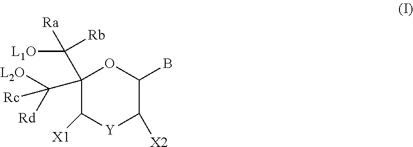

wherein:
B is a heterocyclic nucleobase;
one of L1 and L2 is an internucleoside linking group linking the compound of formula (I) to a polynucleotide and the other of L1 and L2 is H, a protecting group, a phosphorus moiety or an internucleoside linking group linking the compound of formula (I) to a polynucleotide,
Y is O, NH, NR1 or N—C(═O)—R1, wherein R1 is:
   a (C1-C20) alkyl group, optionally substituted by one or more groups selected from an halogen atom, a (C1-C6) alkyl group, a (C3-C8) cycloalkyl group, a (C3-C14) heterocycle, a (C6-C14) aryl group, a (C5-C14) heteroaryl group, —O—Z1, —N(Z1)(Z2), —S—Z1, —CN, —C(=J)-O—Z1, —O—C(=J)-Z1, —C(=J)-N(Z1)(Z2), and —N(Z1)-C(=J)-Z2, wherein
      J is O or S,
      each of Z1 and Z2 is, independently, H, a (C1-C6) alkyl group, optionally substituted by one or more groups selected from a halogen atom and a (C1-C6) alkyl group,
   a (C3-C8) cycloalkyl group, optionally substituted by one or more groups selected from a halogen atom and a (C1-C6) alkyl group,
   a group —[C(═O)]m-R2-(O—CH2-CH2)p-R3, wherein
      m is an integer meaning 0 or 1,
      p is an integer ranging from 0 to 10,
      R2 is a (C1-C20) alkylene group optionally substituted by a (C1-C6) alkyl group, —O—Z3, —N(Z3)(Z4), —S—Z3, —CN, —C(═K)—O—Z3, —O—C(═K)—Z3, —C(═K)—N(Z3)(Z4), or —N(Z3)-C(═K)—Z4, wherein
         K is O or S,
         each of Z3 and Z4 is, independently, H, a (C1-C6) alkyl group, optionally substituted by one or more groups selected from a halogen atom and a (C1-C6) alkyl group, and
      R3 is selected from the group consisting of a hydrogen atom, a (C1-C6) alkyl group, a (C1-C6) alkoxy group, a (C3-C8) cycloalkyl group, a (C3-C14) heterocycle, a (C6-C14) aryl group or a (C5-C14) heteroaryl group, or R3 is a cell targeting moiety,
X1 and X2 are each, independently, a hydrogen atom, a (C1-C6) alkyl group, and
each of Ra, Rb, Rc and Rd is, independently, H or a (C1-C6) alkyl group,
   or is a pharmaceutically acceptable salt thereof.

In some embodiments, the dsRNA comprises one or more compounds of formula (I) wherein Y is:
a) NR1, and R1 is a non-substituted (C1-C20) alkyl group;
b) NR1, and R1 is a non-substituted (C1-C16) alkyl group, which includes an alkyl group selected from a group comprising methyl, isopropyl, butyl, octyl, and hexadecyl;
c) NR1, and R1 is a (C3-C8) cycloalkyl group, optionally substituted by one or more groups selected from a halogen atom and a (C1-C6) alkyl group;
d) NR1, and R1 is a cyclohexyl group;
e) NR1, and R1 is a (C1-C20) alkyl group substituted by a (C6-C14) aryl group;
f) NR1, and R1 is a methyl group substituted by a phenyl group;
g) N—C(═O)—R1, and R1 is an optionally substituted (C1-C20) alkyl group; or
h) N—C(═O)—R1, and R1 is methyl or pentadecyl.

In some embodiments, the dsRNA comprises one or more compounds of formula (I) wherein B is selected from a group consisting of a pyrimidine, a substituted pyrimidine, a purine and a substituted purine, or a pharmaceutically acceptable salt thereof.

In some embodiments, R3 is of formula (II)

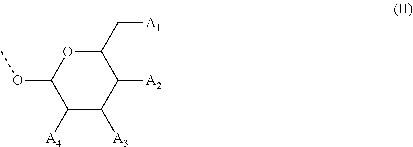

wherein A1, A2 and A3 are OH,
A4 is OH or NHC(═O)—R5, wherein R5 is a (C1-C6) alkyl group, optionally substituted by an halogen atom, or a pharmaceutically acceptable salt thereof

In some embodiments, R3 is N-acetyl-galactosamine, or a pharmaceutically acceptable salt thereof.

In some embodiments, the dsRNA comprises one or more nucleotides from Table A.

In some embodiments, the dsRNA comprises from 2 to 10 compounds of formula (I), or a pharmaceutically acceptable salt thereof. In some embodiments, the 2 to 10 compounds of formula (I) are on the sense strand.

In some embodiments, the sense strand comprises two to five compounds of formula (I) at the 5′ end, and/or comprises one to three compounds of formula (I) at the 3′ end.

In some embodiments,

a) the two to five compounds of formula (I) at the 5′ end of the sense strand comprise lgT3, optionally comprising three consecutive lgT3 nucleotides; and/or
b) the one to three compounds of formula (I) at the 3′ end of the sense strand comprise lT4; optionally comprising two consecutive lT4.

In some embodiments, the dsRNA comprises one or more internucleoside linking groups independently selected from the group consisting of phosphodiester, phosphotriester, phosphorothioate, phosphorodithioate, alkyl-phosphonate and phosphoramidate backbone linking groups, or a pharmaceutically acceptable salt thereof.

In some embodiments, the dsRNA is selected from the dsRNAs in Tables 2-4.

In some embodiments,

a) the sense strand comprises a nucleotide sequence selected from the group consisting of SEQ ID NOs: 578, 585, 587, 620, 621, 622, and 627; and/or
b) the antisense strand comprises a nucleotide sequence selected from the group consisting of SEQ ID NOs: 589, 591, 631, 632, 634, 635 and 639.

In some embodiments, the sense strand and antisense strand of the dsRNA respectively comprise the nucleotide sequences of:

a) SEQ ID NOs: 578 and 589; [C027.001]

b) SEQ ID NOs: 620 and 631; [C027.003]

c) SEQ ID NOs: 585 and 591; [C027.001#40]

d) SEQ ID NOs: 587 and 591; [C027.001#58]

e) SEQ ID NOs: 621 and 634; [C027.003#03]

f) SEQ ID NOs: 622 and 632; [C027.003#06]

g) SEQ ID NOs: 622 and 635; and [C027.003#08]

h) SEQ ID NOs: 627 and 639. [C027.003#47]

In some embodiments that may be combined with any of the preceding embodiments, the dsRNA inhibits expression of a Proprotein Convertase Subtilisin Kexin 9 (PCSK9) gene. In some embodiments, the PCSK9 gene is a human PCSK9 gene (e.g., comprising the polynucleotide sequence of SEQ ID NO:1). In some embodiments, the PCSK9 gene is a non-human PCSK9 gene. In some embodiments, the PCSK9 gene is a non-human primate PCSK9 gene (e.g., cynomolgus monkey PCSK9, such as that represented by UniprotKB Accession No. G7NVZ1).

In another aspect, the present disclosure relates to a vector encoding one or more dsRNAs described herein.

In another aspect, the present disclosure relates to an isolated host cell comprising one or more dsRNAs and/or vectors described herein.

In another aspect, the present disclosure relates to an article of manufacture or kit comprising one or more dsRNAs and/or vectors described herein.

In another aspect, the present disclosure relates to a composition comprising one or more dsRNAs and/or vectors described herein. In some embodiments, the composition is a pharmaceutical composition. In some embodiments, the composition comprises a pharmaceutically acceptable carrier. In some embodiments, the composition comprises a delivery vehicle. In some embodiments, the delivery vehicle is selected from a liposome, lipoplex, complex, and nanoparticle.

In another aspect, the present disclosure relates to a method of inhibiting expression of a PCSK9 gene in a subject, comprising administering to the subject an effective amount of one or more dsRNAs described herein and/or one or more compositions described herein. In another aspect, the present disclosure relates to the use of one or more dsRNAs described herein and/or one or more compositions described herein in a method of inhibiting expression of a PCSK9 gene in a subject. In another aspect, the present disclosure relates to one or more dsRNAs described herein and/or one or more compositions described herein for use in the manufacture of a medicament for inhibiting expression of a PCSK9 gene in a subject. In another aspect, the present disclosure relates to a method of treating or preventing a PCSK9-mediated disease in a subject in need thereof, comprising administering to the subject an effective amount of one or more dsRNAs described herein and/or one or more compositions described herein. In another aspect, the present disclosure relates to the use of one or more dsRNAs described herein and/or one or more compositions described herein in a method of treating or preventing a PCSK9-mediated disease in a subject in need thereof. In another aspect, the present disclosure relates to one or more dsRNAs described herein and/or one or more compositions described herein for use in the manufacture of a medicament for treating or preventing a PCSK9-mediated disease in a subject in need thereof. In some embodiments that may be combined with any of the preceding embodiments, expression of the PCSK9 gene in the liver of the subject is inhibited by the dsRNA. In some embodiments that may be combined with any of the preceding embodiments, the PCSK9-mediated disorder is hypercholesterolemia. In some embodiments that may be combined with any of the preceding embodiments, the administration is subcutaneous, intravenous, or pulmonary administration. In some embodiments that may be combined with any of the preceding embodiments, the subject is a human. In some embodiments that may be combined with any of the preceding embodiments, the administration results in a decrease in serum cholesterol in the subject. In some embodiments that may be combined with any of the preceding embodiments, the methods further comprise administering to the subject an effective amount of one or more additional therapeutic agents for treating or preventing a PCSK9-mediated disease.

It is to be understood that one, some, or all of the properties of the various embodiments described herein may be combined to form other embodiments of the present disclosure. These and other aspects of the present disclosure will become apparent to one of skill in the art.

### BRIEF DESCRIPTION OF THE DRAWINGS

FIG. 1 shows qPCR analysis of PCSK9 mRNA expression in untransfected human Hep3B cells, or in human Hep3B cells transfected with increasing concentrations of 14 different test siRNAs targeting PCSK9, as compared to positive and negative control treatments. * indicates siRNAs that showed most potent reduction of PCSK9 expression in this assay.

FIG. 2 shows qPCR analysis of PCSK9 mRNA expression in untransfected human C3A cells, or in human C3A cells transfected with increasing concentrations of 14 different test siRNAs targeting PCSK9, as compared to positive and negative control treatments. * indicates siRNAs that showed most potent reduction of PCSK9 expression in this assay.

FIGS. 3A and 3B show the results of cytotoxicity assays for cells transfected with siRNAs targeting PCSK9. FIG. 3A shows the results of the cytotoxicity assay for human Hep3B cells transfected with siRNAs targeting PCSK9. FIG. 3B shows the results of the cytotoxicity assay for human C3A cells transfected with siRNAs targeting PCSK9.

FIG. 4 shows qPCR analysis of PCSK9 mRNA expression in untransfected human Hep3B cells, or in human Hep3B cells transfected with increasing concentrations of 60 different test siRNAs targeting PCSK9, as compared to positive and negative control treatments. * indicates siRNAs that showed most potent reduction of PCSK9 expression in this assay.

FIG. 5 shows qPCR analysis of PCSK9 mRNA expression in human C3A cells transfected with increasing concentrations of five different test siRNAs targeting PCSK9, as compared to positive and negative control treatments. * indicates siRNAs that showed most potent reduction of PCSK9 expression in this assay.

FIGS. 6A and 6B show the results of cytotoxicity assays for cells transfected with siRNAs targeting PCSK9. FIG. 6A shows the results of the cytotoxicity assay for human Hep3B cells transfected with siRNAs targeting PCSK9. FIG. 6B shows the results of the cytotoxicity assay for human C3A cells transfected with siRNAs targeting PCSK9.

FIG. 7 shows the amount of PCSK9 protein secreted into the supernatant of human C3A cell cultures for cells transfected with increasing concentrations of ten test siRNAs targeting PCSK9, as determined by ELISA assay.

FIG. 8 shows the amount of PCSK9 protein secreted into the supernatant of human C3A cell cultures transfected with three different concentrations of the siRNAs targeting PCSK9, as determined by ELISA.

FIG. 9 shows the results of cytotoxicity assays during free uptake of three different concentrations of the siRNAs targeting PCSK9 in primary human hepatocytes.

FIG. 10 shows the amount of interferon α (IFNα) protein released into the supernatant of human peripheral blood mononuclear cells (PBMCs) isolated from three donors and transfected with the siRNAs targeting PCSK9, as determined by ELISA.

FIG. 11 shows the in vitro serum stability and relative half-life of siRNAs targeting PCSK9 in 50% mouse serum.

FIG. 12 shows a summary of the results of the in vitro analysis of the siRNAs targeting PCSK9.

FIG. 13A shows serum PCSK9 levels over a time course in human PCSK9 transgenic mice treated with a single 10 mg/kg subcutaneous dose at day 0 of the indicated siRNAs targeting PCSK9, as measured by ELISA. FIG. 13B shows serum total cholesterol levels in these same mice, as determined with a COBAS INTEGRA instrument.

FIG. 13C shows the results of acute toxicity measurements in serum samples at day 3, as determined with a COBAS INTEGRA instrument. FIG. 13D shows the results of acute toxicity measurements in serum samples at day 10, as determined with a COBAS INTEGRA instrument. AST=aspartate aminotransferase; ALT=alanine aminotransferase; BUN=blood urea nitrogen.

FIG. 14A shows qPCR analysis of PCSK9 mRNA expression in untransfected human Hep3B cells, or in human Hep3B cells transfected with two different concentrations of additional test siRNAs targeting PCSK9, as compared to positive and negative control treatments. FIG. 14B shows qPCR analysis of PCSK9 mRNA expression in untransfected human C3A cells, or in human C3A cells transfected with two different concentrations of additional test siRNAs targeting PCSK9, as compared to positive and negative control treatments. An arrow indicates siRNAs that showed >50% knockdown of PCSK9 at concentration of 0.1 nM, or >85% knockdown of PCSK9 at a concentration of 1 nM in both Hep3B and C3A cell lines.

FIG. 15 shows the results of cytotoxicity assays in Hep3B and C3A cells transfected with two different concentrations of the additional test siRNAs targeting PCSK9. An X indicates siRNAs with >50% toxicity at a concentration of 50 nM as compared to the LV2 negative control.

FIG. 16A shows the correlation between the calculated IC₅₀ values in human Hep3B and C3A cells for the tested siRNAs. FIG. 16B shows the correlation between the calculated Iₘₐₓ values in human Hep3B and C3A cells for the additional test siRNAs targeting PCSK9.

FIG. 17 shows a graph depicting residual PCSK9 mRNA expression levels normalized to a LV2 non-silencing control in primary human hepatocytes treated with 100 nM and 1000 nM GalNAc-siRNAs from optimization libraries based on parent sequences C027.001, C027.002, and C027.003.

FIG. 18 shows the amount of interferon α2a (IFNα2a) protein (in pg/mL) released into the supernatant of human peripheral blood mononuclear cells (PBMCs) isolated from three donors and transfected with the siRNAs targeting PCSK9, as determined by ELISA.

FIG. 19A-C are graphs showing relative amounts of serum PCSK9 levels in human PCSK9 transgenic mice treated subcutaneously with a single dose of 42 optimized PCSK9 GalNAc-siRNAs and respective parent molecules at 6 mg/kg at day 0. FIGS. 19A-C represent data for optimized PCSK9 GalNAc-siRNAs based on parent sequences C027.001, C027.002, and C027.003, respectively. Protein expression is represented relative to animals treated with a PBS vehicle control. Human PCSK9 levels were quantified by ELISA, error bars indicate SEM. FIGS. 19D and E show serum LDL cholesterol levels in these same mice at days 14 (19D) and 28 (19E), after siRNA dosing, as determined with a COBAS INTEGRA instrument.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### 1. Definitions

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure described herein include “comprising,” “consisting,” and “consisting essentially of” aspects and embodiments. It should be understood that disclosures of embodiments using the term “comprises” or equivalent also encompass embodiments where “comprises” is replaced with “consists in”.

As used herein, the term “ribonucleotide” or “nucleotide” includes naturally occurring or modified nucleotide, as further detailed below, or a surrogate replacement moiety. One of ordinary skill in the art would understand that guanine, cytosine, adenine, uracil, or thymine in a nucleotide may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the present disclosure by a nucleotide containing, for example, inosine. Sequences comprising such replacement moieties are included as embodiments of the present disclosure.

As used herein, the term “PCSK9” refers to the proprotein convertase subtilisin kexin 9 gene or protein (also known as FH3, HCHOLA3, NARC-1, and NARC1). As used herein, the term “PCSK9” includes human PCSK9, the amino acid and nucleotide sequence of which may be found in, for example, NCBI Reference Sequence: NM_174936.3; mouse PCSK9, the amino acid and nucleotide sequence of which may be found in, for example, NCBI Reference Sequence: NM_153565.2; rat PCSK9, the amino acid and nucleotide sequence of which may be found in, for example, NCBI Reference Sequence: NM_199253.2. Additional examples of PCSK9 mRNA sequences are readily available using, e.g., GenBank.

As used herein, “target sequence” refers to a contiguous portion of the nucleotide sequence of an mRNA molecule formed during the transcription of the target gene, e.g., the PCSK9 gene, or portions thereof, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary”, when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by one of ordinary skill in the art. This includes base-pairing of the oligonucleotide or polynucleotide comprising the first nucleotide sequence to the oligonucleotide or polynucleotide comprising the second nucleotide sequence over the entire length of the first or second nucleotide sequence. Such sequences can be referred to as “fully complementary” with respect to each other herein. Where a first sequence is referred to as “substantially complementary” with respect to a second sequence herein, the two sequences can be fully complementary or they may form one or more, but not more than 4, 3, or 2 mismatched base pairs upon hybridization, while retaining the ability to hybridize under conditions most relevant to their ultimate application. However, where two oligonucleotides are designed to form, upon hybridization, one or more single stranded overhangs, such overhangs shall not be regarded as mismatches with regard to the determination of complementarity. For example, a double-stranded RNA (dsRNA) comprising a first oligonucleotide 21 nucleotides in length and a second oligonucleotide 23 nucleotides in length, wherein the second oligonucleotide comprises a sequence of 21 nucleotides that is fully complementary to the first oligonucleotide, may yet be referred to as “fully complementary” for the purpose of the present disclosure. “Complementary” sequences may also include, or be formed entirely from, non-Watson-Crick base pairs and/or base pairs formed from non-natural and modified nucleotides, in as far as the above requirements with respect to their ability to hybridize are fulfilled. The terms “complementary”, “fully complementary”, and “substantially complementary” may be used with respect to the base matching between the sense strand and the antisense strand of a dsRNA, or between the antisense strand of a dsRNA and a target sequence, as it will be understood from the context of their use. As used herein, a polynucleotide which is “substantially complementary to at least part of” an mRNA refers to a polynucleotide which is substantially complementary to a contiguous portion of the mRNA of interest (e.g., an mRNA encoding PCSK9). For example, a polynucleotide is substantially complementary to at least part of a PCSK9 mRNA if the sequence is substantially complementary to a non-interrupted portion of an mRNA encoding PCSK9.

As used herein, the term “double-stranded RNA” or “dsRNA” refers to a complex of ribonucleic acid molecule(s), having a duplex structure comprising two anti-parallel and substantially complementary, as defined above, nucleic acid strands. The two strands forming the duplex structure may be different portions of one larger RNA molecule, or they may be separate RNA molecules. Where separate RNA molecules, such dsRNA are often referred to in the literature as short interfering RNA (siRNA). Where two strands are part of one larger molecule, and therefore are connected by an uninterrupted chain of nucleotides between the 3′-end of a first strand and the 5′-end of a second strand forming the duplex structure, the connecting RNA chain is referred to as a “hairpin loop”, “short hairpin RNA”, or “shRNA”. Where the two strands are connected covalently by means other than an uninterrupted chain of nucleotides between the 3′-end of a first strand and the 5′-end of a second strand forming the duplex structure, the connecting structure is referred to as a “linker”. The RNA strands may have the same or a different number of nucleotides. The maximum number of base pairs is the number of oligonucleotides in the shortest strand of the dsRNA minus any overhangs that are present in the duplex. In addition to the duplex structure, a dsRNA may comprise one or more nucleotide overhangs. In addition, as used herein, the term “dsRNA” may include chemical modifications to ribonucleotides, including substantial modifications at multiple nucleotides and including all types of modifications disclosed herein or known in the art. Any such modifications, as used in an siRNA type molecule, are encompassed by “dsRNA” for the purposes of the present disclosure.

In some embodiments, the dsRNA comprises a modified ribonucleoside including a deoxyribonucleoside, including, for example, a deoxyribonucleoside overhang(s), one or more deoxyribonucleosides within the double stranded portion of a dsRNA, and the like. However, it is self-evident that under no circumstances is a double-stranded DNA molecule encompassed by the term “dsRNA”.

As used herein, the term “nucleotide overhang” refers to the unpaired nucleotide or nucleotides that protrude from the duplex structure of a dsRNA when a 3′-end of a first strand of the dsRNA extends beyond the 5′end of a second strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule. For clarity, chemical caps or non-nucleotide chemical moieties conjugated to the 3′ end and/or the 5′ end of a dsRNA are not considered in determining whether a dsRNA has an overhang or is blunt ended.

As used herein, the term “antisense strand” refers to the strand of a dsRNA which includes a sequence that is substantially complementary to a target sequence.

As used herein, the term “sense strand” refers to the strand of a dsRNA that includes a sequence that is substantially complementary to a region of the antisense strand.

As used herein, the term “introducing into a cell” means facilitating uptake or absorption into the cell, as would be understood by one of ordinary skill in the art. Absorption or uptake of dsRNA can occur through unaided diffusive or active cellular processes, or by auxiliary agents or devices. The meaning of this term is not to be limited to cell in vitro; a dsRNA may also be “introduced into a cell”, wherein the cell is part of a living organism. In such an instance, introduction into the cell will include delivery to the organism. For example, for in vivo delivery, dsRNA can be injected into a tissue site or administered systemically. In vivo delivery can also be mediated by a beta-glucan delivery system (See e.g., Tesz, G. J. et al. (2011) Biochem J. 436(2): 351-62). In vitro introduction into a cell includes methods known in the art such as electroporation and lipofection. Further approaches are described herein below or known in the art.

As used herein, the term “target gene” refers to a gene of interest, e.g., PCSK9, targeted for inhibition of expression by a dsRNA of the present disclosure.

As used herein, the term “PCSK9-associated disease” is intended to include any disease associated with the PCSK9 gene or protein. Such a disease may be caused, for example, by excess production of the PCSK9 protein, by PCSK9 gene mutations, by abnormal cleavage of the PCSK9 protein, by abnormal interactions between PCSK9 and other proteins or other endogenous or exogenous substances. Exemplary PCSK9-associated diseases include, without limitation, lipidemias, e.g., a hyperlipidemia, and other forms of lipid imbalance such as hypercholesterolemia, hypertriglyceridemia, and the pathological conditions associated with these disorders such as heart and circulatory diseases.

As used herein, the terms “inhibit the expression of” or “inhibiting expression of” in as far as they refer to the PCSK9 gene, refer to the at least partial suppression of the expression of the PCSK9 gene, as manifested by a reduction of the amount of mRNA transcribed from the PCSK9 gene which may be isolated from a first cell or group of cells in which the PCSK9 gene is transcribed and which has or have been treated such that the expression of the PCSK9 gene is inhibited, as compared to a second cell or group of cells substantially identical to the first cell or group of cells but which has or have not been so treated (control cells). As used herein, the term “inhibiting” is used interchangeably with “reducing”, “silencing”, “downregulating”, “suppressing”, and other similar terms, and include any level of inhibition. The degree of inhibition is usually expressed in terms of (((mRNA in control cells)−(mRNA in treated cells))/(mRNA in control cells))·100%.

Alternatively, the degree of inhibition may be given in terms of a reduction of a parameter that is functionally linked to PCSK9 gene transcription, e.g., the amount of protein encoded by the PCSK9 gene which is secreted by a cell, or the number of cells displaying a certain phenotype, e.g., apoptosis. In principle, PCSK9 gene silencing may be determined in any cell expressing the target, either constitutively or by genomic engineering, and by any appropriate assay. However, when a reference is needed in order to determine whether a given dsRNA inhibits the expression of the PCSK9 gene by a certain degree and therefore is encompassed by the present disclosure, the assays provided in the Examples below shall serve as such a reference.

As used herein, in the context of PCSK9 expression, the terms “treat”, “treatment” and the like refer to relief from or alleviation of pathological processes mediated by target gene expression. In the context of the present disclosure, insofar as it relates to any of the other conditions recited herein below (other than pathological processes mediated by target expression), the terms “treat”, “treatment”, and the like refer to relieving or alleviating one or more symptoms associated with such condition. For example, in the context of hyperlipidemia, treatment will involve a decrease in serum lipid levels.

As used herein, the terms “prevent” or “delay progression of” (and grammatical variants thereof) with respect to a disease or disorder relate to prophylactic treatment of a disease, e.g., in an individual suspected to have the disease, or at risk for developing the disease. Prevention may include, but is not limited to, preventing or delaying onset or progression of the disease and/or maintaining one or more symptoms of the disease at a desired or sub-pathological level. For example, in the context of hyperlipidemia, prevention may involve maintaining serum lipid levels at a desired level in an individual suspected to have or at risk for developing hyperlipidemia.

As used herein, the terms “therapeutically effective amount” and “prophylactically effective amount” refer to an amount that provides a therapeutic benefit in the treatment, prevention, or management of pathological processes mediated by target gene expression, e.g., PCSK9 gene expression, or an overt symptom of pathological processes mediated by target gene expression, e.g., PCSK9 gene expression. The specific amount that is therapeutically effective can be readily determined by an ordinary medical practitioner, and may vary depending on factors known in the art, such as, for example, the type of pathological processes mediated by target gene expression, e.g., PCSK9 gene expression, the patient's history and age, the stage of pathological processes mediated by target gene expression, e.g., PCSK9 gene expression, and the administration of other agents that inhibit processes mediated by target gene expression e.g., PCSK9 gene expression.

As used herein, the term “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the individual or subject is a human.

### 2. Double-Stranded RNAs (dsRNAs)

Certain aspects of the present disclosure relate to double-stranded ribonucleic acid (dsRNA) molecules targeting PCSK9. In some embodiments, the dsRNA comprises two strands, a sense strand comprising a first sequence and an antisense strand comprising a second sequence, wherein the first strand and the second strand are sufficiently complementary to hybridize to form a duplex structure. In some embodiments, the sense strand comprises a first sequence that is substantially complementary or fully complementary to the second sequence in the antisense strand. In some embodiments, the second sequence in the antisense strand is substantially complementary or fully complementary to a target sequence. In some embodiments, the target sequence is derived from the sequence of an mRNA formed during the expression of a target gene (e.g., an mRNA formed during the expression of a PCSK9 gene). In some embodiments, the PCSK9 gene is a human PCSK9 gene, e.g., as described herein. In some embodiments, the PCSK9 gene is a non-human PCSK9 gene. In some embodiments, the PCSK9 gene is a non-human primate PCSK9 gene (e.g., cynomolgus monkey PCSK9 (UniprotKB Accession No. G7NVZ1)). In some embodiments, the dsRNA inhibits expression of the PCSK9 gene. In some embodiments, the dsRNA is a small interfering RNA (siRNA). In some embodiments, the dsRNA is a short hairpin RNA (shRNA).

In some embodiments, the sense strand and the antisense strand of the dsRNA are in two separate molecules. In some embodiments, the duplex region is formed between the first sequence in the sense strand and the second sequence in the antisense strand of the two separate molecules. In some embodiments, the dsRNA is an siRNA. In some embodiments, the two separate molecules are not covalently linked to one another. In some embodiments, the two separate molecules are covalently linked to one another. In some embodiments, the two separate molecules are covalently linked to one another by means other than a hairpin loop. In some embodiments, the two separate molecules are covalently linked to one another via a connecting structure (herein referred to as a “covalent linker”).

In some embodiments, each of the first sequence (in the sense strand) and the second sequence (in the antisense strand) may range from 9-30 nucleotides in length. For example, each sequence may be between 12-30 nucleotides in length, 14-30 nucleotides in length, 15-30 nucleotides in length, 25-30 nucleotides in length, 27-30 nucleotides in length, 15-26 nucleotides in length, 15-23 nucleotides in length, 15-22 nucleotides in length, 15-21 nucleotides in length, 15-20 nucleotides in length, 15-19 nucleotides in length, 15-18 nucleotides in length, 15-17 nucleotides in length, 17-30 nucleotides in length, 17-23 nucleotides in length, 17-21 nucleotides in length, 17-19 nucleotides in length, 18-30 nucleotides in length, 18-26 nucleotides in length, 18-25 nucleotides in length, 18-23 nucleotides in length, 18-22 nucleotides in length, 18-21 nucleotides in length, 18-20 nucleotides in length, 19-30 nucleotides in length, 19-25 nucleotides in length, 19-24 nucleotides in length, 19-23 nucleotides in length, 19-22 nucleotides in length, 19-21 nucleotides in length, 19-20 nucleotides in length, 20-30 nucleotides in length, 20-26 nucleotides in length, 20-25 nucleotides in length, 20-24 nucleotides in length, 20-23 nucleotides in length, 20-22 nucleotides in length, 20-21 nucleotides in length, 21-30 nucleotides in length, 21-26 nucleotides in length, 21-25 nucleotides in length, 21-24 nucleotides in length, 21-23 nucleotides in length, or 21-22 nucleotides in length. In some embodiments, each sequence is greater than or equal to 9, 10, 11, 12, 13, 14, 15, 16, 17, 18, 19, 20, 21, 22, 23, 24, 25, 26, 27, 28, or 29 nucleotides in length. In some embodiments, each sequence is less than or equal to 10, 11, 12, 13, 14, 15, 16, 17, 18, 19, 20, 21, 22, 23, 24, 25, 26, 27, 28, 29, or 30 nucleotides in length. That is, each sequence can be any of a range of nucleotide lengths having an upper limit of 10, 11, 12, 13, 14, 15, 16, 17, 18, 19, 20, 21, 22, 23, 24, 25, 26, 27, 28, 29, or 30 and an independently selected lower limit of 9, 10, 11, 12, 13, 14, 15, 16, 17, 18, 19, 20, 21, 22, 23, 24, 25, 26, 27, 28, or 29, wherein the lower limit is less than the upper limit. In some embodiments, each sequence is 9, 10, 11, 12, 13, 14, 15, 16, 17, 18, 19, 20, 21, 22, 23, 24, 25, 26, 27, 28, 29, or 30 nucleotides in length. In some embodiments, the first and second sequences are each less than or equal to 30 nucleotides in length. In some embodiments, the first and second sequences are each at least 19 and less than or equal to 23 nucleotides in length. In some embodiments, the first sequence and the second sequence are a different number of nucleotides in length. In some embodiments, the first sequence is any of 1, 2, 3, 4, 5, 6, 7, 8, 9, or 10 nucleotides longer than the second sequence. In some embodiments, the second sequence is any of 1, 2, 3, 4, 5, 6, 7, 8, 9, or 10 nucleotides longer than the first sequence. In some embodiments, the first sequence and the second sequence are the same number of nucleotides in length.

In some embodiments, each of the sense and antisense strands may range from 9-36 nucleotides in length. For example, each strand may be between 12-30 nucleotides in length, 14-30 nucleotides in length, 15-30 nucleotides in length, 25-30 nucleotides in length, 27-30 nucleotides in length, 15-26 nucleotides in length, 15-23 nucleotides in length, 15-22 nucleotides in length, 15-21 nucleotides in length, 15-20 nucleotides in length, 15-19 nucleotides in length, 15-18 nucleotides in length, 15-17 nucleotides in length, 17-30 nucleotides in length, 17-23 nucleotides in length, 17-21 nucleotides in length, 17-19 nucleotides in length, 18-30 nucleotides in length, 18-26 nucleotides in length, 18-25 nucleotides in length, 18-23 nucleotides in length, 18-22 nucleotides in length, 18-21 nucleotides in length, 18-20 nucleotides in length, 19-30 nucleotides in length, 19-25 nucleotides in length, 19-24 nucleotides in length, 19-23 nucleotides in length, 19-22 nucleotides in length, 19-21 nucleotides in length, 19-20 nucleotides in length, 20-30 nucleotides in length, 20-26 nucleotides in length, 20-25 nucleotides in length, 20-24 nucleotides in length, 20-23 nucleotides in length, 20-22 nucleotides in length, 20-21 nucleotides in length, 21-30 nucleotides in length, 21-26 nucleotides in length, 21-25 nucleotides in length, 21-24 nucleotides in length, 21-23 nucleotides in length, or 21-22 nucleotides in length. In some embodiments, each strand is greater than or equal to 9, 10, 11, 12, 13, 14, 15, 16, 17, 18, 19, 20, 21, 22, 23, 24, 25, 26, 27, 28, 29, 30, 31, 32, 33, 34, or 35 nucleotides in length. In some embodiments, each strand is less than or equal to 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. That is, each strand can be any of a range of nucleotide lengths having an upper limit of 10, 11, 12, 13, 14, 15, 16, 17, 18, 19, 20, 21, 22, 23, 24, 25, 26, 27, 28, 29, 30, 31, 32, 33, 34, 35, or 36, and an independently selected lower limit of 9, 10, 11, 12, 13, 14, 15, 16, 17, 18, 19, 20, 21, 22, 23, 24, 25, 26, 27, 28, 29, 30, 31, 32, 33, 34, or 35, wherein the lower limit is less than the upper limit. In some embodiments, each strand is 9, 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. In some embodiments, the sense strand and antisense strand are the same number of nucleotides in length. In some embodiments, the sense strand and antisense strand are a different number of nucleotides in length.

In some embodiments, the first sequence (in the sense strand) and the second sequence (in the antisense strand) comprise less than 30% GC. By “less than 30% GC” is meant that, compared to the total nucleotide content of the first and/or second sequence, less than 30% of the nucleotides of said sequences are G (guanine) or C (cytosine). G (guanine) and C (cytosine) nucleotide contents also include modified G and C nucleotides. Such modifications are described below and include, for example, 2′-O-methylguanosine (mG), 2′-O-methylcytidine (mC), 2′-fluoro-guanosine (fG), 2′-fluoro-cytidine (fC), or locked guanine and cytosine (lG and lC). Without wanting to be bound by any theory, the inventors have noted that dsRNA of the present disclosure, comprising less than 30% of GC content exhibit higher efficacy in knocking down the expression of human PCSK9.

Overhangs

In some embodiments, a dsRNA of the present disclosure comprises one or more overhangs at the 3′-end, 5′-end, or both ends of one or both of the sense and antisense strands. In some embodiments, the one or more overhangs improve the stability and/or inhibitory activity of the dsRNA.

In some embodiments, the overhang comprises one or more, two or more, three or more, four or more, five or more, or six or more nucleotides. For example, the overhang may comprise 1-6 nucleotides, 2-6 nucleotides, 3-6 nucleotides, 4-6 nucleotides, 5-6 nucleotides, 1-5 nucleotides, 2-5 nucleotides, 3-5 nucleotides, 4-5 nucleotides, 1-4 nucleotides, 2-4 nucleotides, 3-4 nucleotides, 1-3 nucleotides, 2-3 nucleotides, or 1-2 nucleotides. In some embodiments, the overhang is one, two, three, four, five, or six nucleotides in length.

In some embodiments, an overhang of the present disclosure comprises one or more ribonucleotides. In some embodiments, an overhang of the present disclosure comprises one or more deoxyribonucleotides. In some embodiments, the overhang comprises one or more thymines.

In some embodiments, the dsRNA comprises an overhang located at the 3′-end of the antisense strand. In some embodiments, the dsRNA comprises a blunt end at the 5′-end of the antisense strand. In some embodiments, the dsRNA comprises an overhang located at the 3′-end of the antisense strand and a blunt end at the 5′-end of the antisense strand. In some embodiments, the dsRNA comprises an overhang located at the 3′-end of the sense strand. In some embodiments, the dsRNA comprises a blunt end at the 5′-end of the sense strand. In some embodiments, the dsRNA comprises an overhang located at the 3′-end of the sense strand and a blunt end at the 5′-end of the sense strand. In some embodiments, the dsRNA comprises overhangs located at both the 3′-end of the sense and antisense strands of the dsRNA.

In some embodiments, the dsRNA comprises an overhang located at the 5′-end of the antisense strand. In some embodiments, the dsRNA comprises a blunt end at the 3′-end of the antisense strand. In some embodiments, the dsRNA comprises an overhang located at the 5′-end of the antisense strand and a blunt end at the 3′-end of the antisense strand. In some embodiments, the dsRNA comprises an overhang located at the 5′-end of the sense strand. In some embodiments, the dsRNA comprises a blunt end at the 3′-end of the sense strand. In some embodiments, the dsRNA comprises an overhang located at the 5′-end of the sense strand and a blunt end at the 3′-end of the sense strand. In some embodiments, the dsRNA comprises overhangs located at both the 5′-end of the sense and antisense strands of the dsRNA.

In some embodiments, the overhang is the result of the sense strand being longer than the antisense strand. In some embodiments, the overhang is the result of the antisense strand being longer than the sense strand. In some embodiments, the overhang is the result of sense and antisense strands of the same length being staggered. In some embodiments, the overhang forms a mismatch with the target mRNA. In some embodiments, the overhang is complementary to the target mRNA.

In some embodiments, a dsRNA of the present disclosure comprises a sense strand comprising a first sequence and an antisense strand comprising a second sequence, wherein the first and second sequences are substantially complementary or complementary. In some embodiments, the first and second sequences are substantially complementary or complementary and form a duplex region of a dsRNA. In some embodiments, the duplex region of the dsRNA is 9-36 nucleotide pairs in length. For example, the duplex region may be between 12-30 nucleotide pairs in length, 14-30 nucleotide pairs in length, 15-30 nucleotide pairs in length, 15-26 nucleotide pairs in length, 15-23 nucleotide pairs in length, 15-22 nucleotide pairs in length, 15-21 nucleotide pairs in length, 15-20 nucleotide pairs in length, 15-19 nucleotide pairs in length, 15-18 nucleotide pairs in length, 15-17 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 18-30 nucleotide pairs in length, 18-26 nucleotide pairs in length, 18-25 nucleotide pairs in length, 18-24 nucleotide pairs in length, 18-23 nucleotide pairs in length, 18-22 nucleotide pairs in length, 18-21 nucleotide pairs in length, 18-20 nucleotide pairs in length, 19-30 nucleotide pairs in length, 19-25 nucleotide pairs in length, 19-24 nucleotide pairs in length, 19-23 nucleotide pairs in length, 19-22 nucleotide pairs in length, 19-21 nucleotide pairs in length, 19-20 nucleotide pairs in length, 20-30 nucleotide pairs in length, 20-26 nucleotide pairs in length, 20-25 nucleotide pairs in length, 20-24 nucleotide pairs in length, 20-23 nucleotide pairs in length, 20-22 nucleotide pairs in length, 20-21 nucleotide pairs in length, 21-30 nucleotide pairs in length, 21-26 nucleotide pairs in length, 21-25 nucleotide pairs in length, 21-24 nucleotide pairs in length, 21-23 nucleotide pairs in length, or 21-22 nucleotide pairs in length. In some embodiments, the duplex region of the dsRNA is greater than or equal to 9, 10, 11, 12, 13, 14, 15, 16, 17, 18, 19, 20, 21, 22, 23, 24, 25, 26, 27, 28, 29, 30, 31, 32, 33, 34, or 35 nucleotide pairs in length. In some embodiments, the duplex region of the dsRNA is less than or equal to 10, 11, 12, 13, 14, 15, 16, 17, 18, 19, 20, 21, 22, 23, 24, 25, 26, 27, 28, 29, 30, 31, 32, 33, 34, 35, or 36 nucleotide pairs in length. That is, the duplex region of the dsRNA can be any of a range of nucleotide pairs in length having an upper limit of 10, 11, 12, 13, 14, 15, 16, 17, 18, 19, 20, 21, 22, 23, 24, 25, 26, 27, 28, 29, 30, 31, 32, 33, 34, 35, or 36, and an independently selected lower limit of 9, 10, 11, 12, 13, 14, 15, 16, 17, 18, 19, 20, 21, 22, 23, 24, 25, 26, 27, 28, 29, 30, 31, 32, 33, 34, or 35, wherein the lower limit is less than the upper limit. In some embodiments, the duplex region is 9, 10, 11, 12, 13, 14, 15, 16, 17, 18, 19, 20, 21, 22, 23, 24, 25, 26, 27, 28, 29, 30, 31, 32, 33, 34, 35, or 36 nucleotide pairs in length. If more than one dsRNA is used, the duplex region of each dsRNA may be the same or different lengths than the one or more additional dsRNAs.

Target Sequences and First and Second Sequences in dsRNAs

In some embodiments, the target sequence is derived from a PCSK9 gene (such as a human PCSK9 gene). The human PCSK9 gene and associated mRNA sequences are known in the art. In some embodiments, the targeted mRNA has the sequence set forth in NCBI Ref. Seq. NM_174936.3. In some embodiments, a human PCSK9 cDNA has the sequence

(SEQ ID NO: 1)
GTCCGATGGGGCTCTGGTGGCGTGATCTGCGCGCCCCAGGCGTCAAGCACC
CACACCCTAGAAGGTTTCCGCAGCGACGTCGAGGCGCTCATGGTTGCAGGC
GGGCGCCGCCGTTCAGTTCAGGGTCTGAGCCTGGAGGAGTGAGCCAGGCAG
TGAGACTGGCTCGGGCGGGCCGGGACGCGTCGTTGCAGCAGCGGCTCCCAG
CTCCCAGCCAGGATTCCGCGCGCCCCTTCACGCGCCCTGCTCCTGAACTTC
AGCTCCTGCACAGTCCTCCCCACCGCAAGGCTCAAGGCGCCGCCGGCGTGG
ACCGCGCACGGCCTCTAGGTCTCCTCGCCAGGACAGCAACCTCTCCCCTGG
CCCTCATGGGCACCGTCAGCTCCAGGCGGTCCTGGTGGCCGCTGCCACTGC
TGCTGCTGCTGCTGCTGCTCCTGGGTCCCGCGGGCGCCCGTGCGCAGGAGG
ACGAGGACGGCGACTACGAGGAGCTGGTGCTAGCCTTGCGTTCCGAGGAGG
ACGGCCTGGCCGAAGCACCCGAGCACGGAACCACAGCCACCTTCCACCGCT
GCGCCAAGGATCCGTGGAGGTTGCCTGGCACCTACGTGGTGGTGCTGAAGG
AGGAGACCCACCTCTCGCAGTCAGAGCGCACTGCCCGCCGCCTGCAGGCCC
AGGCTGCCCGCCGGGGATACCTCACCAAGATCCTGCATGTCTTCCATGGCC
TTCTTCCTGGCTTCCTGGTGAAGATGAGTGGCGACCTGCTGGAGCTGGCCT
TGAAGTTGCCCCATGTCGACTACATCGAGGAGGACTCCTCTGTCTTTGCCC
AGAGCATCCCGTGGAACCTGGAGCGGATTACCCCTCCACGGTACCGGGCGG
ATGAATACCAGCCCCCCGACGGAGGCAGCCTGGTGGAGGTGTATCTCCTAG
ACACCAGCATACAGAGTGACCACCGGGAAATCGAGGGCAGGGTCATGGTCA
CCGACTTCGAGAATGTGCCCGAGGAGGACGGGACCCGCTTCCACAGACAGG
CCAGCAAGTGTGACAGTCATGGCACCCACCTGGCAGGGGTGGTCAGCGGCC
GGGATGCCGGCGTGGCCAAGGGTGCCAGCATGCGCAGCCTGCGCGTGCTCA
ACTGCCAAGGGAAGGGCACGGTTAGCGGCACCCTCATAGGCCTGGAGTTTA
TTCGGAAAAGCCAGCTGGTCCAGCCTGTGGGGCCACTGGTGGTGCTGCTGC
CCCTGGCGGGTGGGTACAGCCGCGTCCTCAACGCCGCCTGCCAGCGCCTGG
CGAGGGCTGGGGTCGTGCTGGTCACCGCTGCCGGCAACTTCCGGGACGATG
CCTGCCTCTACTCCCCAGCCTCAGCTCCCGAGGTCATCACAGTTGGGGCCA
CCAATGCCCAAGACCAGCCGGTGACCCTGGGGACTTTGGGGACCAACTTTG
GCCGCTGTGTGGACCTCTTTGCCCCAGGGGAGGACATCATTGGTGCCTCCA
GCGACTGCAGCACCTGCTTTGTGTCACAGAGTGGGACATCACAGGCTGCTG
CCCACGTGGCTGGCATTGCAGCCATGATGCTGTCTGCCGAGCCGGAGCTCA
CCCTGGCCGAGTTGAGGCAGAGACTGATCCACTTCTCTGCCAAAGATGTCA
TCAATGAGGCCTGGTTCCCTGAGGACCAGCGGGTACTGACCCCCAACCTGG
TGGCCGCCCTGCCCCCCAGCACCCATGGGGCAGGTTGGCAGCTGTTTTGCA
GGACTGTATGGTCAGCACACTCGGGGCCTACACGGATGGCCACAGCCGTCG
CCCGCTGCGCCCCAGATGAGGAGCTGCTGAGCTGCTCCAGTTTCTCCAGGA
GTGGGAAGCGGCGGGGCGAGCGCATGGAGGCCCAAGGGGGCAAGCTGGTCT
GCCGGGCCCACAACGCTTTTGGGGGTGAGGGTGTCTACGCCATTGCCAGGT
GCTGCCTGCTACCCCAGGCCAACTGCAGCGTCCACACAGCTCCACCAGCTG
AGGCCAGCATGGGGACCCGTGTCCACTGCCACCAACAGGGCCACGTCCTCA
CAGGCTGCAGCTCCCACTGGGAGGTGGAGGACCTTGGCACCCACAAGCCGC
CTGTGCTGAGGCCACGAGGTCAGCCCAACCAGTGCGTGGGCCACAGGGAGG
CCAGCATCCACGCTTCCTGCTGCCATGCCCCAGGTCTGGAATGCAAAGTCA
AGGAGCATGGAATCCCGGCCCCTCAGGAGCAGGTGACCGTGGCCTGCGAGG
AGGGCTGGACCCTGACTGGCTGCAGTGCCCTCCCTGGGACCTCCCACGTCC
TGGGGGCCTACGCCGTAGACAACACGTGTGTAGTCAGGAGCCGGGACGTCA
GCACTACAGGCAGCACCAGCGAAGGGGCCGTGACAGCCGTTGCCATCTGCT
GCCGGAGCCGGCACCTGGCGCAGGCCTCCCAGGAGCTCCAGTGACAGCCCC
ATCCCAGGATGGGTGTCTGGGGAGGGTCAAGGGCTGGGGCTGAGCTTTAAA
ATGGTTCCGACTTGTCCCTCTCTCAGCCCTCCATGGCCTGGCACGAGGGGA
TGGGGATGCTTCCGCCTTTCCGGGGCTGCTGGCCTGGCCCTTGAGTGGGGC
AGCCTCCTTGCCTGGAACTCACTCACTCTGGGTGCCTCCTCCCCAGGTGGA
GGTGCCAGGAAGCTCCCTCCCTCACTGTGGGGCATTTCACCATTCAAACAG
GTCGAGCTGTGCTCGGGTGCTGCCAGCTGCTCCCAATGTGCCGATGTCCGT
GGGCAGAATGACTTTTATTGAGCTCTTGTTCCGTGCCAGGCATTCAATCCT
CAGGTCTCCACCAAGGAGGCAGGATTCTTCCCATGGATAGGGGAGGGGGCG
GTAGGGGCTGCAGGGACAAACATCGTTGGGGGGTGAGTGTGAAAGGTGCTG
ATGGCCCTCATCTCCAGCTAACTGTGGAGAAGCCCCTGGGGGCTCCCTGAT
TAATGGAGGCTTAGCTTTCTGGATGGCATCTAGCCAGAGGCTGGAGACAGG
TGCGCCCCTGGTGGTCACAGGCTGTGCCTTGGTTTCCTGAGCCACCTTTAC
TCTGCTCTATGCCAGGCTGTGCTAGCAACACCCAAAGGTGGCCTGCGGGGA
GCCATCACCTAGGACTGACTCGGCAGTGTGCAGTGGTGCATGCACTGTCTC
AGCCAACCCGCTCCACTACCCGGCAGGGTACACATTCGCACCCCTACTTCA
CAGAGGAAGAAACCTGGAACCAGAGGGGGCGTGCCTGCCAAGCTCACACAG
CAGGAACTGAGCCAGAAACGCAGATTGGGCTGGCTCTGAAGCCAAGCCTCT
TCTTACTTCACCCGGCTGGGCTCCTCATTTTTACGGGTAACAGTGAGGCTG
GGAAGGGGAACACAGACCAGGAAGCTCGGTGAGTGATGGCAGAACGATGCC
TGCAGGCATGGAACTTTTTCCGTTATCACCCAGGCCTGATTCACTGGCCTG
GCGGAGATGCTTCTAAGGCATGGTCGGGGGAGAGGGCCAACAACTGTCCCT
CCTTGAGCACCAGCCCCACCCAAGCAAGCAGACATTTATCTTTTGGGTCTG
TCCTCTCTGTTGCCTTTTTACAGCCAACTTTTCTAGACCTGTTTTGCTTTT
GTAACTTGAAGATATTTATTCTGGGTTTTGTAGCATTTTTATTAATATGGT
GACTTTTTAAAATAAAAACAAACAAACGTTGTCCTAACAAAAAAAAAAAAA
AAAAAAAA.

In some embodiments, the dsRNA antisense strand comprises a sequence that is substantially complementary or complementary to between 12 and 30 nucleotides of a target sequence. For example, the sequence in the antisense strand may be substantially complementary or complementary to between 12-30 nucleotides, 14-30 nucleotides, 15-30 nucleotides, 15-26 nucleotides, 15-23 nucleotides, 15-22 nucleotides, 15-21 nucleotides, 15-20 nucleotides, 15-19 nucleotides, 15-18 nucleotides, 15-17 nucleotides, 17-30 nucleotides, 27-30 nucleotides, 17-23 nucleotides, 17-21 nucleotides, 17-19 nucleotides, 18-30 nucleotides, 18-26 nucleotides, 18-25 nucleotides, 18-23 nucleotides, 18-22 nucleotides, 18-21 nucleotides, 18-20 nucleotides, 19-30 nucleotides, 19-25 nucleotides, 19-24 nucleotides, 19-23 nucleotides, 19-22 nucleotides, 19-21 nucleotides, 19-20 nucleotides, 20-30 nucleotides, 20-26 nucleotides, 20-25 nucleotides, 20-24 nucleotides, 20-23 nucleotides, 20-22 nucleotides, 20-21 nucleotides, 21-30 nucleotides, 21-26 nucleotides, 21-25 nucleotides, 21-24 nucleotides, 21-23 nucleotides, or 21-22 nucleotides of a target sequence. In some embodiments, the sequence in the antisense strand may be substantially complementary or complementary to greater than or equal to 12, 13, 14, 15, 16, 17, 18, 19, 20, 21, 22, 23, 24, 25, 26, 27, 28, or 29 nucleotides of a target sequence. In some embodiments, the sequence in the antisense strand may be substantially complementary or complementary to less than or equal to 13, 14, 15, 16, 17, 18, 19, 20, 21, 22, 23, 24, 25, 26, 27, 28, 29, or 30 nucleotides of a target sequence. That is, the sequence in the antisense strand may be substantially complementary or complementary to any of a range of nucleotides of a target sequence having an upper limit of 13, 14, 15, 16, 17, 18, 19, 20, 21, 22, 23, 24, 25, 26, 27, 28, 29, or 30, and an independently selected lower limit of 12, 13, 14, 15, 16, 17, 18, 19, 20, 21, 22, 23, 24, 25, 26, 27, 28, or 29, wherein the lower limit is less than the upper limit. In some embodiments, the sequence in the antisense strand may be substantially complementary or complementary to 12, 13, 14, 15, 16, 17, 18, 19, 20, 21, 22, 23, 24, 25, 26, 27, 28, 29, or 30 nucleotides. If more than one dsRNA is used, the region of complementarity of each dsRNA may be the same or different lengths than the one or more additional dsRNAs.

In some embodiments, the target sequence comprises UUGUAGCAUUUUUAUUAAUAUGGUGACUUUUUAAAAUAAAAACAAACA (SEQ ID NO:2). In some embodiments, the target sequence comprises GAGUGUGAAAGGUGCUGAUGGCCCUCAUCU (SEQ ID NO:12). In some embodiments, the target sequence (e.g., a first sequence of a sense strand of a dsRNA of the present disclosure) is a sequence described in Table IA.

TABLE lA
siRNA sequence information.

| | | Target start position in |
|---|---|---|
| | | human PCSK9 mRNA |
| siRNA | Target sequence (5′→3′) | (NM_174936.3) |
| B001 | UUGUAGCAUUUUUAUUAAU (SEQ ID NO: 3) | 3649 |
| B003 | GUAGCAUUUUUAUUAAUAU (SEQ ID NO: 4) | 3651 |
| B006 | GCAUUUUUAUUAAUAUGGU (SEQ ID NO: 5) | 3654 |
| B008 | UUUUAUUAAUAUGGUGACU (SEQ ID NO: 6) | 3658 |
| B010 | UAUUAAUAUGGUGACUUUU (SEQ ID NO: 7) | 3661 |
| B011 | AUUAAUAUGGUGACUUUUU (SEQ ID NO: 8) | 3662 |
| B012 | UUAAUAUGGUGACUUUUUA (SEQ ID NO: 9) | 3663 |
| B013 | UAAUAUGGUGACUUUUUAA (SEQ ID NO: 10) | 3664 |
| B014 | UAUGGUGACUUUUUAAAAU (SEQ ID NO: 11) | 3667 |
| C051 | GAGUGUGAAAGGUGCUGAU (SEQ ID NO: 13) | 2891 |
| C209.016 | UUAUUAAUAUGGUGACUUU (SEQ ID NO: 310) | 3660 |
| C217.013 | AUAUGGUGACUUUUUAAAA (SEQ ID NO: 311) | 3656 |
| C218.003 | AUUUUUAUUAAUAUGGUGACU (SEQ ID NO: 312) | 3656 |
| C218.005 | UUUUAUUAAUAUGGUGACUUU (SEQ ID NO: 313) | 3658 |
| C218.006 | UUUAUUAAUAUGGUGACUUUU (SEQ ID NO: 314) | 3659 |
| C218.008 | UAUUAAUAUGGUGACUUUUUA (SEQ ID NO: 315) | 3661 |
| C218.012 | AAUAUGGUGACUUUUUAAAAU (SEQ ID NO: 316) | 3665 |
| C219.001 | GCAUUUUUAUUAAUAUGGUGACU (SEQ ID NO: 317) | 3654 |
| C219.003 | AUUUUUAUUAAUAUGGUGACUUU (SEQ ID NO: 318) | 3656 |
| C219.004 | UUUUUAUUAAUAUGGUGACUUUU (SEQ ID NO: 319) | 3657 |
| C219.006 | UUUAUUAAUAUGGUGACUUUUUA (SEQ ID NO: 320) | 3659 |
| C219.007 | UUAUUAAUAUGGUGACUUUUUAA (SEQ ID NO: 321) | 3660 |

In some embodiments, a dsRNA of the present disclosure comprises a sense strand comprising a first sequence. In some embodiments, the first sequence comprises a target sequence shown in Table IA. In some embodiments, the first sequence is a target sequence shown in Table IA. In some embodiments, the first sequence comprises a sequence selected from SEQ ID NOS: 3-11, 13, and 310-321. In some embodiments, the first sequence comprises a sequence selected from SEQ ID NOS: 6-11, and 310-321. In some embodiments, the first sequence is a sequence selected from SEQ ID NOS: 3-11, 13, and 310-321. In some embodiments, the first sequence is a sequence selected from SEQ ID NOS: 3, 4, and 13. In some embodiments, the first sequence is not one of GCAUUUUUAUUAAUAUGGU (SEQ ID NO: 5), UUUGUAGCAUUUUUAUUAAUAUGGU (SEQ ID NO: 576), or AUUUUUAUUAAUAUGGUGA (SEQ ID NO: 577).

In some embodiments, a dsRNA of the present disclosure comprises a first sequence comprising a sequence selected from the group consisting of SEQ ID NOS: 6-11 and 310-321, wherein the sequence selected from the group consisting of SEQ ID NOS: 6-11 and 310-321 comprises less than 30% GC. In some embodiments, a dsRNA of the present disclosure comprises a first sequence comprising a sequence selected from the group consisting of SEQ ID NOS: 6-11 and 310-321, wherein the first sequence comprises less than 30% GC. In some embodiments, a dsRNA of the present disclosure comprises a first sequence that is one of SEQ ID NOS: 3, 4, and 13, wherein the first sequence comprises less than 30% GC.

In some embodiments, a dsRNA of the present disclosure comprises an antisense strand comprising a second sequence. In some embodiments, the second sequence is substantially complementary or fully complementary to the first sequence (i.e., in the sense strand). In some embodiments, the second sequence is substantially complementary to the first sequence (i.e., in the sense strand), and the second strand comprises at least one mismatch (e.g., one mismatch, two mismatches, three mismatches, or four mismatches) to the first strand. In some embodiments, the second sequence is substantially complementary to the first sequence (i.e., in the sense strand), and the second strand comprises one or two mismatches to the first strand. In some embodiments, the second sequence is substantially complementary to a sequence selected from SEQ ID NOS: 3-11, 13, and 310-321, wherein the second strand comprises at least one mismatch (e.g., one mismatch, two mismatches, three mismatches, or four mismatches) to a sequence selected from SEQ ID NOS: 3-11, 13, and 310-321. In some embodiments, the second sequence is substantially complementary to a sequence selected from SEQ ID NOS: 3-11, 13, and 310-321, wherein the second strand comprises one or two mismatches to a sequence selected from SEQ ID NOS: 3-11, 13, and 310-321. In some embodiments, the second sequence is fully complementary to the first sequence (i.e., in the sense strand). In some embodiments, the second sequence is fully complementary to a sequence selected from SEQ ID NOS: 3-11, 13, and 310-321.

In some embodiments, a dsRNA of the present disclosure comprises a second sequence that is substantially complementary to a sequence selected from the group consisting of SEQ ID NOS: 3-11 and 310-321, wherein the second sequence comprises less than 30% GC. In some embodiments, a dsRNA of the present disclosure comprises a second sequence that is fully complementary to a sequence selected from the group consisting of SEQ ID NOS: 3-11 and 310-321, wherein the second sequence comprises less than 30% GC.

In some embodiments, the second sequence is substantially complementary or fully complementary to a sequence within SEQ ID NO:2 or SEQ ID NO:12. In some embodiments, the second sequence is substantially complementary or fully complementary to at least 15 contiguous nucleotides of SEQ ID NO:2 or SEQ ID NO:12. In some embodiments, the second sequence is substantially complementary or fully complementary to at least 19 contiguous nucleotides of SEQ ID NO:2 or SEQ ID NO:12. In some embodiments, the second sequence is substantially complementary or fully complementary to less than or equal to 30 contiguous nucleotides of SEQ ID NO:2 or SEQ ID NO:12. In some embodiments, the second sequence is substantially complementary or fully complementary to at least 19 and less than or equal to 23 contiguous nucleotides of SEQ ID NO:2 or SEQ ID NO:12. In some embodiments, the second sequence comprises a sequence shown in Table 1B. In some embodiments, the second sequence is a sequence shown in Table 1B.

TABLE 1B
siRNA second sequence information.

| siRNA | Second sequence (5′→3′) |
|---|---|
| B001 | AUUAAUAAAAAUGCUACAA (SEQ ID NO: 370) |
| B003 | AUAUUAAUAAAAAUGCUAC (SEQ ID NO: 371) |
| B006 | ACCAUAUUAAUAAAAAUGC (SEQ ID NO: 372) |
| B008 | AGUCACCAUAUUAAUAAAA (SEQ ID NO: 373) |
| B010 | AAAAGUCACCAUAUUAAUA (SEQ ID NO: 374) |
| B011 | AAAAAGUCACCAUAUUAAU (SEQ ID NO: 375) |
| B012 | UAAAAAGUCACCAUAUUAA (SEQ ID NO: 376) |
| B013 | UUAAAAAGUCACCAUAUUA (SEQ ID NO: 377) |
| B014 | AUUUUAAAAAGUCACCAUA (SEQ ID NO: 378) |
| C051 | AUCAGCACCUUUCACACUC (SEQ ID NO: 379) |
| C209.016 | AAAGUCACCAUAUUAAUAA (SEQ ID NO: 380) |
| C217.013 | UUUUAAAAAGUCACCAUAU (SEQ ID NO: 381) |
| C218.003 | AGUCACCAUAUUAAUAAAAAU (SEQ ID NO: 382) |
| C218.005 | AAAGUCACCAUAUUAAUAAAA (SEQ ID NO: 383) |
| C218.006 | AAAAGUCACCAUAUUAAUAAA (SEQ ID NO: 384) |
| C218.008 | UAAAAAGUCACCAUAUUAAUA (SEQ ID NO: 385) |
| C218.012 | AUUUUAAAAAGUCACCAUAUU (SEQ ID NO: 386) |
| C219.001 | AGUCACCAUAUUAAUAAAAAUGC (SEQ ID NO: 387) |
| C219.003 | AAAGUCACCAUAUUAAUAAAAAU (SEQ ID NO: 388) |
| C219.004 | AAAAGUCACCAUAUUAAUAAAAA (SEQ ID NO: 389) |
| C219.006 | UAAAAAGUCACCAUAUUAAUAAA (SEQ ID NO: 390) |
| C219.007 | UUAAAAAGUCACCAUAUUAAUAA (SEQ ID NO: 391) |

In some embodiments, a dsRNA or the second sequence in the antisense strand of a dsRNA of the present disclosure comprises one or more mismatches to the target sequence. In some embodiments, the target sequence is SEQ ID NO:2 or SEQ ID NO:12. In some embodiments, the dsRNA or the second sequence in the antisense strand of the dsRNA comprises no more than 4, 3, or 2 mismatches to the target sequence. In some embodiments, the dsRNA or the second sequence in the antisense strand of the dsRNA comprises no more than 1 mismatch to the target sequence. In some embodiments, the one or more mismatches is/are not located in the center of the region of complementarity. In some embodiments, the one or more mismatches is located within five, within four, within three, within two or within one nucleotide of the 5′ and/or 3′ ends of the region of complementarity. For example, for a 23 nucleotides dsRNA strand which is complementary to a region of the PCSK9 gene, the dsRNA generally does not contain any mismatch within the central 13 nucleotides of the region of complementarity between the dsRNA strand and the PCSK9 mRNA.

In some embodiments, a dsRNA of the present disclosure comprises a sense strand and/or an antisense strand described in Table 2 or Table 3. While the exemplary siRNAs shown in Table 2 include modifications, siRNAs having the same sequence but a different number/pattern/type of modifications, are also contemplated. siRNAs having the same sequences with no 2′-O-Me and 2′-Fluoro modifications are shown in Table 3. In some embodiments, a dsRNA comprises a sense strand shown in Table 3 but lacking the 5′ CCA and/or 3′ invdT. In some embodiments, a dsRNA comprises an antisense strand shown in Table 3 but lacking the 3′ dTdT.

TABLE 2
siRNA sequences (with modifications).

| | Sequence sense strand 5′-->3′ | Sequence antisense strand 5′-->3′ |
|---|---|---|
| siRNA ID | (SEQ ID NO) | (SEQ ID NO) |
| B001 | mCmCmAfUmUfGmUfAmGfCmAfUmU | fAfUmUfAmAfUmAfAmAfAmAfUm |
| | fUmUfUmAfUmUfAmAfUinvdT | GfCmUfAmCfAmAdTdT |
| | (SEQ ID NO: 14) | (SEQ ID NO: 15) |
| B002 | mCmCmAfUmGfUmAfGmCfAmUfUmU | fUfAmUfUmAfAmUfAmAfAmAfAm |
| | fUmUfAmUfUmAfAmUfAinvdT | UfGmCfUmAfCmAdTdT |
| | (SEQ ID NO: 16) | (SEQ ID NO: 17) |
| B003 | mCmCmAfGmUfAmGfCmAfUmUfUmU | fAfUmAfUmUfAmAfUmAfAmAfAm |
| | fUmAfUmUfAmAfUmAfUinvdT | AfUmGfCmUfAmCdTdT |
| | (SEQ ID NO: 18) | (SEQ ID NO: 19) |
| B004 | mCmCmAfUmAfGmCfAmUfUmUfUmU | fCfAmUfAmUfUmAfAmUfAmAfAm |
| | fAmUfUmAfAmUfAmUfGinvdT | AfAmUfGmCfUmAdTdT |
| | (SEQ ID NO: 20) | (SEQ ID NO: 21) |
| B005 | mCmCmAfAmGfCmAfUmUfUmUfUmA | fCfCmAfUmAfUmUfAmAfUmAfAm |
| | fUmUfAmAfUmAfUmGfGinvdT | AfAmAfUmGfCmUdTdT |
| | (SEQ ID NO: 22) | (SEQ ID NO: 23) |
| B006 | mCmCmAfGmCfAmUfUmUfUmUfAmU | fAfCmCfAmUfAmUfUmAfAmUfAm |
| | fUmAfAmUfAmUfGmGfUinvdT | AfAmAfAmUfGmCdTdT |
| | (SEQ ID NO: 24) | (SEQ ID NO: 25) |
| B007 | mCmCmAfUmUfUmUfUmAfUmUfAmA | fGfUmCfAmCfCmAfUmAfUmUfAm |
| | fUmAfUmGfGmUfGmAfCinvdT | AfUmAfAmAfAmAdTdT |
| | (SEQ ID NO: 26) | (SEQ ID NO: 27) |
| B008 | mCmCmAfUmUfUmUfAmUfUmAfAmU | fAfGmUfCmAfCmCfAmUfAmUfUm |
| | fAmUfGmGfUmGfAmCfUinvdT | AfAmUfAmAfAmAdTdT |
| | (SEQ ID NO: 28) | (SEQ ID NO: 29) |
| B009 | mCmCmAfUmUfUmAfUmUfAmAfUmA | fAfAmGfUmCfAmCfCmAfUmAfUm |
| | fUmGfGmUfGmAfCmUfUinvdT | UfAmAfUmAfAmAdTdT |
| | (SEQ ID NO: 30) | (SEQ ID NO: 31) |
| B010 | mCmCmAfUmAfUmUfAmAfUmAfUmG | fAfAmAfAmGfUmCfAmCfCmAfUm |
| | fGmUfGmAfCmUfUmUfUinvdT | AfUmUfAmAfUmAdTdT |
| | (SEQ ID NO: 32) | (SEQ ID NO: 33) |
| B011 | mCmCmAfAmUfUmAfAmUfAmUfGmG | fAfAmAfAmAfGmUfCmAfCmCfAm |
| | fUmGfAmCfUmUfUmUfUinvdT | UfAmUfUmAfAmUdTdT |
| | (SEQ ID NO: 34) | (SEQ ID NO: 35) |
| B012 | mCmCmAfUmUfAmAfUmAfUmGfGmU | fUfAmAfAmAfAmGfUmCfAmCfCm |
| | fGmAfCmUfUmUfUmUfAinvdT | AfUmAfUmUfAmAdTdT |
| | (SEQ ID NO: 36) | (SEQ ID NO: 37) |
| B013 | mCmCmAfUmAfAmUfAmUfGmGfUmG | fUfUmAfAmAfAmAfGmUfCmAfCm |
| | fAmCfUmUfUmUfUmAfAinvdT | CfAmUfAmUfUmAdTdT |
| | (SEQ ID NO: 38) | (SEQ ID NO: 39) |
| B014 | mCmCmAfUmAfUmGfGmUfGmAfCmU | fAfUmUfUmUfAmAfAmAfAmGfUm |
| | fUmUfUmUfAmAfAmAfUinvdT | CfAmCfCmAfUmAdTdT |
| | (SEQ ID NO: 40) | (SEQ ID NO: 41) |
| C001 | mCmCmAfGmGfCmUfCmUfGmGfUmGf | fAfGmAfUmCfAmCfGmCfCmAfCmC |
| | GmCfGmUfGmAfUmCfUinvdT | fAmGfAmGfCmCdTdT |
| | (SEQ ID NO: 42) | (SEQ ID NO: 43) |
| C002 | mCmCmAfGmCfUmCfUmGfGmUfGmGf | fCfAmGfAmUfCmAfCmGfCmCfAmC |
| | CmGfUmGfAmUfCmUfGinvdT | fCmAfGmAfGmCdTdT |
| | (SEQ ID NO: 44) | (SEQ ID NO: 45) |
| C003 | mCmCmAfCmUfCmUfGmGfUmGfGmCf | fGfCmAfGmAfUmCfAmCfGmCfCmA |
| | GmUfGmAfUmCfUmGfCinvdT | fCmCfAmGfAmGdTdT |
| | (SEQ ID NO: 46) | (SEQ ID NO: 47) |
| C004 | mCmCmAfUmCfUmGfGmUfGmGfCmGf | fCfGmCfAmGfAmUfCmAfCmGfCmC |
| | UmGfAmUfCmUfGmCfGinvdT | fAmCfCmAfGmAdTdT |
| | (SEQ ID NO: 48) | (SEQ ID NO: 49) |
| C005 | mCmCmAfCmAfGmGfCmGfUmCfAmAf | fUfGmUfGmGfGmUfGmCfUmUfGm |
| | GmCfAmCfCmCfAmCfAinvdT | AfCmGfCmCfUmGdTdT |
| | (SEQ ID NO: 50) | (SEQ ID NO: 51) |
| C006 | mCmCmAfAmGfGmCfGmUfCmAfAmGf | fGfUmGfUmGfGmGfUmGfCmUfUm |
| | CmAfCmCfCmAfCmAfCinvdT | GfAmCfGmCfCmUdTdT |
| | (SEQ ID NO: 52) | (SEQ ID NO: 53) |
| C007 | mCmCmAfCmGfUmCfAmAfGmCfAmCf | fAfGmGfGmUfGmUfGmGfGmUfGm |
| | CmCfAmCfAmCfCmCfUinvdT | CfUmUfGmAfCmGdTdT |
| | (SEQ ID NO: 54) | (SEQ ID NO: 55) |
| C008 | mCmCmAfGmUfCmAfAmGfCmAfCmCf | fUfAmGfGmGfUmGfUmGfGmGfUm |
| | CmAfCmAfCmCfCmUfAinvdT | GfCmUfUmGfAmCdTdT |
| | (SEQ ID NO: 56) | (SEQ ID NO: 57) |
| C009 | mCmCmAfUmCfAmAfGmCfAmCfCmCf | fCfUmAfGmGfGmUfGmUfGmGfGm |
| | AmCfAmCfCmCfUmAfGinvdT | UfGmCfUmUfGmAdTdT |
| | (SEQ ID NO: 58) | (SEQ ID NO: 59) |
| C010 | mCmCmAfCmAfAmGfCmAfCmCfCmAf | fUfCmUfAmGfGmGfUmGfUmGfGm |
| | CmAfCmCfCmUfAmGfAinvdT | GfUmGfCmUfUmGdTdT |
| | (SEQ ID NO: 60) | (SEQ ID NO: 61) |
| C011 | mCmCmAfAmAfGmCfAmCfCmCfAmCf | fUfUmCfUmAfGmGfGmUfGmUfGm |
| | AmCfCmCfUmAfGmAfAinvdT | GfGmUfGmCfUmUdTdT |
| | (SEQ ID NO: 62) | (SEQ ID NO: 63) |
| C012 | mCmCmAfAmGfCmAfCmCfCmAfCmAf | fCfUmUfCmUfAmGfGmGfUmGfUm |
| | CmCfCmUfAmGfAmAfGinvdT | GfGmGfUmGfCmUdTdT |
| | (SEQ ID NO: 64) | (SEQ ID NO: 65) |
| C013 | mCmCmAfGmCfAmCfCmCfAmCfAmCf | fCfCmUfUmCfUmAfGmGfGmUfGm |
| | CmCfUmAfGmAfAmGfGinvdT | UfGmGfGmUfGmCdTdT |
| | (SEQ ID NO: 66) | (SEQ ID NO: 67) |
| C014 | mCmCmAfCmAfCmCfCmAfCmAfCmCf | fAfCmCfUmUfCmUfAmGfGmGfUm |
| | CmUfAmGfAmAfGmGfUinvdT | GfUmGfGmGfUmGdTdT |
| | (SEQ ID NO: 68) | (SEQ ID NO: 69) |
| C015 | mCmCmAfAmCfCmCfAmCfAmCfCmCf | fAfAmCfCmUfUmCfUmAfGmGfGm |
| | UmAfGmAfAmGfGmUfUinvdT | UfGmUfGmGfGmUdTdT |
| | (SEQ ID NO: 70) | (SEQ ID NO: 71) |
| C016 | mCmCmAfCmCfCmAfCmAfCmCfCmUf | fAfAmAfCmCfUmUfCmUfAmGfGm |
| | AmGfAmAfGmGfUmUfUinvdT | GfUmGfUmGfGmGdTdT |
| | (SEQ ID NO: 72) | (SEQ ID NO: 73) |
| C017 | mCmCmAfCmCfAmCfAmCfCmCfUmAf | fGfAmAfAmCfCmUfUmCfUmAfGm |
| | GmAfAmGfGmUfUmUfCinvdT | GfGmUfGmUfGmGdTdT |
| | (SEQ ID NO: 74) | (SEQ ID NO: 75) |
| C018 | mCmCmAfCmAfCmAfCmCfCmUfAmGf | fGfGmAfAmAfCmCfUmUfCmUfAm |
| | AmAfGmGfUmUfUmCfCinvdT | GfGmGfUmGfUmGdTdT |
| | (SEQ ID NO: 76) | (SEQ ID NO: 77) |
| C019 | mCmCmAfAmCfAmCfCmCfUmAfGmAf | fCfGmGfAmAfAmCfCmUfUmCfUmA |
| | AmGfGmUfUmUfCmCfGinvdT | fGmGfGmUfGmUdTdT |
| | (SEQ ID NO: 78) | (SEQ ID NO: 79) |
| C020 | mCmCmAfCmAfCmCfCmUfAmGfAmAf | fGfCmGfGmAfAmAfCmCfUmUfCmU |
| | GmGfUmUfUmCfCmGfCinvdT | fAmGfGmGfUmGdTdT |
| | (SEQ ID NO: 80) | (SEQ ID NO: 81) |
| C021 | mCmCmAfAmCfCmCfUmAfGmAfAmGf | fUfGmCfGmGfAmAfAmCfCmUfUmC |
| | GmUfUmUfCmCfGmCfAinvdT | fUmAfGmGfGmUdTdT |
| | (SEQ ID NO: 82) | (SEQ ID NO: 83) |
| C022 | mCmCmAfCmCfCmUfAmGfAmAfGmGf | fCfUmGfCmGfGmAfAmAfCmCfUmU |
| | UmUfUmCfCmGfCmAfGinvdT | fCmUfAmGfGmGdTdT |
| | (SEQ ID NO: 84) | (SEQ ID NO: 85) |
| C023 | mCmCmAfCmCfUmAfGmAfAmGfGmUf | fGfCmUfGmCfGmGfAmAfAmCfCmU |
| | UmUfCmCfGmCfAmGfCinvdT | fUmCfUmAfGmGdTdT |
| | (SEQ ID NO: 86) | (SEQ ID NO: 87) |
| C024 | mCmCmAfCmUfAmGfAmAfGmGfUmU | fCfGmCfUmGfCmGfGmAfAmAfCmC |
| | fUmCfCmGfCmAfGmCfGinvdT | fUmUfCmUfAmGdTdT |
| | (SEQ ID NO: 88) | (SEQ ID NO: 89) |
| C025 | mCmCmAfUmAfGmAfAmGfGmUfUmU | fUfCmGfCmUfGmCfGmGfAmAfAmC |
| | fCmCfGmCfAmGfCmGfAinvdT | fCmUfUmCfUmAdTdT |
| | (SEQ ID NO: 90) | (SEQ ID NO: 91) |
| C026 | mCmCmAfAmGfAmAfGmGfUmUfUmC | fGfUmCfGmCfUmGfCmGfGmAfAm |
| | fCmGfCmAfGmCfGmAfCinvdT | AfCmCfUmUfCmUdTdT |
| | (SEQ ID NO: 92) | (SEQ ID NO: 93) |
| C027 | mCmCmAfGmAfAmGfGmUfUmUfCmCf | fCfGmUfCmGfCmUfGmCfGmGfAmA |
| | GmCfAmGfCmGfAmCfGinvdT | fAmCfCmUfUmCdTdT |
| | (SEQ ID NO: 94) | (SEQ ID NO: 95) |
| C028 | mCmCmAfAmAfGmGfUmUfUmCfCmGf | fAfCmGfUmCfGmCfUmGfCmGfGmA |
| | CmAfGmCfGmAfCmGfUinvdT | fAmAfCmCfUmUdTdT |
| | (SEQ ID NO: 96) | (SEQ ID NO: 97) |
| C029 | mCmCmAfAmGfGmUfUmUfCmCfGmCf | fGfAmCfGmUfCmGfCmUfGmCfGmG |
| | AmGfCmGfAmCfGmUfCinvdT | fAmAfAmCfCmUdTdT |
| | (SEQ ID NO: 98) | (SEQ ID NO: 99) |
| C030 | mCmCmAfGmUfUmUfCmCfGmCfAmGf | fUfCmGfAmCfGmUfCmGfCmUfGmC |
| | CmGfAmCfGmUfCmGfAinvdT | fGmGfAmAfAmCdTdT |
| | (SEQ ID NO: 100) | (SEQ ID NO: 101) |
| C031 | mCmCmAfUmUfUmCfCmGfCmAfGmCf | fCfUmCfGmAfCmGfUmCfGmCfUmG |
| | GmAfCmGfUmCfGmAfGinvdT | fCmGfGmAfAmAdTdT |
| | (SEQ ID NO: 102) | (SEQ ID NO: 103) |
| C032 | mCmCmAfCmCfUmGfAmAfCmUfUmCf | fGfCmAfGmGfAmGfCmUfGmAfAm |
| | AmGfCmUfCmCfUmGfCinvdT | GfUmUfCmAfGmGdTdT |
| | (SEQ ID NO: 104) | (SEQ ID NO: 105) |
| C033 | mCmCmAfCmUfGmAfAmCfUmUfCmAf | fUfGmCfAmGfGmAfGmCfUmGfAm |
| | GmCfUmCfCmUfGmCfAinvdT | AfGmUfUmCfAmGdTdT |
| | (SEQ ID NO: 106) | (SEQ ID NO: 107) |
| C034 | mCmCmAfUmGfAmAfCmUfUmCfAmGf | fGfUmGfCmAfGmGfAmGfCmUfGm |
| | CmUfCmCfUmGfCmAfCinvdT | AfAmGfUmUfCmAdTdT |
| | (SEQ ID NO: 108) | (SEQ ID NO: 109) |
| C035 | mCmCmAfGmGfAmCfAmGfCmAfAmCf | fAfGmGfGmGfAmGfAmGfGmUfUm |
| | CmUfCmUfCmCfCmCfUinvdT | GfCmUfGmUfCmCdTdT |
| | (SEQ ID NO: 110) | (SEQ ID NO: 111) |
| C036 | mCmCmAfGmAfCmAfGmCfAmAfCmCf | fCfAmGfGmGfGmAfGmAfGmGfUm |
| | UmCfUmCfCmCfCmUfGinvdT | UfGmCfUmGfUmCdTdT |
| | (SEQ ID NO: 112) | (SEQ ID NO: 113) |
| C037 | mCmCmAfAmCfAmGfCmAfAmCfCmUf | fCfCmAfGmGfGmGfAmGfAmGfGm |
| | CmUfCmCfCmCfUmGfGinvdT | UfUmGfCmUfGmUdTdT |
| | (SEQ ID NO: 114) | (SEQ ID NO: 115) |
| C038 | mCmCmAfAmUfGmGfGmCfAmCfCmGf | fUfGmGfAmGfCmUfGmAfCmGfGm |
| | UmCfAmGfCmUfCmCfAinvdT | UfGmCfCmCfAmUdTdT |
| | (SEQ ID NO: 116) | (SEQ ID NO: 117) |
| C039 | mCmCmAfGmGfAmUfGmAfAmUfAmC | fGfGmGfGmGfGmCfUmGfGmUfAm |
| | fCmAfGmCfCmCfCmCfCinvdT | UfUmCfAmUfCmCdTdT |
| | (SEQ ID NO: 118) | (SEQ ID NO: 119) |
| C040 | mCmCmAfGmAfUmGfAmAfUmAfCmCf | fCfGmGfGmGfGmGfCmUfGmGfUm |
| | AmGfCmCfCmCfCmCfGinvdT | AfUmUfCmAfUmCdTdT |
| | (SEQ ID NO: 120) | (SEQ ID NO: 121) |
| C041 | mCmCmAfAmUfGmAfAmUfAmCfCmAf | fUfCmGfGmGfGmGfGmCfUmGfGm |
| | GmCfCmCfCmCfCmGfAinvdT | UfAmUfUmCfAmUdTdT |
| | (SEQ ID NO: 122) | (SEQ ID NO: 123) |
| C042 | mCmCmAfCmAfAmGfGmGfGmGfCmAf | fAfGmAfCmCfAmGfCmUfUmGfCmC |
| | AmGfCmUfGmGfUmCfUinvdT | fCmCfCmUfUmGdTdT |
| | (SEQ ID NO: 124) | (SEQ ID NO: 125) |
| C043 | mCmCmAfAmAfGmGfGmGfGmCfAmA | fCfAmGfAmCfCmAfGmCfUmUfGmC |
| | fGmCfUmGfGmUfCmUfGinvdT | fCmCfCmCfUmUdTdT |
| | (SEQ ID NO: 126) | (SEQ ID NO: 127) |
| C044 | mCmCmAfUmGfGmAfCmCfCmUfGmAf | fUfGmCfAmGfCmCfAmGfUmCfAmG |
| | CmUfGmGfCmUfGmCfAinvdT | fGmGfUmCfCmAdTdT |
| | (SEQ ID NO: 128) | (SEQ ID NO: 129) |
| C045 | mCmCmAfGmAfCmCfCmUfGmAfCmUf | fAfCmUfGmCfAmGfCmCfAmGfUmC |
| | GmGfCmUfGmCfAmGfUinvdT | fAmGfGmGfUmCdTdT |
| | (SEQ ID NO: 130) | (SEQ ID NO: 131) |
| C046 | mCmCmAfUmGfAmCfUmGfGmCfUmGf | fAfGmGfGmCfAmCfUmGfCmAfGmC |
| | CmAfGmUfGmCfCmCfUinvdT | fCmAfGmUfCmAdTdT |
| | (SEQ ID NO: 132) | (SEQ ID NO: 133) |
| C047 | mCmCmAfUmCfUmUfCmCfCmAfUmGf | fUfCmCfCmCfUmAfUmCfCmAfUmG |
| | GmAfUmAfGmGfGmGfAinvdT | fGmGfAmAfGmAdTdT |
| | (SEQ ID NO: 134) | (SEQ ID NO: 135) |
| C048 | mCmCmAfCmUfUmCfCmCfAmUfGmGf | fCfUmCfCmCfCmUfAmUfCmCfAmU |
| | AmUfAmGfGmGfGmAfGinvdT | fGmGfGmAfAmGdTdT |
| | (SEQ ID NO: 136) | (SEQ ID NO: 137) |
| C049 | mCmCmAfUmUfCmCfCmAfUmGfGmAf | fCfCmUfCmCfCmCfUmAfUmCfCmA |
| | UmAfGmGfGmGfAmGfGinvdT | fUmGfGmGfAmAdTdT |
| | (SEQ ID NO: 138) | (SEQ ID NO: 139) |
| C050 | mCmCmAfGmUfAmGfGmGfGmCfUmG | fUfUmGfUmCfCmCfUmGfCmAfGmC |
| | fCmAfGmGfGmAfCmAfAinvdT | fCmCfCmUfAmCdTdT |
| | (SEQ ID NO: 140) | (SEQ ID NO: 141) |
| C051 | mCmCmAfGmAfGmUfGmUfGmAfAmA | fAfUmCfAmGfCmAfCmCfUmUfUmC |
| | fGmGfUmGfCmUfGmAfUinvdT | fAmCfAmCfUmCdTdT |
| | (SEQ ID NO: 142) | (SEQ ID NO: 143) |
| C052 | mCmCmAfAmGfUmGfUmGfAmAfAmG | fCfAmUfCmAfGmCfAmCfCmUfUmU |
| | fGmUfGmCfUmGfAmUfGinvdT | fCmAfCmAfCmUdTdT |
| | (SEQ ID NO: 144) | (SEQ ID NO: 145) |
| C053 | mCmCmAfGmUfGmUfGmAfAmAfGmG | fCfCmAfUmCfAmGfCmAfCmCfUmU |
| | fUmGfCmUfGmAfUmGfGinvdT | fUmCfAmCfAmCdTdT |
| | (SEQ ID NO: 146) | (SEQ ID NO: 147) |
| C054 | mCmCmAfUmGfUmGfAmAfAmGfGmU | fGfCmCfAmUfCmAfGmCfAmCfCmU |
| | fGmCfUmGfAmUfGmGfCinvdT | fUmUfCmAfCmAdTdT |
| | (SEQ ID NO: 148) | (SEQ ID NO: 149) |
| C055 | mCmCmAfGmUfGmAfAmAfGmGfUmG | fGfGmCfCmAfUmCfAmGfCmAfCmC |
| | fCmUfGmAfUmGfGmCfCinvdT | fUmUfUmCfAmCdTdT |
| | (SEQ ID NO: 150) | (SEQ ID NO: 151) |
| C056 | mCmCmAfGmCfUmGfUmGfCmCfUmUf | fCfAmGfGmAfAmAfCmCfAmAfGm |
| | GmGfUmUfUmCfCmUfGinvdT | GfCmAfCmAfGmCdTdT |
| | (SEQ ID NO: 152) | (SEQ ID NO: 153) |
| C057 | mCmCmAfUmGfUmGfCmCfUmUfGmGf | fCfUmCfAmGfGmAfAmAfCmCfAmA |
| | UmUfUmCfCmUfGmAfGinvdT | fGmGfCmAfCmAdTdT |
| | (SEQ ID NO: 154) | (SEQ ID NO: 155) |
| C058 | mCmCmAfAmAfCmAfCmCfCmAfAmAf | fCfAmGfGmCfCmAfCmCfUmUfUmG |
| | GmGfUmGfGmCfCmUfGinvdT | fGmGfUmGfUmUdTdT |
| | (SEQ ID NO: 156) | (SEQ ID NO: 157) |
| C059 | mCmCmAfAmCfAmCfCmCfAmAfAmGf | fGfCmAfGmGfCmCfAmCfCmUfUmU |
| | GmUfGmGfCmCfUmGfCinvdT | fGmGfGmUfGmUdTdT |
| | (SEQ ID NO: 158) | (SEQ ID NO: 159) |
| C060 | mCmCmAfGmAfAmGfAmAfAmCfCmUf | fUfCmUfGmGfUmUfCmCfAmGfGm |
| | GmGfAmAfCmCfAmGfAinvdT | UfUmUfCmUfUmCdTdT |
| | (SEQ ID NO: 160) | (SEQ ID NO: 161) |
| C209.016 | mCmCmAfUmUfAmUfUmAfAmUfAmU | fAfAmAfGmUfCmAfCmCfAmUfAm |
| | fGmGfUmGfAmCfUmUfUinvdT (SEQ ID | UfUmAfAmUfAmAdTdT (SEQ ID |
| | NO: 346) | NO: 347) |
| C217.013 | mCmCmAfAmUfAmUfGmGfUmGfAmC | fUfUmUfUmAfAmAfAmAfGmUfCm |
| | fUmUfUmUfUmAfAmAfAinvdT (SEQ | AfCmCfAmUfAmUdtdt (SEQ ID NO: |
| | ID NO: 348) | 349) |
| C218.003 | mCmCmAfAmUfUmUfUmUfAmUfUmA | fAfGmUfCmAfCmCfAmUfAmUfUm |
| | fAmUfAmUfGmGfUmGfAmCfUinvdT | AfAmUfAmAfAmAfAmUdTdT (SEQ |
| | (SEQ ID NO: 350) | ID NO: 351) |
| C218.005 | mCmCmAfUmUfUmUfAmUfUmAfAmU | fAfAmAfGmUfCmAfCmCfAmUfAm |
| | fAmUfGmGfUmGfAmCfUmUfUinvdT | UfUmAfAmUfAmAfAmAdTdT (SEQ |
| | (SEQ ID NO: 352) | ID NO: 353) |
| C218.006 | mCmCmAfUmUfUmAfUmUfAmAfUmA | fAfAmAfAmGfUmCfAmCfCmAfUm |
| | fUmGfGmUfGmAfCmUfUmUfUinvdT | AfUmUfAmAfUmAfAmAdTdT (SEQ |
| | (SEQ ID NO: 354) | ID NO: 355) |
| C218.008 | mCmCmAfUmAfUmUfAmAfUmAfUmG | fUfAmAfAmAfAmGfUmCfAmCfCm |
| | fGmUfGmAfCmUfUmUfUmUfAinvdT | AfUmAfUmUfAmAfUmAdTdT |
| | (SEQ ID NO: 356) | (SEQ ID NO: 357) |
| C218.012 | mCmCmAfAmAfUmAfUmGfGmUfGmA | fAfUmUfUmUfAmAfAmAfAmGfUm |
| | fCmUfUmUfUmUfAmAfAmAfUinvdT | CfAmCfCmAfUmAfUmUdTdT (SEQ |
| | (SEQ ID NO: 358) | ID NO: 359) |
| C219.001 | mCmCmAfGmCfAmUfUmUfUmUfAmUfUm | fAfGmUfCmAfCmCfAmUfAmUfUmAfA |
| | AfAmUfAmUfGmGfUmGfAmCfUinvdT | mUfAmAfAmAfAmUfGmCdTdT (SEQ |
| | (SEQ ID NO: 360) | ID NO: 361) |
| C219.003 | mCmCmAfAmUfUmUfUmUfAmUfUmAfAm | fAfAmAfGmUfCmAfCmCfAmUfAmUfU |
| | UfAmUfGmGfUmGfAmCfUmUfUinvdT | mAfAmUfAmAfAmAfAmUdTdT (SEQ |
| | (SEQ ID NO: 362) | ID NO: 363) |
| C219.004 | mCmCmAfUmUfUmUfUmAfUmUfAmAfUm | fAfAmAfAmGfUmCfAmCfCmAfUmAfU |
| | AfUmGfGmUfGmAfCmUfUmUfUinvdT | mUfAmAfUmAfAmAfAmAdTdT (SEQ |
| | (SEQ ID NO: 364) | ID NO: 365) |
| C219.006 | mCmCmAfUmUfUmAfUmUfAmAfUmAfUm | fUfAmAfAmAfAmGfUmCfAmCfCmAfU |
| | GfGmUfGmAfCmUfUmUfUmUfAinvdT | mAfUmUfAmAfUmAfAmAdTdT (SEQ |
| | (SEQ ID NO: 366) | ID NO: 367) |
| C219.007 | mCmCmAfUmUfAmUfUmAfAmUfAmUfGm | fUfUmAfAmAfAmAfGmUfCmAfCmCfA |
| | GfUmGfAmCfUmUfUmUfUmAfAinvdT | mUfAmUfUmAfAmUfAmAdTdT (SEQ |
| | (SEQ ID NO: 368) | ID NO: 369) |
| C209.001 | mCmCmAfCmAfGmGfAmCfAmGfCmAf | fGfGmGfAmGfAmGfGmUfUmGfCm |
| | AmCfCmUfCmUfCmCfCinvdT (SEQ ID | UfGmUfCmCfUmGdTdT (SEQ ID |
| | NO: 484) | NO: 485) |
| C209.002 | mCmCmAfAmGfGmAfCmAfGmCfAmAf | fGfGmGfGmAfGmAfGmGfUmUfGm |
| | CmCfUmCfUmCfCmCfCinvdT (SEQ ID | CfUmGfUmCfCmUdTdT (SEQ ID NO: |
| | NO: 486) | 487) |
| C209.003 | mCmCmAfUmGfCmCfAmCfUmGfCmUf | fAfGmCfAmGfCmAfGmCfAmGfCmA |
| | GmCfUmGfCmUfGmCfUinvdT (SEQ ID | fGmUfGmGfCmAdTdT (SEQ ID NO: |
| | NO: 488) | 489) |
| C209.004 | mCmCmAfCmAfCmUfGmCfUmGfCmUf | fAfGmCfAmGfCmAfGmCfAmGfCmA |
| | GmCfUmGfCmUfGmCfUinvdT (SEQ ID | fGmCfAmGfUmGdTdT (SEQ ID NO: |
| | NO: 490) | 491) |
| C209.005 | mCmCmAfUmGfCmUfGmCfUmGfCmUf | fAfGmGfAmGfCmAfGmCfAmGfCm |
| | GmCfUmGfCmUfCmCfUinvdT (SEQ ID | AfGmCfAmGfCmAdTdT (SEQ ID |
| | NO: 492) | NO: 493) |
| C209.006 | mCmCmAfAmCfCmCfUmGfAmCfUmGf | fCfAmCfUmGfCmAfGmCfCmAfGmU |
| | GmCfUmGfCmAfGmUfGinvdT (SEQ ID | fCmAfGmGfGmUdTdT (SEQ ID NO: |
| | NO: 494) | 495) |
| C209.007 | mCmCmAfUmGfAmAfAmGfGmUfGmC | fGfGmGfCmCfAmUfCmAfGmCfAmC |
| | fUmGfAmUfGmGfCmCfCinvdT (SEQ ID | fCmUfUmUfCmAdTdT (SEQ ID NO: |
| | NO: 496) | 497) |
| C209.008 | mCmCmAfGmGfCmUfGmUfGmCfCmUf | fAfGmGfAmAfAmCfCmAfAmGfGm |
| | UmGfGmUfUmUfCmCfUinvdT (SEQ ID | CfAmCfAmGfCmCdTdT (SEQ ID NO: |
| | NO: 498) | 499) |
| C209.009 | mCmCmAfCmUfGmUfGmCfCmUfUmGf | fUfCmAfGmGfAmAfAmCfCmAfAm |
| | GmUfUmUfCmCfUmGfAinvdT (SEQ ID | GfGmCfAmCfAmGdTdT (SEQ ID |
| | NO: 500) | NO: 501) |
| C209.010 | mCmCmAfGmGfAmAfGmAfAmAfCmCf | fCfUmGfGmUfUmCfCmAfGmGfUm |
| | UmGfGmAfAmCfCmAfGinvdT (SEQ ID | UfUmCfUmUfCmCdTdT (SEQ ID NO: |
| | NO: 502) | 503) |
| C209.011 | mCmCmAfAmAfGmAfAmAfCmCfUmGf | fCfUmCfUmGfGmUfUmCfCmAfGmG |
| | GmAfAmCfCmAfGmAfGinvdT (SEQ ID | fUmUfUmCfUmUdTdT (SEQ ID NO: |
| | NO: 504) | 505) |
| C209.012 | mCmCmAfAmGfAmAfAmCfCmUfGmGf | fCfCmUfCmUfGmGfUmUfCmCfAmG |
| | AmAfCmCfAmGfAmGfGinvdT (SEQ ID | fGmUfUmUfCmUdTdT (SEQ ID NO: |
| | NO: 506) | 507) |
| C209.013 | mCmCmAfAmAfCmCfUmGfGmAfAmCf | fCfCmCfCmCfUmCfUmGfGmUfUmC |
| | CmAfGmAfGmGfGmGfGinvdT (SEQ ID | fCmAfGmGfUmUdTdT (SEQ ID NO: |
| | NO: 508) | 509) |
| C209.014 | mCmCmAfCmAfUmUfUmUfUmAfUmU | fCfAmCfCmAfUmAfUmUfAmAfUm |
| | fAmAfUmAfUmGfGmUfGinvdT (SEQ | AfAmAfAmAfUmGdTdT (SEQ ID |
| | ID NO: 510) | NO: 511) |
| C209.015 | mCmCmAfAmUfUmUfUmUfAmUfUmA | fUfCmAfCmCfAmUfAmUfUmAfAm |
| | fAmUfAmUfGmGfUmGfAinvdT (SEQ | UfAmAfAmAfAmUdTdT (SEQ ID |
| | ID NO: 512) | NO: 513) |
| C209.017 | mCmCmAfAmAfUmAfUmGfGmUfGmA | fUfUmUfAmAfAmAfAmGfUmCfAm |
| | fCmUfUmUfUmUfAmAfAinvdT (SEQ ID | CfCmAfUmAfUmUdTdT (SEQ ID |
| | NO: 514) | NO: 515) |
| C209.018 | mCmCmAfAmUfAmUfGmGfUmGfAmC | fUfUmUfUmAfAmAfAmAfGmUfCm |
| | fUmUfUmUfUmAfAmAfAinvdT (SEQ | AfCmCfAmUfAmUdTdT (SEQ ID |
| | ID NO: 516) | NO: 517) |
| C209.019 | mCmCmAfAmUfGmGfUmGfAmCfUmU | fUfAmUfUmUfUmAfAmAfAmAfGm |
| | fUmUfUmAfAmAfAmUfAinvdT (SEQ | UfCmAfCmCfAmUdTdT (SEQ ID NO: |
| | ID NO: 518) | 519) |
| C209.020 | mCmCmAfUmGfGmUfGmAfCmUfUmU | fUfUmAfUmUfUmUfAmAfAmAfAm |
| | fUmUfAmAfAmAfUmAfAinvdT (SEQ | GfUmCfAmCfCmAdTdT (SEQ ID NO: |
| | ID NO: 520) | 521) |
| C209.021 | mCmCmAfGmGfUmGfAmCfUmUfUmU | fUfUmUfAmUfUmUfUmAfAmAfAm |
| | fUmAfAmAfAmUfAmAfAinvdT (SEQ | AfGmUfCmAfCmCdTdT (SEQ ID NO: |
| | ID NO: 522) | 523) |
| C209.022 | mCmCmAfGmUfGmAfCmUfUmUfUmU | fUfUmUfUmAfUmUfUmUfAmAfAm |
| | fAmAfAmAfUmAfAmAfAinvdT (SEQ | AfAmGfUmCfAmCdTdT (SEQ ID |
| | ID NO: 524) | NO: 525) |
| C209.023 | mCmCmAfUmGfAmCfUmUfUmUfUmA | fUfUmUfUmUfAmUfUmUfUmAfAm |
| | fAmAfAmUfAmAfAmAfAinvdT (SEQ | AfAmAfGmUfCmAdTdT (SEQ ID |
| | ID NO: 526) | NO: 527) |
| C209.024 | mCmCmAfGmAfCmUfUmUfUmUfAmA | fGfUmUfUmUfUmAfUmUfUmUfAm |
| | fAmAfUmAfAmAfAmAfCinvdT (SEQ ID | AfAmAfAmGfUmCdTdT (SEQ ID |
| | NO: 528) | NO: 529) |
| C209.025 | mCmCmAfAmCfUmUfUmUfUmAfAmA | fUfGmUfUmUfUmUfAmUfUmUfUm |
| | fAmUfAmAfAmAfAmCfAinvdT (SEQ ID | AfAmAfAmAfGmUdTdT (SEQ ID |
| | NO: 530) | NO: 531) |
| C209.026 | mCmCmAfCmUfUmUfUmUfAmAfAmA | fUfUmGfUmUfUmUfUmAfUmUfUm |
| | fUmAfAmAfAmAfCmAfAinvdT (SEQ ID | UfAmAfAmAfAmGdTdT (SEQ ID |
| | NO: 532) | NO: 533) |
| C209.027 | mCmCmAfUmUfUmUfUmAfAmAfAmU | fUfUmUfGmUfUmUfUmUfAmUfUm |
| | fAmAfAmAfAmCfAmAfAinvdT (SEQ ID | UfUmAfAmAfAmAdTdT (SEQ ID |
| | NO: 534) | NO: 535) |
| C209.028 | mCmCmAfUmUfUmUfAmAfAmAfUmA | fGfUmUfUmGfUmUfUmUfUmAfUm |
| | fAmAfAmAfCmAfAmAfCinvdT (SEQ ID | UfUmUfAmAfAmAdTdT (SEQ ID |
| | NO: 536) | NO: 537) |
| C209.029 | mCmCmAfUmUfUmAfAmAfAmUfAmA | fUfGmUfUmUfGmUfUmUfUmUfAm |
| | fAmAfAmCfAmAfAmCfAinvdT (SEQ ID | UfUmUfUmAfAmAdTdT (SEQ ID |
| | NO: 538) | NO: 539) |
| C217.002 | mCmCmAfCmAfUmUfUmUfUmAfUmU | fCfAmCfCmAfUmAfUmUfAmAfUm |
| | fAmAfUmAfUmGfGmUfGinvdT (SEQ | AfAmAfAmAfUmGdTdT (SEQ ID |
| | ID NO: 540) | NO: 541) |
| C217.003 | mCmCmAfAmUfUmUfUmUfAmUfUmA | fUfCmAfCmCfAmUfAmUfUmAfAm |
| | fAmUfAmUfGmGfUmGfAinvdT (SEQ | UfAmAfAmAfAmUdTdT (SEQ ID |
| | ID NO: 542) | NO: 543) |
| C217.004 | mCmCmAfUmUfUmUfUmAfUmUfAmA | fGfUmCfAmCfCmAfUmAfUmUfAm |
| | fUmAfUmGfGmUfGmAfCinvdT (SEQ ID | AfUmAfAmAfAmAdTdT (SEQ ID |
| | NO: 544) | NO: 545) |
| C217.006 | mCmCmAfUmUfUmUfAmUfUmAfAmU | fAfGmUfCmAfCmCfAmUfAmUfUm |
| | fAmUfGmGfUmGfAmCfUinvdT (SEQ ID | AfAmUfAmAfAmAdTdT (SEQ ID |
| | NO: 546) | NO: 547) |
| C217.007 | mCmCmAfUmUfUmAfUmUfAmAfUmA | fAfAmGfUmCfAmCfCmAfUmAfUm |
| | fUmGfGmUfGmAfCmUfUinvdT (SEQ ID | UfAmAfUmAfAmAdTdT (SEQ ID |
| | NO: 548) | NO: 549) |
| C217.012 | mCmCmAfAmAfUmAfUmGfGmUfGmA | fUfUmUfAmAfAmAfAmGfUmCfAm |
| | fCmUfUmUfUmUfAmAfAinvdT (SEQ ID | CfCmAfUmAfUmUdTdT (SEQ ID |
| | NO: 550) | NO: 551) |
| C218.001 | mCmCmAfGmCfAmUfUmUfUmUfAmU | fUfCmAfCmCfAmUfAmUfUmAfAm |
| | fUmAfAmUfAmUfGmGfUmGfAinvdT | UfAmAfAmAfAmUfGmCdTdT (SEQ |
| | (SEQ ID NO: 552) | ID NO: 553) |
| C218.002 | mCmCmAfCmAfUmUfUmUfUmAfUmU | fGfUmCfAmCfCmAfUmAfUmUfAm |
| | fAmAfUmAfUmGfGmUfGmAfCinvdT | AfUmAfAmAfAmAfUmGdTdT (SEQ |
| | (SEQ ID NO: 554) | ID NO: 555) |
| C218.004 | mCmCmAfUmUfUmUfUmAfUmUfAmA | fAfAmGfUmCfAmCfCmAfUmAfUm |
| | fUmAfUmGfGmUfGmAfCmUfUinvdT | UfAmAfUmAfAmAfAmAdTdT (SEQ |
| | (SEQ ID NO: 556) | ID NO: 557) |
| C218.007 | mCmCmAfUmUfAmUfUmAfAmUfAmU | fAfAmAfAmAfGmUfCmAfCmCfAm |
| | fGmGfUmGfAmCfUmUfUmUfUinvdT | UfAmUfUmAfAmUfAmAdTdT (SEQ |
| | (SEQ ID NO: 558) | ID NO: 559) |
| C218.009 | mCmCmAfAmUfUmAfAmUfAmUfGmG | fUfUmAfAmAfAmAfGmUfCmAfCm |
| | fUmGfAmCfUmUfUmUfUmAfAinvdT | CfAmUfAmUfUmAfAmUdTdT (SEQ |
| | (SEQ ID NO: 560) | ID NO: 561) |
| C218.010 | mCmCmAfUmUfAmAfUmAfUmGfGmU | fUfUmUfAmAfAmAfAmGfUmCfAm |
| | fGmAfCmUfUmUfUmUfAmAfAinvdT | CfCmAfUmAfUmUfAmAdTdT (SEQ |
| | (SEQ ID NO: 562) | ID NO: 563) |
| C218.011 | mCmCmAfUmAfAmUfAmUfGmGfUmG | fUfUmUfUmAfAmAfAmAfGmUfCm |
| | fAmCfUmUfUmUfUmAfAmAfAinvdT | AfCmCfAmUfAmUfUmAdTdT (SEQ |
| | (SEQ ID NO: 564) | ID NO: 565) |
| C219.002 | mCmCmAfCmAfUmUfUmUfUmAfUmU | fAfAmGfUmCfAmCfCmAfUmAfUm |
| | fAmAfUmAfUmGfGmUfGmAfCmUfUin | UfAmAfUmAfAmAfAmAfUmGdTdT |
| | vdT (SEQ ID NO: 566) | (SEQ ID NO: 567) |
| C219.005 | mCmCmAfUmUfUmUfAmUfUmAfAmU | fAfAmAfAmAfGmUfCmAfCmCfAm |
| | fAmUfGmGfUmGfAmCfUmUfUmUfUin | UfAmUfUmAfAmUfAmAfAmAdTdT |
| | vdT (SEQ ID NO: 568) | (SEQ ID NO: 569) |
| C219.008 | mCmCmAfUmAfUmUfAmAfUmAfUmG | fUfUmUfAmAfAmAfAmGfUmCfAm |
| | fGmUfGmAfCmUfUmUfUmUfAmAfAin | CfCmAfUmAfUmUfAmAfUmAdTdT |
| | vdT (SEQ ID NO: 570) | (SEQ ID NO: 571) |
| C219.009 | mCmCmAfAmUfUmAfAmUfAmUfGmG | fUfUmUfUmAfAmAfAmAfGmUfCm |
| | fUmGfAmCfUmUfUmUfUmAfAmAfAin | AfCmCfAmUfAmUfUmAfAmUdTdT |
| | vdT (SEQ ID NO: 572) | (SEQ ID NO: 573) |
| C219.010 | mCmCmAfUmUfAmAfUmAfUmGfGmU | fAfUmUfUmUfAmAfAmAfAmGfUm |
| | fGmAfCmUfUmUfUmUfAmAfAmAfUin | CfAmCfCmAfUmAfUmUfAmAdTdT |
| | vdT (SEQ ID NO: 574) | (SEQ ID NO: 575) |

mX = 2′-O-Me nucleotide
fX = 2′-F nucleotide
dX = DNA nucleotide
invdX = inverted dX

TABLE 3
siRNA sequences (without O-Me and F modifications).

| | Sequence sense strand 5′-->3′ | Sequence antisense strand 5′-->3′ |
|---|---|---|
| siRNA ID | (SEQ ID NO) | (SEQ ID NO) |
| B001 | CCAUUGUAGCAUUUUUAUUAAUinvdT | AUUAAUAAAAAUGCUACAAdTdT |
| | (SEQ ID NO: 162) | (SEQ ID NO: 163) |
| B002 | CCAUGUAGCAUUUUUAUUAAUAinvdT | UAUUAAUAAAAAUGCUACAdTdT |
| | (SEQ ID NO: 164) | (SEQ ID NO: 165) |
| B003 | CCAGUAGCAUUUUUAUUAAUAUinvdT | AUAUUAAUAAAAAUGCUACdTdT |
| | (SEQ ID NO: 166) | (SEQ ID NO: 167) |
| B004 | CCAUAGCAUUUUUAUUAAUAUGinvdT | CAUAUUAAUAAAAAUGCUAdTdT |
| | (SEQ ID NO: 168) | (SEQ ID NO: 169) |
| B005 | CCAAGCAUUUUUAUUAAUAUGGinvdT | CCAUAUUAAUAAAAAUGCUdTdT |
| | (SEQ ID NO: 170) | (SEQ ID NO: 171) |
| B006 | CCAGCAUUUUUAUUAAUAUGGUinvdT | ACCAUAUUAAUAAAAAUGCdTdT |
| | (SEQ ID NO: 172) | (SEQ ID NO: 173) |
| B007 | CCAUUUUUAUUAAUAUGGUGACinvdT | GUCACCAUAUUAAUAAAAAdTdT |
| | (SEQ ID NO: 174) | (SEQ ID NO: 175) |
| B008 | CCAUUUUAUUAAUAUGGUGACUinvdT | AGUCACCAUAUUAAUAAAAdTdT |
| | (SEQ ID NO: 176) | (SEQ ID NO: 177) |
| B009 | CCAUUUAUUAAUAUGGUGACUUinvdT | AAGUCACCAUAUUAAUAAAdTdT |
| | (SEQ ID NO: 178) | (SEQ ID NO: 179) |
| B010 | CCAUAUUAAUAUGGUGACUUUUinvdT | AAAAGUCACCAUAUUAAUAdTdT |
| | (SEQ ID NO: 180) | (SEQ ID NO: 181) |
| B011 | CCAAUUAAUAUGGUGACUUUUUinvdT | AAAAAGUCACCAUAUUAAUdTdT |
| | (SEQ ID NO: 182) | (SEQ ID NO: 183) |
| B012 | CCAUUAAUAUGGUGACUUUUUAinvdT | UAAAAAGUCACCAUAUUAAdTdT |
| | (SEQ ID NO: 184) | (SEQ ID NO: 185) |
| B013 | CCAUAAUAUGGUGACUUUUUAAinvdT | UUAAAAAGUCACCAUAUUAdTdT |
| | (SEQ ID NO: 186) | (SEQ ID NO: 187) |
| B014 | CCAUAUGGUGACUUUUUAAAAUinvdT | AUUUUAAAAAGUCACCAUAdTdT |
| | (SEQ ID NO: 188) | (SEQ ID NO: 189) |
| C001 | CCAGGCUCUGGUGGCGUGAUCUinvdT | AGAUCACGCCACCAGAGCCdTdT |
| | (SEQ ID NO: 190) | (SEQ ID NO: 191) |
| C002 | CCAGCUCUGGUGGCGUGAUCUGinvdT | CAGAUCACGCCACCAGAGCdTdT |
| | (SEQ ID NO: 192) | (SEQ ID NO: 193) |
| C003 | CCACUCUGGUGGCGUGAUCUGCinvdT | GCAGAUCACGCCACCAGAGdTdT |
| | (SEQ ID NO: 194) | (SEQ ID NO: 195) |
| C004 | CCAUCUGGUGGCGUGAUCUGCGinvdT | CGCAGAUCACGCCACCAGAdTdT |
| | (SEQ ID NO: 196) | (SEQ ID NO: 197) |
| C005 | CCACAGGCGUCAAGCACCCACAinvdT | UGUGGGUGCUUGACGCCUGdTdT |
| | (SEQ ID NO: 198) | (SEQ ID NO: 199) |
| C006 | CCAAGGCGUCAAGCACCCACACinvdT | GUGUGGGUGCUUGACGCCUdTdT |
| | (SEQ ID NO: 200) | (SEQ ID NO: 201) |
| C007 | CCACGUCAAGCACCCACACCCUinvdT | AGGGUGUGGGUGCUUGACGdTdT |
| | (SEQ ID NO: 202) | (SEQ ID NO: 203) |
| C008 | CCAGUCAAGCACCCACACCCUAinvdT | UAGGGUGUGGGUGCUUGACdTdT |
| | (SEQ ID NO: 204) | (SEQ ID NO: 205) |
| C009 | CCAUCAAGCACCCACACCCUAGinvdT | CUAGGGUGUGGGUGCUUGAdTdT |
| | (SEQ ID NO: 206) | (SEQ ID NO: 207) |
| C010 | CCACAAGCACCCACACCCUAGAinvdT | UCUAGGGUGUGGGUGCUUGdTdT |
| | (SEQ ID NO: 208) | (SEQ ID NO: 209) |
| C011 | CCAAAGCACCCACACCCUAGAAinvdT | UUCUAGGGUGUGGGUGCUUdTdT |
| | (SEQ ID NO: 210) | (SEQ ID NO: 211) |
| C012 | CCAAGCACCCACACCCUAGAAGinvdT | CUUCUAGGGUGUGGGUGCUdTdT |
| | (SEQ ID NO: 212) | (SEQ ID NO: 213) |
| C013 | CCAGCACCCACACCCUAGAAGGinvdT | CCUUCUAGGGUGUGGGUGCdTdT |
| | (SEQ ID NO: 214) | (SEQ ID NO: 215) |
| C014 | CCACACCCACACCCUAGAAGGUinvdT | ACCUUCUAGGGUGUGGGUGdTdT |
| | (SEQ ID NO: 216) | (SEQ ID NO: 217) |
| C015 | CCAACCCACACCCUAGAAGGUUinvdT | AACCUUCUAGGGUGUGGGUdTdT |
| | (SEQ ID NO: 218) | (SEQ ID NO: 219) |
| C016 | CCACCCACACCCUAGAAGGUUUinvdT | AAACCUUCUAGGGUGUGGGdTdT |
| | (SEQ ID NO: 220) | (SEQ ID NO: 221) |
| C017 | CCACCACACCCUAGAAGGUUUCinvdT | GAAACCUUCUAGGGUGUGGdTdT |
| | (SEQ ID NO: 222) | (SEQ ID NO: 223) |
| C018 | CCACACACCCUAGAAGGUUUCCinvdT | GGAAACCUUCUAGGGUGUGdTdT |
| | (SEQ ID NO: 224) | (SEQ ID NO: 225) |
| C019 | CCAACACCCUAGAAGGUUUCCGinvdT | CGGAAACCUUCUAGGGUGUdTdT |
| | (SEQ ID NO: 226) | (SEQ ID NO: 227) |
| C020 | CCACACCCUAGAAGGUUUCCGCinvdT | GCGGAAACCUUCUAGGGUGdTdT |
| | (SEQ ID NO: 228) | (SEQ ID NO: 229) |
| C021 | CCAACCCUAGAAGGUUUCCGCAinvdT | UGCGGAAACCUUCUAGGGUdTdT |
| | (SEQ ID NO: 230) | (SEQ ID NO: 231) |
| C022 | CCACCCUAGAAGGUUUCCGCAGinvdT | CUGCGGAAACCUUCUAGGGdTdT |
| | (SEQ ID NO: 232) | (SEQ ID NO: 233) |
| C023 | CCACCUAGAAGGUUUCCGCAGCinvdT | GCUGCGGAAACCUUCUAGGdTdT |
| | (SEQ ID NO: 234) | (SEQ ID NO: 235) |
| C024 | CCACUAGAAGGUUUCCGCAGCGinvdT | CGCUGCGGAAACCUUCUAGdTdT |
| | (SEQ ID NO: 236) | (SEQ ID NO: 237) |
| C025 | CCAUAGAAGGUUUCCGCAGCGAinvdT | UCGCUGCGGAAACCUUCUAdTdT |
| | (SEQ ID NO: 238) | (SEQ ID NO: 239) |
| C026 | CCAAGAAGGUUUCCGCAGCGACinvdT | GUCGCUGCGGAAACCUUCUdTdT |
| | (SEQ ID NO: 240) | (SEQ ID NO: 241) |
| C027 | CCAGAAGGUUUCCGCAGCGACGinvdT | CGUCGCUGCGGAAACCUUCdTdT |
| | (SEQ ID NO: 242) | (SEQ ID NO: 243) |
| C028 | CCAAAGGUUUCCGCAGCGACGUinvdT | ACGUCGCUGCGGAAACCUUdTdT |
| | (SEQ ID NO: 244) | (SEQ ID NO: 245) |
| C029 | CCAAGGUUUCCGCAGCGACGUCinvdT | GACGUCGCUGCGGAAACCUdTdT |
| | (SEQ ID NO: 246) | (SEQ ID NO: 247) |
| C030 | CCAGUUUCCGCAGCGACGUCGAinvdT | UCGACGUCGCUGCGGAAACdTdT |
| | (SEQ ID NO: 248) | (SEQ ID NO: 249) |
| C031 | CCAUUUCCGCAGCGACGUCGAGinvdT | CUCGACGUCGCUGCGGAAAdTdT |
| | (SEQ ID NO: 250) | (SEQ ID NO: 251) |
| C032 | CCACCUGAACUUCAGCUCCUGCinvdT | GCAGGAGCUGAAGUUCAGGdTdT |
| | (SEQ ID NO: 252) | (SEQ ID NO: 253) |
| C033 | CCACUGAACUUCAGCUCCUGCAinvdT | UGCAGGAGCUGAAGUUCAGdTdT |
| | (SEQ ID NO: 254) | (SEQ ID NO: 255) |
| C034 | CCAUGAACUUCAGCUCCUGCACinvdT | GUGCAGGAGCUGAAGUUCAdTdT |
| | (SEQ ID NO: 256) | (SEQ ID NO: 257) |
| C035 | CCAGGACAGCAACCUCUCCCCUinvdT | AGGGGAGAGGUUGCUGUCCdTdT |
| | (SEQ ID NO: 258) | (SEQ ID NO: 259) |
| C036 | CCAGACAGCAACCUCUCCCCUGinvdT | CAGGGGAGAGGUUGCUGUCdTdT |
| | (SEQ ID NO: 260) | (SEQ ID NO: 261) |
| C037 | CCAACAGCAACCUCUCCCCUGGinvdT | CCAGGGGAGAGGUUGCUGUdTdT |
| | (SEQ ID NO: 262) | (SEQ ID NO: 263) |
| C038 | CCAAUGGGCACCGUCAGCUCCAinvdT | UGGAGCUGACGGUGCCCAUdTdT |
| | (SEQ ID NO: 264) | (SEQ ID NO: 265) |
| C039 | CCAGGAUGAAUACCAGCCCCCCinvdT | GGGGGGCUGGUAUUCAUCCdTdT |
| | (SEQ ID NO: 266) | (SEQ ID NO: 267) |
| C040 | CCAGAUGAAUACCAGCCCCCCGinvdT | CGGGGGGCUGGUAUUCAUCdTdT |
| | (SEQ ID NO: 268) | (SEQ ID NO: 269) |
| C041 | CCAAUGAAUACCAGCCCCCCGAinvdT | UCGGGGGGCUGGUAUUCAUdTdT |
| | (SEQ ID NO: 270) | (SEQ ID NO: 271) |
| C042 | CCACAAGGGGGCAAGCUGGUCUinvdT | AGACCAGCUUGCCCCCUUGdTdT |
| | (SEQ ID NO: 272) | (SEQ ID NO: 273) |
| C043 | CCAAAGGGGGCAAGCUGGUCUGinvdT | CAGACCAGCUUGCCCCCUUdTdT |
| | (SEQ ID NO: 274) | (SEQ ID NO: 275) |
| C044 | CCAUGGACCCUGACUGGCUGCAinvdT | UGCAGCCAGUCAGGGUCCAdTdT |
| | (SEQ ID NO: 276) | (SEQ ID NO: 277) |
| C045 | CCAGACCCUGACUGGCUGCAGUinvdT | ACUGCAGCCAGUCAGGGUCdTdT |
| | (SEQ ID NO: 278) | (SEQ ID NO: 279) |
| C046 | CCAUGACUGGCUGCAGUGCCCUinvdT | AGGGCACUGCAGCCAGUCAdTdT |
| | (SEQ ID NO: 280) | (SEQ ID NO: 281) |
| C047 | CCAUCUUCCCAUGGAUAGGGGAinvdT | UCCCCUAUCCAUGGGAAGAdTdT |
| | (SEQ ID NO: 282) | (SEQ ID NO: 283) |
| C048 | CCACUUCCCAUGGAUAGGGGAGinvdT | CUCCCCUAUCCAUGGGAAGdTdT |
| | (SEQ ID NO: 284) | (SEQ ID NO: 285) |
| C049 | CCAUUCCCAUGGAUAGGGGAGGinvdT | CCUCCCCUAUCCAUGGGAAdTdT |
| | (SEQ ID NO: 286) | (SEQ ID NO: 287) |
| C050 | CCAGUAGGGGCUGCAGGGACAAinvdT | UUGUCCCUGCAGCCCCUACdTdT |
| | (SEQ ID NO: 288) | (SEQ ID NO: 289) |
| C051 | CCAGAGUGUGAAAGGUGCUGAUinvdT | AUCAGCACCUUUCACACUCdTdT |
| | (SEQ ID NO: 290) | (SEQ ID NO: 291) |
| C052 | CCAAGUGUGAAAGGUGCUGAUGinvdT | CAUCAGCACCUUUCACACUdTdT |
| | (SEQ ID NO: 292) | (SEQ ID NO: 293) |
| C053 | CCAGUGUGAAAGGUGCUGAUGGinvdT | CCAUCAGCACCUUUCACACdTdT |
| | (SEQ ID NO: 294) | (SEQ ID NO: 295) |
| C054 | CCAUGUGAAAGGUGCUGAUGGCinvdT | GCCAUCAGCACCUUUCACAdTdT |
| | (SEQ ID NO: 296) | (SEQ ID NO: 297) |
| C055 | CCAGUGAAAGGUGCUGAUGGCCinvdT | GGCCAUCAGCACCUUUCACdTdT |
| | (SEQ ID NO: 298) | (SEQ ID NO: 299) |
| C056 | CCAGCUGUGCCUUGGUUUCCUGinvdT | CAGGAAACCAAGGCACAGCdTdT |
| | (SEQ ID NO: 300) | (SEQ ID NO: 301) |
| C057 | CCAUGUGCCUUGGUUUCCUGAGinvdT | CUCAGGAAACCAAGGCACAdTdT |
| | (SEQ ID NO: 302) | (SEQ ID NO: 303) |
| C058 | CCAAACACCCAAAGGUGGCCUGinvdT | CAGGCCACCUUUGGGUGUUdTdT |
| | (SEQ ID NO: 304) | (SEQ ID NO: 305) |
| C059 | CCAACACCCAAAGGUGGCCUGCinvdT | GCAGGCCACCUUUGGGUGUdTdT |
| | (SEQ ID NO: 306) | (SEQ ID NO: 307) |
| C060 | CCAGAAGAAACCUGGAACCAGAinvdT | UCUGGUUCCAGGUUUCUUCdTdT |
| | (SEQ ID NO: 308) | (SEQ ID NO: 309) |
| C209.016 | CCAUUAUUAAUAUGGUGACUUUinvdT | AAAGUCACCAUAUUAAUAAdTdT |
| | (SEQ ID NO: 322) | (SEQ ID NO: 323) |
| C217.013 | CCAAUAUGGUGACUUUUUAAAAinvdT | UUUUAAAAAGUCACCAUAUdtdt |
| | (SEQ ID NO: 324) | (SEQ ID NO: 325) |
| C218.003 | CCAAUUUUUAUUAAUAUGGUGACUin | AGUCACCAUAUUAAUAAAAAUdT |
| | vdT (SEQ ID NO: 326) | dT (SEQ ID NO: 327) |
| C218.005 | CCAUUUUAUUAAUAUGGUGACUUUin | AAAGUCACCAUAUUAAUAAAAdT |
| | vdT (SEQ ID NO: 328) | dT (SEQ ID NO: 329) |
| C218.006 | CCAUUUAUUAAUAUGGUGACUUUUin | AAAAGUCACCAUAUUAAUAAAdT |
| | vdT (SEQ ID NO: 330) | dT (SEQ ID NO: 331) |
| C218.008 | CCAUAUUAAUAUGGUGACUUUUUAin | UAAAAAGUCACCAUAUUAAUAdT |
| | vdT (SEQ ID NO: 332) | dT (SEQ ID NO: 333) |
| C218.012 | CCAAAUAUGGUGACUUUUUAAAAUin | AUUUUAAAAAGUCACCAUAUUdT |
| | vdT (SEQ ID NO: 334) | dT (SEQ ID NO: 335) |
| C219.001 | CCAGCAUUUUUAUUAAUAUGGUGAC | AGUCACCAUAUUAAUAAAAAUG |
| | UmvdT (SEQ ID NO: 336) | CdTdT (SEQ ID NO: 337) |
| C219.003 | CCAAUUUUUAUUAAUAUGGUGACUU | AAAGUCACCAUAUUAAUAAAAA |
| | UmvdT (SEQ ID NO: 338) | UdTdT (SEQ ID NO: 339) |
| C219.004 | CCAUUUUUAUUAAUAUGGUGACUUU | AAAAGUCACCAUAUUAAUAAAA |
| | UmvdT (SEQ ID NO: 340) | AdTdT (SEQ ID NO: 341) |
| C219.006 | CCAUUUAUUAAUAUGGUGACUUUUU | UAAAAAGUCACCAUAUUAAUAA |
| | AtnvdT (SEQ ID NO: 342) | AdTdT (SEQ ID NO: 343) |
| C219.007 | CCAUUAUUAAUAUGGUGACUUUUUA | UUAAAAAGUCACCAUAUUAAUA |
| | AtnvdT (SEQ ID NO: 344) | AdTdT (SEQ ID NO: 345) |
| C209.001 | CCACAGGACAGCAACCUCUCCCinvdT | GGGAGAGGUUGCUGUCCUGdTdT |
| | (SEQ ID NO: 392) | (SEQ ID NO: 393) |
| C209.002 | CCAAGGACAGCAACCUCUCCCCinvdT | GGGGAGAGGUUGCUGUCCUdTdT |
| | (SEQ ID NO: 394) | (SEQ ID NO: 395) |
| C209.003 | CCAUGCCACUGCUGCUGCUGCUinvdT | AGCAGCAGCAGCAGUGGCAdTdT |
| | (SEQ ID NO: 396) | (SEQ ID NO: 397) |
| C209.004 | CCACACUGCUGCUGCUGCUGCUinvdT | AGCAGCAGCAGCAGCAGUGdTdT |
| | (SEQ ID NO: 398) | (SEQ ID NO: 399) |
| C209.005 | CCAUGCUGCUGCUGCUGCUCCUinvdT | AGGAGCAGCAGCAGCAGCAdTdT |
| | (SEQ ID NO: 400) | (SEQ ID NO: 401) |
| C209.006 | CCAACCCUGACUGGCUGCAGUGinvdT | CACUGCAGCCAGUCAGGGUdTdT |
| | (SEQ ID NO: 402) | (SEQ ID NO: 403) |
| C209.007 | CCAUGAAAGGUGCUGAUGGCCCinvdT | GGGCCAUCAGCACCUUUCAdTdT |
| | (SEQ ID NO: 404) | (SEQ ID NO: 405) |
| C209.008 | CCAGGCUGUGCCUUGGUUUCCUinvdT | AGGAAACCAAGGCACAGCCdTdT |
| | (SEQ ID NO: 406) | (SEQ ID NO: 407) |
| C209.009 | CCACUGUGCCUUGGUUUCCUGAinvdT | UCAGGAAACCAAGGCACAGdTdT |
| | (SEQ ID NO: 408) | (SEQ ID NO: 409) |
| C209.010 | CCAGGAAGAAACCUGGAACCAGinvdT | CUGGUUCCAGGUUUCUUCCdTdT |
| | (SEQ ID NO: 410) | (SEQ ID NO: 411) |
| C209.011 | CCAAAGAAACCUGGAACCAGAGinvdT | CUCUGGUUCCAGGUUUCUUdTdT |
| | (SEQ ID NO: 412) | (SEQ ID NO: 413) |
| C209.012 | CCAAGAAACCUGGAACCAGAGGinvdT | CCUCUGGUUCCAGGUUUCUdTdT |
| | (SEQ ID NO: 414) | (SEQ ID NO: 415) |
| C209.013 | CCAAACCUGGAACCAGAGGGGGinvdT | CCCCCUCUGGUUCCAGGUUdTdT |
| | (SEQ ID NO: 416) | (SEQ ID NO: 417) |
| C209.014 | CCACAUUUUUAUUAAUAUGGUGinvdT | CACCAUAUUAAUAAAAAUGdTdT |
| | (SEQ ID NO: 418) | (SEQ ID NO: 419) |
| C209.015 | CCAAUUUUUAUUAAUAUGGUGAinvdT | UCACCAUAUUAAUAAAAAUdTdT |
| | (SEQ ID NO: 420) | (SEQ ID NO: 421) |
| C209.017 | CCAAAUAUGGUGACUUUUUAAAinvdT | UUUAAAAAGUCACCAUAUUdTdT |
| | (SEQ ID NO: 422) | (SEQ ID NO: 423) |
| C209.018 | CCAAUAUGGUGACUUUUUAAAAinvdT | UUUUAAAAAGUCACCAUAUdTdT |
| | (SEQ ID NO: 424) | (SEQ ID NO: 425) |
| C209.019 | CCAAUGGUGACUUUUUAAAAUAinvdT | UAUUUUAAAAAGUCACCAUdTdT |
| | (SEQ ID NO: 426) | (SEQ ID NO: 427) |
| C209.020 | CCAUGGUGACUUUUUAAAAUAAinvdT | UUAUUUUAAAAAGUCACCAdTdT |
| | (SEQ ID NO: 428) | (SEQ ID NO: 429) |
| C209.021 | CCAGGUGACUUUUUAAAAUAAAinvdT | UUUAUUUUAAAAAGUCACCdTdT |
| | (SEQ ID NO: 430) | (SEQ ID NO: 431) |
| C209.022 | CCAGUGACUUUUUAAAAUAAAAinvdT | UUUUAUUUUAAAAAGUCACdTdT |
| | (SEQ ID NO: 432) | (SEQ ID NO: 433) |
| C209.023 | CCAUGACUUUUUAAAAUAAAAAinvdT | UUUUUAUUUUAAAAAGUCAdTdT |
| | (SEQ ID NO: 434) | (SEQ ID NO: 435) |
| C209.024 | CCAGACUUUUUAAAAUAAAAACinvdT | GUUUUUAUUUUAAAAAGUCdTdT |
| | (SEQ ID NO: 436) | (SEQ ID NO: 437) |
| C209.025 | CCAACUUUUUAAAAUAAAAACAinvdT | UGUUUUUAUUUUAAAAAGUdTdT |
| | (SEQ ID NO: 438) | (SEQ ID NO: 439) |
| C209.026 | CCACUUUUUAAAAUAAAAACAAinvdT | UUGUUUUUAUUUUAAAAAGdTdT |
| | (SEQ ID NO: 440) | (SEQ ID NO: 441) |
| C209.027 | CCAUUUUUAAAAUAAAAACAAAinvdT | UUUGUUUUUAUUUUAAAAAdTdT |
| | (SEQ ID NO: 442) | (SEQ ID NO: 443) |
| C209.028 | CCAUUUUAAAAUAAAAACAAACinvdT | GUUUGUUUUUAUUUUAAAAdTdT |
| | (SEQ ID NO: 444) | (SEQ ID NO: 445) |
| C209.029 | CCAUUUAAAAUAAAAACAAACAinvdT | UGUUUGUUUUUAUUUUAAAdTdT |
| | (SEQ ID NO: 446) | (SEQ ID NO: 447) |
| C217.002 | CCACAUUUUUAUUAAUAUGGUGinvdT | CACCAUAUUAAUAAAAAUGdTdT |
| | (SEQ ID NO: 448) | (SEQ ID NO: 449) |
| C217.003 | CCAAUUUUUAUUAAUAUGGUGAinvdT | UCACCAUAUUAAUAAAAAUdTdT |
| | (SEQ ID NO: 450) | (SEQ ID NO: 451) |
| C217.004 | CCAUUUUUAUUAAUAUGGUGACinvdT | GUCACCAUAUUAAUAAAAAdTdT |
| | (SEQ ID NO: 452) | (SEQ ID NO: 453) |
| C217.006 | CCAUUUAUUAAUAUGGUGACUUinvdT | AAGUCACCAUAUUAAUAAAdTdT |
| | (SEQ ID NO: 454) | (SEQ ID NO: 455) |
| C217.007 | CCAUUAUUAAUAUGGUGACUUUinvdT | AAAGUCACCAUAUUAAUAAdTdT |
| | (SEQ ID NO: 456) | (SEQ ID NO: 457) |
| C217.012 | CCAAAUAUGGUGACUUUUUAAAinvdT | UUUAAAAAGUCACCAUAUUdTdT |
| | (SEQ ID NO: 458) | (SEQ ID NO: 459) |
| C218.001 | CCAGCAUUUUUAUUAAUAUGGUGAin | UCACCAUAUUAAUAAAAAUGCdT |
| | vdT (SEQ ID NO: 460) | dT (SEQ ID NO: 461) |
| C218.002 | CCACAUUUUUAUUAAUAUGGUGACin | GUCACCAUAUUAAUAAAAAUGdT |
| | vdT (SEQ ID NO: 462) | dT (SEQ ID NO: 463) |
| C218.004 | CCAUUUUUAUUAAUAUGGUGACUUin | AAGUCACCAUAUUAAUAAAAAdT |
| | vdT (SEQ ID NO: 464) | dT (SEQ ID NO: 465) |
| C218.007 | CCAUUAUUAAUAUGGUGACUUUUUin | AAAAAGUCACCAUAUUAAUAAdT |
| | vdT (SEQ ID NO: 466) | dT (SEQ ID NO: 467) |
| C218.009 | CCAAUUAAUAUGGUGACUUUUUAAin | UUAAAAAGUCACCAUAUUAAUdT |
| | vdT (SEQ ID NO: 468) | dT (SEQ ID NO: 469) |
| C218.010 | CCAUUAAUAUGGUGACUUUUUAAAin | UUUAAAAAGUCACCAUAUUAAdT |
| | vdT (SEQ ID NO: 470) | dT (SEQ ID NO: 471) |
| C218.011 | CCAUAAUAUGGUGACUUUUUAAAAin | UUUUAAAAAGUCACCAUAUUAdT |
| | vdT (SEQ ID NO: 472) | dT (SEQ ID NO: 473) |
| C219.002 | CCACAUUUUUAUUAAUAUGGUGACU | AAGUCACCAUAUUAAUAAAAAU |
| | UmvdT (SEQ ID NO: 474) | GdTdT (SEQ ID NO: 475) |
| C219.005 | CCAUUUUAUUAAUAUGGUGACUUUU | AAAAAGUCACCAUAUUAAUAAA |
| | UmvdT (SEQ ID NO: 476) | AdTdT (SEQ ID NO: 477) |
| C219.008 | CCAUAUUAAUAUGGUGACUUUUUAA | UUUAAAAAGUCACCAUAUUAAU |
| | AncivdT (SEQ ID NO: 478) | AdTdT (SEQ ID NO: 479) |
| C219.009 | CCAAUUAAUAUGGUGACUUUUUAAA | UUUUAAAAAGUCACCAUAUUAA |
| | AncivdT (SEQ ID NO: 480) | UdTdT (SEQ ID NO: 481) |
| C219.010 | CCAUUAAUAUGGUGACUUUUUAAAA | AUUUUAAAAAGUCACCAUAUUA |
| | UmvdT (SEQ ID NO: 482) | AdTdT (SEQ ID NO: 483) |

invdX = inverted dX nucleotide

In some embodiments, the dsRNA comprises one or more modified nucleotides described in PCT Publication WO 2019/170731, the disclosure of which is incorporated herein in its entirety. In such modified nucleotides, the ribose ring has been replaced by a six-membered heterocyclic ring. Such a modified nucleotide has the structure of formula (I):

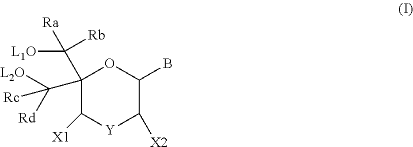

wherein:
B is a heterocyclic nucleobase;
one of L1 and L2 is an internucleoside linking group linking the compound of formula (I) to a polynucleotide and the other of L1 and L2 is H, a protecting group, a phosphorus moiety or an internucleoside linking group linking the compound of formula (I) to a polynucleotide,
Y is O, NH, NR1 or N—C(═O)—R1, wherein R1 is:
   a (C1-C20) alkyl group, optionally substituted by one or more groups selected from an halogen atom, a (C1-C6) alkyl group, a (C3-C8) cycloalkyl group, a (C3-C14) heterocycle, a (C6-C14) aryl group, a (C5-C14) heteroaryl group, —O—Z1, —N(Z1)(Z2), —S—Z1, —CN, —C(=J)-O—Z1, —O—C(=J)-Z1, —C(=J)-N(Z1)(Z2), and —N(Z1)-C(=J)-Z2, wherein
      J is O or S,
      each of Z1 and Z2 is, independently, H, a (C1-C6) alkyl group, optionally substituted by one or more groups selected from a halogen atom and a (C1-C6) alkyl group,
   a (C3-C8) cycloalkyl group, optionally substituted by one or more groups selected from a halogen atom and a (C1-C6) alkyl group,
   a group —[C(═O)]m-R2-(O—CH2-CH2)p-R3, wherein
      m is an integer meaning 0 or 1,
      p is an integer ranging from 0 to 10,
      R2 is a (C1-C20) alkylene group optionally substituted by a (C1-C6) alkyl group, —O—Z3, —N(Z3)(Z4), —S—Z3, —CN, —C(═K)—O—Z3, —O—C(═K)—Z3, —C(═K)—N(Z3)(Z4), or —N(Z3)-C(═K)—Z4, wherein
         K is O or S,
         each of Z3 and Z4 is, independently, H, a (C1-C6) alkyl group, optionally substituted by one or more groups selected from a halogen atom and a (C1-C6) alkyl group, and
      R3 is selected from the group consisting of a hydrogen atom, a (C1-C6) alkyl group, a (C1-C6) alkoxy group, a (C3-C8) cycloalkyl group, a (C3-C14) heterocycle, a (C6-C14) aryl group or a (C5-C14) heteroaryl group, or R3 is a cell targeting moiety,
X1 and X2 are each, independently, a hydrogen atom, a (C1-C6) alkyl group, and
each of Ra, Rb, Rc and Rd is, independently, H or a (C1-C6) alkyl group,
   or is a pharmaceutically acceptable salt thereof.

In some embodiments, Y is NR1, R1 is a non-substituted (C1-C20) alkyl group, and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a non-substituted (C1-C16) alkyl group, which includes an alkyl group selected from a group comprising methyl, isopropyl, butyl, octyl, hexadecyl, and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a (C3-C8) cycloalkyl group, optionally substituted by one or more groups selected from a halogen atom and a (C1-C6) alkyl group, and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a cyclohexyl group, and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a (C1-C20) alkyl group substituted by a (C6-C14) aryl group and L1, L2, Ra, Rb, Rc, Rd, X1, X2, R2, R3 and B have the same meaning as defined for the general formula (I), or a pharmaceutically acceptable salt thereof.

In some embodiments, Y is NR1, R1 is a methyl group substituted by a phenyl group, and L1, L2, Ra, Rb, Rc, Rd, X1, X2, R2, R3 and B have the same meaning as defined for the general formula (I), or a pharmaceutically acceptable salt thereof.

In some embodiments, Y is N—C(═O)—R1, R1 is an optionally substituted (C1-C20) alkyl group, and L1, L2, Ra, Rb, Rc, Rd, X1, X2, R2, R3 and B have the same meaning as defined for the general formula (I), or a pharmaceutically acceptable salt thereof.

In some embodiments, Y is N—C(═O)—R1, R1 is selected from a group comprising methyl and pentadecyl and L1, L2, Ra, Rb, Rc, Rd, X1, X2, R2, R3 and B have the same meaning as defined for the general formula (I), or a pharmaceutically acceptable salt thereof.

In some embodiments, the dsRNA comprises one or more compounds of formula (I) wherein Y is

a) NR1, wherein R1 is a non-substituted (C1-C20) alkyl group;
b) NR1, wherein R1 is a non-substituted (C1-C16) alkyl group, which includes an alkyl group selected from a group comprising methyl, isopropyl, butyl, octyl, and hexadecyl;
c) NR1, wherein R1 is a (C3-C8) cycloalkyl group, optionally substituted by one or more groups selected from a halogen atom and a (C1-C6) alkyl group;
d) NR1, wherein R1 is a cyclohexyl group;
e) NR1, wherein R1 is a (C1-C20) alkyl group substituted by a (C6-C14) aryl group;
f) NR1, wherein R1 is a methyl group substituted by a phenyl group;
g) N—C(═O)—R1, wherein R1 is an optionally substituted (C1-C20) alkyl group; or
h) N—C(═O)—R1, wherein R1 is methyl or pentadecyl.

In some embodiments, B is selected from a group comprising a pyrimidine, a substituted pyrimidine, a purine and a substituted purine, or a pharmaceutically acceptable salt thereof.

In some embodiments, the internucleoside linking group in the dsRNA is independently selected from the group consisting of phosphodiester, phosphotriester, phosphorothioate, phosphorodithioate, alkyl-phosphonate and phosphoramidate backbone linking groups, or a pharmaceutically acceptable salt thereof. In some embodiments, the dsRNA comprises one or more internucleoside linking groups independently selected from the group consisting of phosphodiester, phosphotriester, phosphorothioate, phosphorodithioate, alkyl-phosphonate and phosphoramidate backbone linking groups, or a pharmaceutically acceptable salt thereof.

In some embodiments, the dsRNA comprises from 2 to 10 compounds of formula (I), or a pharmaceutically acceptable salt thereof. In an embodiment, the 2 to 10 compounds of formula (I) are on the sense strand.

In further embodiments, the dsRNA comprises one or more targeted nucleotides or a pharmaceutically acceptable salt thereof.

In some embodiments, R3 is of the formula (II):

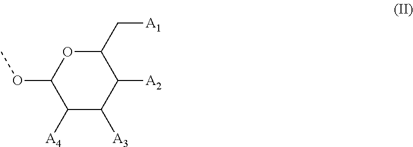

wherein A1, A2 and A3 are OH,
A4 is OH or NHC(═O)—R5, wherein R5 is a (C1-C6) alkyl group, optionally substituted by a halogen atom. or a pharmaceutically acceptable salt thereof

In some embodiments, R3 is N-acetyl-galactosamine, or a pharmaceutically acceptable salt thereof

The precursors that can be used to make modified siRNAs having nucleotides of formula (I) are exemplified in Table A below. Table A shows examples of phosphoramidite nucleotide analogs for oligonucleotide synthesis. In the (2S,6R) diastereomeric series, the phosphoramidites as nucleotide precursors are abbreviated with a “pre-l”, the nucleotide analogs are abbreviated with an “l”, followed by the nucleobase and a number, which specifies the group Y in formula (I). To distinguish both stereochemistries, the analogues (2R,6R)-diastereoisomers are indicated with an additional “b.” Targeted nucleotide precursors, targeted nucleotide analogs and solid supports are abbreviated as described above, but with an “lg” instead of the “l.”

TABLE A

| | | | Name in | |
|---|---|---|---|---|
| | | Precursor | oligo- | Stereo- |
| No | Structure | name | sequence | chemistry |
| 1 | 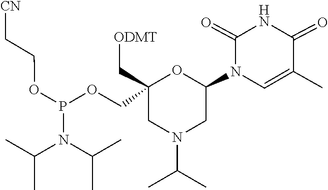 | pre-1T3 | 1T3 | (2S,6R) |
| 2 | 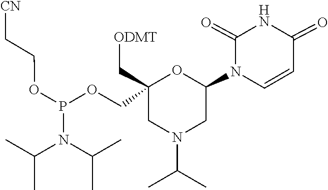 | pre-1U3 | 1U3 | (2S,6R) |
| 3 | 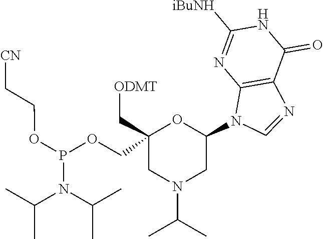 | pre-1G3 | 1G3 | (2S,6R) |
| 4 | 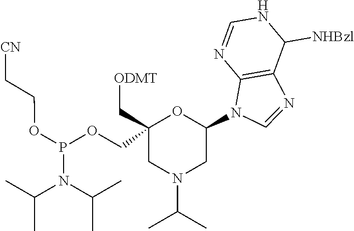 | pre-1A3 | 1A3 | (2S,6R) |
| 5 | 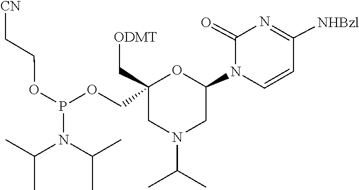 | pre-1C3 | 1C3 | (2S,6R) |
| 6 | 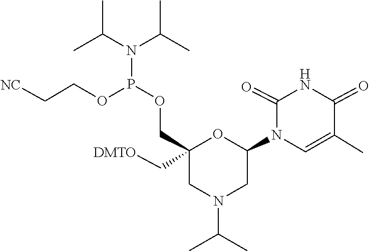 | pre-1T3b | 1T3b | (2R,6R) |
| 7 | 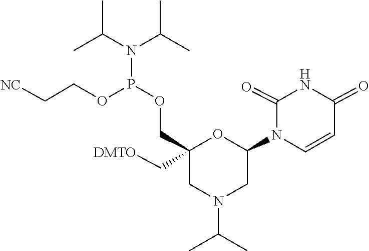 | pre-1U3b | 1U3b | (2R,6R) |
| 8 | 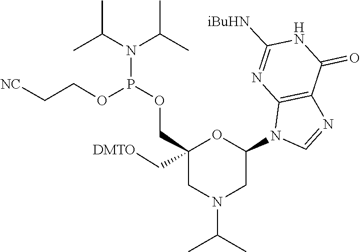 | pre-1G3b | 1G3b | (2R,6R) |
| 9 | 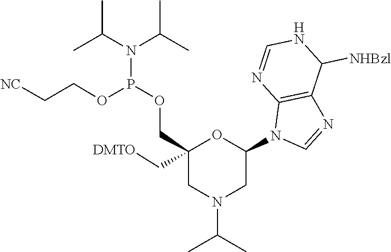 | pre-1A3b | 1A3b | (2R,6R) |
| 10 | 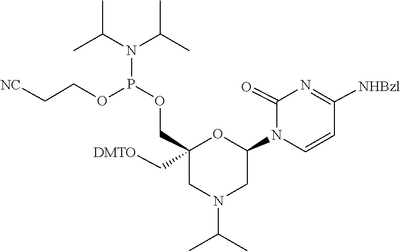 | pre-1C3b | 1C3b | (2R,6R) |
| 11 | 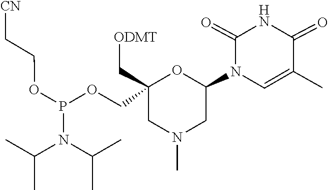 | pre-1T2 | 1T2 | (2S,6R) |
| 12 | 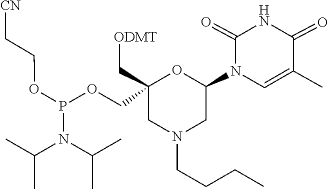 | pre-1T6 | 1T6 | (2S,6R) |
| 13 | 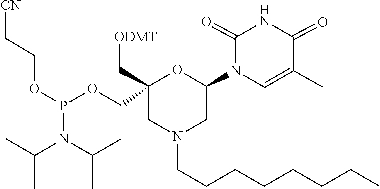 | pre-1T7 | 1T7 | (2S,6R) |
| 14 | 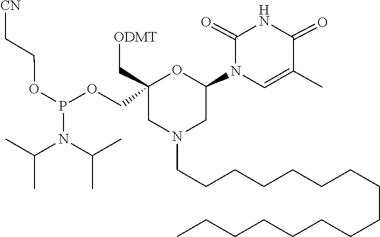 | pre-1T8 | 1T8 | (2S,6R) |
| 15 | 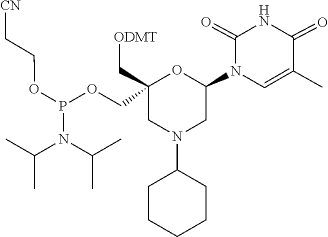 | pre-1T4 | 1T4 | (2S,6R) |
| 16 | 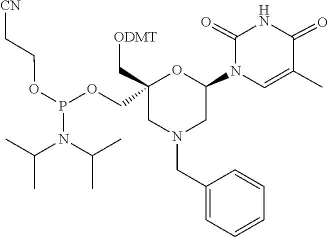 | pre-1T5 | 1T5 | (2S,6R) |
| 17 | 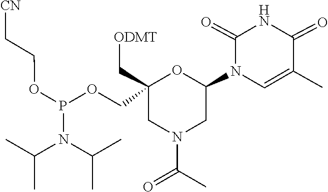 | pre-1T9 | 1T9 | (2S,6R) |
| 18 | 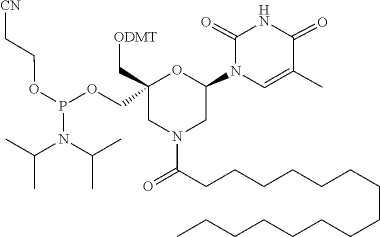 | pre-1T10 | 1T10 | (2S,6R) |
| 19 | 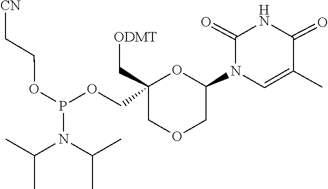 | pre-1T1 | 1T1 | (2S,6R) |
| 20 | 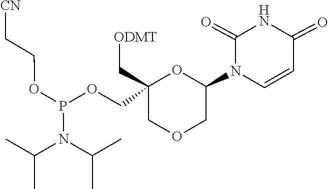 | pre-1U1 | 1U1 | (2S,6R) |
| 21 | 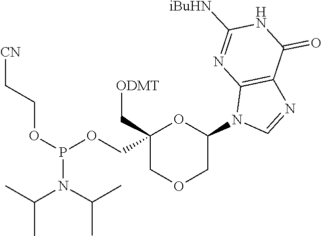 | pre-1G1 | 1G1 | (2S,6R) |
| 22 | 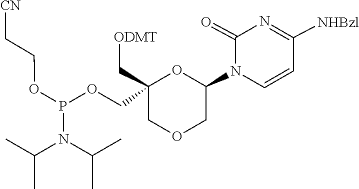 | pre-1C1 | 1C1 | (2S,6R) |
| 23 | 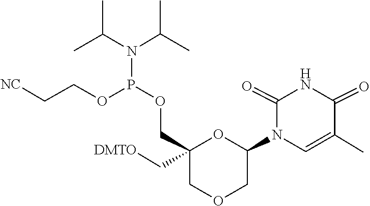 | pre-1T1b | 1T1b | (2R,6R) |
| 24 | 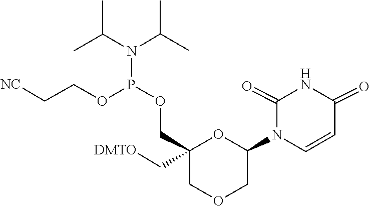 | pre-1U1b | 1U1b | (2R,6R) |
| 25 | 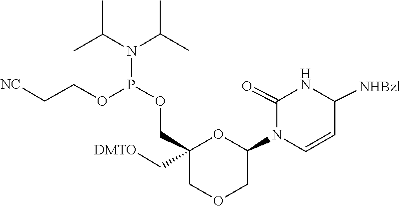 | pre-1C1b | 1C1b | (2R,6R) |
| 26 | 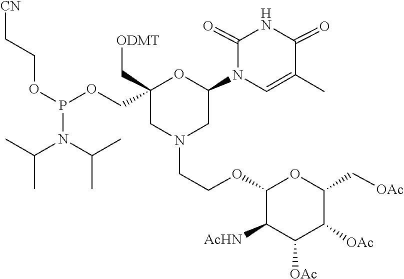 | pre-1gT9 | 1gT9 | (2S,6R) |
| 27 | 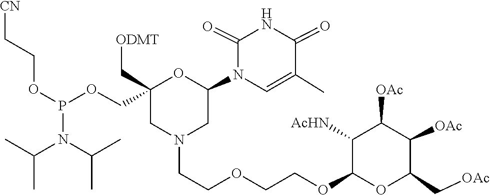 | pre-1gT8 | 1gT8 | (2S,6R) |
| 28 | 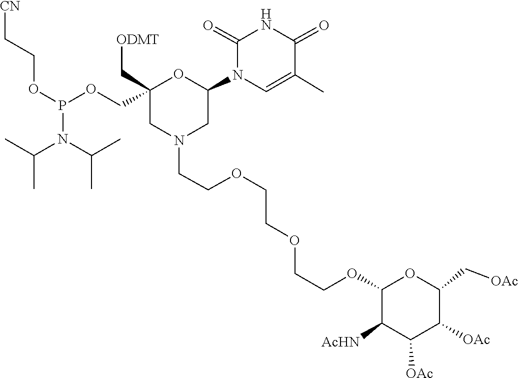 | pre-1gT7 | 1gT7 | (2S,6R) |
| 29 | 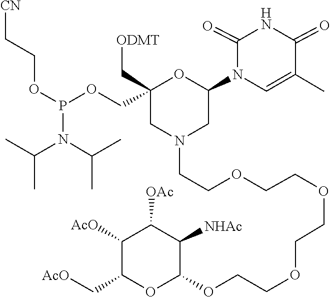 | pre-1gT6 | 1gT6 | (2S,6R) |
| 30 | 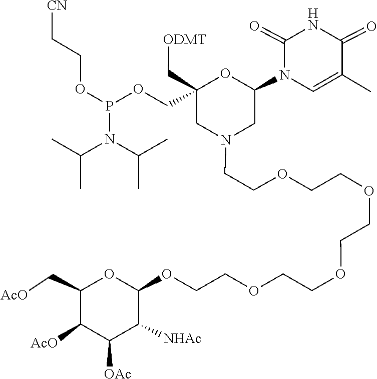 | pre-1gT5 | 1gT5 | (2S,6R) |
| 31 | 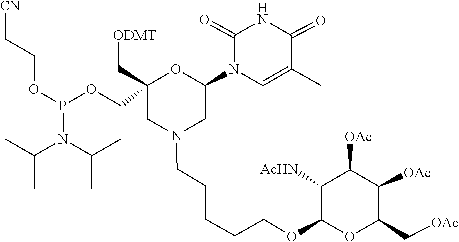 | pre-1gT3 | 1gT3 | (2S,6R) |
| 32 | 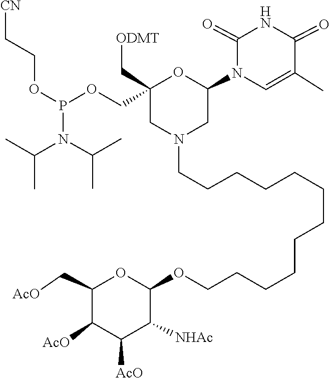 | pre-1gT4 | 1gT4 | (2S,6R) |
| 33 | 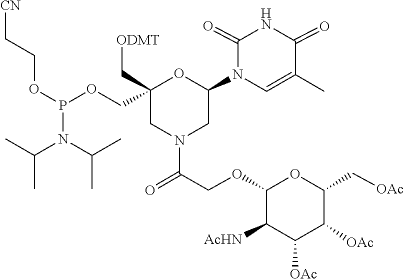 | pre-1gT12 | 1gT12 | (2S,6R) |
| 34 | 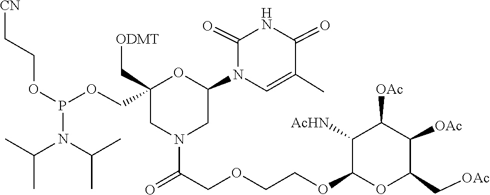 | pre-1gT11 | 1gT11 | (2S,6R) |
| 35 | 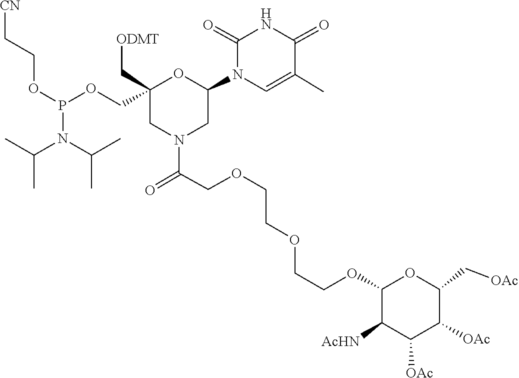 | pre-1gT10 | 1gT10 | (2S,6R) |
| 36 | 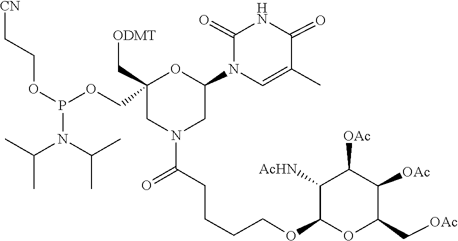 | pre-1gT1 | 1gT1 | (2S,6R) |
| 37 | 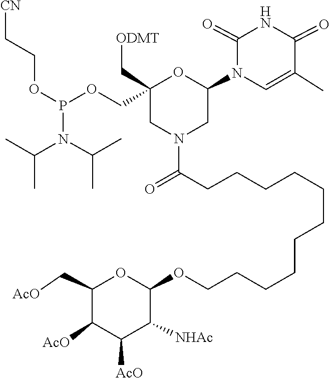 | pre-1gT2 | 1gT2 | (2S,6R) |
| 38 | 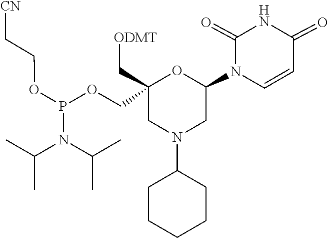 | pre-1U4 | 1U4 | (2S,6R) |
| 39 | 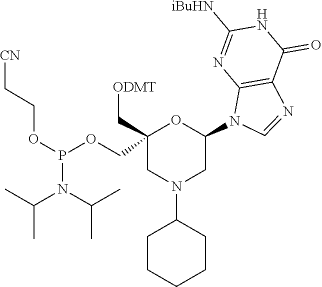 | pre-1G4 | 1G4 | (2S,6R) |
| 40 | 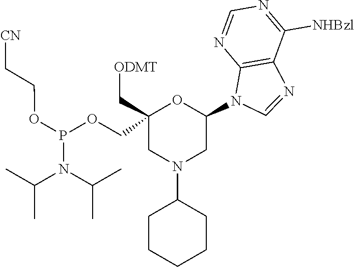 | pre-1A4 | 1A4 | (2S,6R) |
| 41 | 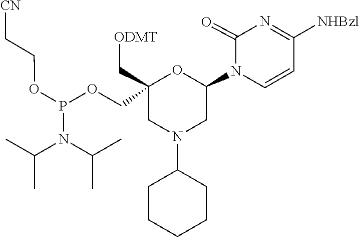 | pre-1C4 | 1C4 | (2S,6R) |
| 42 | 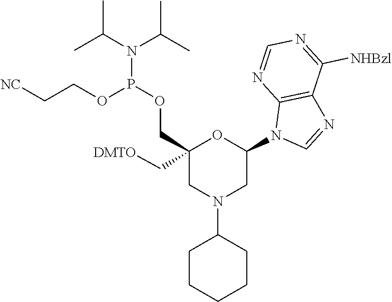 | pre-1A4b | 1A4b | (2R,6R) |
| 43 | 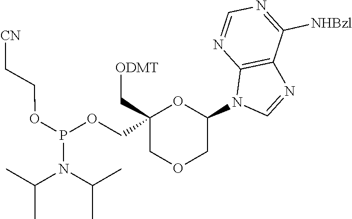 | pre-1A1 | 1A1 | (2S,6R) |
| 44 | 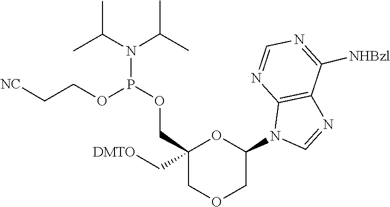 | pre-1A1b | 1A1b | (2R,6R) |
| 45 | 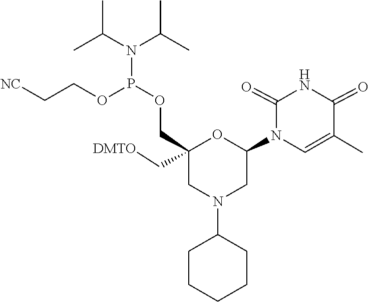 | pre-1T4b | 1T4b | (2R,6R) |
| 46 | 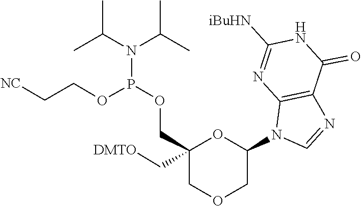 | pre-1G1b | 1G1b | (2R,6R) |

The modified nucleotides of formula (I) may be incorporated at the 5′, 3′, or both ends of the sense strand and/or antisense strand of the dsRNA. By way of example, one or more (e.g., 1, 2, 3, 4, or 5 or more) modified nucleotides may be incorporated at the 5′ end of the sense strand of the dsRNA. In some embodiments, one or more (e.g., 1, 2, 3, or more) modified nucleotides are positioned in the 5′ end of the sense strand, where the modified nucleotides do not complement the antisense sequence but may be optionally paired with an equal or smaller number of complementary nucleotides at the corresponding 3′ end of the antisense strand.

In some embodiments, the dsRNA may comprise a sense strand having a sense sequence of 17, 18, or 19 nucleotides in length, where three to five nucleotides of formula (I) (e.g., three consecutive lgT3 or lgT7 with or without additional nucleotides of formula (I)) are placed in the 5′ end of the sense sequence, making the sense strand 20, 21, or 22 nucleotides in length. In such embodiments, the sense strand may additionally comprise two consecutive nucleotides of formula (I) (e.g., lT4 or lT3) at the 3′ of the sense sequence, making the sense strand 22, 23, or 24 nucleotides in length. The dsRNA may comprise an antisense sequence of 19 nucleotides in length, where the antisense sequence may additionally be linked to 2 modified nucleotides or deoxyribonucleotides (e.g., dT) at its 3′ end, making the antisense strand 21 nucleotides in length. In further embodiments, the sense strand of the dsRNA contains only naturally occurring internucleotide bonds (phosphodiester bond), where the antisense strand may optionally contain non-naturally occurring internucleotide bonds. For example, the antisense strand may contain phosphorothioate bonds in the backbone near or at its 5′ and/or 3′ ends.

In some embodiments, the use of modified nucleotides of formula (I) circumvents the need for other RNA modifications such as the use of non-naturally occurring internucleotide bonds, thereby simplifying the chemical synthesis of dsRNAs. Moreover, the modified nucleotides of formula (I) can be readily made to contain cell targeted moieties such as GalNAc derivatives (which include GalNAc itself), enhancing the delivery efficiency of dsRNAs incorporating such nucleotides. Further, it has been shown that dsRNAs incorporating modified nucleotides of formula (I), e.g., at the sense strand, significantly improve the stability and therapeutic potency of the dsRNAs.

In some embodiments, a dsRNA of the present disclosure comprises a sense strand and/or an antisense strand described in double-stranded ribonucleic acid (dsRNA), wherein the dsRNA comprises a sense strand comprising a first sequence and an antisense strand comprising a second sequence, wherein the first sequence and the second sequence are complementary, wherein the first sequence comprises a sequence selected from the group consisting of SEQ ID NOS: 6-11 and 310-321. The siRNAs in table 4 may comprise any one or more of the following modifications: mX=2′-O-methyl-nucleotide, fX=2′-fluoro-nucleotide, lX=locked nucleotide, dT=deoxythymidine, lgT3=lgT3 nucleotide analog, lT4=lT4 nucleotide analog, PO=phosphodiester linkage; and PS=phosphorothioate linkage.

TABLE 4
Optimized PCSK9 GalNAc siRNAs

| | | Sequence antisense strand |
|---|---|---|
| siRNA ID | Sequence sense strand 5′-->3′ | 5′-->3′ |
| C027.001 | lgT3-PO-lgT3-PO-lgT3-PO-fU- | fA-PS4U-PS-mU-PO-fU- |
| | PO-mA-PO-fU-PO-mG-PO-fG- | PO-mU-PO-fA-PO-mA- |
| | PO-mU-PO-fG-PO-mA-PO-fC- | PO-fA-PO-mA-PO-fA-PO- |
| | PO-mU-PO-fU-PO-mU-PO-fU- | mG-PO-fU-PO-mC-PO-fA- |
| | PO-mU-PO-fA-PO-mA-PO-fA- | PO-mC-PO-fC-PO-mA- |
| | PS-mA-PS-fU (SEQ ID NO: 578) | PO4U-PO-mA-PS-dT-PS- |
| | | dT (SEQ ID NO: 589) |
| C027.002 | lgT3-PO-lgT3-PO-lgT3-PO-fG- | fA-PS-fC-PS-mC-PO-fA- |
| | PO-mC-PO-fA-PO-mU-PO-fU- | PO-mU-PO-fA-PO-mU- |
| | PO-mU-PO-fU-PO-mU-PO-fA- | PO4U-PO-mA-PO-fA-PO- |
| | PO-mU-PO-fU-PO-mA-PO-fA- | mU-PO-fA-PO-mA-PO-fA- |
| | PO-mU-PO-fA-PO-mU-PO-fG- | PO-mA-PO-fA-PO-mU- |
| | PS-mG-PS-fU (SEQ ID NO: 599) | PO-fG-PO-mC-PS-dT-PS- |
| | | dT (SEQ ID NO: 610) |
| C027.003 | lgT3-PO-lgT3-PO-lgT3-PO-fU- | fA-PS-fA-PS-mA-PO-fG- |
| | PO-mU-PO-fA-PO-mU-PO-fU- | PO-mU-PO-fC-PO-mA- |
| | PO-mA-PO-fA-PO-mU-PO-fA- | PO-fC-PO-mC-PO-fA-PO- |
| | PO-mU-PO-fG-PO-mG-PO-fU- | mU-PO-fA-PO-mU-PO-fU- |
| | PO-mG-PO-fA-PO-mC-PO-fU- | PO-mA-PO-fA-PO-mU- |
| | PS-mU-PS-fU (SEQ ID NO: 620) | PO-fA-PO-mA-PS-dT-PS- |
| | | dT (SEQ ID NO: 631) |
| C027.001#01 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PS-mA-PS-mU (SEQ ID NO: 579) | mA-PO-mU-PO-mA-PS- |
| | | mA-PS-mA (SEQ ID |
| | | NO: 590) |
| C027.001#02 | lgT3-PO-lgT3-PO-lgT3-PO-mU- | mA-PS-fU-PS-mU-PO- |
| | PO-mA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PS-mA-PS-mU (SEQ ID NO: 579) | PO-mA-PO-mU-PO-mA- |
| | | PS-mA-PS-mA (SEQ ID |
| | | NO: 591) |
| C027.001#03 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PS-mA-PS-mU (SEQ ID NO: 579) | PO-mU-PO-mA-PS-mA- |
| | | PS-mA (SEQ ID NO: 592) |
| C027.001#04 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PS-mA-PS-mU (SEQ ID NO: 579) | mA-PO-mU-PO-mA-PS- |
| | | mA-PS-mA (SEQ ID |
| | | NO: 593) |
| C027.001#05 | lgT3-PO-lgT3-PO-lgT3-PO-mU- | fA-PS4U-PS-mU-PO-fU- |
| | PO-mA-PO-mU-PO-mG-PO-fG- | PO-mU-PO-fA-PO-mA- |
| | PO-mU-PO-fG-PO-fA-PO-fC- | PO-fA-PO-mA-PO-fA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PS-mA-PS-mU (SEQ ID NO: 579) | PO4U-PO-mA-PS-dT-PS- |
| | | dT (SEQ ID NO: 594) |
| C027.001#06 | lgT3-PO-lgT3-PO-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PS-mA-PS-mU (SEQ ID NO: 580) | mA-PO-mU-PO-mA-PS- |
| | | mA-PS-mA (SEQ ID |
| | | NO: 590) |
| C027.001#07 | lgT3-PO-lgT3-PO-lgT3-PO-lU- | mA-PS-fU-PS-mU-PO- |
| | PO-lA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PS-mA-PS-mU (SEQ ID NO: 580) | PO-mA-PO-mU-PO-mA- |
| | | PS-mA-PS-mA (SEQ ID |
| | | NO: 591) |
| C027.001#08 | lgT3-PO-lgT3-PO-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PS-mA-PS-mU (SEQ ID NO: 580) | PO-mU-PO-mA-PS-mA- |
| | | PS-mA (SEQ ID NO: 592) |
| C027.001#09 | lgT3-PO-lgT3-PO-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PS-mA-PS-mU (SEQ ID NO: 580) | mA-PO-mU-PO-mA-PS- |
| | | mA-PS-mA (SEQ ID |
| | | NO: 593) |
| C027.001#10 | lgT3-PO-lgT3-PO-lgT3-PO-lU- | fA-PS4U-PS-mU-PO-fU- |
| | PO-lA-PO-mU-PO-mG-PO-fG- | PO-mU-PO-fA-PO-mA- |
| | PO-mU-PO-fG-PO-fA-PO-fC- | PO-fA-PO-mA-PO-fA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PS-mA-PS-mU (SEQ ID NO: 580) | PO4U-PO-mA-PS-dT-PS- |
| | | dT (SEQ ID NO: 594) |
| C027.001#11 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-lT4-PO-lT4 (SEQ ID | mA-PO-mU-PO-mA-PS- |
| | NO: 581) | mA-PS-mA (SEQ ID |
| | | NO: 590) |
| C027.001#12 | lgT3-PO-lgT3-PO-lgT3-PO-mU- | mA-PS-fU-PS-mU-PO- |
| | PO-mA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PO-lT4-PO-lT4 (SEQ ID | PO-mA-PO-mU-PO-mA- |
| | NO: 581) | PS-mA-PS-mA (SEQ ID |
| | | NO: 591) |
| C027.001#13 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-lT4-PO-lT4 (SEQ ID | PO-mU-PO-mA-PS-mA- |
| | NO: 581) | PS-mA (SEQ ID NO: 592) |
| C027.001#14 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-lT4-PO-lT4 (SEQ ID | mA-PO-mU-PO-mA-PS- |
| | NO: 581) | mA-PS-mA (SEQ ID |
| | | NO: 593) |
| C027.001#15 | lgT3-PO-lgT3-PO-lgT3-PO-mU- | fA-PS4U-PS-mU-PO-fU- |
| | PO-mA-PO-mU-PO-mG-PO-fG- | PO-mU-PO-fA-PO-mA- |
| | PO-mU-PO-fG-PO-fA-PO-fC- | PO-fA-PO-mA-PO-fA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-lT4-PO-lT4 (SEQ ID | PO4U-PO-mA-PS-dT-PS- |
| | NO: 581) | dT (SEQ ID NO: 594) |
| C027.001#16 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-lT4-PO-lT4 (SEQ ID | mA-PO-mU-PO-mA-PS- |
| | NO: 581) | lA-PS-lA (SEQ ID |
| | | NO: 595) |
| C027.001#17 | lgT3-PO-lgT3-PO-lgT3-PO-mU- | mA-PS-fU-PS-mU-PO- |
| | PO-mA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PO-lT4-PO-lT4 (SEQ ID | PO-mA-PO-mU-PO-mA- |
| | NO: 581) | PS-lA-PS-lA (SEQ ID |
| | | NO: 596) |
| C027.001#18 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-lT4-PO-lT4 (SEQ ID | PO-mU-PO-mA-PS-lA-PS- |
| | NO: 581) | lA (SEQ ID NO: 597) |
| C027.001#19 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-lT4-PO-lT4 (SEQ ID | mA-PO-mU-PO-mA-PS- |
| | NO: 581) | lA-PS-lA (SEQ ID |
| | | NO: 598) |
| C027.001#20 | lgT3-PS-lgT3-PS-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PS-lT4-PS-lT4 (SEQ ID NO: 582) | mA-PO-mU-PO-mA-PS- |
| | | mA-PS-mA (SEQ ID |
| | | NO: 590) |
| C027.001#21 | lgT3-PS-lgT3-PS-lgT3-PO-mU- | mA-PS-fU-PS-mU-PO- |
| | PO-mA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PS-lT4-PS-lT4 (SEQ ID NO: 582) | PO-mA-PO-mU-PO-mA- |
| | | PS-mA-PS-mA (SEQ ID |
| | | NO: 591) |
| C027.001#22 | lgT3-PS-lgT3-PS-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PS-lT4-PS-lT4 (SEQ ID NO: 582) | PO-mU-PO-mA-PS-mA- |
| | | PS-mA (SEQ ID NO: 592) |
| C027.001#23 | lgT3-PS-lgT3-PS-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PS-lT4-PS-lT4 (SEQ ID NO: 582) | mA-PO-mU-PO-mA-PS- |
| | | mA-PS-mA (SEQ ID |
| | | NO: 593) |
| C027.001#24 | lgT3-PS-lgT3-PS-lgT3-PO-mU- | fA-PS4U-PS-mU-PO-fU- |
| | PO-mA-PO-mU-PO-mG-PO-fG- | PO-mU-PO-fA-PO-mA- |
| | PO-mU-PO-fG-PO-fA-PO-fC- | PO-fA-PO-mA-PO-fA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PS-lT4-PS-lT4 (SEQ ID NO: 582) | PO4U-PO-mA-PS-dT-PS- |
| | | dT (SEQ ID NO: 594) |
| C027.001#25 | lgT3-PS-lgT3-PS-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PS-lT4-PS-lT4 (SEQ ID NO: 582) | mA-PO-mU-PO-mA-PS- |
| | | lA-PS-lA (SEQ ID |
| | | NO: 595) |
| C027.001#26 | lgT3-PS-lgT3-PS-lgT3-PO-mU- | mA-PS-fU-PS-mU-PO- |
| | PO-mA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PS-lT4-PS-lT4 (SEQ ID NO: 582) | PO-mA-PO-mU-PO-mA- |
| | | PS-lA-PS-lA (SEQ ID |
| | | NO: 596) |
| C027.001#27 | lgT3-PS-lgT3-PS-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PS-lT4-PS-lT4 (SEQ ID NO: 582) | PO-mU-PO-mA-PS-lA-PS- |
| | | lA (SEQ ID NO: 597) |
| C027.001#28 | lgT3-PS-lgT3-PS-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PS-lT4-PS-lT4 (SEQ ID NO: 582) | mA-PO-mU-PO-mA-PS- |
| | | lA-PS-lA (SEQ ID |
| | | NO: 598) |
| C027.001#29 | lgT3-PO-lgT3-PO-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-lT4-PO-lT4 (SEQ ID | mA-PO-mU-PO-mA-PS- |
| | NO: 583) | mA-PS-mA (SEQ ID |
| | | NO: 590) |
| C027.001#30 | lgT3-PO-lgT3-PO-lgT3-PO-lU- | mA-PS-fU-PS-mU-PO- |
| | PO-lA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PO-lT4-PO-lT4 (SEQ ID | PO-mA-PO-mU-PO-mA- |
| | NO: 583) | PS-mA-PS-mA (SEQ ID |
| | | NO: 591) |
| C027.001#31 | lgT3-PO-lgT3-PO-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-lT4-PO-lT4 (SEQ ID | PO-mU-PO-mA-PS-mA- |
| | NO: 583) | PS-mA (SEQ ID NO: 592) |
| C027.001#32 | lgT3-PO-lgT3-PO-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-lT4-PO-lT4 (SEQ ID | mA-PO-mU-PO-mA-PS- |
| | NO: 583) | mA-PS-mA (SEQ ID |
| | | NO: 593) |
| C027.001#33 | lgT3-PO-lgT3-PO-lgT3-PO-lU- | fA-PS4U-PS-mU-PO-fU- |
| | PO-lA-PO-mU-PO-mG-PO-fG- | PO-mU-PO-fA-PO-mA- |
| | PO-mU-PO-fG-PO-fA-PO-fC- | PO-fA-PO-mA-PO-fA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-lT4-PO-lT4 (SEQ ID | PO4U-PO-mA-PS-dT-PS- |
| | NO: 583) | dT (SEQ ID NO: 594) |
| C027.001#34 | lgT3-PS-lgT3-PS-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PS-lT4-PS-lT4 (SEQ ID NO: 584) | mA-PO-mU-PO-mA-PS- |
| | | mA-PS-mA (SEQ ID |
| | | NO: 590) |
| C027.001#35 | lgT3-PS-lgT3-PS-lgT3-PO-lU- | mA-PS-fU-PS-mU-PO- |
| | PO-lA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PS-lT4-PS-lT4 (SEQ ID NO: 584) | PO-mA-PO-mU-PO-mA- |
| | | PS-mA-PS-mA (SEQ ID |
| | | NO: 591) |
| C027.001#36 | lgT3-PS-lgT3-PS-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PS-lT4-PS-lT4 (SEQ ID NO: 584) | PO-mU-PO-mA-PS-mA- |
| | | PS-mA (SEQ ID NO: 592) |
| C027.001#37 | lgT3-PS-lgT3-PS-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PS-lT4-PS-lT4 (SEQ ID NO: 584) | mA-PO-mU-PO-mA-PS- |
| | | mA-PS-mA (SEQ ID |
| | | NO: 593) |
| C027.001#38 | lgT3-PS-lgT3-PS-lgT3-PO-lU- | fA-PS4U-PS-mU-PO-fU- |
| | PO-lA-PO-mU-PO-mG-PO-fG- | PO-mU-PO-fA-PO-mA- |
| | PO-mU-PO-fG-PO-fA-PO-fC- | PO-fA-PO-mA-PO-fA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PS-lT4-PS-lT4 (SEQ ID NO: 584) | PO4U-PO-mA-PS-dT-PS- |
| | | dT (SEQ ID NO: 594) |
| C027.001#39 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-mA-PO-mU-PO-lT4-PO-lT4 | mA-PO-mU-PO-mA-PS- |
| | (SEQ ID NO: 585) | mA-PS-mA (SEQ ID |
| | | NO: 590) |
| C027.001#40 | lgT3-PO-lgT3-PO-lgT3-PO-mU- | mA-PS-fU-PS-mU-PO- |
| | PO-mA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PO-mA-PO-mU-PO-lT4-PO-lT4 | PO-mA-PO-mU-PO-mA- |
| | (SEQ ID NO: 585) | PS-mA-PS-mA (SEQ ID |
| | | NO: 591) |
| C027.001#41 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-mA-PO-mU-PO-lT4-PO-lT4 | PO-mU-PO-mA-PS-mA- |
| | (SEQ ID NO: 585) | PS-mA (SEQ ID NO: 592) |
| C027.001#42 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-mA-PO-mU-PO-lT4-PO-lT4 | mA-PO-mU-PO-mA-PS- |
| | (SEQ ID NO: 585) | mA-PS-mA (SEQ ID |
| | | NO: 593) |
| C027.001#43 | lgT3-PO-lgT3-PO-lgT3-PO-mU- | fA-PS4U-PS-mU-PO-fU- |
| | PO-mA-PO-mU-PO-mG-PO-fG- | PO-mU-PO-fA-PO-mA- |
| | PO-mU-PO-fG-PO-fA-PO-fC- | PO-fA-PO-mA-PO-fA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-mA-PO-mU-PO-lT4-PO-lT4 | PO4U-PO-mA-PS-dT-PS- |
| | (SEQ ID NO: 585) | dT (SEQ ID NO: 594) |
| C027.001#44 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-mA-PO-mU-PO-lT4-PO-lT4 | mA-PO-mU-PO-mA-PS- |
| | (SEQ ID NO: 585) | lA-PS-lA (SEQ ID |
| | | NO: 595) |
| C027.001#45 | lgT3-PO-lgT3-PO-lgT3-PO-mU- | mA-PS-fU-PS-mU-PO- |
| | PO-mA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PO-mA-PO-mU-PO-lT4-PO-lT4 | PO-mA-PO-mU-PO-mA- |
| | (SEQ ID NO: 585) | PS-lA-PS-lA (SEQ ID |
| | | NO: 596) |
| C027.001#46 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-mA-PO-mU-PO-lT4-PO-lT4 | PO-mU-PO-mA-PS-lA-PS- |
| | (SEQ ID NO: 585) | lA (SEQ ID NO: 597) |
| C027.001#47 | lgT3-PO-lgT3-PO-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-mA-PO-mU-PO-lT4-PO-lT4 | mA-PO-mU-PO-mA-PS- |
| | (SEQ ID NO: 585) | lA-PS-lA (SEQ ID |
| | | NO: 598) |
| C027.001#48 | lgT3-PS-lgT3-PS-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-mA-PO-mU-PS-lT4-PS-lT4 | mA-PO-mU-PO-mA-PS- |
| | (SEQ ID NO: 586) | mA-PS-mA (SEQ ID |
| | | NO: 590) |
| C027.001#49 | lgT3-PS-lgT3-PS-lgT3-PO-mU- | mA-PS-fU-PS-mU-PO- |
| | PO-mA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PO-mA-PO-mU-PS-lT4-PS-lT4 | PO-mA-PO-mU-PO-mA- |
| | (SEQ ID NO: 586) | PS-mA-PS-mA (SEQ ID |
| | | NO: 591) |
| C027.001#50 | lgT3-PS-lgT3-PS-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-mA-PO-mU-PS-lT4-PS-lT4 | PO-mU-PO-mA-PS-mA- |
| | (SEQ ID NO: 586) | PS-mA (SEQ ID NO: 592) |
| C027.001#51 | lgT3-PS-lgT3-PS-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-mA-PO-mU-PS-lT4-PS-lT4 | mA-PO-mU-PO-mA-PS- |
| | (SEQ ID NO: 586) | mA-PS-mA (SEQ ID |
| | | NO: 593) |
| C027.001#52 | lgT3-PS-lgT3-PS-lgT3-PO-mU- | fA-PS4U-PS-mU-PO-fU- |
| | PO-mA-PO-mU-PO-mG-PO-fG- | PO-mU-PO-fA-PO-mA- |
| | PO-mU-PO-fG-PO-fA-PO-fC- | PO-fA-PO-mA-PO-fA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-mA-PO-mU-PS-lT4-PS-lT4 | PO4U-PO-mA-PS-dT-PS- |
| | (SEQ ID NO: 586) | dT (SEQ ID NO: 594) |
| C027.001#53 | lgT3-PS-lgT3-PS-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-mA-PO-mU-PS-lT4-PS-lT4 | mA-PO-mU-PO-mA-PS- |
| | (SEQ ID NO: 586) | lA-PS-lA (SEQ ID |
| | | NO: 595) |
| C027.001#54 | lgT3-PS-lgT3-PS-lgT3-PO-mU- | mA-PS-fU-PS-mU-PO- |
| | PO-mA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PO-mA-PO-mU-PS-lT4-PS-lT4 | PO-mA-PO-mU-PO-mA- |
| | (SEQ ID NO: 586) | PS-lA-PS-lA (SEQ ID |
| | | NO: 596) |
| C027.001#55 | lgT3-PS-lgT3-PS-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-mA-PO-mU-PS-lT4-PS-lT4 | PO-mU-PO-mA-PS-lA-PS- |
| | (SEQ ID NO: 586) | lA (SEQ ID NO: 597) |
| C027.001#56 | lgT3-PS-lgT3-PS-lgT3-PO-mU- | mA-PS-fU-PS-mU-PO-fU- |
| | PO-mA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-mA-PO-mU-PS-lT4-PS-lT4 | mA-PO-mU-PO-mA-PS- |
| | (SEQ ID NO: 586) | lA-PS-lA (SEQ ID |
| | NO: 598) | |
| C027.001#57 | lgT3-PO-lgT3-PO-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-mA-PO-mU-PO-lT4-PO-lT4 | mA-PO-mU-PO-mA-PS- |
| | (SEQ ID NO: 587) | mA-PS-mA (SEQ ID |
| | | NO: 590) |
| C027.001#58 | lgT3-PO-lgT3-PO-lgT3-PO-lU- | mA-PS-fU-PS-mU-PO- |
| | PO-lA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PO-mA-PO-mU-PO-lT4-PO-lT4 | PO-mA-PO-mU-PO-mA- |
| | (SEQ ID NO: 587) | PS-mA-PS-mA (SEQ ID |
| | | NO: 591) |
| C027.001#59 | lgT3-PO-lgT3-PO-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-mA-PO-mU-PO-lT4-PO-lT4 | PO-mU-PO-mA-PS-mA- |
| | (SEQ ID NO: 587) | PS-mA (SEQ ID NO: 592) |
| C027.001#60 | lgT3-PO-lgT3-PO-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-mA-PO-mU-PO-lT4-PO-lT4 | mA-PO-mU-PO-mA-PS- |
| | (SEQ ID NO: 587) | mA-PS-mA (SEQ ID |
| | | NO: 593) |
| C027.001#61 | lgT3-PO-lgT3-PO-lgT3-PO-lU- | fA-PS4U-PS-mU-PO-fU- |
| | PO-lA-PO-mU-PO-mG-PO-fG- | PO-mU-PO-fA-PO-mA- |
| | PO-mU-PO-fG-PO-fA-PO-fC- | PO-fA-PO-mA-PO-fA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-mA-PO-mU-PO-lT4-PO-lT4 | PO4U-PO-mA-PS-dT-PS- |
| | (SEQ ID NO: 587) | dT (SEQ ID NO: 594) |
| C027.001#62 | lgT3-PS-lgT3-PS-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-mA-PO-mA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-mA-PO-mU-PS-lT4-PS-lT4 | mA-PO-mU-PO-mA-PS- |
| | (SEQ ID NO: 588) | mA-PS-mA (SEQ ID |
| | | NO: 590) |
| C027.001#63 | lgT3-PS-lgT3-PS-lgT3-PO-lU- | mA-PS-fU-PS-mU-PO- |
| | PO-lA-PO-mU-PO-mG-PO-fG- | mU-PO-mU-PO-fA-PO- |
| | PO-mU-PO-fG-PO-fA-PO-fC- | mA-PO-mA-PO-mA-PO- |
| | PO-mU-PO-mU-PO-mU-PO-mU- | mA-PO-mG-PO-mU-PO- |
| | PO-mU-PO-mA-PO-mA-PO-mA- | mC-PO-fA-PO-mC-PO-fC- |
| | PO-mA-PO-mU-PS-lT4-PS-lT4 | PO-mA-PO-mU-PO-mA- |
| | (SEQ ID NO: 588) | PS-mA-PS-mA (SEQ ID |
| | | NO: 591) |
| C027.001#64 | lgT3-PS-lgT3-PS-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-fA-PO-fA-PO-mA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-mA-PO-mU-PS-lT4-PS-lT4 | PO-mU-PO-mA-PS-mA- |
| | (SEQ ID NO: 588) | PS-mA (SEQ ID NO: 592) |
| C027.001#65 | lgT3-PS-lgT3-PS-lgT3-PO-lU- | mA-PS-fU-PS-mU-PO-fU- |
| | PO-lA-PO-mU-PO-mG-PO-fG- | PO-mU-PO-mA-PO-mA- |
| | PO-mU-PO-fG-PO-fA-PO-fC- | PO-mA-PO-fA-PO-mA- |
| | PO-mU-PO-mU-PO-mU-PO-mU- | PO-mG-PO-mU-PO-mC- |
| | PO-mU-PO-mA-PO-mA-PO-mA- | PO-fA-PO-mC-PO-fC-PO- |
| | PO-mA-PO-mU-PS-lT4-PS-lT4 | mA-PO-mU-PO-mA-PS- |
| | (SEQ ID NO: 588) | mA-PS-mA (SEQ ID |
| | | NO: 593) |
| C027.001#66 | lgT3-PS-lgT3-PS-lgT3-PO-lU- | fA-PS4U-PS-mU-PO-fU- |
| | PO-lA-PO-mU-PO-mG-PO-fG- | PO-mU-PO-fA-PO-mA- |
| | PO-mU-PO-fG-PO-fA-PO-fC- | PO-fA-PO-mA-PO-fA-PO- |
| | PO-mU-PO-mU-PO-mU-PO-mU- | mG-PO-mU-PO-mC-PO- |
| | PO-mU-PO-mA-PO-mA-PO-mA- | fA-PO-mC-PO-fC-PO-mA- |
| | PO-mA-PO-mU-PS-lT4-PS-lT4 | PO4U-PO-mA-PS-dT-PS- |
| | (SEQ ID NO: 588) | dT (SEQ ID NO: 594) |
| C027.002#01 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-mG-PS-mU (SEQ ID NO: 600) | mU-PO-mG-PO-mC-PS- |
| | | mA-PS-mA (SEQ ID |
| | | NO: 611) |
| C027.002#02 | lgT3-PO-lgT3-PO-lgT3-PO-mG- | mA-PS-fC-PS-mC-PO-mA- |
| | PO-mC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-mG-PS-mU (SEQ ID NO: 600) | mU-PO-mG-PO-mC-PS- |
| | | mA-PS-mA (SEQ ID |
| | | NO: 612) |
| C027.002#03 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PS-mG-PS-mU (SEQ ID NO: 600) | PO-mG-PO-mC-PS-mA- |
| | | PS-mA (SEQ ID NO: 613) |
| C027.002#04 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-mG-PS-mU (SEQ ID NO: 600) | mU-PO-mG-PO-mC-PS- |
| | | mA-PS-mA (SEQ ID |
| | | NO: 614) |
| C027.002#05 | lgT3-PO-lgT3-PO-lgT3-PO-mG- | fA-PS-fC-PS-mC-PO-fA- |
| | PO-mC-PO-mA-PO-mU-PO-fU- | PO-mU-PO-fA-PO-mU- |
| | PO-mU-PO-fU-PO-fU-PO-fA- | PO4U-PO-mA-PO-fA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PS-mG-PS-mU (SEQ ID NO: 600) | PO-fG-PO-mC-PS-dT-PS- |
| | | dT (SEQ ID NO: 615) |
| C027.002#06 | lgT3-PO-lgT3-PO-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-mG-PS-mU (SEQ ID NO: 601) | mU-PO-mG-PO-mC-PS- |
| | | mA-PS-mA (SEQ ID |
| | | NO: 611) |
| C027.002#07 | lgT3-PO-lgT3-PO-lgT3-PO-lG- | mA-PS-fC-PS-mC-PO-mA- |
| | PO-lC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-mG-PS-mU (SEQ ID NO: 601) | mU-PO-mG-PO-mC-PS- |
| | | mA-PS-mA (SEQ ID |
| | | NO: 612) |
| C027.002#08 | lgT3-PO-lgT3-PO-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PS-mG-PS-mU (SEQ ID NO: 601) | PO-mG-PO-mC-PS-mA- |
| | | PS-mA (SEQ ID NO: 613) |
| C027.002#09 | lgT3-PO-lgT3-PO-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-mG-PS-mU (SEQ ID NO: 601) | mU-PO-mG-PO-mC-PS- |
| | | mA-PS-mA (SEQ ID |
| | | NO: 614 ) |
| C027.002#10 | lgT3-PO-lgT3-PO-lgT3-PO-lG- | fA-PS-fC-PS-mC-PO-fA- |
| | PO-lC-PO-mA-PO-mU-PO-fU- | PO-mU-PO-fA-PO-mU- |
| | PO-mU-PO-fU-PO-fU-PO-fA- | PO4U-PO-mA-PO-fA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PS-mG-PS-mU (SEQ ID NO: 601) | PO-fG-PO-mC-PS-dT-PS- |
| | | dT (SEQ ID NO: 615) |
| C027.002#11 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-lT4-PO-lT4 (SEQ ID | mU-PO-mG-PO-mC-PS- |
| | NO: 602) | mA-PS-mA (SEQ ID |
| | | NO: 611) |
| C027.002#12 | lgT3-PO-lgT3-PO-lgT3-PO-mG- | mA-PS-fC-PS-mC-PO-mA- |
| | PO-mC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-lT4-PO-lT4 (SEQ ID | mU-PO-mG-PO-mC-PS- |
| | NO: 602) | mA-PS-mA (SEQ ID |
| | | NO: 612) |
| C027.002#13 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-lT4-PO-lT4 (SEQ ID | PO-mG-PO-mC-PS-mA- |
| | NO: 602) | PS-mA (SEQ ID NO: 613) |
| C027.002#14 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-lT4-PO-lT4 (SEQ ID | mU-PO-mG-PO-mC-PS- |
| | NO: 602) | mA-PS-mA (SEQ ID |
| | | NO: 614) |
| C027.002#15 | lgT3-PO-lgT3-PO-lgT3-PO-mG- | fA-PS-fC-PS-mC-PO-fA- |
| | PO-mC-PO-mA-PO-mU-PO-fU- | PO-mU-PO-fA-PO-mU- |
| | PO-mU-PO-fU-PO-fU-PO-fA- | PO4U-PO-mA-PO-fA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-lT4-PO-lT4 (SEQ ID | PO-fG-PO-mC-PS-dT-PS- |
| | NO: 602) | dT (SEQ ID NO: 615) |
| C027.002#16 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-lT4-PO-lT4 (SEQ ID | mU-PO-mG-PO-mC-PS- |
| | NO: 602) | lA-PS-lA (SEQ ID |
| | | NO: 616) |
| C027.002#17 | lgT3-PO-lgT3-PO-lgT3-PO-mG- | mA-PS-fC-PS-mC-PO-mA- |
| | PO-mC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-lT4-PO-lT4 (SEQ ID | mU-PO-mG-PO-mC-PS- |
| | NO: 602) | lA-PS-lA (SEQ ID |
| | | NO: 617) |
| C027.002#18 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-lT4-PO-lT4 (SEQ ID | PO-mG-PO-mC-PS-lA-PS- |
| | NO: 602) | lA (SEQ ID NO: 618) |
| C027.002#19 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-lT4-PO-lT4 (SEQ ID | mU-PO-mG-PO-mC-PS- |
| | NO: 602) | lA-PS-lA (SEQ ID |
| | | NO: 619) |
| C027.002#20 | lgT3-PS-lgT3-PS-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-lT4-PS-lT4 (SEQ ID NO: 603) | mU-PO-mG-PO-mC-PS- |
| | | mA-PS-mA (SEQ ID |
| | | NO: 611) |
| C027.002#21 | lgT3-PS-lgT3-PS-lgT3-PO-mG- | mA-PS-fC-PS-mC-PO-mA- |
| | PO-mC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-lT4-PS-lT4 (SEQ ID NO: 603) | mU-PO-mG-PO-mC-PS- |
| | | mA-PS-mA (SEQ ID |
| | | NO: 612) |
| C027.002#22 | lgT3-PS-lgT3-PS-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PS-lT4-PS-lT4 (SEQ ID NO: 603) | PO-mG-PO-mC-PS-mA- |
| | | PS-mA (SEQ ID NO: 613) |
| C027.002#23 | lgT3-PS-lgT3-PS-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-lT4-PS-lT4 (SEQ ID NO: 603) | mU-PO-mG-PO-mC-PS- |
| | | mA-PS-mA (SEQ ID |
| | | NO: 614) |
| C027.002#24 | lgT3-PS-lgT3-PS-lgT3-PO-mG- | fA-PS-fC-PS-mC-PO-fA- |
| | PO-mC-PO-mA-PO-mU-PO-fU- | PO-mU-PO-fA-PO-mU- |
| | PO-mU-PO-fU-PO-fU-PO-fA- | PO4U-PO-mA-PO-fA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PS-lT4-PS-lT4 (SEQ ID NO: 603) | PO-fG-PO-mC-PS-dT-PS- |
| | | dT (SEQ ID NO: 615) |
| C027.002#25 | lgT3-PS-lgT3-PS-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-lT4-PS-lT4 (SEQ ID NO: 603) | mU-PO-mG-PO-mC-PS- |
| | | lA-PS-lA (SEQ ID |
| | | NO: 616) |
| C027.002#26 | lgT3-PS-lgT3-PS-lgT3-PO-mG- | mA-PS-fC-PS-mC-PO-mA- |
| | PO-mC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-lT4-PS-lT4 (SEQ ID NO: 603) | mU-PO-mG-PO-mC-PS- |
| | | lA-PS-lA (SEQ ID |
| | | NO: 617) |
| C027.002#27 | lgT3-PS-lgT3-PS-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PS-lT4-PS-lT4 (SEQ ID NO: 603) | PO-mG-PO-mC-PS-lA-PS- |
| | | lA (SEQ ID NO: 618) |
| C027.002#28 | lgT3-PS-lgT3-PS-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-lT4-PS-lT4 (SEQ ID NO: 603) | mU-PO-mG-PO-mC-PS- |
| | | lA-PS-lA (SEQ ID |
| | | NO: 619) |
| C027.002#29 | lgT3-PO-lgT3-PO-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-lT4-PO-lT4 (SEQ ID | mU-PO-mG-PO-mC-PS- |
| | NO: 604) | mA-PS-mA (SEQ ID |
| | | NO: 611) |
| C027.002#30 | lgT3-PO-lgT3-PO-lgT3-PO-lG- | mA-PS-fC-PS-mC-PO-mA- |
| | PO-lC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-lT4-PO-lT4 (SEQ ID | mU-PO-mG-PO-mC-PS- |
| | NO: 604) | mA-PS-mA (SEQ ID |
| | | NO: 612) |
| C027.002#31 | lgT3-PO-lgT3-PO-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-lT4-PO-lT4 (SEQ ID | PO-mG-PO-mC-PS-mA- |
| | NO: 604) | PS-mA (SEQ ID NO: 613) |
| C027.002#32 | lgT3-PO-lgT3-PO-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-lT4-PO-lT4 (SEQ ID | mU-PO-mG-PO-mC-PS- |
| | NO: 604) | mA-PS-mA (SEQ ID |
| | | NO: 614) |
| C027.002#33 | lgT3-PO-lgT3-PO-lgT3-PO-lG- | fA-PS-fC-PS-mC-PO-fA- |
| | PO-lC-PO-mA-PO-mU-PO-fU- | PO-mU-PO-fA-PO-mU- |
| | PO-mU-PO-fU-PO-fU-PO-fA- | PO4U-PO-mA-PO-fA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-lT4-PO-lT4 (SEQ ID | PO-fG-PO-mC-PS-dT-PS- |
| | NO: 604) | dT (SEQ ID NO: 615) |
| C027.002#34 | lgT3-PS-lgT3-PS-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-lT4-PS-lT4 (SEQ ID NO: 605) | mU-PO-mG-PO-mC-PS- |
| | | mA-PS-mA (SEQ ID |
| | | NO: 611) |
| C027.002#35 | lgT3-PS-lgT3-PS-lgT3-PO-lG- | mA-PS-fC-PS-mC-PO-mA- |
| | PO-lC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-lT4-PS-lT4 (SEQ ID NO: 605) | mU-PO-mG-PO-mC-PS- |
| | | mA-PS-mA (SEQ ID |
| | | NO: 612) |
| C027.002#36 | lgT3-PS-lgT3-PS-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PS-lT4-PS-lT4 (SEQ ID NO: 605) | PO-mG-PO-mC-PS-mA- |
| | | PS-mA (SEQ ID NO: 613) |
| C027.002#37 | lgT3-PS-lgT3-PS-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PS-lT4-PS-lT4 (SEQ ID NO: 605) | mU-PO-mG-PO-mC-PS- |
| | | mA-PS-mA (SEQ ID |
| | | NO: 614) |
| C027.002#38 | lgT3-PS-lgT3-PS-lgT3-PO-lG- | fA-PS-fC-PS-mC-PO-fA- |
| | PO-lC-PO-mA-PO-mU-PO-fU- | PO-mU-PO-fA-PO-mU- |
| | PO-mU-PO-fU-PO-fU-PO-fA- | PO4U-PO-mA-PO-fA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PS-lT4-PS-lT4 (SEQ ID NO: 605) | PO-fG-PO-mC-PS-dT-PS- |
| | | dT (SEQ ID NO: 615) |
| C027.002#39 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PO-lT4-PO-lT4 | mU-PO-mG-PO-mC-PS- |
| | (SEQ ID NO: 606) | mA-PS-mA (SEQ ID |
| | | NO: 611) |
| C027.002#40 | lgT3-PO-lgT3-PO-lgT3-PO-mG- | mA-PS-fC-PS-mC-PO-mA- |
| | PO-mC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PO-lT4-PO-lT4 | mU-PO-mG-PO-mC-PS- |
| | (SEQ ID NO: 606) | mA-PS-mA (SEQ ID |
| | | NO: 612) |
| C027.002#41 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-mG-PO-mU-PO-lT4-PO-lT4 | PO-mG-PO-mC-PS-mA- |
| | (SEQ ID NO: 606) | PS-mA (SEQ ID NO: 613) |
| C027.002#42 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PO-lT4-PO-lT4 | mU-PO-mG-PO-mC-PS- |
| | (SEQ ID NO: 606) | mA-PS-mA (SEQ ID |
| | | NO: 614) |
| C027.002#43 | lgT3-PO-lgT3-PO-lgT3-PO-mG- | fA-PS-fC-PS-mC-PO-fA- |
| | PO-mC-PO-mA-PO-mU-PO-fU- | PO-mU-PO-fA-PO-mU- |
| | PO-mU-PO-fU-PO-fU-PO-fA- | PO4U-PO-mA-PO-fA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-mG-PO-mU-PO-lT4-PO-lT4 | PO-fG-PO-mC-PS-dT-PS- |
| | (SEQ ID NO: 606) | dT (SEQ ID NO: 615) |
| C027.002#44 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PO-lT4-PO-lT4 | mU-PO-mG-PO-mC-PS- |
| | (SEQ ID NO: 606) | lA-PS-lA (SEQ ID |
| | | NO: 616) |
| C027.002#45 | lgT3-PO-lgT3-PO-lgT3-PO-mG- | mA-PS-fC-PS-mC-PO-mA- |
| | PO-mC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PO-lT4-PO-lT4 | mU-PO-mG-PO-mC-PS- |
| | (SEQ ID NO: 606) | lA-PS-lA (SEQ ID |
| | | NO: 617) |
| C027.002#46 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-mG-PO-mU-PO-lT4-PO-lT4 | PO-mG-PO-mC-PS-lA-PS- |
| | (SEQ ID NO: 606) | lA (SEQ ID NO: 618) |
| C027.002#47 | lgT3-PO-lgT3-PO-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PO-lT4-PO-lT4 | mU-PO-mG-PO-mC-PS- |
| | (SEQ ID NO: 606) | lA-PS-lA (SEQ ID |
| | | NO: 619) |
| C027.002#48 | lgT3-PS-lgT3-PS-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PS-lT4-PS-lT4 | mU-PO-mG-PO-mC-PS- |
| | (SEQ ID NO: 607) | mA-PS-mA (SEQ ID |
| | | NO: 611) |
| C027.002#49 | lgT3-PS-lgT3-PS-lgT3-PO-mG- | mA-PS-fC-PS-mC-PO-mA- |
| | PO-mC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PS-lT4-PS-lT4 | mU-PO-mG-PO-mC-PS- |
| | (SEQ ID NO: 607) | mA-PS-mA (SEQ ID |
| | | NO: 612) |
| C027.002#50 | lgT3-PS-lgT3-PS-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-mG-PO-mU-PS-lT4-PS-lT4 | PO-mG-PO-mC-PS-mA- |
| | (SEQ ID NO: 607) | PS-mA (SEQ ID NO: 613) |
| C027.002#51 | lgT3-PS-lgT3-PS-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PS-lT4-PS-lT4 | mU-PO-mG-PO-mC-PS- |
| | (SEQ ID NO: 607) | mA-PS-mA (SEQ ID |
| | | NO: 614) |
| C027.002#52 | lgT3-PS-lgT3-PS-lgT3-PO-mG- | fA-PS-fC-PS-mC-PO-fA- |
| | PO-mC-PO-mA-PO-mU-PO-fU- | PO-mU-PO-fA-PO-mU- |
| | PO-mU-PO-fU-PO-fU-PO-fA- | PO4U-PO-mA-PO-fA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-mG-PO-mU-PS-lT4-PS-lT4 | PO-fG-PO-mC-PS-dT-PS- |
| | (SEQ ID NO: 607) | dT (SEQ ID NO: 615) |
| C027.002#53 | lgT3-PS-lgT3-PS-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PS-lT4-PS-lT4 | mU-PO-mG-PO-mC-PS- |
| | (SEQ ID NO: 607) | lA-PS-lA (SEQ ID |
| | | NO: 616) |
| C027.002#54 | lgT3-PS-lgT3-PS-lgT3-PO-mG- | mA-PS-fC-PS-mC-PO-mA- |
| | PO-mC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PS-lT4-PS-lT4 | mU-PO-mG-PO-mC-PS- |
| | (SEQ ID NO: 607) | lA-PS-lA (SEQ ID |
| | | NO: 617) |
| C027.002#55 | lgT3-PS-lgT3-PS-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-mG-PO-mU-PS-lT4-PS-lT4 | PO-mG-PO-mC-PS-lA-PS- |
| | (SEQ ID NO: 607) | lA (SEQ ID NO: 618) |
| C027.002#56 | lgT3-PS-lgT3-PS-lgT3-PO-mG- | mA-PS-fC-PS-mC-PO-fA- |
| | PO-mC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PS-lT4-PS- | mU-PO-mG-PO-mC-PS- |
| | lT4 (SEQ ID NO: 607) | lA-PS-lA (SEQ ID |
| | | NO: 619) |
| C027.002#57 | lgT3-PO-lgT3-PO-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PO-lT4-PO-lT4 | mU-PO-mG-PO-mC-PS- |
| | (SEQ ID NO: 608) | mA-PS-mA (SEQ ID |
| | | NO: 611) |
| C027.002#58 | lgT3-PO-lgT3-PO-lgT3-PO-lG- | mA-PS-fC-PS-mC-PO-mA- |
| | PO-lC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PO-lT4-PO-lT4 | mU-PO-mG-PO-mC-PS- |
| | (SEQ ID NO: 608) | mA-PS-mA (SEQ ID |
| | | NO: 612) |
| C027.002#59 | lgT3-PO-lgT3-PO-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-mG-PO-mU-PO-lT4-PO-lT4 | PO-mG-PO-mC-PS-mA- |
| | (SEQ ID NO: 608) | PS-mA (SEQ ID NO: 613) |
| C027.002#60 | lgT3-PO-lgT3-PO-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PO-lT4-PO-lT4 | mU-PO-mG-PO-mC-PS- |
| | (SEQ ID NO: 608) | mA-PS-mA (SEQ ID |
| | | NO: 614) |
| C027.002#61 | lgT3-PO-lgT3-PO-lgT3-PO-lG- | fA-PS-fC-PS-mC-PO-fA- |
| | PO-lC-PO-mA-PO-mU-PO-fU- | PO-mU-PO-fA-PO-mU- |
| | PO-mU-PO-fU-PO-fU-PO-fA- | PO4U-PO-mA-PO-fA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-mG-PO-mU-PO-lT4-PO-lT4 | PO-fG-PO-mC-PS-dT-PS- |
| | (SEQ ID NO: 608) | dT (SEQ ID NO: 615) |
| C027.002#62 | lgT3-PS-lgT3-PS-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PS-lT4-PS-lT4 | mU-PO-mG-PO-mC-PS- |
| | (SEQ ID NO: 609) | mA-PS-mA (SEQ ID |
| | | NO: 611) |
| C027.002#63 | lgT3-PS-lgT3-PS-lgT3-PO-lG- | mA-PS-fC-PS-mC-PO-mA- |
| | PO-lC-PO-mA-PO-mU-PO-fU- | PO-mU-PO-fA-PO-mU- |
| | PO-mU-PO-fU-PO-fU-PO-fA- | PO-mU-PO-mA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PS-lT4-PS-lT4 | mU-PO-mG-PO-mC-PS- |
| | (SEQ ID NO: 609) | mA-PS-mA (SEQ ID |
| | | NO: 612) |
| C027.002#64 | lgT3-PS-lgT3-PS-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO4U-PO-fA-PO-mA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-mG-PO-mU-PS-lT4-PS-lT4 | PO-mG-PO-mC-PS-mA- |
| | (SEQ ID NO: 609) | PS-mA (SEQ ID NO: 613) |
| C027.002#65 | lgT3-PS-lgT3-PS-lgT3-PO-lG- | mA-PS-fC-PS-mC-PO-fA- |
| | PO-lC-PO-mA-PO-mU-PO-fU- | PO-mU-PO-mA-PO-mU- |
| | PO-mU-PO-fU-PO-fU-PO-fA- | PO-mU-PO-fA-PO-mA- |
| | PO-mU-PO-mU-PO-mA-PO-mA- | PO-mU-PO-mA-PO-mA- |
| | PO-mU-PO-mA-PO-mU-PO-mG- | PO-fA-PO-mA-PO-fA-PO- |
| | PO-mG-PO-mU-PS-lT4-PS-lT4 | mU-PO-mG-PO-mC-PS- |
| | (SEQ ID NO: 609) | mA-PS-mA (SEQ ID |
| | | NO: 614) |
| C027.002#66 | lgT3-PS-lgT3-PS-lgT3-PO-lG- | fA-PS-fC-PS-mC-PO-fA- |
| | PO-lC-PO-mA-PO-mU-PO-fU- | PO-mU-PO-fA-PO-mU- |
| | PO-mU-PO-fU-PO-fU-PO-fA- | PO4U-PO-mA-PO-fA-PO- |
| | PO-mU-PO-mU-PO-mA-PO-mA- | mU-PO-mA-PO-mA-PO- |
| | PO-mU-PO-mA-PO-mU-PO-mG- | fA-PO-mA-PO-fA-PO-mU- |
| | PO-mG-PO-mU-PS-lT4-PS-lT4 | PO-fG-PO-mC-PS-dT-PS- |
| | (SEQ ID NO: 609) | dT (SEQ ID NO: 615) |
| C027.003#01 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PS-mU-PS-mU (SEQ ID NO: 621) | mU-PO-mA-PO-mA-PS- |
| | | mA-PS-mA (SEQ ID |
| | | NO: 632) |
| C027.003#02 | lgT3-PO-lgT3-PO-lgT3-PO-mU- | mA-PS-fA-PS-mA-PO- |
| | PO-mU-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PS-mU-PS-mU (SEQ ID NO: 621) | PO-mU-PO-mA-PO-mA- |
| | | PS-mA-PS-mA (SEQ ID |
| | | NO: 633) |
| C027.003#03 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PS-mU-PS-mU (SEQ ID NO: 621) | PO-mA-PO-mA-PS-mA- |
| | | PS-mA (SEQ ID NO: 634) |
| C027.003#04 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PS-mU-PS-mU (SEQ ID NO: 621) | mU-PO-mA-PO-mA-PS- |
| | | mA-PS-mA (SEQ ID |
| | | NO: 635) |
| C027.003#05 | lgT3-PO-lgT3-PO-lgT3-PO-mU- | fA-PS-fA-PS-mA-PO-fG- |
| | PO-mU-PO-mA-PO-mU-PO-fU- | PO-mU-PO-fC-PO-mA- |
| | PO-mA-PO-fA-PO-fU-PO-fA- | PO-fC-PO-mC-PO-fA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PS-mU-PS-mU (SEQ ID NO: 621) | PO-fA-PO-mA-PS-dT-PS- |
| | | dT (SEQ ID NO: 636) |
| C027.003#06 | lgT3-PO-lgT3-PO-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PS-mU-PS-mU (SEQ ID NO: 622) | mU-PO-mA-PO-mA-PS- |
| | | mA-PS-mA (SEQ ID |
| | | NO: 632) |
| C027.003#07 | lgT3-PO-lgT3-PO-lgT3-PO-lT- | mA-PS-fA-PS-mA-PO- |
| | PO-lT-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PS-mU-PS-mU (SEQ ID NO: 622) | PO-mU-PO-mA-PO-mA- |
| | | PS-mA-PS-mA (SEQ ID |
| | | NO: 633) |
| C027.003#08 | lgT3-PO-lgT3-PO-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PS-mU-PS-mU (SEQ ID NO: 622) | PO-mA-PO-mA-PS-mA- |
| | | PS-mA (SEQ ID NO: 634) |
| C027.003#09 | lgT3-PO-lgT3-PO-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PS-mU-PS-mU (SEQ ID NO: 622) | mU-PO-mA-PO-mA-PS- |
| | | mA-PS-mA (SEQ ID |
| | | NO: 635) |
| C027.003#10 | lgT3-PO-lgT3-PO-lgT3-PO-lT- | fA-PS-fA-PS-mA-PO-fG- |
| | PO-lT-PO-mA-PO-mU-PO-fU- | PO-mU-PO-fC-PO-mA- |
| | PO-mA-PO-fA-PO-fU-PO-fA- | PO-fC-PO-mC-PO-fA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PS-mU-PS-mU (SEQ ID NO: 622) | PO-fA-PO-mA-PS-dT-PS- |
| | | dT (SEQ ID NO: 636) |
| C027.003#11 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-lT4-PO-lT4 (SEQ ID | mU-PO-mA-PO-mA-PS- |
| | NO: 623) | mA-PS-mA (SEQ ID |
| | | NO: 632) |
| C027.003#12 | lgT3-PO-lgT3-PO-lgT3-PO-mU- | mA-PS-fA-PS-mA-PO- |
| | PO-mU-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PO-lT4-PO-lT4 (SEQ ID | PO-mU-PO-mA-PO-mA- |
| | NO: 623) | PS-mA-PS-mA (SEQ ID |
| | | NO: 633) |
| C027.003#13 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-lT4-PO-lT4 (SEQ ID | PO-mA-PO-mA-PS-mA- |
| | NO: 623) | PS-mA (SEQ ID NO: 634) |
| C027.003#14 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-lT4-PO-lT4 (SEQ ID | mU-PO-mA-PO-mA-PS- |
| | NO: 623) | mA-PS-mA (SEQ ID |
| | | NO: 635) |
| C027.003#15 | lgT3-PO-lgT3-PO-lgT3-PO-mU- | fA-PS-fA-PS-mA-PO-fG- |
| | PO-mU-PO-mA-PO-mU-PO-fU- | PO-mU-PO-fC-PO-mA- |
| | PO-mA-PO-fA-PO-fU-PO-fA- | PO-fC-PO-mC-PO-fA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-lT4-PO-lT4 (SEQ ID | PO-fA-PO-mA-PS-dT-PS- |
| | NO: 623) | dT (SEQ ID NO: 636) |
| C027.003#16 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-lT4-PO-lT4 (SEQ ID | mU-PO-mA-PO-mA-PS- |
| | NO: 623) | lA-PS-lA (SEQ ID |
| | | NO: 637) |
| C027.003#17 | lgT3-PO-lgT3-PO-lgT3-PO-mU- | mA-PS-fA-PS-mA-PO- |
| | PO-mU-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PO-lT4-PO-lT4 (SEQ ID | PO-mU-PO-mA-PO-mA- |
| | NO: 623) | PS-lA-PS-lA (SEQ ID |
| | | NO: 638) |
| C027.003#18 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-lT4-PO-lT4 (SEQ ID | PO-mA-PO-mA-PS-lA-PS- |
| | NO: 623) | lA (SEQ ID NO: 639) |
| C027.003#19 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-lT4-PO-lT4 (SEQ ID | mU-PO-mA-PO-mA-PS- |
| | NO: 623) | lA-PS-lA (SEQ ID |
| | | NO: 640) |
| C027.003#20 | lgT3-PS-lgT3-PS-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PS-lT4-PS-lT4 (SEQ ID NO: 624) | mU-PO-mA-PO-mA-PS- |
| | | mA-PS-mA (SEQ ID |
| | | NO: 632) |
| C027.003#21 | lgT3-PS-lgT3-PS-lgT3-PO-mU- | mA-PS-fA-PS-mA-PO- |
| | PO-mU-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PS-lT4-PS-lT4 (SEQ ID NO: 624) | PO-mU-PO-mA-PO-mA- |
| | | PS-mA-PS-mA (SEQ ID |
| | | NO: 633) |
| C027.003#22 | lgT3-PS-lgT3-PS-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PS-lT4-PS-lT4 (SEQ ID NO: 624) | PO-mA-PO-mA-PS-mA- |
| | | PS-mA (SEQ ID NO: 634) |
| C027.003#23 | lgT3-PS-lgT3-PS-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PS-lT4-PS-lT4 (SEQ ID NO: 624) | mU-PO-mA-PO-mA-PS- |
| | | mA-PS-mA (SEQ ID |
| | | NO: 635) |
| C027.003#24 | lgT3-PS-lgT3-PS-lgT3-PO-mU- | fA-PS-fA-PS-mA-PO-fG- |
| | PO-mU-PO-mA-PO-mU-PO-fU- | PO-mU-PO-fC-PO-mA- |
| | PO-mA-PO-fA-PO-fU-PO-fA- | PO-fC-PO-mC-PO-fA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PS-lT4-PS-lT4 (SEQ ID NO: 624) | PO-fA-PO-mA-PS-dT-PS- |
| | | dT (SEQ ID NO: 636) |
| C027.003#25 | lgT3-PS-lgT3-PS-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PS-lT4-PS-lT4 (SEQ ID NO: 624) | mU-PO-mA-PO-mA-PS- |
| | | lA-PS-lA (SEQ ID |
| | | NO: 637) |
| C027.003#26 | lgT3-PS-lgT3-PS-lgT3-PO-mU- | mA-PS-fA-PS-mA-PO- |
| | PO-mU-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PS-lT4-PS-lT4 (SEQ ID NO: 624) | PO-mU-PO-mA-PO-mA- |
| | | PS-lA-PS-lA (SEQ ID |
| | | NO: 638) |
| C027.003#27 | lgT3-PS-lgT3-PS-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PS-lT4-PS-lT4 (SEQ ID NO: 624) | PO-mA-PO-mA-PS-lA-PS- |
| | | lA (SEQ ID NO: 639) |
| C027.003#28 | lgT3-PS-lgT3-PS-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PS-lT4-PS-lT4 (SEQ ID NO: 624) | mU-PO-mA-PO-mA-PS- |
| | | lA-PS-lA (SEQ ID |
| | | NO: 640) |
| C027.003#29 | lgT3-PO-lgT3-PO-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-lT4-PO-lT4 (SEQ ID | mU-PO-mA-PO-mA-PS- |
| | NO: 625) | mA-PS-mA (SEQ ID |
| | | NO: 632) |
| C027.003#30 | lgT3-PO-lgT3-PO-lgT3-PO-lT- | mA-PS-fA-PS-mA-PO- |
| | PO-lT-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PO-lT4-PO-lT4 (SEQ ID | PO-mU-PO-mA-PO-mA- |
| | NO: 625) | PS-mA-PS-mA (SEQ ID |
| | | NO: 633) |
| C027.003#31 | lgT3-PO-lgT3-PO-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-lT4-PO-lT4 (SEQ ID | PO-mA-PO-mA-PS-mA- |
| | NO: 625) | PS-mA (SEQ ID NO: 634) |
| C027.003#32 | lgT3-PO-lgT3-PO-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-lT4-PO-lT4 (SEQ ID | mU-PO-mA-PO-mA-PS- |
| | NO: 625) | mA-PS-mA (SEQ ID |
| | | NO: 635) |
| C027.003#33 | lgT3-PO-lgT3-PO-lgT3-PO-lT- | fA-PS-fA-PS-mA-PO-fG- |
| | PO-lT-PO-mA-PO-mU-PO-fU- | PO-mU-PO-fC-PO-mA- |
| | PO-mA-PO-fA-PO-fU-PO-fA- | PO-fC-PO-mC-PO-fA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-lT4-PO-lT4 (SEQ ID | PO-fA-PO-mA-PS-dT-PS- |
| | NO: 625) | dT (SEQ ID NO: 636) |
| C027.003#34 | lgT3-PS-lgT3-PS-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PS-lT4-PS-lT4 (SEQ ID NO: 626) | mU-PO-mA-PO-mA-PS- |
| | | mA-PS-mA (SEQ ID |
| | | NO: 632) |
| C027.003#35 | lgT3-PS-lgT3-PS-lgT3-PO-lT- | mA-PS-fA-PS-mA-PO- |
| | PO-lT-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PS-lT4-PS-lT4 (SEQ ID NO: 626) | PO-mU-PO-mA-PO-mA- |
| | | PS-mA-PS-mA (SEQ ID |
| | | NO: 633) |
| C027.003#36 | lgT3-PS-lgT3-PS-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PS-lT4-PS-lT4 (SEQ ID NO: 626) | PO-mA-PO-mA-PS-mA- |
| | | PS-mA (SEQ ID NO: 634) |
| C027.003#37 | lgT3-PS-lgT3-PS-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PS-lT4-PS-lT4 (SEQ ID NO: 626) | mU-PO-mA-PO-mA-PS- |
| | | mA-PS-mA (SEQ ID |
| | | NO: 635) |
| C027.003#38 | lgT3-PS-lgT3-PS-lgT3-PO-lT- | fA-PS-fA-PS-mA-PO-fG- |
| | PO-lT-PO-mA-PO-mU-PO-fU- | PO-mU-PO-fC-PO-mA- |
| | PO-mA-PO-fA-PO-fU-PO-fA- | PO-fC-PO-mC-PO-fA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PS-lT4-PS-lT4 (SEQ ID NO: 626) | PO-fA-PO-mA-PS-dT-PS- |
| | | dT (SEQ ID NO: 636) |
| C027.003#39 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-mU-PO-mU-PO-lT4-PO-lT4 | mU-PO-mA-PO-mA-PS- |
| | (SEQ ID NO: 627) | mA-PS-mA (SEQ ID |
| | | NO: 632) |
| C027.003#40 | lgT3-PO-lgT3-PO-lgT3-PO-mU- | mA-PS-fA-PS-mA-PO- |
| | PO-mU-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PO-mU-PO-mU-PO-lT4-PO-lT4 | PO-mU-PO-mA-PO-mA- |
| | (SEQ ID NO: 627) | PS-mA-PS-mA (SEQ ID |
| | | NO: 633) |
| C027.003#41 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-mU-PO-mU-PO-lT4-PO-lT4 | PO-mA-PO-mA-PS-mA- |
| | (SEQ ID NO: 627) | PS-mA (SEQ ID NO: 634) |
| C027.003#42 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-mU-PO-mU-PO-lT4-PO-lT4 | mU-PO-mA-PO-mA-PS- |
| | (SEQ ID NO: 627) | mA-PS-mA (SEQ ID |
| | | NO: 635) |
| C027.003#43 | lgT3-PO-lgT3-PO-lgT3-PO-mU- | fA-PS-fA-PS-mA-PO-fG- |
| | PO-mU-PO-mA-PO-mU-PO-fU- | PO-mU-PO-fC-PO-mA- |
| | PO-mA-PO-fA-PO-fU-PO-fA- | PO-fC-PO-mC-PO-fA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-mU-PO-mU-PO-lT4-PO-lT4 | PO-fA-PO-mA-PS-dT-PS- |
| | (SEQ ID NO: 627) | dT (SEQ ID NO: 636) |
| C027.003#44 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-mU-PO-mU-PO-lT4-PO-lT4 | mU-PO-mA-PO-mA-PS- |
| | (SEQ ID NO: 627) | lA-PS-lA (SEQ ID |
| | | NO: 637) |
| C027.003#45 | lgT3-PO-lgT3-PO-lgT3-PO-mU- | mA-PS-fA-PS-mA-PO- |
| | PO-mU-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PO-mU-PO-mU-PO-lT4-PO-lT4 | PO-mU-PO-mA-PO-mA- |
| | (SEQ ID NO: 627) | PS-lA-PS-lA (SEQ ID |
| | | NO: 638) |
| C027.003#46 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-mU-PO-mU-PO-lT4-PO-lT4 | PO-mA-PO-mA-PS-lA-PS- |
| | (SEQ ID NO: 627) | lA (SEQ ID NO: 639) |
| C027.003#47 | lgT3-PO-lgT3-PO-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-mU-PO-mU-PO-lT4-PO-lT4 | mU-PO-mA-PO-mA-PS- |
| | (SEQ ID NO: 627) | lA-PS-lA (SEQ ID |
| | | NO: 640) |
| C027.003#48 | lgT3-PS-lgT3-PS-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-mU-PO-mU-PS-lT4-PS-lT4 | mU-PO-mA-PO-mA-PS- |
| | (SEQ ID NO: 628) | mA-PS-mA (SEQ ID |
| | NO: 632) | |
| C027.003#49 | lgT3-PS-lgT3-PS-lgT3-PO-mU- | mA-PS-fA-PS-mA-PO- |
| | PO-mU-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PO-mU-PO-mU-PS-lT4-PS-lT4 | PO-mU-PO-mA-PO-mA- |
| | (SEQ ID NO: 628) | PS-mA-PS-mA (SEQ ID |
| | | NO: 633) |
| C027.003#50 | lgT3-PS-lgT3-PS-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-mU-PO-mU-PS-lT4-PS-lT4 | PO-mA-PO-mA-PS-mA- |
| | (SEQ ID NO: 628) | PS-mA (SEQ ID NO: 634) |
| C027.003#51 | lgT3-PS-lgT3-PS-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-mU-PO-mU-PS-lT4-PS-lT4 | mU-PO-mA-PO-mA-PS- |
| | (SEQ ID NO: 628) | mA-PS-mA (SEQ ID |
| | | NO: 635) |
| C027.003#52 | lgT3-PS-lgT3-PS-lgT3-PO-mU- | fA-PS-fA-PS-mA-PO-fG- |
| | PO-mU-PO-mA-PO-mU-PO-fU- | PO-mU-PO-fC-PO-mA- |
| | PO-mA-PO-fA-PO-fU-PO-fA- | PO-fC-PO-mC-PO-fA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-mU-PO-mU-PS-lT4-PS-lT4 | PO-fA-PO-mA-PS-dT-PS- |
| | (SEQ ID NO: 628) | dT (SEQ ID NO: 636) |
| C027.003#53 | lgT3-PS-lgT3-PS-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-mU-PO-mU-PS-lT4-PS-lT4 | mU-PO-mA-PO-mA-PS- |
| | (SEQ ID NO: 628) | lA-PS-lA (SEQ ID |
| | | NO: 637) |
| C027.003#54 | lgT3-PS-lgT3-PS-lgT3-PO-mU- | mA-PS-fA-PS-mA-PO- |
| | PO-mU-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PO-mU-PO-mU-PS-lT4-PS-lT4 | PO-mU-PO-mA-PO-mA- |
| | (SEQ ID NO: 628) | PS-lA-PS-lA (SEQ ID |
| | | NO: 638) |
| C027.003#55 | lgT3-PS-lgT3-PS-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-mU-PO-mU-PS-lT4-PS-lT4 | PO-mA-PO-mA-PS-lA-PS- |
| | (SEQ ID NO: 628) | lA (SEQ ID NO: 639) |
| C027.003#56 | lgT3-PS-lgT3-PS-lgT3-PO-mU- | mA-PS-fA-PS-mA-PO-fG- |
| | PO-mU-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-mU-PO-mU-PS-lT4-PS-lT4 | mU-PO-mA-PO-mA-PS- |
| | (SEQ ID NO: 628) | lA-PS-lA (SEQ ID |
| | | NO: 640) |
| C027.003#57 | lgT3-PO-lgT3-PO-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-mU-PO-mU-PO-lT4-PO-lT4 | mU-PO-mA-PO-mA-PS- |
| | (SEQ ID NO: 629) | mA-PS-mA (SEQ ID |
| | | NO: 632) |
| C027.003#58 | lgT3-PO-lgT3-PO-lgT3-PO-lT- | mA-PS-fA-PS-mA-PO- |
| | PO-lT-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PO-mU-PO-mU-PO-lT4-PO-lT4 | PO-mU-PO-mA-PO-mA- |
| | (SEQ ID NO: 629) | PS-mA-PS-mA (SEQ ID |
| | | NO: 633) |
| C027.003#59 | lgT3-PO-lgT3-PO-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-mU-PO-mU-PO-lT4-PO-lT4 | PO-mA-PO-mA-PS-mA- |
| | (SEQ ID NO: 629) | PS-mA (SEQ ID NO: 634) |
| C027.003#60 | lgT3-PO-lgT3-PO-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-mU-PO-mU-PO-lT4-PO-lT4 | mU-PO-mA-PO-mA-PS- |
| | (SEQ ID NO: 629) | mA-PS-mA (SEQ ID |
| | | NO: 635) |
| C027.003#61 | lgT3-PO-lgT3-PO-lgT3-PO-lT- | fA-PS-fA-PS-mA-PO-fG- |
| | PO-lT-PO-mA-PO-mU-PO-fU- | PO-mU-PO-fC-PO-mA- |
| | PO-mA-PO-fA-PO-fU-PO-fA- | PO-fC-PO-mC-PO-fA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-mU-PO-mU-PO-lT4-PO-lT4 | PO-fA-PO-mA-PS-dT-PS- |
| | (SEQ ID NO: 629) | dT (SEQ ID NO: 636) |
| C027.003#62 | lgT3-PS-lgT3-PS-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-mC-PO-mC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-mU-PO-mU-PS-lT4-PS-lT4 | mU-PO-mA-PO-mA-PS- |
| | (SEQ ID NO: 630) | mA-PS-mA (SEQ ID |
| | | NO: 632) |
| C027.003#63 | lgT3-PS-lgT3-PS-lgT3-PO-lT- | mA-PS-fA-PS-mA-PO- |
| | PO-lT-PO-mA-PO-mU-PO-fU- | mG-PO-mU-PO-fC-PO- |
| | PO-mA-PO-fA-PO-fU-PO-fA- | mA-PO-mC-PO-mC-PO- |
| | PO-mU-PO-mG-PO-mG-PO-mU- | mA-PO-mU-PO-mA-PO- |
| | PO-mG-PO-mA-PO-mC-PO-mU- | mU-PO4U-PO-mA-PO-fA- |
| | PO-mU-PO-mU-PS-lT4-PS-lT4 | PO-mU-PO-mA-PO-mA- |
| | (SEQ ID NO: 630) | PS-mA-PS-mA (SEQ ID |
| | | NO: 633) |
| C027.003#64 | lgT3-PS-lgT3-PS-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-fC-PO-fC-PO-mA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-mU-PO-mU-PS-lT4-PS-lT4 | PO-mA-PO-mA-PS-mA- |
| | (SEQ ID NO: 630) | PS-mA (SEQ ID NO: 634) |
| C027.003#65 | lgT3-PS-lgT3-PS-lgT3-PO-lT- | mA-PS-fA-PS-mA-PO-fG- |
| | PO-lT-PO-mA-PO-mU-PO-fU- | PO-mU-PO-mC-PO-mA- |
| | PO-mA-PO-fA-PO-fU-PO-fA- | PO-mC-PO-fC-PO-mA- |
| | PO-mU-PO-mG-PO-mG-PO-mU- | PO-mU-PO-mA-PO-mU- |
| | PO-mG-PO-mA-PO-mC-PO-mU- | PO4U-PO-mA-PO-fA-PO- |
| | PO-mU-PO-mU-PS-lT4-PS-lT4 | mU-PO-mA-PO-mA-PS- |
| | (SEQ ID NO: 630) | mA-PS-mA (SEQ ID |
| | | NO: 635) |
| C027.003#66 | lgT3-PS-lgT3-PS-lgT3-PO-lT- | fA-PS-fA-PS-mA-PO-fG- |
| | PO-lT-PO-mA-PO-mU-PO-fU- | PO-mU-PO-fC-PO-mA- |
| | PO-mA-PO-fA-PO-fU-PO-fA- | PO-fC-PO-mC-PO-fA-PO- |
| | PO-mU-PO-mG-PO-mG-PO-mU- | mU-PO-mA-PO-mU-PO- |
| | PO-mG-PO-mA-PO-mC-PO-mU- | fU-PO-mA-PO-fA-PO-mU- |
| | PO-mU-PO-mU-PS-lT4-PS-lT4 | PO-fA-PO-mA-PS-dT-PS- |
| | (SEQ ID NO: 630) | dT (SEQ ID NO: 636) |

In some embodiments, a dsRNA of the present disclosure comprises:
a) a sense strand comprising a nucleotide sequence selected from the group consisting of SEQ ID NOs: 578, 585, 587, 620, 621, 622, and 627; and/or
b) an antisense strand comprising a nucleotide sequence selected from the group consisting of SEQ ID NOs: 589, 591, 631, 632, 634, 635 and 639.

In some embodiments, the sense strand and the antisense strand of the dsRNA respectively comprise the nucleotide sequences of:

a) SEQ ID NOs: 578 and 589; [C027.001]

b) SEQ ID NOs: 620 and 631; [C027.003]

c) SEQ ID NOs: 585 and 591; [C027.001#40]

d) SEQ ID NOs: 587 and 591; [C027.001#58]

e) SEQ ID NOs: 621 and 634; [C027.003#03]

f) SEQ ID NOs: 622 and 632; [C027.003#06]

g) SEQ ID NOs: 622 and 635; and [C027.003#08]

h) SEQ ID NOs: 627 and 639; [C027.003#47]

In some embodiments, a dsRNA of the present disclosure (e.g., a first dsRNA) is used in a method or composition (e.g., a pharmaceutical composition) with one or more additional dsRNAs (e.g., at least a second dsRNA). In some embodiments, the second dsRNA also targets PCSK9. In some embodiments, the second dsRNA targets a region of PCSK9 that is different from the region targeted by the first dsRNA. In some embodiments, the second dsRNA targets a sequence derived from the sequence of an mRNA formed during the expression of a target gene other than the PCSK9 gene. In some embodiments, the second dsRNA targets a gene that interacts with PCSK9 and/or a gene that is involved in lipid metabolism or cholesterol metabolism.

dsRNA Modifications

In some embodiments, a dsRNA of the present disclosure comprises one or more modifications. Modifications may include any modification known in the art, including, for example, end modifications, base modifications, sugar modifications/replacements, and backbone modifications. End modifications may include, e.g., 5′ end modifications (such as phosphorylation, conjugation, inverted linkages, and the like) and 3′ end modifications (such as conjugates, DNA nucleotides, inverted linkages, and the like). Base modifications may include, e.g., replacement with stabilizing bases, destabilizing bases, bases that base pair with an expanded repertoire of partners, removal of bases (abasic nucleotides), or conjugated bases. Sugar modifications/replacements may include, e.g., modifications at the 2′ or 4′ position, or replacement of the sugar. Backbone modifications may include, e.g., modification or replacement of the phosphodiester linkages.

dsRNAs of the present disclosure may include one or more of modified nucleotides known in the art, including, 2′-O-methyl modified nucleotides, 2′-fluoro modified nucleotides, 2′-deoxy modified nucleotides, 2′-O-methoxyethyl modified nucleotides, modified nucleotides comprising alternate internucleotide linkages such as thiophosphates and phosphorothioates (e.g., 5′-phosphorothioate), phosphotriester modified nucleotides, modified nucleotides terminally linked to a cholesterol derivative or lipophilic moiety, peptide nucleic acids (PNAs; see Nielsen et al. (1991) Science 254, 1497-1500), constrained ethyl (cEt) modified nucleotides, inverted deoxy modified nucleotides, inverted dideoxy modified nucleotides, locked nucleic acid modified nucleotides, abasic modified nucleotides, 2′-amino modified nucleotides, 2′-alkyl modified nucleotides, morpholino-modified nucleotides, phosphoramidate modified nucleotides, modified nucleotides comprising modifications at other sites of the sugar or base of an oligonucleotide, and non-natural base-containing modified nucleotides. In some embodiments, at least one of the one or more modified nucleotides is a 2′-O-methyl nucleotide, 5′-phosphorothioate nucleotide, or a terminal nucleotide linked to a cholesterol derivative or lipophilic moiety. The incorporation of 2′-O-methyl, 2′-O-ethyl, 2′-O-propyl, 2′-O-allyl, 2′-O-aminoalkyl, or 2′-deoxy-2′-fluoro group in nucleosides of an oligonucleotide may confer enhanced hybridization properties and/or enhanced nuclease stability to the oligonucleotide. Further, oligonucleotides containing phosphorothioate backbones may have enhanced nuclease stability.

In some embodiments, a dsRNA of the present disclosure comprises one or more 2′-O-methyl nucleotides and one or more 2′-fluoro nucleotides. In some embodiments, the dsRNA comprises two or more 2′-O-methyl nucleotides and two or more 2′-fluoro nucleotides. In some embodiments, the dsRNA comprises two or more 2′-O-methyl nucleotides (OMe) and two or more 2′-fluoro nucleotides (F) in an alternating pattern, e.g., the pattern OMe-F-OMe-F or the pattern F-OMe-F-OMe. In some embodiments, the dsRNA comprises up to 10 contiguous nucleotides that are each a 2′-O-methyl nucleotide. In some embodiments, the dsRNA comprises up to 10 contiguous nucleotides that are each a 2′-fluoro nucleotide.

In some embodiments, a dsRNA of the present disclosure comprises one or more phosphorothioate groups. In some embodiments, a dsRNA of the present disclosure comprises two or more, three or more, four or more, five or more, six or more, seven or more, eight or more, nine or more, or 10 or more phosphorothioate groups. In some embodiments, the dsRNA does not comprise a phosphorothioate group.

In some embodiments, the dsRNA comprises one or more phosphotriester groups. In some embodiments, the dsRNA comprises two or more, three or more, four or more, five or more, six or more, seven or more, eight or more, nine or more, or 10 or more phosphotriester groups. In some embodiments, the dsRNA does not comprise a phosphotriester group.

In some embodiments, the dsRNA comprises two or more, three or more, four or more, five or more, six or more, seven or more, eight or more, nine or more, or 10 or more different modified nucleotides described herein. In some embodiments, the dsRNA comprises up to two contiguous modified nucleotides, up to three contiguous modified nucleotides, up to four contiguous modified nucleotides, up to five contiguous modified nucleotides, up to six contiguous modified nucleotides, up to seven contiguous modified nucleotides, up to eight contiguous modified nucleotides, up to nine contiguous modified nucleotides, or up to 10 contiguous modified nucleotides. In some embodiments, the contiguous modified nucleotides are the same modified nucleotide. In some embodiments, the contiguous modified nucleotides are two or more, three or more, four or more, five or more, six or more, seven or more, eight or more, nine or more, or ten or more different modified nucleotides.

dsRNA Conjugates

dsRNAs of the present disclosure may be chemically/physically linked to one or more ligands, moieties, or conjugates. In some embodiments, the dsRNA is conjugated/attached to one or more ligands via a linker. Any linker known in the art may be used, including, for example, trivalent branched linkers. Conjugating a ligand to a dsRNA may alter its distribution, enhance its cellular absorption and/or targeting to a particular tissue and/or uptake by one or more specific cell types (e.g., liver cells), and/or enhance the lifetime of the dsRNA agent. In some embodiments, a hydrophobic ligand is conjugated to the dsRNA to facilitate direct permeation of the cellular membrane and/or uptake across the cells (e.g., liver cells).

In some embodiments, the dsRNA is attached to one or more N-acetylgalactosamine (GalNAc) derivatives via a linker. In some embodiments, the dsRNA is attached to three or more GalNAc derivatives via a linker. In some embodiments, the linker is a trivalent branched linker. In some embodiments, the dsRNA is attached to three or more GalNAc derivatives via a trivalent branched linker. In some embodiments, the one or more GalNAc derivatives is attached to the 3′-end of the sense strand, the 3′-end of the antisense strand, the 5′-end of the sense strand, and/or the 5′-end of the antisense strand of the dsRNA.

Exemplary and non-limiting conjugates and linkers are described, e.g., in Biessen et al., Bioconjugate Chem. 13(2):295-302 (2002); Cedillo et al., Molecules 22(8):E1356 (2017); Grijalvo et al., Genes 9(2):E74 (2018); Huang et al., Molecular Therapy: Nucleic Acids 6:116-132 (2017); Nair et al., J. Am. Chem. Soc. 136:16958-16961 (2014); Ostergaard et al., Bioconjugate Chem. 26:1451-1455 (2015); Springer et al., Nucleic Acid Therapeutics 28(3):109-118 (2018); and U.S. Pat. Nos. 8,106,022, 9,127,276, and 8,927,705. GalNAc conjugation can be readily performed by methods well known in the art (e.g., as described in the above documents).

In some embodiments, a dsRNA of the present disclosure is attached to the compound below.

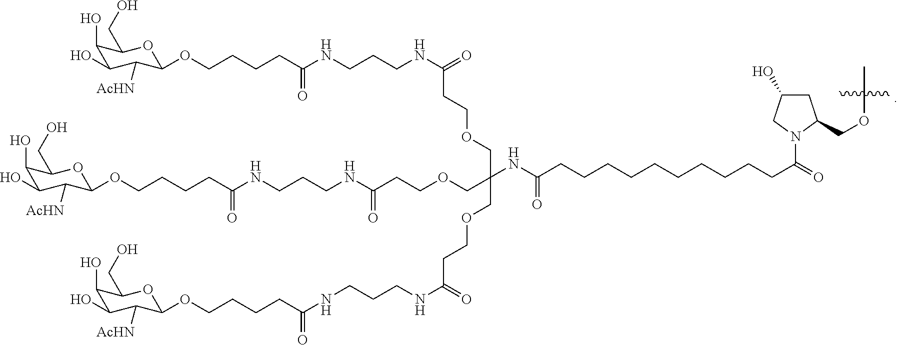 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some embodiments, the ligand is one or more targeting groups (e.g., a cell or tissue targeting agent), e.g., one or more proteins, glycoproteins, peptides, or molecules having a specific affinity for a co-ligand. Such ligands may include without limitation a lectin, glycoprotein, lipid or protein, e.g., an antibody, which binds to a specified cell type such as a liver cell. A targeting group may be a thyrotropin, melanotropin, lectin, glycoprotein, surfactant protein A, mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, or biotin.

Ligands may include, for example, a naturally occurring substance, such as a protein, carbohydrate (e.g., N-acetyl-glucosamine or N-acetyl-galactosamine), lipopolysaccharide, lipid, recombinant or synthetic molecule such as a synthetic polymer, polyamine, an alpha helical peptide, lectins, vitamins, and cofactors. In some embodiments, the ligand is one or more dyes, crosslinkers, polycyclic aromatic hydrocarbons, peptide conjugates (e.g., antennapedia peptide, Tat peptide), polyethylene glycol (PEG), enzymes, haptens, transport/absorption facilitators, synthetic ribonucleases (e.g., imidazole, bisimidazole, histamine, or imidazole clusters), human serum albumin (HSA), or LDL.

In some embodiments, the dsRNA is conjugated to one or more cholesterol derivatives or lipophilic moieties. Any lipophilic compound known in the art may be conjugated to the dsRNA, including, without limitation, cholesterol or a cholesterol derivative; cholic acid; a vitamin (such as folate, vitamin A, vitamin E (tocopherol), biotin, pyridoxal); bile or fatty acid conjugates, including both saturated and non-saturated (such as lauroyl (C₁₂), myristoyl (C₁₄) and palmitoyl (C₁₆), stearoyl (Cis) and docosanyl (C₂₂), lithocholic acid and/or lithocholic acid oleylamine conjugate (lithocholic-oleyl, C₄₃)); polymeric backbones or scaffolds (such as PEG, triethylene glycol (TEG), hexaethylene glycol (HEG), poly(lactic-co-glycolic acid) (PLGA), poly(lactide-co-glycolide) (PLG), hydrodynamic polymers); steroids (such as dihydrotestosterone); terpene (such as triterpene); cationic lipids or peptides; and/or a lipid or lipid-based molecule. Such a lipid or lipid-based molecule may bind a serum protein, e.g., human serum albumin (HSA). A lipid-based ligand may be used to modulate (e.g., control) the binding of the conjugate to a target tissue. For example, a lipid or lipid-based ligand that binds to HSA more strongly will be less likely to be targeted to the kidney and therefore less likely to be cleared from the body. The target tissue may be the liver, including parenchymal cells of the liver.

I. Compositions

Certain aspects of the present disclosure relate to compositions (e.g., pharmaceutical compositions) comprising a dsRNA as described herein. In some embodiments, the composition (e.g., pharmaceutical composition) further comprises a pharmaceutically acceptable carrier. In some embodiments, the composition (e.g., pharmaceutical composition) is useful for treating a disease or disorder associated with the expression or activity of the PCSK9 gene. In some embodiments, the disease or disorder associated with the expression of the PCSK9 gene is lipidemia (e.g., hyperlipidemia) and/or other forms of lipid imbalances such as hypercholesterolemia, hypertriglyceridemia, and pathological conditions associated with these disorders such as heart and circulatory diseases. Compositions (e.g., pharmaceutical compositions) of the present disclosure are formulated based upon the mode of delivery, including, for example, compositions formulated for delivery to the liver via parenteral delivery.

The compositions (e.g., pharmaceutical composition) of the present disclosure may be administered in dosages sufficient to inhibit expression of the PCSK9 gene. In some embodiments, a suitable dose of a dsRNA is in the range of 0.01 mg/kg-200 mg/kg body weight of the recipient.

One of ordinary skill in the art will appreciate that certain factors may influence the dosage and timing required to effectively treat a subject, including, but not limited to, severity of the disease or disorder, previous treatments, the general health and/or age of the subject, and one or more other diseases being present. Moreover, treatment of a subject with a therapeutically effective amount of a pharmaceutical composition can include a single treatment or a series of treatments. Estimates of effective dosages and in vivo half-lives for dsRNAs as disclosed herein may be made using conventional methodologies or on the basis of in vivo testing using an appropriate animal model.

dsRNA molecules of the present disclosure can be formulated in a pharmaceutically acceptable carrier or diluent. Pharmaceutically acceptable carriers can be liquid or solid, and may be selected with the planned manner of administration in mind so as to provide for the desired bulk, consistency, and other pertinent transport and chemical properties. Any known pharmaceutically acceptable carrier or diluent may be used, including, for example, water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), calcium salts (e.g., calcium sulfate, calcium chloride, calcium phosphate, etc.) and wetting agents (e.g., sodium lauryl sulfate).

dsRNA molecules of the present disclosure can be formulated into compositions (e.g., pharmaceutical compositions) containing the dsRNA admixed, encapsulated, conjugated, or otherwise associated with other molecules, molecular structures, or mixtures of nucleic acids. For example, a composition comprising one or more dsRNAs as described herein can contain other therapeutic agents such as other lipid lowering agents (e.g., statins). In some embodiments, the composition (e.g., pharmaceutical composition) further comprises a delivery vehicle (as described herein).

II. Methods of Making dsRNAs

A dsRNA of the present disclosure may be synthesized by any method known in the art. For example, a dsRNA may be synthesized by use of an automated synthesizer, by in vitro transcription and purification (e.g., using commercially available in vitro RNA synthesis kits), by transcription and purification from cells (e.g., cells comprising an expression cassette/vector encoding the dsRNA), and the like.

Preparation of Modified dsRNAs

Ligand-conjugated dsRNAs and ligand-molecule bearing sequence-specific linked nucleosides of the present disclosure may be assembled by any method known in the art, including, for example, by assembly on a suitable DNA synthesizer utilizing standard nucleotide or nucleoside precursors, or nucleotide or nucleoside conjugate precursors that already bear the linking moiety, ligand-nucleotide, or nucleoside-conjugated precursors that already bear the ligand molecule, or non-nucleoside ligand-bearing building blocks.

Ligand-conjugated dsRNAs of the present disclosure may be synthesized by any method known in the art, including, for example, by the use of a dsRNA bearing a pendant reactive functionality such as that derived from the attachment of a linking molecule onto the dsRNA. In some embodiments, this reactive oligonucleotide may be reacted directly with commercially-available ligands, ligands that are synthesized bearing any of a variety of protecting groups, or ligands that have a linking moiety attached thereto. In some embodiments, the methods facilitate the synthesis of ligand-conjugated dsRNA by the use of nucleoside monomers that have been appropriately conjugated with ligands and that may further be attached to a solid support material. In some embodiments, a dsRNA bearing an aralkyl ligand attached to the 3′-end of the dsRNA is prepared by first covalently attaching a monomer building block to a controlled-pore-glass support via a long-chain aminoalkyl group; then, nucleotides are bonded via standard solid-phase synthesis techniques to the monomer building-block bound to the solid support. The monomer building-block may be a nucleoside or other organic compound that is compatible with solid-phase synthesis.

In some embodiments, functionalized nucleoside sequences of the present disclosure possessing an amino group at the 5′-terminus are prepared using DNA synthesizer, and then reacted with an active ester derivative of a selected ligand. Active ester derivatives are well known to one of ordinary skill in the art. The reaction of the amino group and the active ester produces an oligonucleotide in which the selected ligand is attached to the 5′-position through a linking group. The amino group at the 5′-terminus can be prepared utilizing a 5′-amino-modifier C6 reagent. In some embodiments, ligand molecules are conjugated to oligonucleotides at the 5′-position by the use of a ligand-nucleoside phosphoramidite wherein the ligand is linked to the 5′-hydroxy group directly or indirectly via a linker. Such ligand-nucleoside phosphoramidites are typically used at the end of an automated synthesis procedure to provide a ligand-conjugated oligonucleotide bearing the ligand at the 5′-terminus.

III. Vectors and dsRNA Delivery

A dsRNA of the present disclosure may be delivered directly or indirectly. In some embodiments, the dsRNA is delivered directly by administering a composition (e.g., pharmaceutical composition) comprising the dsRNA to a subject. In some embodiments, the dsRNA is delivered indirectly by administering one or more vectors described herein.

Delivery

A dsRNA of the present disclosure may be delivered by any method known in the art, including, for example, by adapting a method of delivering a nucleic acid molecule for use with a dsRNA (See e.g., Akhtar, S. et al. (1992) Trends Cell. Biol. 2(5): 139-144; WO 94/02595), or via additional methods known in the art (See e.g., Kanasty, R. et al. (2013) Nature Materials 12: 967-977; Wittrup, A. and Lieberman, J. (2015) Nature Reviews Genetics 16: 543-552; Whitehead, K. et al. (2009) Nature Reviews Drug Discovery 8: 129-138; Gary, D. et al. (2007) 121 (1-2): 64-73; Wang. J. et al. (2010) AAPS J. 12(4): 492-503; Draz, M. et al. (2014) Theranostics 4(9): 872-892; Wan, C. et al. (2013) Drug Deliv. And Transl. Res. 4(1): 74-83; Erdmann, V. A. and Barciszewski, J. (eds.) (2010) “RNA Technologies and Their Applications”, Springer-Verlag Berlin Heidelberg, DOI 10.1007/978-3-642-12168-5; Xu, C. and Wang, J. (2015) Asian Journal of Pharmaceutical Sciences 10(1): 1-12).

In some embodiments, a dsRNA of the present disclosure is delivered by a delivery vehicle comprising the dsRNA. In some embodiments, the delivery vehicle is a liposome, lipoplex, complex, or nanoparticle.

Liposomal Formulations

Liposomes are unilamellar or multilamellar vesicles which have a membrane formed from a lipophilic material and an aqueous interior. In some embodiments, a liposome is a vesicle composed of amphiphilic lipids arranged in a spherical bilayer or bilayers. The aqueous portion contains the composition to be delivered. Cationic liposomes possess the advantage of being able to fuse to the cell wall. Advantages of liposomes include, e.g., liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes. For example, engineered cationic liposomes and sterically stabilized liposomes can be used to deliver the dsRNA. See, e.g., Podesta et al. (2009) Methods Enzymol. 464, 343-54; U.S. Pat. No. 5,665,710.

Nucleic Acid-Lipid Particles

In some embodiments, a dsRNA of the present disclosure is fully encapsulated in a lipid formulation, e.g., to form a nucleic acid-lipid particle, e.g., a SPLP, pSPLP, or SNALP. As used herein, the term “SNALP” refers to a stable nucleic acid-lipid particle, including SPLP. As used herein, the term “SPLP” refers to a nucleic acid-lipid particle comprising plasmid DNA encapsulated within a lipid vesicle. Nucleic acid-lipid particles, e.g., SNALPs, typically contain a cationic lipid, a non-cationic lipid, and a lipid that prevents aggregation of the particle (e.g., a PEG-lipid conjugate). SNALPs and SPLPs are useful for systemic applications, as they exhibit extended circulation lifetimes following intravenous (i.v.) injection and accumulate at distal sites (e.g., sites physically separated from the administration site). SPLPs include “pSPLP”, which include an encapsulated condensing agent-nucleic acid complex as set forth in PCT Publication No. WO 00/03683.

In some embodiments, dsRNAs when present in the nucleic acid-lipid particles are resistant in aqueous solution to degradation with a nuclease. Nucleic acid-lipid particles and their methods of preparation are disclosed in, e.g., U.S. Pat. Nos. 5,976,567; 6,534,484; 6,815,432; and PCT Publication No. WO 96/40964.

In some embodiments, the nucleic acid-lipid particles comprise a cationic lipid. Any cationic lipid or mixture thereof known in the art may be used. In some embodiments, the nucleic acid-lipid particles comprise a non-cationic lipid. Any non-cationic lipid or mixture thereof known in the art may be used. In some embodiments, the nucleic acid-lipid particle comprises a conjugated lipid (e.g., to prevent aggregation). Any conjugated lipid known in the art may be used.

Additional Formulations

Factors that are important to consider in order to successfully deliver a dsRNA molecule in vivo include: (1) biological stability of the delivered molecule, (2) preventing nonspecific effects, and (3) accumulation of the delivered molecule in the target tissue. The nonspecific effects of a dsRNA can be minimized by local administration, for example by direct injection or implantation into a tissue or topically administering the preparation. For administering a dsRNA systemically for the treatment of a disease, the dsRNA may be modified or alternatively delivered using a drug delivery system; both methods act to prevent the rapid degradation of the dsRNA by endo and exo-nucleases in vivo. Modification of the RNA or the pharmaceutical carrier may also permit targeting of the dsRNA composition to the target tissue and avoid undesirable off-target effects. As described above, dsRNA molecules may be modified by chemical conjugation to lipophilic groups such as cholesterol to enhance cellular uptake and prevent degradation. In some embodiments, the dsRNA is delivered using drug delivery systems such as a nanoparticle, a dendrimer, a polymer, liposomes, or a cationic delivery system. Positively charged cationic delivery systems facilitate binding of a dsRNA molecule (negatively charged) and also enhance interactions at the negatively charged cell membrane to permit efficient uptake of a dsRNA by the cell. Cationic lipids, dendrimers, or polymers can either be bound to a dsRNA, or induced to form a vesicle or micelle (See e.g., Kim S. H. et al. (2008) Journal of Controlled Release 129(2):107-116) that encases a dsRNA. The formation of vesicles or micelles further prevents degradation of the dsRNA when administered systemically. Methods for making and administering cationic-dsRNA complexes are known in the art. In some embodiments, a dsRNA forms a complex with cyclodextrin for systemic administration.

Vector Encoded dsRNAs

A dsRNA of the present disclosure may be encoded by a recombinant vector. In some embodiments, the vector is a DNA vector or an RNA vector. In some embodiments, the vector is a plasmid, cosmid, or viral vector. In some embodiments, the vector is compatible with expression in prokaryotic cells. In some embodiments, the vector is compatible with expression in E. coli. In some embodiments, the vector is compatible with expression in eukaryotic cells. In some embodiments, the vector is compatible with expression in yeast cells. In some embodiments, the vector is compatible with expression in vertebrate cells. Any expression vector capable of encoding the dsRNA known in the art may be used, including, for example, vectors derived from adenovirus (AV), adeno-associated virus (AAV), retroviruses (e.g., lentiviruses (LV), Rhabdoviruses, murine leukemia virus, etc.), herpes virus, SV40 virus, polyoma virus, papilloma virus, picornavirus, pox virus (e.g., orthopox or avipox), and the like. The tropism of viral vectors or viral-derived vectors may be modified by pseudotyping the vectors with envelope proteins or other surface antigens from one or more other viruses, or by substituting different viral capsid proteins, as appropriate. For example, lentiviral vectors may be pseudotypes with surface proteins from vesicular stomatitis virus (VSV), rabies, Ebola, Mokola, and the like. AAV vectors may be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes have been described previously, e.g., Rabinowitz et al. (2002) J. Virol. 76:791-801.

Selection of recombinant vectors, methods for inserting nucleic acid sequences into the vector for expressing a dsRNA, and methods of delivering vectors into one or more cells of interest are known in the art. See, e.g., Domburg (1995) Gene Therap. 2:301-310; Eglitis (1998) Biotechniques 6:608-614; Miller (1990) Hum. Gene Therap. 1:5-14; Anderson (1998) Nature 392:25-30; Xia et al. (2002) Nat. Biotech. 20:1006-1010; Robinson et al. (2003) Nat. Genet. 33:401-406; Samulski et al. (1987) J. Virol. 61:3096-3101; Fisher et al. (1996) J. Virol. 70:520-532; Samulski et al. (1989) J. Virol. 63-3822-3826; U.S. Pat. Nos. 5,252,479; 5,139,941; WO 94/13788; and WO 93/24641.

Vectors useful for the delivery of a dsRNA as described herein may include regulatory elements (e.g., heterologous promoter, enhancer, etc.) sufficient for expression of the dsRNA in the desired target cell or tissue. In some embodiments, the vector comprises one or more sequences encoding the dsRNA linked to one or more heterologous promoters. Any heterologous promoter known in the art capable of expressing a dsRNA may be used, including, for example, the U6 or H1 RNA pol III promoters, the T7 promoter, and the cytomegalovirus promoter. The one or more heterologous promoters may be an inducible promoter, a repressible promoter, a regulatable promoter, and/or a tissue-specific promoter. Selection of additional promoters is within the abilities of one of ordinary skill in the art. In some embodiments, the regulatory elements are selected to provide constitutive expression. In some embodiments, the regulatory elements are selected to provide regulated/inducible/repressible expression. In some embodiments, the regulatory elements are selected to provide tissue-specific expression. In some embodiments, the regulatory elements and sequence encoding the dsRNA form a transcription unit.

A dsRNA of the present disclosure may be expressed from transcription units inserted into DNA or RNA vectors (See, e.g., Couture, A, et al. (1996) TIG 12:5-10; WO 00/22113; WO 00/22114; and U.S. Pat. No. 6,054,299). Expression may be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al. (1995) PNAS 92:1292).

In some embodiments, the sense and antisense strands of a dsRNA are encoded on separate expression vectors. In some embodiments, the sense and antisense strands are expressed on two separate expression vectors that are co-introduced (e.g., by transfection or infection) into the same target cell. In some embodiments, the sense and antisense strands are encoded on the same expression vector. In some embodiments, the sense and antisense strands are transcribed from separate promoters which are located on the same expression vector. In some embodiments, the sense and antisense strands are transcribed from the same promoter on the same expression vector. In some embodiments, the sense and antisense strands are transcribed from the same promoter as an inverted repeat joined by a linker polynucleotide sequence such that the dsRNA has a stem and loop structure.

IV. Cells

Certain aspects of the present disclosure relate to one or more isolated cells comprising a dsRNA as described herein, or one or more cells comprising a vector encoding a dsRNA as described herein. In some embodiments, the one or more cells are prokaryotic cells. In some embodiments, the one or more cells are E. coli cells. In some embodiments, the one or more cells are eukaryotic cells. Any eukaryotic cell known in the art may comprise a dsRNA or vector described herein, including, for example, yeast cells, monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); Hep3B cells; C3A cells; mouse mammary tumor (MMT 060562, ATCC CCL51); CHO cells (such as DHFR-CHO cells, e.g., ATCC CRL-9096); TRI cells (Mather et al., Annals N.Y. Acad. Sci. 383:44-68 (1982)); MRC 5 cells; FS4 cells; myeloma cell lines (such as NS0 and Sp2/0); and primary cells from a subject (such as primary cells isolated from a human or a non-human primate).

V. Methods of Using dsRNA

Certain aspects of the present disclosure relate to methods for inhibiting the expression of the PCSK9 gene in a mammal comprising administering an effective amount of one or more dsRNAs of the present disclosure, one or more vectors of the present disclosure, or a composition (e.g., pharmaceutical composition) of the present disclosure comprising one or more dsRNAs of the present disclosure. Certain aspects of the present disclosure relate to methods of treating and/or preventing one or more PCSK9-mediated diseases or disorders comprising administering one or more dsRNAs of the present disclosure and/or one or more vectors of the present disclosure and/or a composition (e.g., pharmaceutical composition) comprising one or more dsRNAs of the present disclosure. In some embodiments, downregulating PCSK9 expression in a subject alleviates one or more symptoms of a PCSK9-mediated disease or disorder in the subject. Examples of dsRNAs are described in Section II.

In some embodiments, expression of the PCSK9 gene in the subject is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or about 100% after treatment as compared to pretreatment levels. In some embodiments, expression of the PCSK9 gene is inhibited by at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 25 fold, at least about 50 fold, at least about 75 fold, or at least about 100 fold after treatment as compared to pretreatment levels. In some embodiments, the PCSK9 gene is inhibited in the liver of the subject.

In some embodiments, the subject is human. In some embodiments, the subject has or has been diagnosed with a PCSK9-mediated disorder or disease. In some embodiments, the subject is suspected to have a PCSK9-mediated disorder or disease. In some embodiments, the subject is at risk for developing a PCSK9-mediated disorder or disease.

The dsRNAs and compositions (e.g., pharmaceutical compositions) described herein may be used to treat lipidemia (e.g., hyperlipidemia) and/or other forms of lipid imbalances such as hypercholesterolemia, hypertriglyceridemia, and pathological conditions associated with these disorders such as heart and circulatory diseases. In some embodiments, the method includes administering an effective amount of the dsRNA to a subject having a heterozygous LDLR genotype.

In some embodiments, the effect of inhibiting PCSK9 gene expression by any of the methods described herein results in a decrease in cholesterol levels in a subject. In some embodiments, the effect of inhibiting PCSK9 gene expression results in a decrease in cholesterol in the blood of a subject. In some embodiments, the effect of inhibiting PCSK9 gene expression results in a decrease in cholesterol in the serum of a subject. In some embodiments, cholesterol levels are decreased by at least about 5%, at least about 10%, at least about 15%, at least about 20%, at least about 25%, at least about 30%, at least about 35%, at least about 40%, at least about 45%, at least about 50%, at least about 55%, at least about 60% or more as compared to pretreatment levels. In some embodiments, cholesterol levels are decreased by at least about 1.1 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 25 fold, at least about 50 fold, at least about 75 fold, at least about 100 fold or more as compared to pretreatment levels.

A dsRNA or composition (e.g., pharmaceutical composition) described herein may be administered by any means known in the art, including, without limitation, oral or parenteral routes, including intravenous, intramuscular, subcutaneous, pulmonary, transdermal, and airway (aerosol) administration. Typically, when treating a mammal with hyperlipidemia, the dsRNA molecules are administered systemically via parenteral means. In some embodiments, the dsRNAs and/or compositions are administered by subcutaneous administration. In some embodiments, the dsRNAs and/or compositions are administered by intravenous administration. In some embodiments, the dsRNAs and/or compositions are administered by pulmonary administration.

A treatment or preventative effect of a dsRNA is evident when there is a statistically significant improvement in one or more parameters of disease status, or by a failure to worsen or to develop symptoms where they would otherwise be anticipated. For example, a favorable change of at least 10%, 15%, 20%, 25%, 30%, 35%, 40%, 45%, 50% or more in a measurable parameter of disease may be indicative of effective treatment. Efficacy for a given dsRNA or composition comprising the dsRNA may also be judged using an experimental animal model for the given disease or disorder known in the art. When using an experimental animal model, efficacy of treatment is evidenced when a statistically significant reduction in a marker or symptom is observed.

Additional Agents

In some embodiments, a dsRNA of the present disclosure is administered in combination with one or more additional therapeutic agents. In some embodiments, the dsRNA and additional therapeutic agent are administered in combination in the same composition. In some embodiments, the dsRNA and additional therapeutic agent are administered as part of separate compositions. In some embodiments, the separate compositions are administered concurrently. In some embodiments, a composition comprising the dsRNA is first administered to the subject, and then the additional therapeutic agent is administered to the subject. In some embodiments, a composition comprising the additional therapeutic agent is first administered to the subject, and then the composition comprising the dsRNA is administered to the subject.

Examples of additional therapeutic agents include any known in the art to treat a lipid disorder, such as hypercholesterolemia, atherosclerosis or dyslipidemia. For example, the additional agent may be one or more of HMG-CoA reductase inhibitor (e.g., a statin), a fibrate, a bile acid sequestrant, niacin, an antiplatelet agent, an angiotensin converting enzyme inhibitor, an angiotensin II receptor antagonist (e.g., losartan potassium), an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, or a peroxisome proliferation activated receptor (PPAR) agonist. Particular examples include, without limitation, atorvastatin, pravastatin, simvastatin, lovastatin, fluvastatin, cerivastatin, rosuvastatin, ezetimibe, bezafibrate, clofibrate, fenofibrate, gemfibrozil, ciprofibrate, cholestyramine, colestipol, colesevelam, and niacin. Exemplary combination therapies suitable for administration with a dsRNA targeting PCSK9 include, e.g., niacin/lovastatin, amlodipine/atorvastatin, and ezetimibe/simvastatin.

In some embodiments, the present disclosure provides a method of instructing an end user (e.g., a caregiver or a subject) how to administer a dsRNA described herein. The method includes, optionally, providing the end user with one or more doses of the dsRNA, and instructing the end user to administer the dsRNA on a regimen described herein, thereby instructing the end user.

Identification of Patients

In some embodiments, the present disclosure provides methods of treating a subject by selecting a subject on the basis that the subject is in need of LDL lowering, LDL lowering without HDL lowering, ApoB lowering, or total cholesterol lowering. In some embodiments, the method comprises administering to the subject a dsRNA in an amount sufficient to lower the subject's LDL levels or ApoB levels (e.g., without substantially lowering HDL levels).

Genetic predisposition plays a role in the development of target gene associated diseases, e.g., hyperlipidemia. Therefore, a subject in need of a dsRNA may be identified by taking a family history, or, for example, screening for one or more genetic markers or variants. Examples of genes involved in hyperlipidemia may include, without limitation, LDL receptor (LDLR), the apoliproteins (ApoAl, ApoB, ApoE, and the like), cholesteryl ester transfer protein (CETP), lipoprotein lipase (LPL), hepatic lipase (LIPC), endothelial lipase (EL), lecithin:cholesteryl acyltransferase (LCAT).

A healthcare provider, such as a doctor, nurse, or family member, can take a family history before prescribing or administering a dsRNA. In addition, a test may be performed to determine a genotype or phenotype. For example, a DNA test may be performed on a sample from the subject, e.g., a blood sample, to identify the PCSK9 genotype and/or phenotype before a PCSK9 dsRNA is administered to the subject. In some embodiments, a test is performed to identify a related genotype and/or phenotype, e.g., a LDLR genotype. Examples of genetic variants with the LDLR gene are known in the art, (Costanza et al. (2005) Am. J. Epidemiol. 15; 161(8):714-24; Yamada et al. (2008) J. Med. Genet. January; 45(1):22-8; and Boes et al. (2009) Exp. Gerontol. 44:136-160).

VI. Kits and Articles of Manufacture

Certain aspects of the present disclosure relate to an article of manufacture or a kit comprising one or more of the dsRNAs, vector(s), or composition(s) (e.g., pharmaceutical composition(s)) as described herein useful for the treatment and/or prevention of a PCSK9-mediated disorder or disease as described above. The article of manufacture or kit may further comprise a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating or preventing the disease and may have a sterile access port (for example the container may be an intravenous solution bag or a vial having a stopper pierceable by a hypodermic injection needle). At least one active agent in the composition is a dsRNA described herein. The label or package insert indicates that the composition is used for treating a PCSK9-mediated disorder or disease. In some embodiments, disease is a lipidemia (e.g., hyperlipidemia) and/or other forms of lipid imbalances such as hypercholesterolemia, hypertriglyceridemia, and pathological conditions associated with these disorders such as heart and circulatory diseases. Moreover, the article of manufacture or kit may comprise (a) a first container with a composition contained therein, wherein the composition comprises a dsRNA described herein; and (b) a second container with a composition contained therein, wherein the composition comprises a second therapeutic agent. The article of manufacture or kit in this embodiment of the present disclosure may further comprise a package insert indicating that the compositions can be used to treat a particular disease. Alternatively, or additionally, the article of manufacture or kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Without limiting the present disclosure, a number of embodiments of the present disclosure are described below for the purpose of illustration.

Item 1: A double-stranded ribonucleic acid (dsRNA), wherein the dsRNA comprises a sense strand comprising a first sequence and an antisense strand comprising a second sequence, wherein the first sequence and the second sequence are complementary, and wherein the first sequence comprises a sequence selected from the group consisting of SEQ ID NOS: 6-11 and 310-321.

Item 2: The dsRNA of item 1, wherein the dsRNA comprises:

(1)
(SEQ ID NO: 176)
CCAUUUUAUUAAUAUGGUGACUinvdT
in the sense strand and
(SEQ ID NO: 177)
AGUCACCAUAUUAAUAAAAdTdT
in the antisense strand,
(2)
(SEQ ID NO: 180)
CCAUAUUAAUAUGGUGACUUUUinvdT
in the sense strand and
(SEQ ID NO: 181)
AAAAGUCACCAUAUUAAUAdTdT
in the antisense strand,
(3)
(SEQ ID NO: 182)
CCAAUUAAUAUGGUGACUUUUUinvdT
in the sense strand and
(SEQ ID NO: 183)
AAAAAGUCACCAUAUUAAUdTdT
in the antisense strand,
(4)
(SEQ ID NO: 184)
CCAUUAAUAUGGUGACUUUUUAinvdT
in the sense strand and
(SEQ ID NO: 185)
UAAAAAGUCACCAUAUUAAdTdT
in the antisense strand,
(5)
(SEQ ID NO: 186)
CCAUAAUAUGGUGACUUUUUAAinvdT
in the sense strand and
(SEQ ID NO: 187)
UUAAAAAGUCACCAUAUUAdTdT
in the antisense strand,
(6)
(SEQ ID NO: 188)
CCAUAUGGUGACUUUUUAAAAUinvdT
in the sense strand and
(SEQ ID NO: 189)
AUUUUAAAAAGUCACCAUAdTdT
in the antisense strand,
(7)
(SEQ ID NO: 322)
CCAUUAUUAAUAUGGUGACUUUinvdT
in the sense strand and
(SEQ ID NO: 323)
AAAGUCACCAUAUUAAUAAdTdT
in the antisense strand,
(8)
(SEQ ID NO: 324)
CCAAUAUGGUGACUUUUUAAAAinvdT
in the sense strand and
(SEQ ID NO: 325)
UUUUAAAAAGUCACCAUAUdtdt
in the antisense strand,
(9)
(SEQ ID NO: 326)
CCAAUUUUUAUUAAUAUGGUGACUinvdT
in the sense strand and
(SEQ ID NO: 327)
AGUCACCAUAUUAAUAAAAAUdTdT
in the antisense strand,
(10)
(SEQ ID NO: 328)
CCAUUUUAUUAAUAUGGUGACUUUinvdT
in the sense strand and
(SEQ ID NO: 329)
AAAGUCACCAUAUUAAUAAAAdTdT
in the antisense strand,
(11)
(SEQ ID NO: 330)
CCAUUUAUUAAUAUGGUGACUUUUinvdT
in the sense strand and
(SEQ ID NO: 331)
AAAAGUCACCAUAUUAAUAAAdTdT
in the antisense strand,
(12)
(SEQ ID NO: 332)
CCAUAUUAAUAUGGUGACUUUUUAinvdT
in the sense strand and
(SEQ ID NO: 333)
UAAAAAGUCACCAUAUUAAUAdTdT
in the antisense strand,
(13)
(SEQ ID NO: 334)
CCAAAUAUGGUGACUUUUUAAAAUinvdT
in the sense strand and
(SEQ ID NO: 335)
AUUUUAAAAAGUCACCAUAUUdTdT
in the antisense strand,
(14)
(SEQ ID NO: 336)
CCAGCAUUUUUAUUAAUAUGGUGACUinvdT
in the sense strand and
(SEQ ID NO: 337)
AGUCACCAUAUUAAUAAAAAUGCdTdT
in the antisense strand,
(15)
(SEQ ID NO: 338)
CCAAUUUUUAUUAAUAUGGUGACUUUinvdT
in the sense strand and
(SEQ ID NO: 339)
AAAGUCACCAUAUUAAUAAAAAUdTdT
in the antisense strand,
(16)
(SEQ ID NO: 340)
CCAUUUUUAUUAAUAUGGUGACUUUUinvdT
in the sense strand and
(SEQ ID NO: 341)
AAAAGUCACCAUAUUAAUAAAAAdTdT
in the antisense strand,
(17)
(SEQ ID NO: 342)
CCAUUUAUUAAUAUGGUGACUUUUUAinvdT
in the sense strand and
(SEQ ID NO: 343)
UAAAAAGUCACCAUAUUAAUAAAdTdT
in the antisense strand, or
(18)
(SEQ ID NO: 344)
CCAUUAUUAAUAUGGUGACUUUUUAAinvdT
in the sense strand and
(SEQ ID NO: 345)
UUAAAAAGUCACCAUAUUAAUAAdTdT
in the antisense strand.

Item 3: A double-stranded ribonucleic acid (dsRNA), wherein the dsRNA comprises a sense strand comprising a first sequence and an antisense strand comprising a second sequence, wherein only the first sequence and the second sequence are complementary, and wherein the first sequence is one of SEQ ID NOS: 3, 4, and 13.

Item 4: The dsRNA of item 3, wherein the dsRNA comprises:

(19)
(SEQ ID NO: 162)
CCAUUGUAGCAUUUUUAUUAAUinvdT
in the sense strand and
(SEQ ID NO: 163)
AUUAAUAAAAAUGCUACAAdTdT
in the antisense strand,
(20)
(SEQ ID NO: 166)
CCAGUAGCAUUUUUAUUAAUAUinvdT
in the sense strand and
(SEQ ID NO: 167)
AUAUUAAUAAAAAUGCUACdTdT
in the antisense strand, or
(21)
(SEQ ID NO: 290)
CCAGAGUGUGAAAGGUGCUGAUinvdT
in the sense strand and
(SEQ ID NO: 291)
AUCAGCACCUUUCACACUCdTdT
in the antisense strand.

Item 5: The dsRNA of any one of items 1-4, wherein the first and second sequences are each less than or equal to 30 nucleotides in length.

Item 6: The dsRNA of any one of items 1-5, wherein the first and second sequences are each at least 19 and less than or equal to 23 nucleotides in length.

Item 7: The dsRNA of any one of items 1-6, wherein the dsRNA is a small interfering RNA (siRNA) or short hairpin RNA (shRNA).

Item 8: The dsRNA of any one of items 1-7, wherein the dsRNA comprises one or more modified nucleotides.

Item 9: the dsRNA of item 8, wherein at least one of the one or more modified nucleotides is a 2′-O-methyl nucleotide, 5′-phosphorothioate nucleotide, or a terminal nucleotide linked to a cholesterol derivative or lipophilic moiety.

Item 10: The dsRNA of item 8, wherein at least one of the one or more modified nucleotides is a 2′-fluoro, 2′-deoxy, 2′-O-methoxyethyl, constrained ethyl (cEt), inverted deoxy, inverted dideoxy, locked nucleic acid, abasic, 2′-amino, 2′-alkyl, morpholino, phosphoramidate, or a non-natural base-containing nucleotide.

Item 11: The dsRNA of item 10, wherein the dsRNA comprises one or more 2′-O-methyl nucleotides and one or more 2′-fluoro nucleotides.

Item 12: The dsRNA of item 11, wherein the dsRNA comprises two or more 2′-O-methyl nucleotides and two or more 2′-fluoro nucleotides in the pattern OMe-F-OMe-F or F-OMe-F-OMe, wherein OMe represents a 2′-O-methyl nucleotide, and wherein F represents a 2′-fluoro nucleotide.

Item 13: The dsRNA of item 11, wherein the dsRNA comprises up to 10 contiguous nucleotides that are each a 2′-O-methyl nucleotide or up to 10 contiguous nucleotides that are each a 2′-fluoro nucleotide.

Item 14: The dsRNA of any one of items 1-13, wherein the dsRNA comprises one or more phosphorothioate groups.

Item 15: The dsRNA of any one of items 1-13, wherein the dsRNA does not comprise a phosphorothioate group.

Item 16: The dsRNA of any one of items 1-15, wherein the dsRNA comprises one or more phosphotriester groups.

Item 17: The dsRNA of any one of items 1-15, wherein the dsRNA does not comprise a phosphotriester group.

Item 18: The dsRNA of any one of items 1-17, wherein the dsRNA is attached to one or more GalNAc derivatives via a linker.

Item 19: The dsRNA of item 18, wherein the dsRNA is attached to three GalNAc derivatives via a trivalent branched linker.

Item 20: The dsRNA of item 18 or item 19, wherein at least one of the one or more GalNAc derivatives is attached to the 3′ end of the sense strand, the 3′ end of the antisense strand, or the 5′ end of the sense strand of the dsRNA.

Item 21: The dsRNA of any one of items 1, 3, and 5-20, wherein one or both of the sense strand and the antisense strand further comprises a 5′ overhang comprising one or more nucleotides.

Item 22: The dsRNA of any one of items 1, 3, and 5-21, wherein one or both of the sense strand and the antisense strand further comprises a 3′ overhang comprising one or more nucleotides.

Item 23: The dsRNA of item 22, wherein the 3′ overhang comprises two nucleotides.

Item 24: The dsRNA of any one of items 21-23, wherein the overhang comprises one or more thymines.

Item 25: The dsRNA of any one of items 1-24, wherein the dsRNA inhibits expression of a Proprotein Convertase Subtilisin Kexin 9 (PCSK9) gene.

Item 26: The dsRNA of item 1, wherein one or both of strands of the dsRNA comprise one or more compounds having the structure of formula (I):

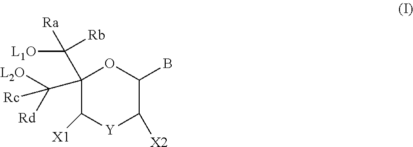

wherein:
B is a heterocyclic nucleobase;
one of L1 and L2 is an internucleoside linking group linking the compound of formula (I) to a polynucleotide and the other of L1 and L2 is H, a protecting group, a phosphorus moiety or an internucleoside linking group linking the compound of formula (I) to a polynucleotide,
Y is O, NH, NR1 or N—C(═O)—R1, wherein R1 is:
   a (C1-C20) alkyl group, optionally substituted by one or more groups selected from an halogen atom, a (C1-C6) alkyl group, a (C3-C8) cycloalkyl group, a (C3-C14) heterocycle, a (C6-C14) aryl group, a (C5-C14) heteroaryl group, —O—Z1, —N(Z1)(Z2), —S—Z1, —CN, —C(=J)-O—Z1, —O—C(=J)-Z1, —C(=J)-N(Z1)(Z2), and —N(Z1)-C(=J)-Z2, wherein
   J is O or S,
   each of Z1 and Z2 is, independently, H, a (C1-C6) alkyl group, optionally substituted by one or more groups selected from a halogen atom and a (C1-C6) alkyl group,
      a (C3-C8) cycloalkyl group, optionally substituted by one or more groups selected from a halogen atom and a (C1-C6) alkyl group,
      a group —[C(═O)]m-R2-(O—CH2-CH2)p-R3, wherein
      m is an integer meaning 0 or 1,
      p is an integer ranging from 0 to 10,
R2 is a (C1-C20) alkylene group optionally substituted by a (C1-C6) alkyl group, —O—Z3, —N(Z3)(Z4), —S—Z3, —CN, —C(═K)—O—Z3, —O—C(═K)—Z3, —C(═K)—N(Z3)(Z4), or —N(Z3)-C(═K)—Z4, wherein
   K is O or S,
   each of Z3 and Z4 is, independently, H, a (C1-C6) alkyl group, optionally substituted by one or more groups selected from a halogen atom and a (C1-C6) alkyl group, and
   R3 is selected from the group consisting of a hydrogen atom, a (C1-C6) alkyl group, a (C1-C6) alkoxy group, a (C3-C8) cycloalkyl group, a (C3-C14) heterocycle, a (C6-C14) aryl group or a (C5-C14) heteroaryl group, or R3 is a cell targeting moiety,
X1 and X2 are each, independently, a hydrogen atom, a (C1-C6) alkyl group, and
each of Ra, Rb, Rc and Rd is, independently, H or a (C1-C6) alkyl group,
   or is a pharmaceutically acceptable salt thereof.

Item 27: The dsRNA of item 26, comprising one or more compounds of formula (I) wherein Y is:
a) NR1, R1 is a non-substituted (C1-C20) alkyl group;
b) NR1, R1 is a non-substituted (C1-C16) alkyl group, which includes an alkyl group selected from a group comprising methyl, isopropyl, butyl, octyl, and hexadecyl;
c) NR1, R1 is a (C3-C8) cycloalkyl group, optionally substituted by one or more groups selected from a halogen atom and a (C1-C6) alkyl group;
d) NR1, R1 is a cyclohexyl group;
e) NR1, R1 is a (C1-C20) alkyl group substituted by a (C6-C14) aryl group;
f) NR1, R1 is a methyl group substituted by a phenyl group; g) N—C(═O)—R1, R1 is an optionally substituted (C1-C20) alkyl group; or h) N—C(═O)—R1, R1 is methyl or pentadecyl.

Item 28: The dsRNA of items 26 or 27, comprising one or more compounds of formula (I) wherein B is selected from a group consisting of a pyrimidine, a substituted pyrimidine, a purine and a substituted purine, or a pharmaceutically acceptable salt thereof.

Item 29: The dsRNA of any one of items 26 to 28, wherein R3 is of formula (II)

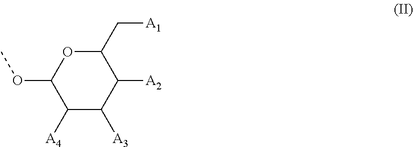

wherein A1, A2 and A3 are OH,
A4 is OH or NHC(═O)—R5, wherein R5 is a (C1-C6) alkyl group, optionally substituted by an halogen atom, or a pharmaceutically acceptable salt thereof

Item 30: The dsRNA of any one of items 26 to 29, wherein R3 is N-acetyl-galactosamine, or a pharmaceutically acceptable salt thereof.

Item 31: The dsRNA of any one of items 26 to 30, comprising one or more nucleotides from Table A.

Item 32: The dsRNA of any one of items 26 to 31, comprising from 2 to 10 compounds of formula (I), or a pharmaceutically acceptable salt thereof.

Item 33: The dsRNA of item 32, wherein the 2 to 10 compounds of formula (I) are on the sense strand.

Item 34: The dsRNA of any one of items 26 to 33, wherein the sense strand comprises two to five compounds of formula (I) at the 5′ end, and/or comprises one to three compounds of formula (I) at the 3′ end.

Item 35: The dsRNA of any one of items 26 to 34, wherein

a) the two to five compounds of formula (I) at the 5′ end of the sense strand comprise lgT3, optionally comprising three consecutive lgT3 nucleotides; and/or
b) the one to three compounds of formula (I) at the 3′ end of the sense strand comprise lT4; optionally comprising two consecutive lT4.

Item 36: The dsRNA of any one of items 26 to 35, comprising one or more internucleoside linking groups independently selected from the group consisting of phosphodiester, phosphotriester, phosphorothioate, phosphorodithioate, alkyl-phosphonate and phosphoramidate backbone linking groups, or a pharmaceutically acceptable salt thereof.

Item 37: The dsRNA of any one of items 26 to 36, selected from the dsRNAs in Tables 2-4.

Item 38: The dsRNA of any one of items 26 to 37, wherein:

a) the sense strand comprises a nucleotide sequence selected from the group consisting of SEQ ID NOs: 578, 585, 587, 620, 621, 622, and 627; and/or
b) the antisense strand comprises a nucleotide sequence selected from the group consisting of SEQ ID NOs: 589, 591, 631, 632, 634, 635 and 639.

Item 39: The dsRNA of item 38, wherein the sense strand and antisense strand of the dsRNA respectively comprise the nucleotide sequences of:

a) SEQ ID NOs: 578 and 589; [C027.001]

b) SEQ ID NOs: 620 and 631; [C027.003]

c) SEQ ID NOs: 585 and 591; [C027.001#40]

d) SEQ ID NOs: 587 and 591; [C027.001#58]

e) SEQ ID NOs: 621 and 634; [C027.003#03]

f) SEQ ID NOs: 622 and 632; [C027.003#06]

g) SEQ ID NOs: 622 and 635; and [C027.003#08]

h) SEQ ID NOs: 627 and 639. [C027.003#47]

Item 40: A vector encoding the dsRNA of any one of items 1-39.

Item 41: An isolated host cell comprising the dsRNA of any one of items 1-39 or the vector of item 40.

Item 42: A kit comprising the dsRNA of any one of items 1-39.

Item 43: A composition comprising the dsRNA of any one of items 1-39.

Item 44: The composition of item 43, further comprising a pharmaceutically acceptable carrier.

Item 45: The composition of item 43 or item 44, further comprising a delivery vehicle.

Item 46: The composition of item 31, wherein the delivery vehicle is selected from the group consisting of a liposome, lipoplex, complex, and nanoparticle.

Item 47: A method of inhibiting expression of a PCSK9 gene in a subject, comprising administering to the subject an effective amount of the dsRNA of any one of items 1-39 or the composition of item 44.

Item 48: A method of treating or preventing a PCSK9-mediated disease in a subject in need thereof, comprising administering to the subject an effective amount of a dsRNA of any one of items 1-39 or the composition of item 44.

Item 49: The method of item 48, wherein the PCSK9-mediated disorder is hypercholesterolemia.

Item 50: The method of any one of items 48-49, wherein the expression of the PCSK9 gene in the liver of the subject is inhibited by the dsRNA.

Item 51: The method of any one of items 48-50, wherein the administration is subcutaneous, intravenous, or pulmonary administration.

Item 52: The method of any one of items 48-51, wherein the subject is a human.

Item 53: The method of any one of items 48-52, wherein the administration results in a decrease in serum cholesterol in the subject.

Item 54: The method of any one of items 48-53, further comprising administering to the subject an effective amount of one or more additional therapeutic agents for treating or preventing a PCSK9-mediated disease.

Although the foregoing disclosure has been described in some detail by way of illustration and example for purposes of clarity of understanding, the descriptions and examples should not be construed as limiting the scope of the present disclosure.

### EXAMPLES

The present disclosure will be more fully understood by reference to the following examples. They should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Identification of siRNAs for Inhibition of Human PCSK9 Expression

Methods

siRNA Production

siRNAs, including negative control siRNAs (“LV2 neg. Control” and “LV2 neg. Control 2”), were produced using solid phase oligonucleotide synthesis. Positive control siRNA s48694 was purchased from Ambion. The sequence of each siRNA, including nucleotide modifications, is shown in Table 2 supra.

Cells and Tissue Culture

Human Hep3B and human C3A cells were cultured as follows. Human Hep3B cells were grown at 37° C., 5% CO₂ and 95% RH, and cultivated in EMEM medium (ATCC, cat. no. 30-2003) supplemented with 10% FBS. Human C3A cells were grown at 37° C., 5% CO₂ and 95% RH, and cultivated in MEM medium (ThermoFisher, cat. no. 41090) supplemented with 10% FBS.

Transfections

For knock-down experiments, 20,000 cells/well of either Hep3B or C3A cells were used in a 96-well plate. The cells were transfected with the indicated concentration of siRNAs using 0.2 μl/well of Lipofectamine® RNAiMAX transfection reagent (ThermoFisher) according to the manufacturer's protocol in a reverse transfection setup and incubated for 48 h without medium change. Usually, N=4 technical replicates were carried out per test sample. For testing siRNA-related toxicities, 15,000 Hep3B or C3A cells were transfected as described above and incubated for 72 h.

mRNA Expression Analysis

48 hours after siRNA transfection, the cellular RNA was harvested by usage of Promega's SV96 total RNA isolation system (cat. no. Z3500) according to the manufacturer's protocol including a DNase step during the procedure.

For cDNA synthesis the Reverse Transcriptase kit (cat. no. N8080234) was used from ThermoFisher. cDNA synthesis from 30 ng RNA was performed using 1.2 μl 10×RT buffer, 2.64 μl MgCl₂ (25 mM), 2.4 μl dNTPs (10 mM), 0.6 μl random hexamers (50 μM), 0.6 μl Oligo(dT)16 (50 μM), 0.24 μl RNase inhibitor (20 U/μl) and 0.3 μl Multiscribe (50 U/μl) in a total volume of 12 μl. Samples were incubated at 25° C. for 10 minutes and 42° C. for 60 minutes. The reaction was stopped by heating to 95° C. for 5 minutes.

PCSK9 mRNA levels were quantified by qPCR using the TaqMan Universal PCR Master Mix (cat. no. 4305719) and the TaqMan® Gene Expression assay Hs00545399_m1 from ThermoFisher. PCR was performed in technical duplicates with the ABI Prism 7900 under the following PCR conditions: 2 minutes at 50° C., 10 minutes at 95° C., 40 cycles with 95° C. for 15 seconds and 1 minute at 60° C. PCR was set up as a simplex PCR detecting the target gene (PCSK9) in one reaction and the housekeeping gene (RPL37A) for normalization in a second reaction. The final volume for the PCR reaction was 12.5 μl in a 1×PCR master mix, RPL37A primers were used in a final concentration of 50 nM and the probe of 200 nM. The ΔΔCt method was applied to calculate relative expression levels of the target transcripts. Percentage of PCSK9 expression was calculated by normalization based on the levels of the LV2 non-silencing siRNA control sequence.

IC₅₀ Measurements

Hep3B or C3A cells were transfected with the indicated siRNAs at concentrations ranging from 10 nM-0.01 pM using 10-fold dilution steps. The half maximal inhibitory concentration (IC₅₀) for each siRNA was calculated by applying a Biostat-Speed statistical calculation tool. Results were obtained using the 4-parameter logistic model according to Ratkovsky and Reedy (1986). The adjustment was obtained by non-linear regression using the Levenberg-Marquardt algorithm in SAS v9.1.3 software.

ELISA Assays

PCSK9 protein concentration was quantified in the supernatant of a culture of 25,000 C3A cells 48 hours after transfection with the indicated concentrations of siRNAs by R&D Systems' human PCSK9 Quantikine ELISA kit (cat. no. DPC900). The ELISA assays were performed using 50 μl of undiluted cell culture supernatant according to the manufacturer's protocol. Percentage of PCSK9 expression was calculated by normalization based on the mean levels of non-silencing siRNA control sequences.

Cytotoxicity

Cytotoxicity of each siRNA was measured 72 hours after transfection of a culture of 15,000 Hep3B or C3A cells by determining the ratio of cellular viability/toxicity in each sample. Cell viability was measured by determination of the intracellular ATP content using the CellTiter-Glo® assay (Promega, cat. no. G7570) according to the manufacturer's protocol. Cell toxicity was measured in the supernatant using the ToxiLight™ assay (Lonza, cat. no. LT07-217) according to the manufacturer's protocol.

Results

In order to identify siRNAs useful in targeting human PCSK9, the following criteria were applied. First, 19mers from the human PCSK9 mRNA sequence as set forth in NM_174936.3 (SEQ ID NO:1) were identified in silico with an overlap of 18 nucleotides. After a first round of filtering, 715 potential siRNAs of interest were identified. Next, all 19mers that overlapped with known SNPs (identified with a prevalence of greater than 10% in a Caucasian population) were excluded, leaving a pool of 692 19mer sequences. All 692 19mers were then aligned to the PCSK9 mRNA sequence of Macaca fascicularis, and all sequences having more than 1 mismatch to Macaca fascicularis PCSK9 were excluded, leaving 130 siRNA sequences with 0 mismatches, and an additional 267 siRNA sequences with 1 mismatch.

An in silico analysis was then carried out to identify any potential off-target transcripts in the human transcriptome (RefSeq RNA version 2015-10-20). Human off-target sequences with RNAseq expression (Illumina Body Atlas) FPKM<0.5 in liver tissue were not considered. All siRNA sequences of interest had either greater than two mismatches to any human transcript other than PCSK9, or had two mismatches with four or fewer human genes; sequences that did not meet one of these two criteria were filtered out. After filtration, 229 potential siRNAs were left. A final filtering step was carried out to identify siRNA sequences with less than 30% GC content, and 14 siRNAs were identified for functional characterization. All 14 of these siRNAs recognized target sequences in the 3′ untranslated region (UTR) of human PCSK9.

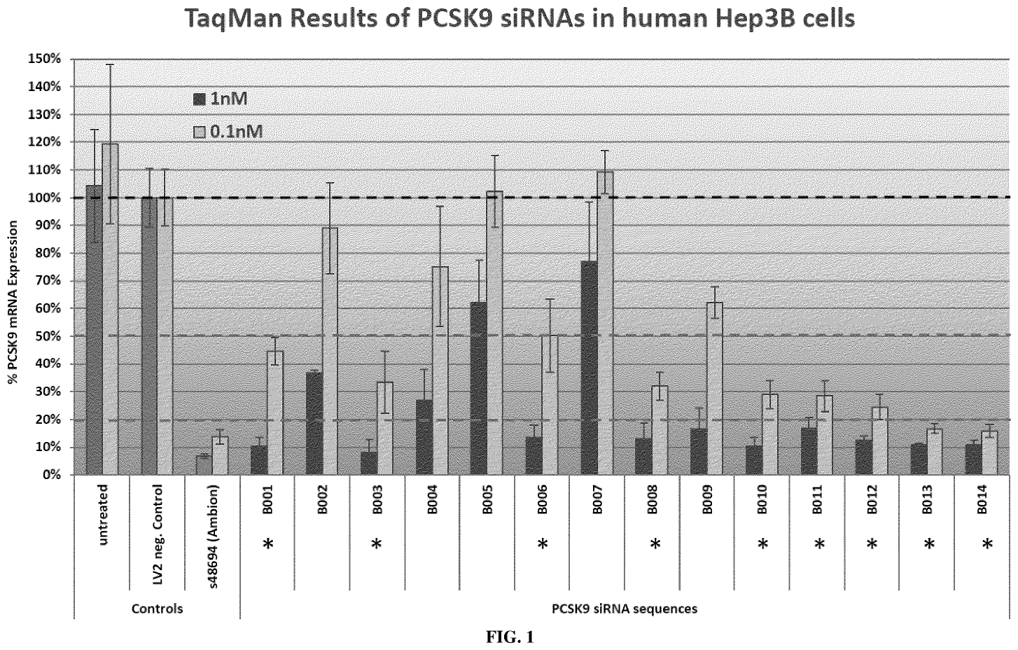

As described above, the 14 siRNAs were produced with nucleotides having 2′O-methyl and 2′-fluoro groups, but without additional modifications such as GalNAc ligands or phosphorothioates. To test the ability of these 14 siRNAs to reduce expression of PCSK9, human Hep3B cells were transfected with 0.1 nM or 1.0 nM of each siRNA and incubated for 48 hours. After incubation, mRNA expression of PCSK9 was measured in each sample and compared to positive and negative controls (FIG. 1). Nine of the 14 siRNAs showed the most potent hPCSK9 inhibition, reducing PCSK9 mRNA expression by at least 80% at a concentration of 1.0 nM, and by at least 50% at a concentration of 0.1 nM.

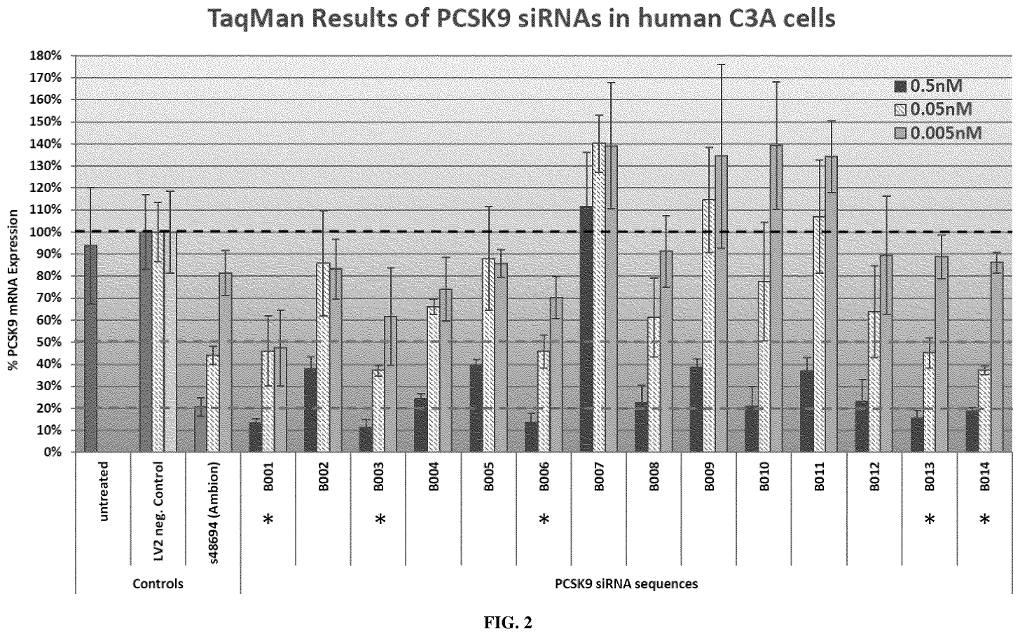

The activity of the 14 siRNAs of interest was further tested in human C3A cells, which are characterized by a higher level of PCSK9 expression than Hep3B cells (FIG. 2). siRNAs were tested at the following concentrations: 0.5 nM, 0.05 nM, and 0.005 nM. Five of the 14 siRNAs showed the most potent inhibition of hPCSK9 expression in this more stringent assay (B001, B003, B006, B013, and B014). These siRNAs reduced PCSK9 mRNA expression by at least 80% at a concentration of 0.5 nM, and by at least 50% at a concentration of 0.05 nM.

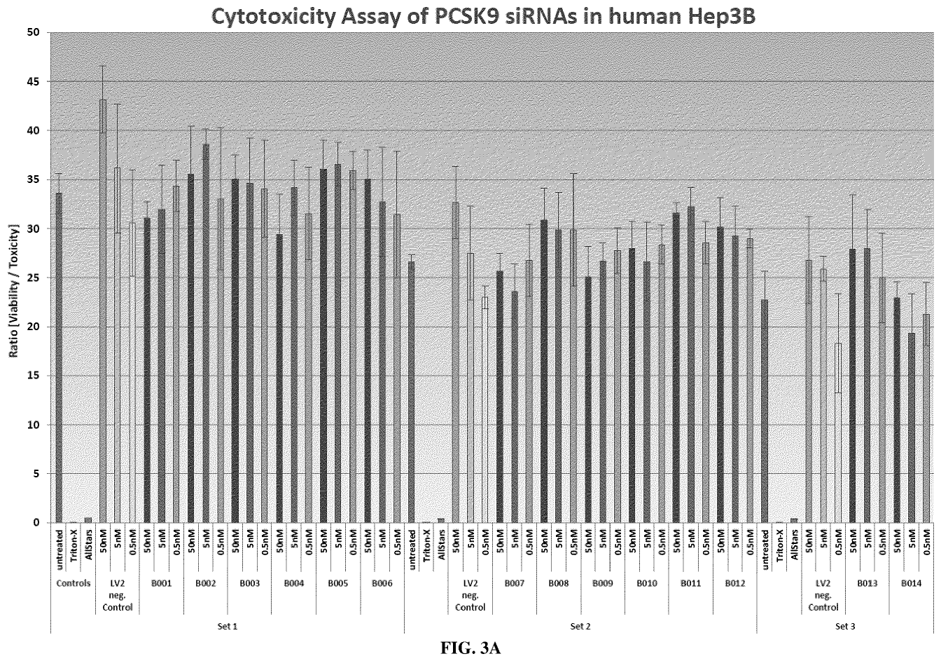

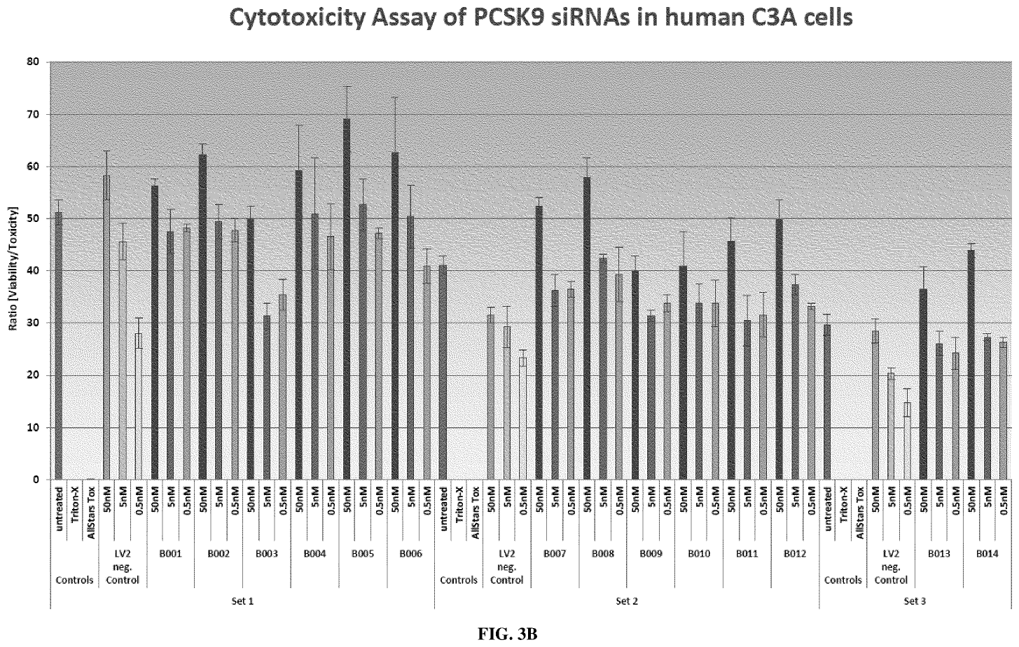

Next, cellular cytotoxicity was measured in Hep3B and C3A cells 72 hours after transfection with the 14 siRNAs of interest. Surprisingly, no obvious cytotoxicity in Hep3B or C3A cells was indicated for any of the siRNAs tested, even when used at a concentration of up to 50 nM (FIGS. 3A and 3B).

Taken together, these results demonstrate the identification of siRNAs capable of potent inhibition of PCSK9 expression in multiple human cell lines without significant cytotoxicity.

### Example 2: Characterization of Additional siRNAs for Inhibition of Human PCSK9 Expression

Additional siRNA sequences were selected as described above, except that the sequences were filtered for those having 30-65% G+C content. 60 siRNAs were produced as described in Example 1. These siRNAs recognize targets distributed throughout the 5′ UTR, 3′ UTR, and open reading frame (ORF) of human PCSK9.

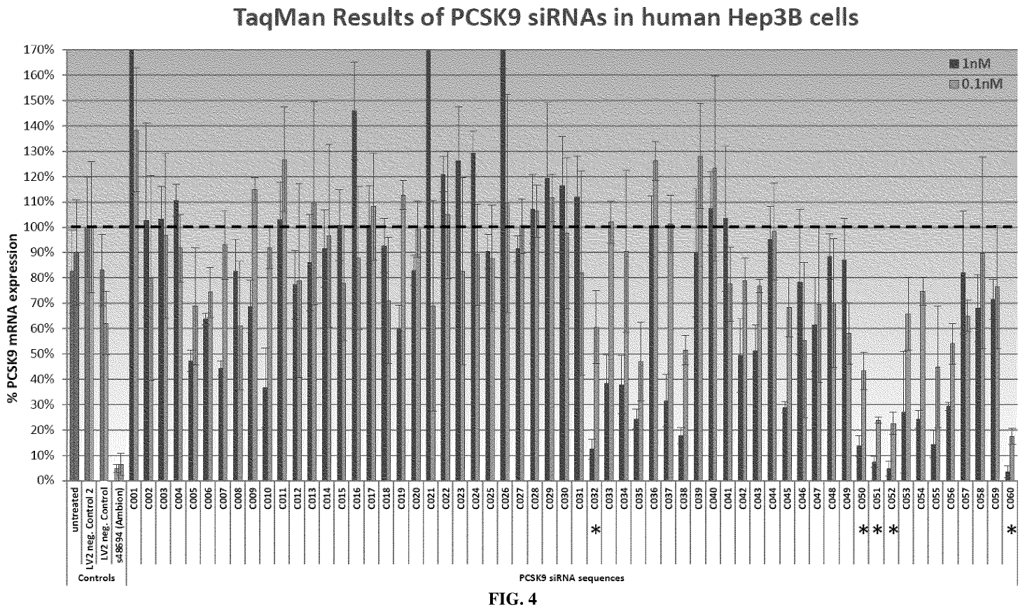

To test the ability of these 60 siRNAs to reduce expression of PCSK9, human Hep3B cells were transfected with 0.1 nM or 1.0 nM of each siRNA and incubated for 48 hours. After incubation, mRNA expression of PCSK9 was measured in each sample and compared to positive and negative controls (FIG. 4). Five of the 60 siRNAs showed potent inhibition of hPCSK9 expression, reducing PCSK9 mRNA expression by at least 86% at a concentration of 1.0 nM.

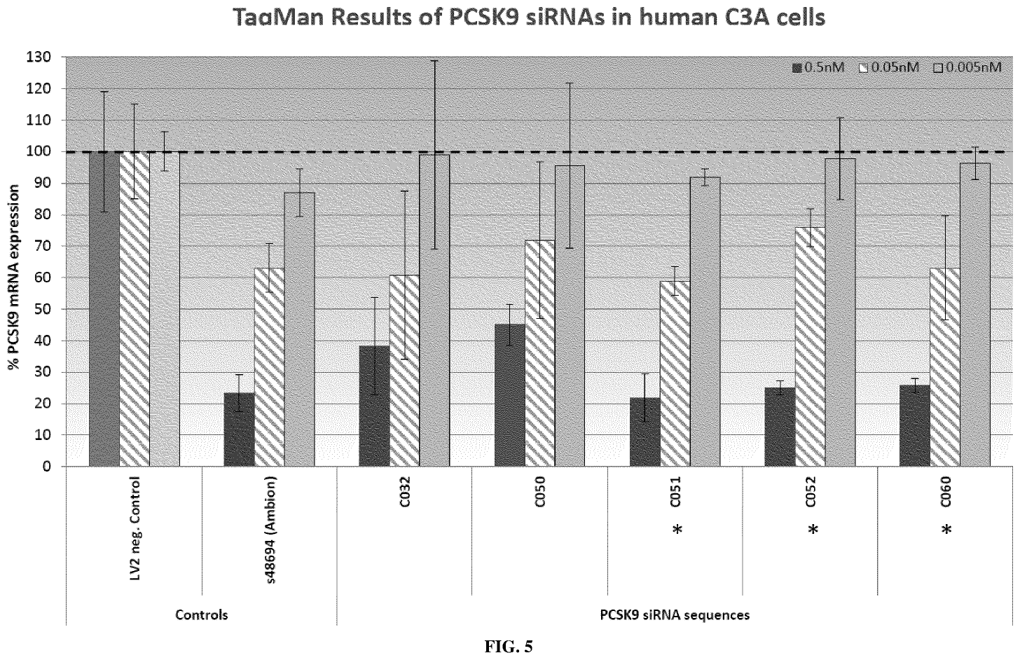

The activity of the five most potent out of 60 siRNAs was further tested in human C3A cells (FIG. 5). siRNAs were tested at the following concentrations: 0.5 nM, 0.05 nM, and 0.005 nM. Three of the siRNAs tested showed potent inhibition of hPCSK9 expression, reducing PCSK9 mRNA expression by at least 75% at a concentration of 0.5 nM.

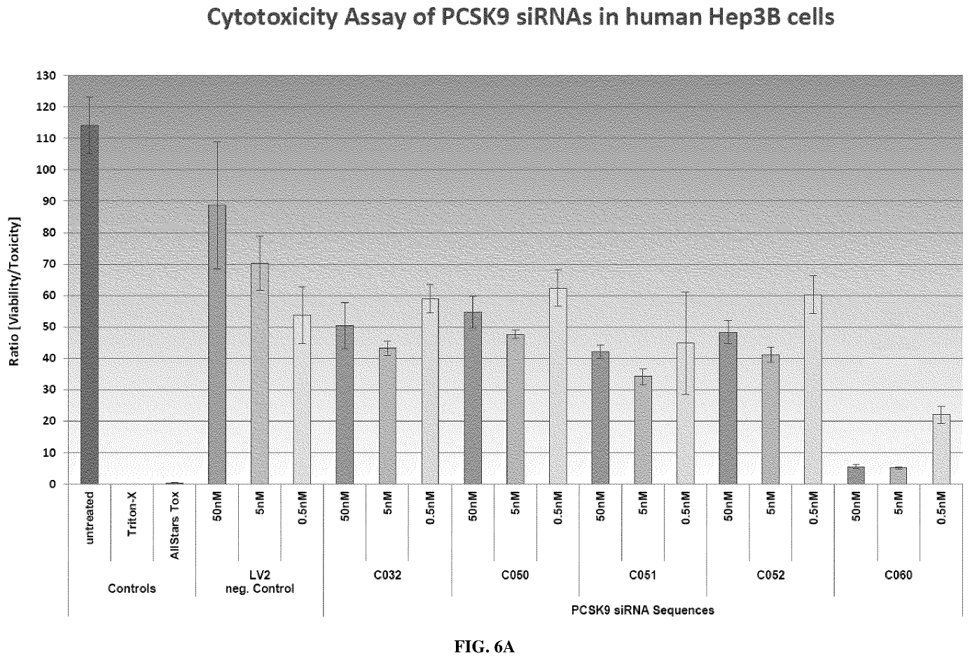

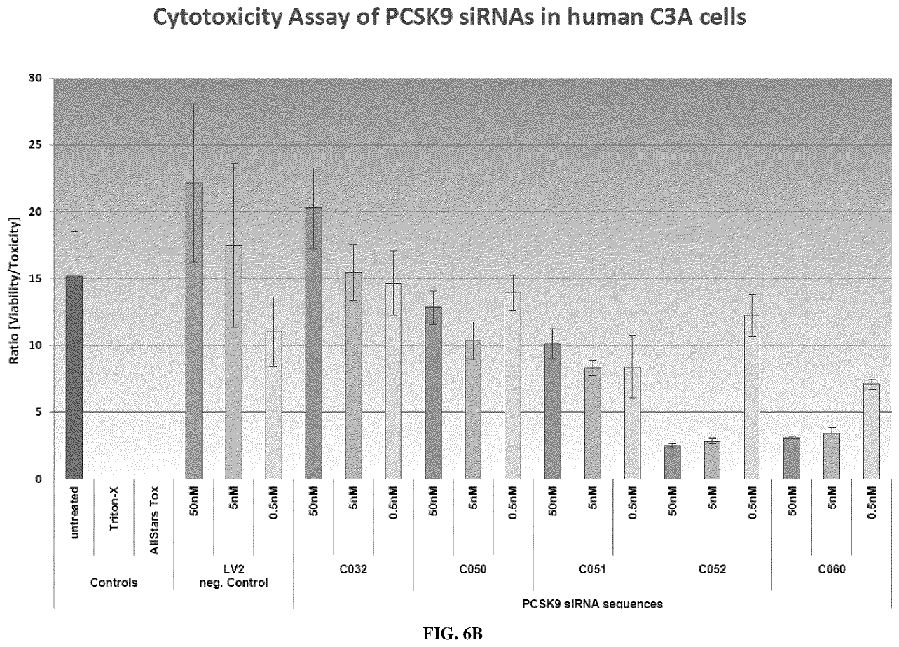

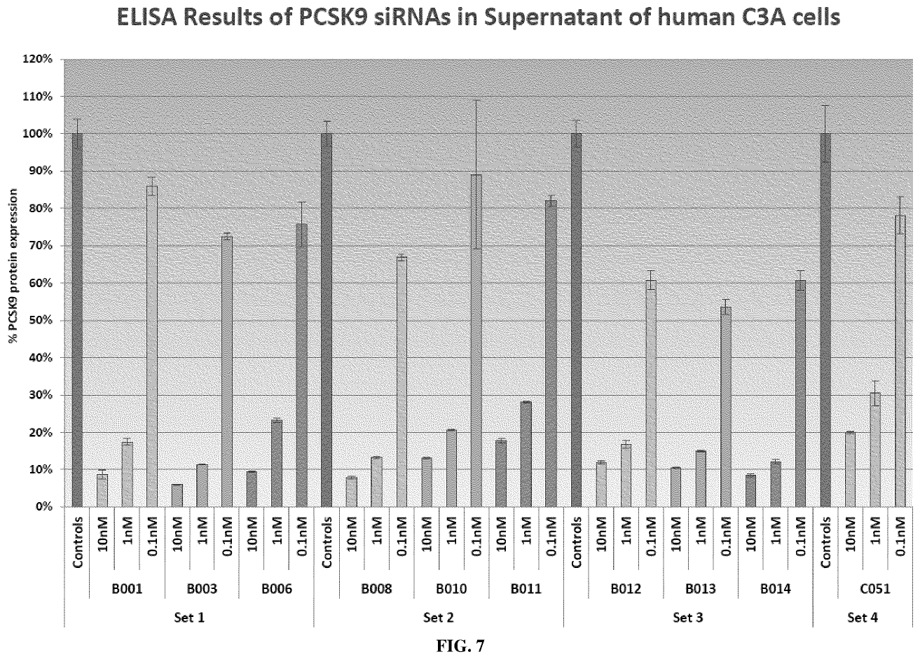

Next, cellular cytotoxicity was measured in Hep3B and C3A cells 72 hours after siRNA transfection (FIGS. 6A and 6B). One siRNA, C060, caused significant cytotoxicity in both cell lines, and another, C052, caused significant cytotoxicity in C3A cells. Based on the results of the activity and cytotoxicity data in C3A and Hep3B cells described above, 10 siRNAs were selected for IC₅₀ measurements (B001, B003, B006, B008, B010, B013, B014, and C051). The ten siRNAs were all of similar potency. The ten siRNAs were further found to reduce hPCSK9 protein in C3A cells as measured by ELISA assay, particularly at higher concentrations tested (FIG. 7).

The results of these experiments are summarized in Table A. The portion of each siRNA that contains its hPCSK9 target sequence is shown in Table B.

TABLE A
Functional activities of siRNAs.

| | | | | | C3A ELISA | | | |
|---|---|---|---|---|---|---|---|---|
| | C3A cells | | Hep3B cells | | (% Knock-down) | | | Cytotox- |
| | IC₅₀ | | IC₅₀ | | | C₅₀ | | icity |
| siRNA | (pM) | Iₘₐₓ % | (pM) | Iₘₐₓ % | siRNA | (pM) | ₘₐₓ % | IC₅₀ (pM) |
| B001 | 177 | 93.0 | 564 | 91.2 | B001 | 177 | 93.0 | 564 |
| B003 | 136 | 88.5 | 101 | 96.1 | B003 | 136 | 88.5 | 101 |
| B006 | 105 | 85.8 | 119 | 95.4 | B006 | 105 | 85.8 | 119 |
| B008 | 93 | 78.0 | 51 | 90.3 | B008 | 93 | 78.0 | 51 |
| B010 | 106 | 86.4 | 55 | 92.8 | B010 | 106 | 86.4 | 55 |
| B011 | 79 | 88.0 | 104 | 88.1 | B011 | 79 | 88.0 | 104 |
| B012 | 118 | 88.4 | 77 | 91.2 | B012 | 118 | 88.4 | 77 |
| B013 | 68 | 88.8 | 58 | 93.4 | B013 | 68 | 88.8 | 58 |
| B014 | 84 | 88.1 | 70 | 93.2 | B014 | 84 | 88.1 | 70 |
| C051 | 170 | 80.1 | 53 | 93.2 | C051 | 170 | 80.1 | 53 |

TABLE B
siRNA sequence information.

| | | | Target start |
|---|---|---|---|
| | | | position in |
| | # of | | human PCSK9 |
| | Mismatches | | mRNA |
| | to macaque | Sense strand target | (NM_174936; |
| siRNA | PCSK9 | sequence (5′→3′) | SEQ ID NO: 1) |
| B001 | 0 | UUGUAGCAUUUUUAUUAAU | 3649 |
| | | (SEQ ID NO: 3) | |
| B003 | 0 | GUAGCAUUUUUAUUAAUAU | 3651 |
| | | (SEQ ID NO: 4) | |
| B006 | 1 | GCAUUUUUAUUAAUAUGGU | 3654 |
| | | (SEQ ID NO: 5) | |
| B008 | 1 | UUUUAUUAAUAUGGUGACU | 3658 |
| | | (SEQ ID NO: 6) | |
| B010 | 1 | UAUUAAUAUGGUGACUUUU | 3661 |
| | | (SEQ ID NO: 7) | |
| B011 | 1 | AUUAAUAUGGUGACUUUUU | 3662 |
| | | (SEQ ID NO: 8) | |
| B012 | 1 | UUAAUAUGGUGACUUUUUA | 3663 |
| | | (SEQ ID NO: 9) | |
| B013 | 1 | UAAUAUGGUGACUUUUUAA | 3664 |
| | | (SEQ ID NO: 10) | |
| B014 | 1 | UAUGGUGACUUUUUAAAAU | 3667 |
| | | (SEQ ID NO: 11) | |
| C051 | 1 | GAGUGUGAAAGGUGCUGAU | 2891 |
| | | (SEQ ID NO: 13) | |

As compared with the 14 siRNAs described in Example 1, these 60 siRNAs had a significantly lower proportion of those effective at knocking down hPCSK9 expression (5/60 were effective in Hep3B cells, as compared to 9/14 from Example 1). Without wishing to be bound to theory, it is thought that the siRNAs from Example 1 may exhibit higher efficacy due to their lower G+C content and/or the specific region of PCSK9 targeted (e.g., the 3′ UTR). Taken together, these results further illustrate the unpredictability of effective siRNA knockdown of human PCSK9 expression. Moreover, the results obtained using the 60 siRNA sequences further underscore the efficacy and low level of cytotoxicity of the siRNAs described in Example 1.

### Example 3: In Vitro and In Vivo Evaluation of PCSK9 siRNA Molecules

Methods

siRNA Production

siRNAs, including negative control siRNAs, were produced using solid phase oligonucleotide synthesis.

Cells and Tissue Culture

Human C3A cells were grown at 37° C., 5% CO₂ and 95% RH, and cultivated in MEM medium (ThermoFisher, cat. no. 41090) supplemented with 10% FBS.

Human peripheral blood mononuclear cells (PBMCs) were isolated from approximately 16 mL of blood from three healthy donors that were collected in Vacutainer tubes coated with sodium heparin (BD, Heidelberg Germany) according to manufacturer's instructions.

Primary human and cynomolgus hepatocytes were cultured as follows: cryopreserved cells were thawed and plated using a Plating and Thawing Kit (PTK-1, Primacyt), and were incubated at 37° C., 5% CO₂ and 95% RH. 6 hours after plating, the medium was changed to maintenance medium (KLC-MM, KaLy-Cell) supplemented with 1% FBS.

Transfections

For knock-down experiments in C3A cells, 25,000 cells/well were used in a 96-well plate. The cells were transfected with the indicated concentration of siRNAs using 0.2 μl/well of Lipofectamine® RNAiMAX transfection reagent (ThermoFisher) according to the manufacturer's protocol in a reverse transfection setup and incubated for 48 h without medium change. Usually, N=4 technical replicates were carried out per test sample.

For transfection of human PBMCs, 100 nM of the siRNAs were reverse transfected into 1×10⁵ PBMCs with 0.3 μL Lipofectamine 2000 per 96-well (n=2) in a total volume of 150 μL serum-free RPMI medium for 24 hours. Single stranded RNA (“R-0006”) and DNA (“CpG ODN”) oligonucleotides, as well as double stranded unmodified and 2′-O-methyl modified siRNA (“132/161”) were applied as controls. mRNA expression analysis

48 hours after siRNA transfection or 72 hours after free siRNA uptake, the cellular RNA was harvested by usage of Promega's SV96 total RNA isolation system (cat. no. Z3500) according to the manufacturer's protocol including a DNase step during the procedure.

For cDNA synthesis the Reverse Transcriptase kit (cat. no. N8080234) was used from ThermoFisher. cDNA synthesis from 30 ng RNA was performed using 1.2 μl 10×RT buffer, 2.64 μl MgCl₂ (25 mM), 2.4 μl dNTPs (10 mM), 0.6 μl random hexamers (50 μM), 0.6 μl Oligo(dT)16 (50 μM), 0.24 μl RNase inhibitor (20 U/μl) and 0.3 μl Multiscribe (50 U/μl) in a total volume of 12 μl. Samples were incubated at 25° C. for 10 minutes and 42° C. for 60 minutes. The reaction was stopped by heating to 95° C. for 5 minutes.

PCSK9 mRNA levels were quantified by qPCR using the ThermoFisher TaqMan Universal PCR Master Mix (cat. no. 4305719) and the TaqMan® Gene Expression assays Hs00545399_m1 and Mf03418189_m1 for human and cynomolgus samples, respectively. PCR was performed in technical duplicates with the ABI Prism 7900 under the following PCR conditions: 2 minutes at 50° C., 10 minutes at 95° C., 40 cycles with 95° C. for 15 seconds and 1 minute at 60° C. PCR was set up as a simplex PCR detecting the target gene (PCSK9) in one reaction and the housekeeping gene (RPL37A) for normalization in a second reaction. The final volume for the PCR reaction was 12.5 μl in a 1×PCR master mix, RPL37A primers were used in a final concentration of 50 nM and the probe of 200 nM. The ΔΔCt method was applied to calculate relative expression levels of the target transcripts. Percentage of PCSK9 expression was calculated by normalization based on the levels of the LV2 non-silencing siRNA control sequence.

IC₅₀ Measurements

C3A cells were transfected with the indicated siRNAs at concentrations ranging from 25 nM-0.1 pM using 8-fold dilution steps. The half maximal inhibitory concentration (IC₅₀) for each siRNA was calculated by applying a Biostat-Speed statistical calculation tool. Results were obtained using the 4-parameter logistic model according to Ratkovsky and Reedy (1986). The adjustment was obtained by non-linear regression using the Levenberg-Marquardt algorithm in SAS v9.1.3 software.

For IC₅₀ measurements in primary human and cynomolgus hepatocytes, 70,000 cells in 96-well plates were incubated for 72 hours under free uptake conditions with the siRNAs at concentrations ranging from 10 μM-0.005 nM using 5-fold dilution steps.

ELISA Assays

PCSK9 protein concentration was quantified in the supernatant of C3A cells 48 hours after transfection with the indicated concentrations of siRNAs by R&D Systems' human PCSK9 Quantikine ELISA kit (cat. no. DPC900). The ELISA assays were performed using 50 μl of undiluted cell culture supernatant according to the manufacturer's protocol. Percentage of PCSK9 expression was calculated by normalization based on the mean levels of non-silencing siRNA control sequences.

IFNα protein concentration was quantified in the supernatant of PBMCs as follows: 25 μL of the cell culture supernatant was used for measurement of IFNα concentration applying a self-established electrochemiluminescence assay based on MesoScale Discovery's technology, and using a pan IFNα monoclonal capture antibody (MT1/3/5, Mabtech).

Cytotoxicity

Cytotoxicity of each siRNA was measured 72 hours after incubation with 50,000 primary human hepatocytes under free uptake conditions by determining the ratio of cellular viability/toxicity in each sample. Cell viability was measured by determination of the intracellular ATP content using the CellTiter-Glo® assay (Promega, cat. no. G7570) according to the manufacturer's protocol. Cell toxicity was measured in the supernatant using the ToxiLight™ assay (Lonza, cat. no. LT07-217) according to the manufacturer's protocol.

Nuclease Stability

The siRNAs were tested for nuclease stability in 50% mouse serum. For this purpose, 160 μL mouse serum (Sigma, cat. No. M5905) was incubated at 37° C. for 0, 8, 24, 32, 48, 56, and 72 hours. At each time point, 21 μL of the reaction was taken out and quenched with 23 μL stop solution (for 3,000 μL stop solution: 1123 μL Tissue & Cell Lysis Solution (Epicentre, cat. No. MTC096H), 183 μL 20 mg/mL Proteinase K (Sigma, cat. No. P2308), 1694 μL water) at 65° C. for 30 minutes. Prior to HPLC analysis on a Waters 2695 Separation Module and a 2487 Dual Absorbance Detector, 33 μL of RNase-free water was added to each sample. 50 μL of the solution was analyzed by HPLC using a DNAPac PA200 analytical column (Thermo Scientific, cat. No. 063000), and the following gradient:

| Time (min) | Flow (mL/min) | % Buffer A* | % Buffer B** |
|---|---|---|---|
| 0 | 1 | 75 | 25 |
| 20 | 1 | 35 | 65 |

*Buffer A: 20 mM sodium phosphate (Sigma, cat. No. 342483), pH 11;
**Buffer B: 20 mM sodium phosphate (Sigma, cat. No. 342483), 1M sodium bromide (Sigma, cat. No. 02119), pH 11.

Mouse Model

The female mice used in the following experiments carried a transgene encoding full-length human PCSK9, and were knockouts for the corresponding mouse PCSK9. The transgenic model, strain “hTg-mKO line #2”, was in-licensed from IRCM (Institut de Recherches Cliniques do Montréal) via Univalor Inc.

In Vivo Measurements

Serum PCSK9 levels in mice treated with siRNAs were determined using the same R&D Systems' human PCSK9 Quantikine ELISA kit (Cat. No. DPC900) with 1:40 pre-dilutions. Relative PCSK9 serum levels were calculated to pre-dosing values.

Serum total and LDL cholesterol levels in transgenic mice treated with PCSK9 siRNAs were determined with a COBAS INTEGRA instrument and Roche's LDLC3 assay or Horiba's ABX Pentra LDL Direct CP assay.

Serum AST, ALT, and BUN levels were determined using standard clinical chemistry assays with a COBAS INTEGRA instrument.

Results

The 10 PCSK9 siRNA sequences as shown in Tables A and B were conjugated and characterized in vitro. IC₅₀ measurements were taken for the siRNAs using human C3A cells (Table C). IC₅₀ values in human C3A cells transfected with the indicated siRNAs ranged from 9.7-125.0 pM.

TABLE C
IC₅₀ activities of 10 siRNAs in human C3A cells

| siRNA: | IC₅₀ (pM): | Iₘₐₓ %: |
|---|---|---|
| C032.001 | 114.0 | 79.6 |
| C032.004 | 76.3 | 86.1 |
| C032.005 | 23.7 | 88.4 |
| C032.006 | 70.9 | 87.4 |
| C032.007 | — | 84.6 |
| C032.008 | 88.1 | 83.2 |
| C032.009 | 125.0 | 86.3 |
| C032.010 | 9.7 | 86.7 |
| C032.011 | 15.4 | 89.6 |
| C032.012 | 12.7 | 90.3 |

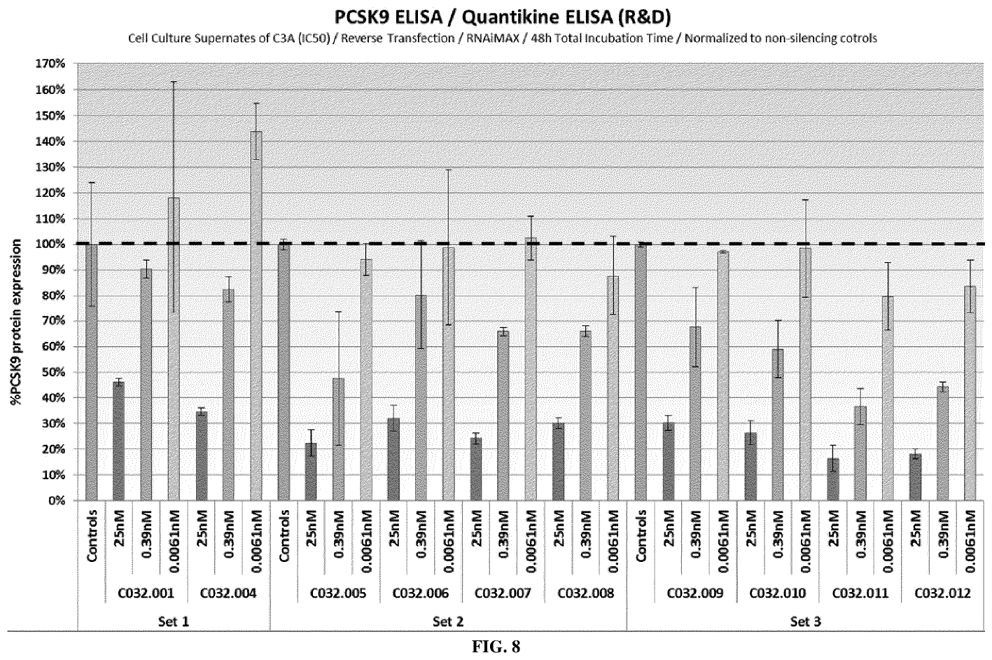

PCSK9 protein knockdown was confirmed by quantification of PCSK9 levels in the supernatants of the C3A cells transfected with three different concentrations (25, 0.39, and 0.0061 nM) of the siRNAs (FIG. 8).

The IC₅₀ of each siRNA was also measured using free uptake into primary cells. Primary cynomolgus monkey hepatocytes were treated with the siRNAs, and the IC₅₀ for each siRNA was calculated (Table D). IC₅₀ values ranged from 94.2-486.0 nM. siRNA sequences C032.004 and C032.005 (without a mismatch to the cynomolgus PCSK9) showed good dose-dependent knockdown activity, and, to a lesser extent, so did sequence C032.012 (with one mismatch to macaque species).

TABLE D
IC₅₀ activities of 10 siRNAs in primary cynomolgus
hepatocytes under free uptake conditions

| siRNA: | IC₅₀ (nM): | Iₘₐₓ %: | Comment: |
|---|---|---|---|
| C032.001 | n.a. | n.a. | 1 MM cyno |
| C032.004 | 94.2 | 55.8 | 0 MM cyno |
| C032.005 | 117.0 | 66.7 | 0 MM cyno |
| C032.006 | n.a. | n.a. | 1 MM cyno |
| C032.007 | n.a. | n.a. | 1 MM cyno |
| C032.008 | n.a. | n.a. | 1 MM cyno |
| C032.009 | n.a. | n.a. | 1 MM cyno |
| C032.010 | n.a. | n.a. | 1 MM cyno |
| C032.011 | n.a. | n.a. | 1 MM cyno |
| C032.012 | 486.0 | 30.9 | 1 MM cyno |

n.a. = not active
MM = mismatch

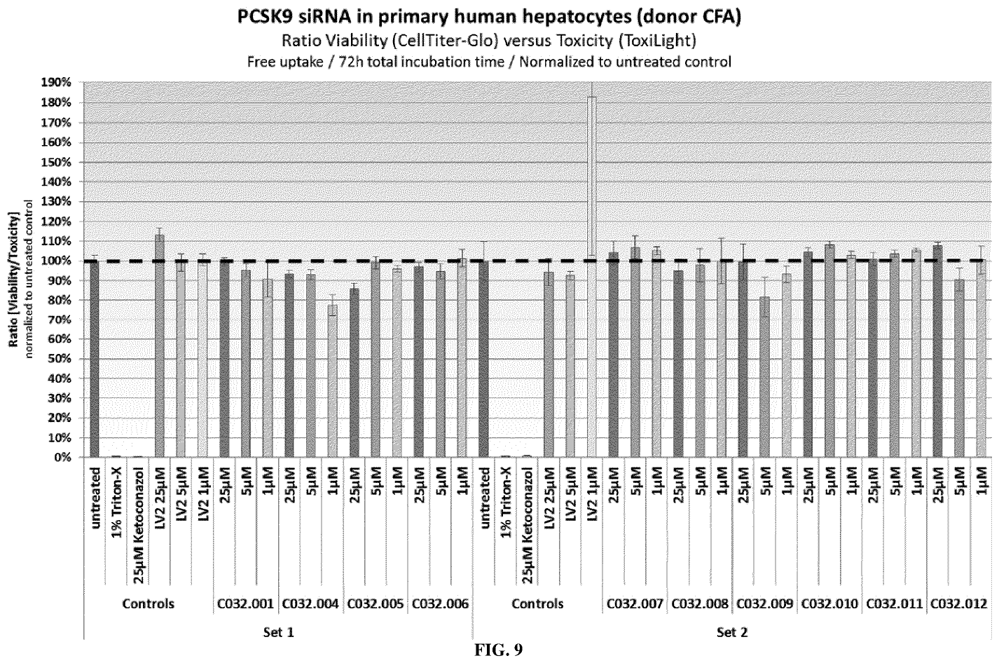

Primary human hepatocytes were also treated with the siRNAs, and the IC₅₀ for each siRNA was calculated in this primary human cell type (Table E). IC₅₀ values ranges from 9.4-189.0 nM. The cytotoxicity of the siRNAs was also examined in this primary human cell type under free uptake conditions (FIG. 9). No dose-dependent cytotoxic effects were observed for any of the tested siRNAs.

TABLE E
IC₅₀ activities of 10 siRNAs in primary human
hepatocytes under free uptake conditions

| siRNA: | IC₅₀ (nM): | Iₘₐₓ %: |
|---|---|---|
| C032.001 | 36.3 | 70.9 |
| C032.004 | 14.6 | 56.7 |
| C032.005 | 16.7 | 64.4 |
| C032.006 | 17.2 | 61.4 |
| C032.007 | 27.7 | 72.6 |
| C032.008 | 9.4 | 54.1 |
| C032.009 | 189.0 | 35.4 |
| C032.010 | 34.7 | 50.6 |
| C032.011 | 17.6 | 61.6 |
| C032.012 | 11.7 | 72.3 |

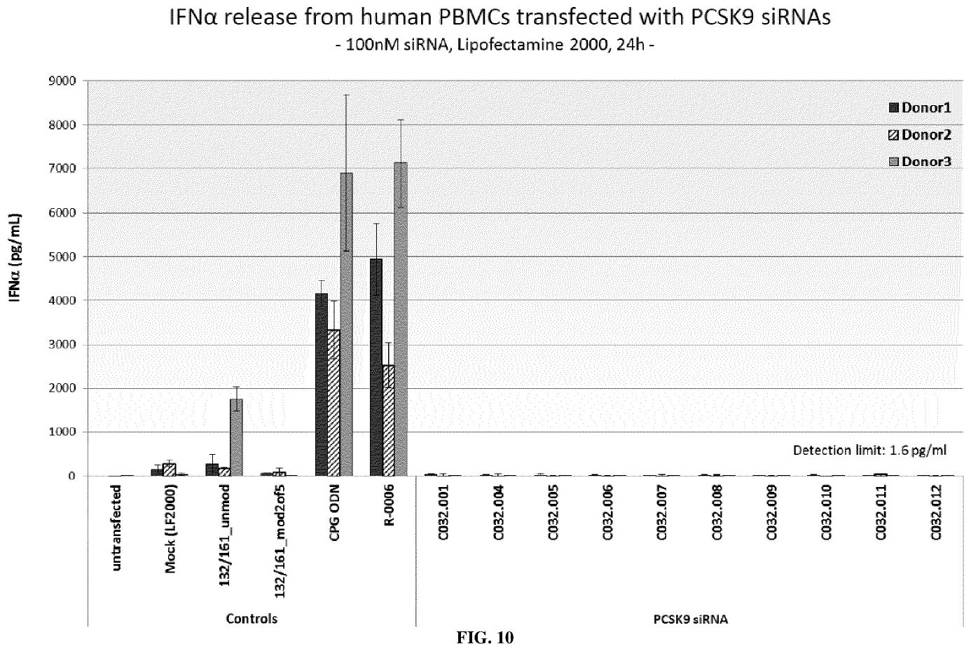

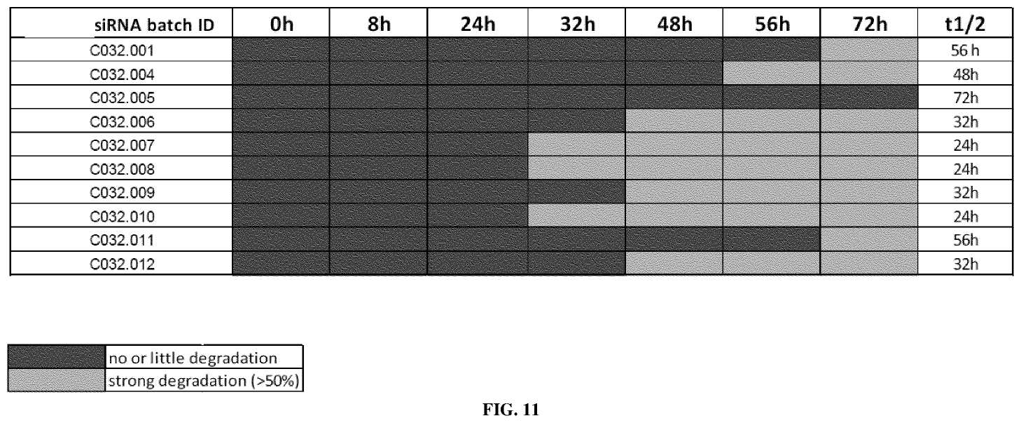

The immune response to the siRNAs was measured in primary human cells by examining the production of interferon α secreted from human primary PMBCs isolated from three different healthy donors (FIG. 10) in response to transfection of the siRNAs. No signs of immune stimulation in human PBMCs were observed for any of the tested siRNAs.

The 10 PCSK9 siRNAs were also tested for their in vitro nuclease stability in 50% murine serum, and the relative stability and half-lives were determined (FIG. 11). Whereas all siRNAs were stable for at least 24 hours, compound C032.005 was identified as most stable with little or no degradation at the latest time point measured (72 hours).

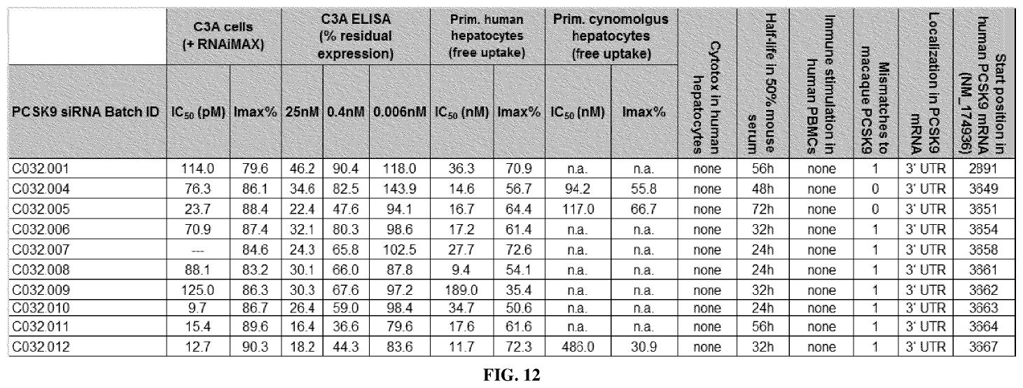

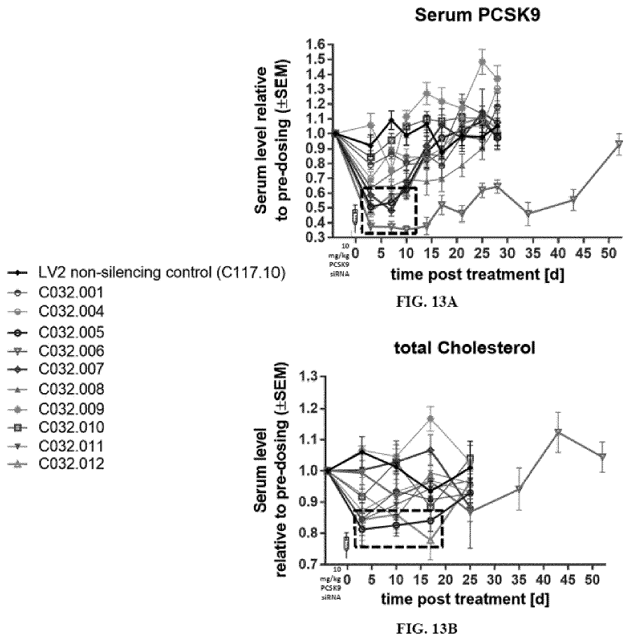

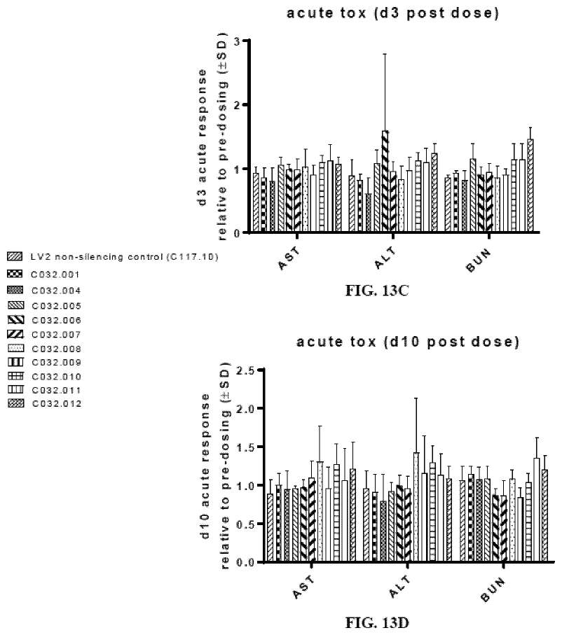

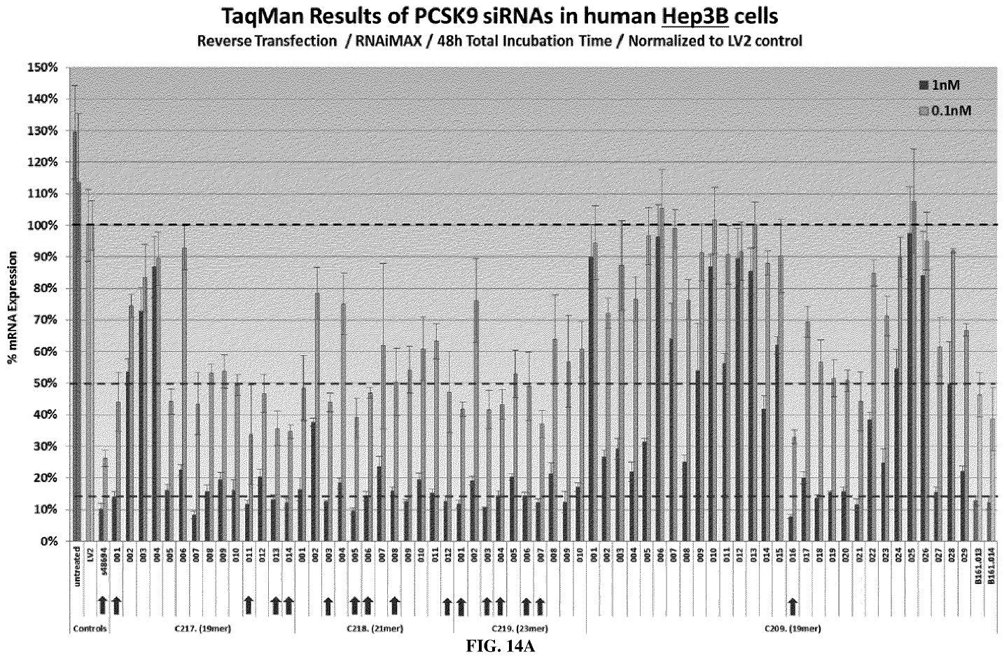

A summary of the results from the in vitro analyses is shown in FIG. 12. Next, the in vivo effects on serum PCSK9 protein levels (FIG. 13A), as well as serum total cholesterol levels (FIG. 13B), were examined for a single subcutaneous injection of 10 mg/kg of the 10 conjugated siRNAs, as compared to non-silencing control siRNA. The mice used in the in vivo efficacy experiments carried a transgene encoding full-length human PCSK9, and were knockouts for the corresponding mouse PCSK9. siRNAs C032.005, C032.007, and C032.012 had a nearly identical pattern on PCSK9 reduction, with maximum PCSK9 knockdown of approximately 49-52% between days 3 and 7, with a return to baseline between days 17 and 21. siRNA C032.006 was most active on PCSK9 levels with a maximum knockdown of 65% at day 10, a return to baseline levels at day 52. The highest reduction of total cholesterol levels was obtained using siRNA C032.005 and C032.012, with a maximum reduction of 19% and 22%, respectively. Interestingly, no major effect on cholesterol levels was observed for siRNA C032.006 even though it has the greatest effect on PCSK9 levels.

At day 3 (FIG. 13C) and 10 (FIG. 13D) of the same in vivo study in the human PCSK9 transgenic mice, acute toxicology parameters were measured in serum samples. No obvious hepatic (as determined by AST and ALT levels) or renal (as determined by BUN levels) toxicities were detected with any of the compounds tested. Taken together, the good in vitro profile of PCSK9 siRNA C032.012 also translated to the in vivo setting in a relevant transgenic mouse model. Furthermore, siRNA C032.005 exhibited a good in vivo profile on PCSK9 and total cholesterol inhibition. Two additional siRNAs, C032.006 and C032.007, were identified with potent PCSK9 inhibition in vivo, but interestingly, these two siRNAs had no major effect on lowering cholesterol levels.

### Example 4: In Vitro Evaluation of Additional Test PCSK9 siRNA Molecules

Methods

Unless indicated otherwise, all experiments were carried out as described in the examples above.

Results

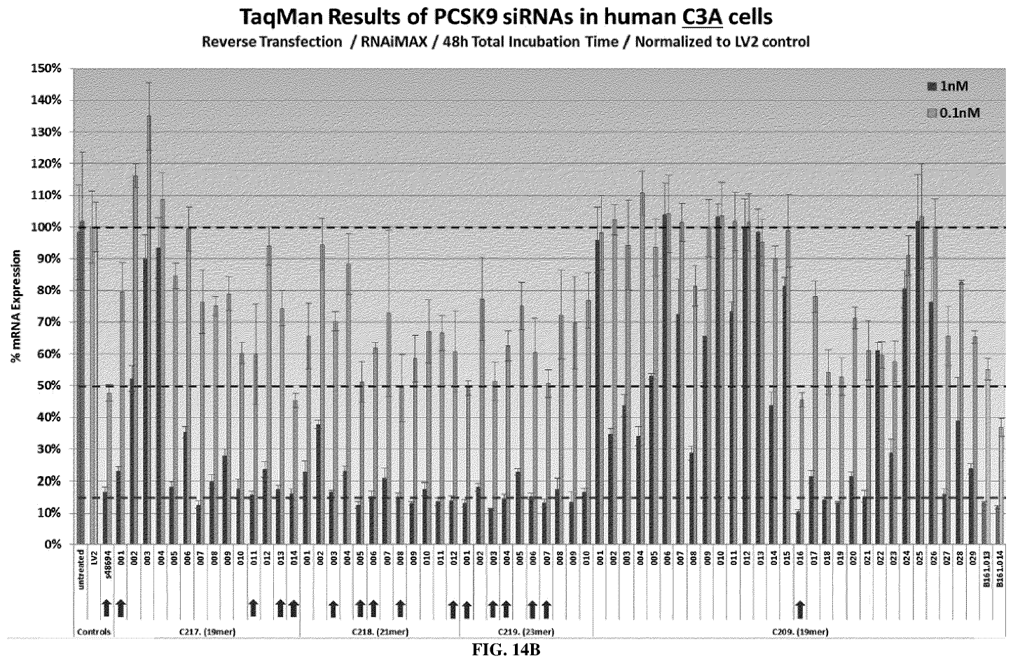

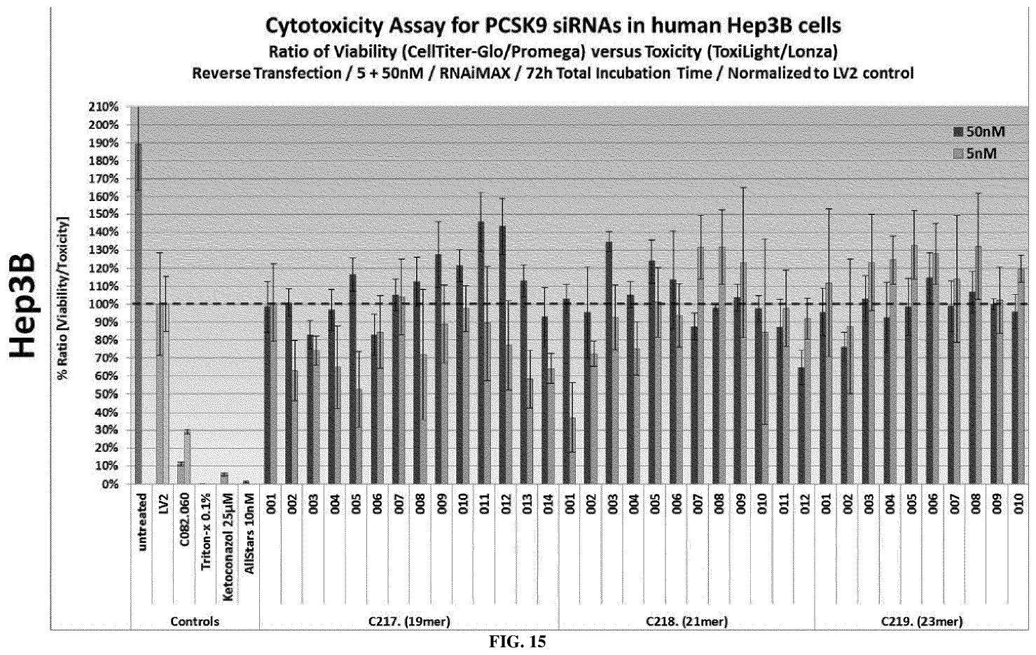

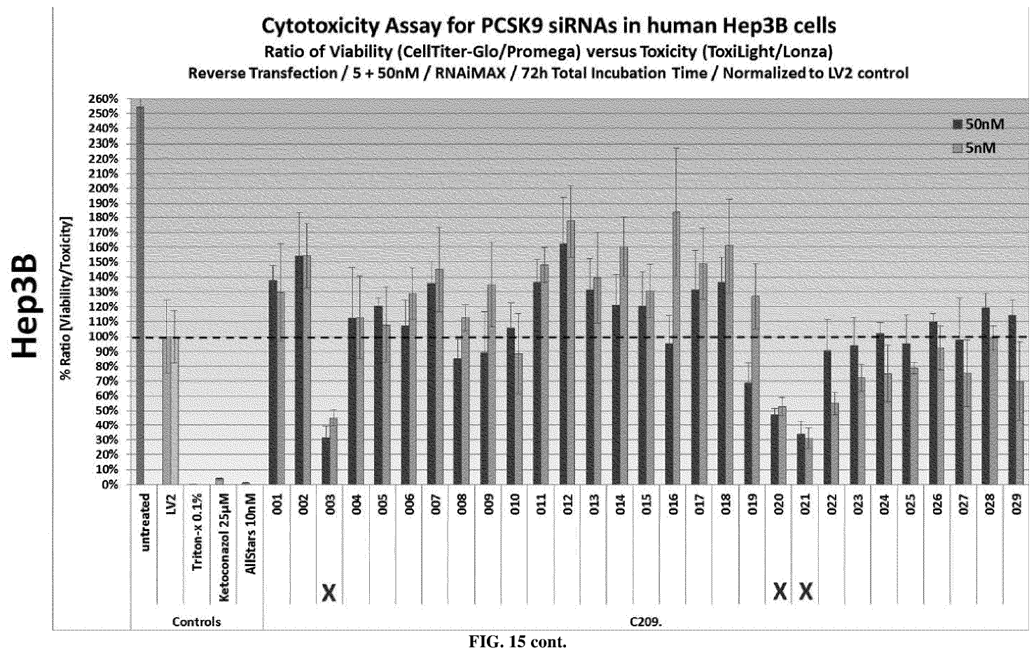

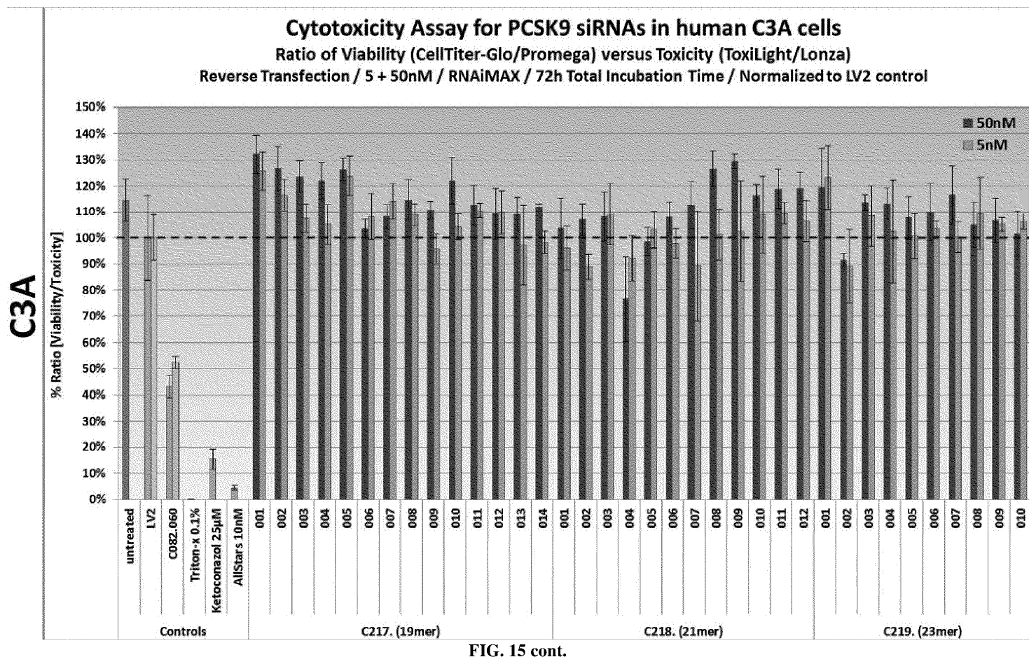

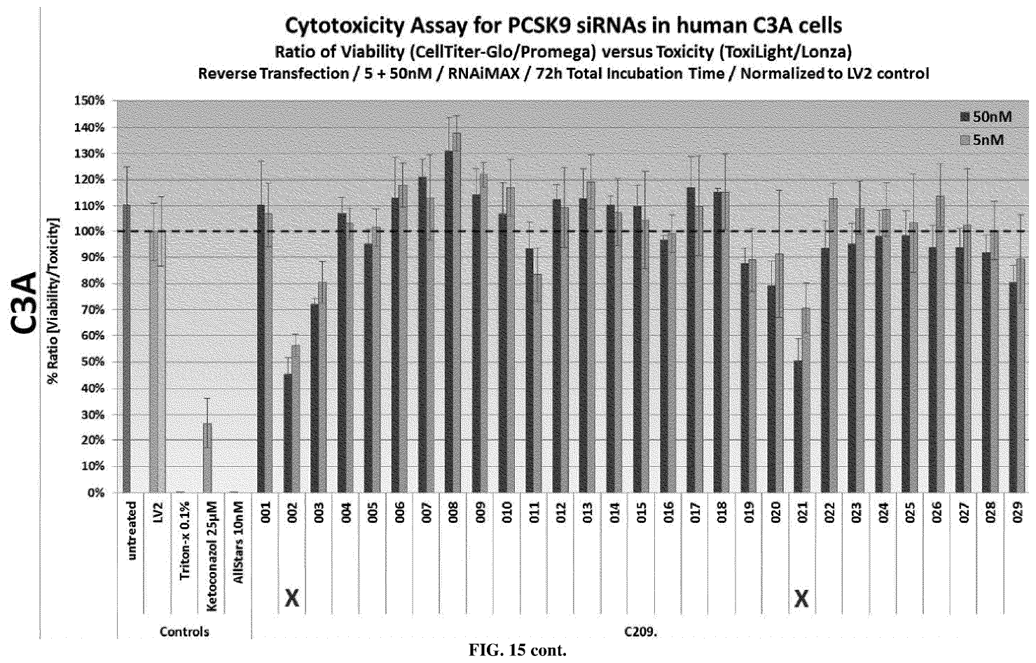

An additional set of siRNAs targeting PCSK9 were designed using a looser off-target filter criterion, as well as allowing for greater variation in siRNA length (19, 21, and 23mers), and these additional siRNAs were synthesized. Their ability to knock down PCSK9 mRNA expression in human Hep3B (FIG. 14A), and human C3A cells (FIG. 14B) was next tested using 0.1 and 1 nM siRNA transfections. The relative cytotoxicity of the transfected siRNAs was also tested in these two human cell types at 5 and 50 nM concentrations (FIG. 15). A toxic effect was observed in both human cell types when treated with the siRNA C209.021. The IC₅₀ values of the siRNAs were calculated for the 15 most active and non-toxic sequences using the human Hep3B cells (Table F) and human C3A cells (Table G). IC₅₀ values in human Hep3B cells ranged from 3.3-45.2 pM, while IC₅₀ values in human C3A cells ranged from 14.1-102.0 pM. The best maximum knockdown in both cell types was obtained using siRNA C209.016.

TABLE F
IC₅₀ activities of additional PCSK9
siRNAs in Hep3B cells

| siRNA: | IC₅₀ (pM): | Iₘₐₓ %: |
|---|---|---|
| C217.001 | 39.8 | 93.0 |
| C217.011 | 26.7 | 95.1 |
| C217.013 | 32.6 | 95.7 |
| C217.014 | 5.2 | 92.4 |
| C218.003 | 32.4 | 89.8 |
| C218.005 | 27.9 | 95.2 |
| C218.006 | 34.3 | 88.9 |
| C218.008 | 3.3 | 91.8 |
| C218.012 | 4.2 | 95.2 |
| C219.001 | 11.8 | 90.4 |
| C219.003 | 21.2 | 95.2 |
| C219.004 | 32.0 | 91.9 |
| C219.006 | 45.2 | 87.9 |
| C219.007 | 32.1 | 93.0 |
| C209.016 | 4.9 | 96.6 |
| S48694 (Ambion) | 6.6 | 95.1 |

TABLE G
IC₅₀ activities of additional PCSK9
siRNAs in C3A cells

| siRNA: | IC₅₀ (pM): | Iₘₐₓ %: |
|---|---|---|
| C217.001 | 102.0 | 94.4 |
| C217.011 | 50.7 | 93.4 |
| C217.013 | 31.0 | 92.0 |
| C217.014 | 17.4 | 93.4 |
| C218.003 | 40.8 | 92.2 |
| C218.005 | 46.7 | 95.7 |
| C218.006 | 49.7 | 90.3 |
| C218.008 | 51.4 | 91.9 |
| C218.012 | 33.2 | 94.4 |
| C219.001 | 33.8 | 88.9 |
| C219.003 | 86.3 | 95.3 |
| C219.004 | 66.1 | 93.5 |
| C219.006 | 73.4 | 93.1 |
| C219.007 | 65.4 | 94.2 |
| C209.016 | 14.1 | 96.8 |
| S48694 (Ambion) | 41.5 | 91.4 |

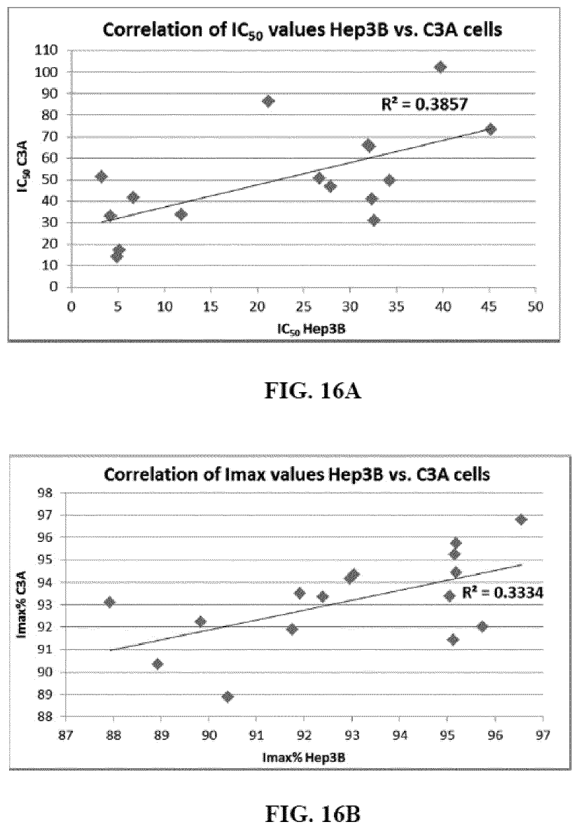

Finally, a comparison was made to understand the correlation between the calculated IC₅₀ values (FIG. 16A) in the Hep3B vs. C3A cells, as well as the correlation between the Iₘₐₓ values (FIG. 16B) in these two cell types.

Taken together, the data provided in this example show that two additional PCSK9 siRNA sequences, C209.016 and C218.012, had a good activity profile in all of the in vitro assays applied. C218.012 represents a nucleotide extended sequence as compared to C032.012.

### Example 5: Lead Optimization of GalNAc-Conjugated PCSK9 siRNA Sequences

Methods

Unless indicated otherwise, all experiments were carried out as described in the examples above. GalNAc-siRNAs, including those comprising nucleotide analogs described above, were generated based on the indicated sequences (see sequence listings above) as described in WO 2019/170731.

Results

Based on the results from Examples 3 and 4, three parent PCSK9 siRNA sequences were selected (siRNA IDs B014/C032.012/C217.014, B006/C032.006/C217.001, and C209.016/C217.007) and each molecule synthesized with three consecutive, GalNAc-conjugated nucleotide analogs at the 5′ end of respective siRNA sense strands (siRNA IDs C027.001, C027.002, and C027.003; Table 4 above). The parent sequences of siRNA IDs C027.001, C027.002, and C027.003 were then used for an optimization campaign that included 66 different chemical modifications per siRNA sequence. The resulting sequences and modification pattern are shown in Table 4, above.

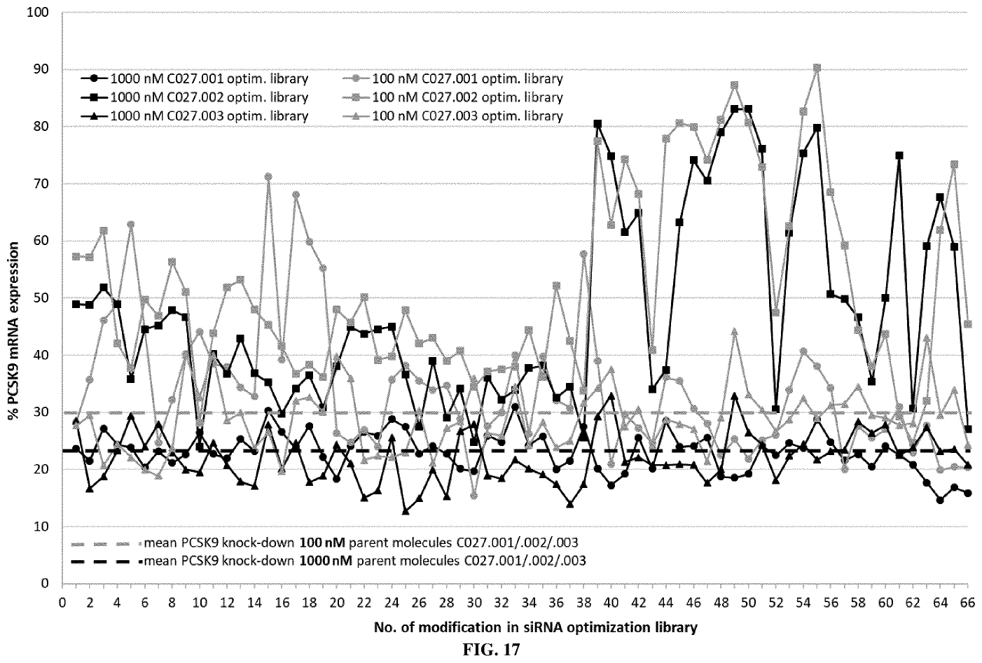

The in vitro activity of these optimization libraries was tested in cryopreserved primary human hepatocytes under free uptake conditions using 10 nM, 100 nM, and 1000 nM concentrations of PCSK9 GalNAc-siRNAs. As depicted in FIG. 17, the optimization libraries based on parent sequences C027.001 and C027.003 were identified to exhibit higher overall in vitro activities as compared to parent sequence C027.002. Of note, numerous modification patterns were identified that strongly impaired the siRNA activity of the molecule, in particular for parent sequence C027.002. On the other hand, a large number of sequence modifications were identified that led to improved knockdown activities as compared to the respective parent molecules.

In order to evaluate improved stability features of the optimized PCSK9 GalNAc-siRNAs, the optimization libraries were assayed for their in vitro half-lives in 50% mouse serum. As demonstrated in Table H, a large number of modifications were identified with improved nuclease stability as compared to the respective parent molecules which was most evident for the optimization library of parent siRNA ID C027.001.

TABLE H
Nuclease stability of optimized PCSK9 GalNAc-siRNA
constructs in 50% mouse serum

| siRNA ID | t_{1/2} |
|---|---|
| C027.001 (parent) | >24 h |
| C027.001#01 | 168 h |
| C027.001#02 | 168 h |
| C027.001#03 | 168 h |
| C027.001#04 | 168 h |
| C027.001#05 | >48 h |
| C027.001#06 | >72 h |
| C027.001#07 | 168 h |
| C027.001#08 | 168 h |
| C027.001#09 | >72 h |
| C027.001#10 | >72 h |
| C027.001#11 | >96 h |
| C027.001#12 | >168 h |
| C027.001#13 | 96 h |
| C027.001#14 | 96 h |
| C027.001#15 | >24 h |
| C027.001#16 | 96 h |
| C027.001#17 | 96 h |
| C027.001#18 | 96 h |
| C027.001#19 | 96 h |
| C027.001#20 | 96 h |
| C027.001#21 | 168 h |
| C027.001#22 | 96 h |
| C027.001#23 | 96 h |
| C027.001#24 | >48 h |
| C027.001#25 | >72 h |
| C027.001#26 | 96 h |
| C027.001#27 | 96 h |
| C027.001#28 | 96 h |
| C027.001#29 | 96 h |
| C027.001#30 | 96 h |
| C027.001#31 | 96 h |
| C027.001#32 | 96 h |
| C027.001#33 | >48 h |
| C027.001#34 | 48 h |
| C027.001#35 | 48 h |
| C027.001#36 | 48 h |
| C027.001#37 | 48 h |
| C027.001#38 | 48 h |
| C027.001#39 | 96 h |
| C027.001#40 | 168 h |
| C027.001#41 | 96 h |
| C027.001#42 | 96 h |
| C027.001#43 | >24 h |
| C027.001#44 | 96 h |
| C027.001#45 | 96 h |
| C027.001#46 | 96 h |
| C027.001#47 | 96 h |
| C027.001#48 | 96 h |
| C027.001#49 | 96 h |
| C027.001#50 | 96 h |
| C027.001#51 | 168 h |
| C027.001#52 | >48 h |
| C027.001#53 | >96 h |
| C027.001#54 | >96 h |
| C027.001#55 | >96 h |
| C027.001#56 | >96 h |
| C027.001#57 | 96 h |
| C027.001#58 | 96 h |
| C027.001#59 | 96 h |
| C027.001#60 | 96 h |
| C027.001#61 | >72 h |
| C027.001#62 | >96 h |
| C027.001#63 | >96 h |
| C027.001#64 | 96 h |
| C027.001#65 | 96 h |
| C027.001#66 | >96 h |
| C027.002 (parent) | >72 h |
| C027.002#01 | 96 h |
| C027.002#02 | 96 h |
| C027.002#03 | 96 h |
| C027.002#04 | 96 h |
| C027.002#05 | >72 h |
| C027.002#06 | >96 h |
| C027.002#07 | >72 h |
| C027.002#08 | >96 h |
| C027.002#09 | 96 h |
| C027.002#10 | >72 h |
| C027.002#11 | 96 h |
| C027.002#12 | >72 h |
| C027.002#13 | >72 h |
| C027.002#14 | >96 h |
| C027.002#15 | >48 h |
| C027.002#16 | >72 h |
| C027.002#17 | 96 h |
| C027.002#18 | 96 h |
| C027.002#19 | >72 h |
| C027.002#20 | 96 h |
| C027.002#21 | 96 h |
| C027.002#22 | 96 h |
| C027.002#23 | 96 h |
| C027.002#24 | 96 h |
| C027.002#25 | 96 h |
| C027.002#26 | 96 h |
| C027.002#27 | 96 h |
| C027.002#28 | 96 h |
| C027.002#29 | >72 h |
| C027.002#30 | >72 h |
| C027.002#31 | 72 h |
| C027.002#32 | 72 h |
| C027.002#33 | 72 h |
| C027.002#34 | 72 h |
| C027.002#35 | >72 h |
| C027.002#36 | >96 h |
| C027.002#37 | 72 h |
| C027.002#38 | 96 h |
| C027.002#39 | 72 h |
| C027.002#40 | 72 h |
| C027.002#41 | 72 h |
| C027.002#42 | >96 h |
| C027.002#43 | 72 h |
| C027.002#44 | >96 h |
| C027.002#45 | >168 h |
| C027.002#46 | >72 h |
| C027.002#47 | >96 h |
| C027.002#48 | 72 h |
| C027.002#49 | >96 h |
| C027.002#50 | 72 h |
| C027.002#51 | >72 h |
| C027.002#52 | >48 h |
| C027.002#53 | 72 h |
| C027.002#54 | 96 h |
| C027.002#55 | >96 h |
| C027.002#56 | >96 h |
| C027.002#57 | 96 h |
| C027.002#58 | >72 h |
| C027.002#59 | >96 h |
| C027.002#60 | >96 h |
| C027.002#61 | >48 h |
| C027.002#62 | >72 h |
| C027.002#63 | 96 h |
| C027.002#64 | >96 h |
| C027.002#65 | >96 h |
| C027.002#66 | >48 h |
| C027.003 (parent) | 48 h |
| C027.003#01 | >48 h |
| C027.003#02 | >48 h |
| C027.003#03 | >48 h |
| C027.003#04 | >48 h |
| C027.003#05 | 24 h |
| C027.003#06 | 72 h |
| C027.003#07 | >48 h |
| C027.003#08 | >72 h |
| C027.003#09 | 48 h |
| C027.003#10 | >48 h |
| C027.003#11 | >48 h |
| C027.003#12 | >48 h |
| C027.003#13 | >48 h |
| C027.003#14 | >48 h |
| C027.003#15 | 24 h |
| C027.003#16 | >48 h |
| C027.003#17 | >48 h |
| C027.003#18 | >48 h |
| C027.003#19 | >48 h |
| C027.003#20 | >48 h |
| C027.003#21 | >48 h |
| C027.003#22 | >48 h |
| C027.003#23 | >48 h |
| C027.003#24 | >24 h |
| C027.003#25 | >24 h |
| C027.003#26 | >24 h |
| C027.003#27 | >48 h |
| C027.003#28 | >24 h |
| C027.003#29 | >48 h |
| C027.003#30 | >48 h |
| C027.003#31 | >48 h |
| C027.003#32 | >48 h |
| C027.003#33 | >48 h |
| C027.003#34 | >48 h |
| C027.003#35 | >48 h |
| C027.003#36 | >48 h |
| C027.003#37 | >24 h |
| C027.003#38 | >24 h |
| C027.003#39 | >48 h |
| C027.003#40 | >24 h |
| C027.003#41 | >48 h |
| C027.003#42 | >24 h |
| C027.003#43 | >24 h |
| C027.003#44 | >48 h |
| C027.003#45 | >24 h |
| C027.003#46 | >48 h |
| C027.003#47 | >48 h |
| C027.003#48 | 48 h |
| C027.003#49 | 48 h |
| C027.003#50 | >48 h |
| C027.003#51 | >24 h |
| C027.003#52 | 24 h |
| C027.003#53 | 48 h |
| C027.003#54 | >24 h |
| C027.003#55 | >48 h |
| C027.003#56 | >48 h |
| C027.003#57 | >48 h |
| C027.003#58 | >48 h |
| C027.003#59 | >48 h |
| C027.003#60 | >48 h |
| C027.003#61 | >48 h |
| C027.003#62 | >48 h |
| C027.003#63 | >48 h |
| C027.003#64 | >48 h |
| C027.003#65 | >48 h |
| C027.003#66 | >48 h |

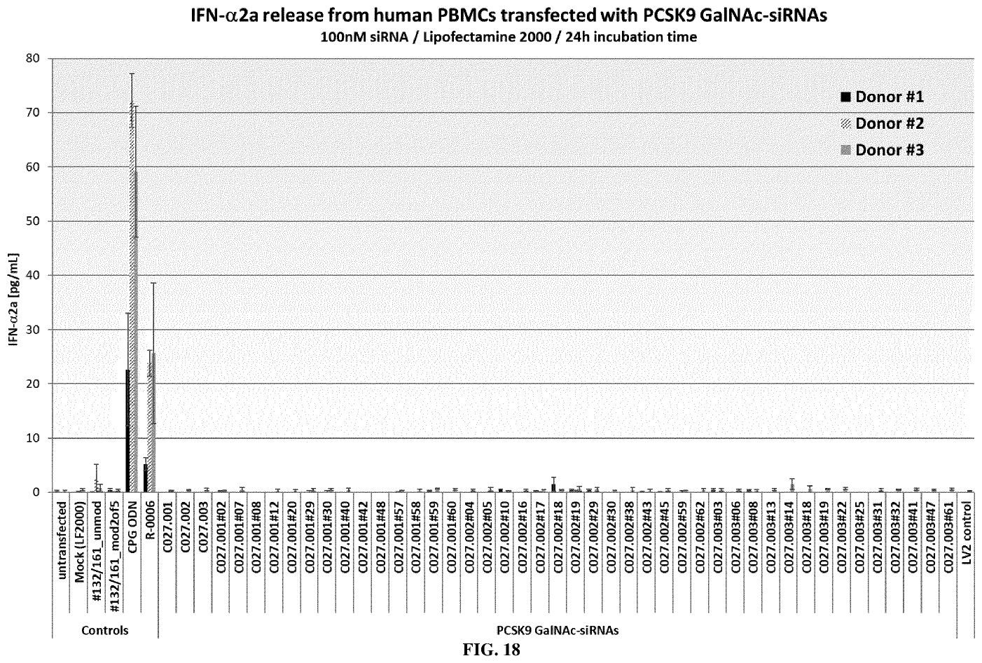

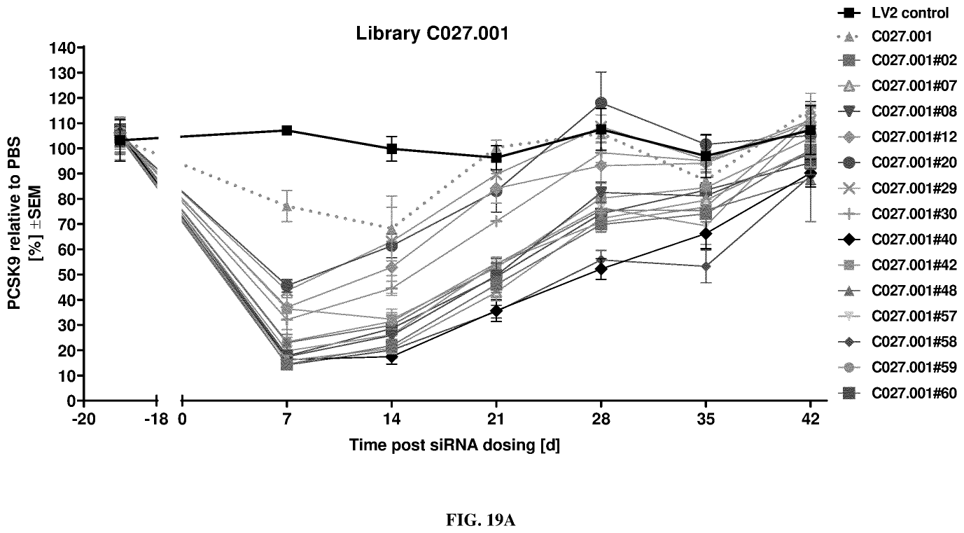

Prior to in vivo activity testing, 14 siRNA modifications were selected for each of the three different parent sequences of siRNA IDs C027.001, C027.002 and C027.003 based on siRNA activity, stability as well as chemical considerations. The immune stimulatory potential was measured in the human PBMC assay using IFNα2a secretion to the supernatant as readout (FIG. 18). No signs of immune stimulation in human PBMCs were observed for any of the tested PCSK9 GalNAc-siRNAs.

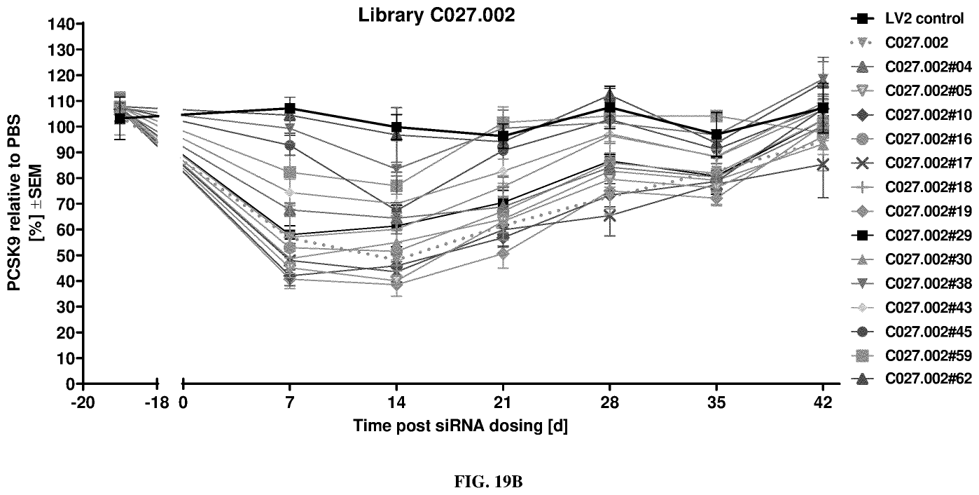

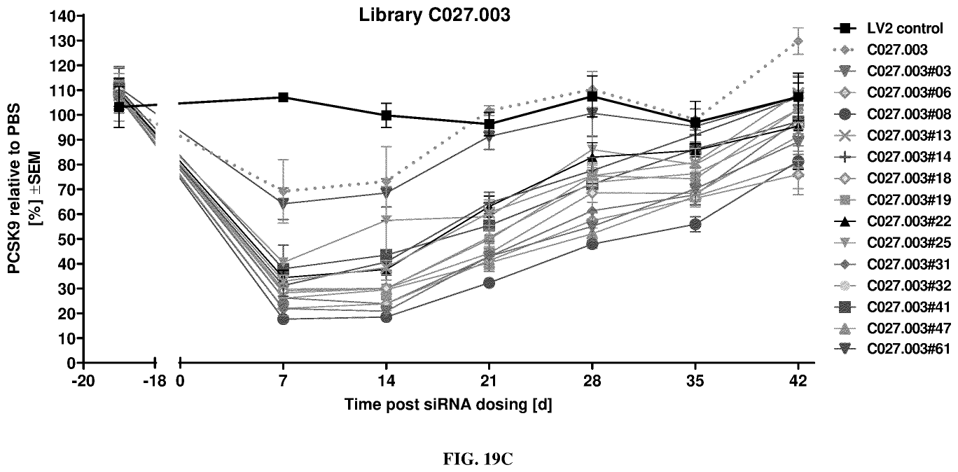

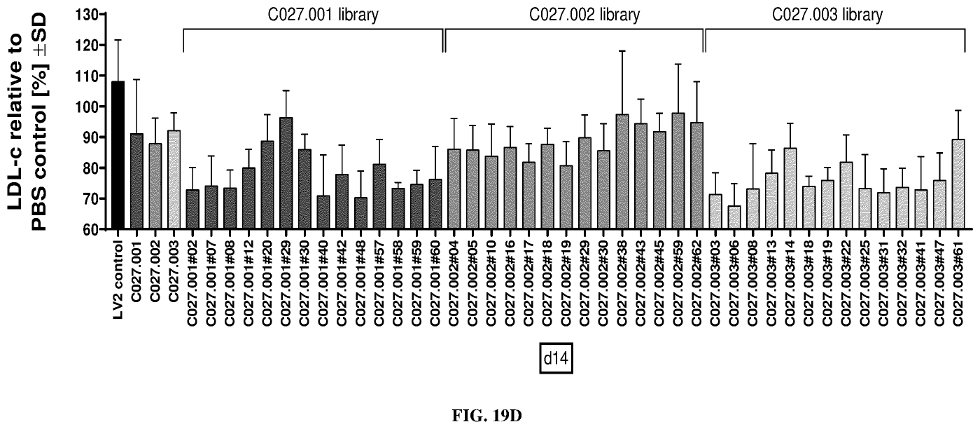

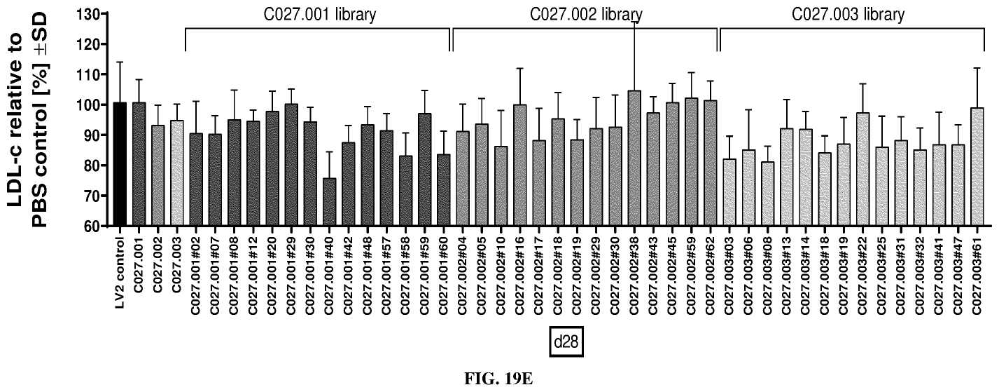

Next, in total 42 out of 198 optimized PCSK9 GalNAc-siRNAs based on the three different parent sequences were used for in vivo pharmacology testing in human PCSK9 transgenic mice and compared to the respective parent molecules C027.001, C027.002 and C027.003 (FIGS. 19A-C). After subcutaneous administration of the selected compounds at a single 6 mg/kg dose, maximum target PCSK9 protein knockdown (KDₘₐₓ) of 86% (C027.001#58), 62% (C027.002#19) and 82% (C027.003#08) were achieved between day 7 and 14 for the three respective optimization libraries when compared to animals treated with PBS vehicle control. The gain of in vivo activity was most striking for the libraries of parent sequences C027.001 and C027.003 which exhibited KDₘₐₓ values of 32% and 31%, respectively and a return to baseline 3 weeks after dosing. Interestingly, the potency gain for the library of parent sequence C027.002 (KDₘₐₓ=52%) was less pronounced. This was also reflected on KD₅₀ level (50% of maximum knockdown) which was reached at day ˜20 for the best molecule of library C027.002 (C027.002#19). Instead, libraries C027.001 and C027.003 reached KD₅₀ at days ˜26 and ˜30, respectively for molecules C027.001#40 and C027.003#08. Molecules identified with the best overall in vivo pharmacology profile on PCSK9 level (KDₘₐₓ and KD₅₀) in this study were C027.001#40, C027.001#58, C027.003#03, C027.003#06, C027.003#08 and C027.003#47.

In the same study serum LDL cholesterol (LDL-c) was measured at days 14 and 28 after siRNA dosing (FIGS. 19D and 19E). This analysis confirmed the identification of a large number of optimized molecules with an improved in vivo pharmacology profile as compared to the respective parent sequences. A maximum LDL-c reduction of 32% was achieved for siRNA ID C027.003#06 at day 14 after dosing.

A summary of selected siRNAs used in the examples is shown in Table I below.

TABLE I
siRNAs used in the examples.

| | | | Target start |
|---|---|---|---|
| | | | position in |
| | | | human PCSK9 |
| siRNA | | SEQ ID | mRNA |
| name(s) | Target sequence (5′→3′) | NO. | (NM_174936.3) |
| B001; | UUGUAGCAUUUUUAUUAAU | 3 | 3649 |
| C032.004 | | | |
| B003; | GUAGCAUUUUUAUUAAUAU | 4 | 3651 |
| C032.005 | | | |
| B006; | GCAUUUUUAUUAAUAUGGU | 5 | 3654 |
| C032.006; | | | |
| C217.001; | | | |
| C027.002 | | | |
| B008; | UUUUAUUAAUAUGGUGACU | 6 | 3658 |
| C032.007 | | | |
| B010; | UAUUAAUAUGGUGACUUUU | 7 | 3661 |
| C032.008 | | | |
| B011; | AUUAAUAUGGUGACUUUUU | 8 | 3662 |
| C032.009 | | | |
| B012; | UUAAUAUGGUGACUUUUUA | 9 | 3663 |
| C032.010 | | | |
| B013; | UAAUAUGGUGACUUUUUAA | 10 | 3664 |
| C032.011; | | | |
| C217.011 | | | |
| B014; | UAUGGUGACUUUUUAAAAU | 11 | 3667 |
| C032.012; | | | |
| C217.014; | | | |
| C027.001 | | | |
| C051; | GAGUGUGAAAGGUGCUGAU | 13 | 2891 |
| C032.001 | | | |
| C209.016; | UUAUUAAUAUGGUGACUUU | 310 | 3660 |
| C217.007; | | | |
| C027.003 | | | |
| C217.013 | AUAUGGUGACUUUUUAAAA | 311 | 3656 |
| C218.003 | AUUUUUAUUAAUAUGGUGACU | 312 | 3656 |
| C218.005 | UUUUAUUAAUAUGGUGACUUU | 313 | 3658 |
| C218.006 | UUUAUUAAUAUGGUGACUUUU | 314 | 3659 |
| C218.008 | UAUUAAUAUGGUGACUUUUUA | 315 | 3661 |
| C218.012 | AAUAUGGUGACUUUUUAAAAU | 316 | 3665 |
| C219.001 | GCAUUUUUAUUAAUAUGGUGACU | 317 | 3654 |
| C219.003 | AUUUUUAUUAAUAUGGUGACUUU | 318 | 3656 |
| C219.004 | UUUUUAUUAAUAUGGUGACUUUU | 319 | 3657 |
| C219.006 | UUUAUUAAUAUGGUGACUUUUUA | 320 | 3659 |
| C219.007 | UUAUUAAUAUGGUGACUUUUUAA | 321 | 3660 |

(1) CCAUUUUAUUAAUAUGGUGACUinvdT (SEQ ID NO: 176) in the sense
strand and AGUCACCAUAUUAAUAAAAdTdT (SEQ ID NO: 177) in the antisense strand,
(2) CCAUAUUAAUAUGGUGACUUUUinvdT (SEQ ID NO: 180) in the sense
strand and AAAAGUCACCAUAUUAAUAdTdT (SEQ ID NO: 181) in the antisense strand,
(3) CCAAUUAAUAUGGUGACUUUUUinvdT (SEQ ID NO: 182) in the sense
strand and AAAAAGUCACCAUAUUAAUdTdT (SEQ ID NO: 183) in the antisense strand,
(4) CCAUUAAUAUGGUGACUUUUUAinvdT (SEQ ID NO: 184) in the sense
strand and UAAAAAGUCACCAUAUUAAdTdT (SEQ ID NO: 185) in the antisense strand,
(5) CCAUAAUAUGGUGACUUUUUAAinvdT (SEQ ID NO: 186) in the sense
strand and UUAAAAAGUCACCAUAUUAdTdT (SEQ ID NO: 187) in the antisense strand,
(6) CCAUAUGGUGACUUUUUAAAAUinvdT (SEQ ID NO: 188) in the sense
strand and AUUUUAAAAAGUCACCAUAdTdT (SEQ ID NO: 189) in the antisense strand,
(7) CCAUUAUUAAUAUGGUGACUUUinvdT (SEQ ID NO: 322) in the sense
strand and AAAGUCACCAUAUUAAUAAdTdT (SEQ ID NO: 323) in the antisense strand,
(8) CCAAUAUGGUGACUUUUUAAAAinvdT (SEQ ID NO: 324) in the sense
strand and UUUUAAAAAGUCACCAUAUdtdt (SEQ ID NO: 325) in the antisense strand,
(9) CCAAUUUUUAUUAAUAUGGUGACUinvdT (SEQ ID NO: 326) in the
sense strand and AGUCACCAUAUUAAUAAAAAUdTdT (SEQ ID NO: 327) in the
antisense strand,
(10) CCAUUUUAUUAAUAUGGUGACUUUinvdT (SEQ ID NO: 328) in the
sense strand and AAAGUCACCAUAUUAAUAAAAdTdT (SEQ ID NO: 329) in the
antisense strand,
(11) CCAUUUAUUAAUAUGGUGACUUUUinvdT (SEQ ID NO: 330) in the
sense strand and AAAAGUCACCAUAUUAAUAAAdTdT (SEQ ID NO: 331) in the
antisense strand,
(12) CCAUAUUAAUAUGGUGACUUUUUAinvdT (SEQ ID NO: 332) in the
sense strand and UAAAAAGUCACCAUAUUAAUAdTdT (SEQ ID NO: 333) in the
antisense strand,
(13) CCAAAUAUGGUGACUUUUUAAAAUinvdT (SEQ ID NO: 334) in the
sense strand and AUUUUAAAAAGUCACCAUAUUdTdT (SEQ ID NO: 335) in the
antisense strand,
(14) CCAGCAUUUUUAUUAAUAUGGUGACUinvdT (SEQ ID NO: 336) in the
sense strand and AGUCACCAUAUUAAUAAAAAUGCdTdT (SEQ ID NO: 337) in the
antisense strand,
(15) CCAAUUUUUAUUAAUAUGGUGACUUUinvdT (SEQ ID NO: 338) in the
sense strand and AAAGUCACCAUAUUAAUAAAAAUdTdT (SEQ ID NO: 339) in the
antisense strand,
(16) CCAUUUUUAUUAAUAUGGUGACUUUUinvdT (SEQ ID NO: 340) in the
sense strand and AAAAGUCACCAUAUUAAUAAAAAdTdT (SEQ ID NO: 341) in the
antisense strand,
(17) CCAUUUAUUAAUAUGGUGACUUUUUAinvdT (SEQ ID NO: 342) in the
sense strand and UAAAAAGUCACCAUAUUAAUAAAdTdT (SEQ ID NO: 343) in the
antisense strand, or
(18) CCAUUAUUAAUAUGGUGACUUUUUAAinvdT (SEQ ID NO: 344) in the
sense strand and UUAAAAAGUCACCAUAUUAAUAAdTdT (SEQ ID NO: 345) in the
antisense strand.

Although the foregoing disclosure has been described in some detail by way of illustration and example for purposes of clarity of understanding, the descriptions and examples should not be construed as limiting the scope of the present disclosure.

What is claimed is:

1. A double-stranded ribonucleic acid (dsRNA), wherein the dsRNA comprises a sense strand comprising a first sequence and an antisense strand comprising a second sequence, wherein the first sequence and the second sequence are complementary, wherein the first sequence comprises a sequence selected from the group consisting of SEQ ID NOS: 6-11 and 310-321, wherein the dsRNA is optionally a small interfering RNA (siRNA) or short hairpin RNA (shRNA), and wherein the dsRNA optionally inhibits expression of a Proprotein Convertase Subtilisin Kexin 9 (PCSK9) gene.

2. The dsRNA of claim 1, wherein the dsRNA comprises:

3. A double-stranded ribonucleic acid (dsRNA), wherein the dsRNA comprises a sense strand comprising a first sequence and an antisense strand comprising a second sequence, wherein only the first sequence and the second sequence are complementary, wherein the first sequence is one of SEQ ID NOS: 3, 4, and 13, wherein the dsRNA is optionally a small interfering RNA (siRNA) or short hairpin RNA (shRNA), and wherein the dsRNA optionally inhibits expression of a Proprotein Convertase Subtilisin Kexin 9 (PCSK9) gene.

4. The dsRNA of claim 3, wherein the dsRNA comprises:
(19)
(SEQ ID NO: 162)
CCAUUGUAGCAUUUUUAUUAAUinvdT
in the sense strand and
(SEQ ID NO: 163)
AUUAAUAAAAAUGCUACAAdTdT
in the antisense strand,
(20)
(SEQ ID NO: 166)
CCAGUAGCAUUUUUAUUAAUAUinvdT
in the sense strand and
(SEQ ID NO: 167)
AUAUUAAUAAAAAUGCUACdTdT
in the antisense strand, or
(21)
(SEQ ID NO: 290)
CCAGAGUGUGAAAGGUGCUGAUinvdT
in the sense strand and
(SEQ ID NO: 291)
AUCAGCACCUUUCACACUCdTdT
in the antisense strand.

5. The dsRNA of any one of claims 1-4, wherein the first and second sequences are each less than or equal to 30 nucleotides in length, and optionally wherein the first and second sequences are each at least 19 and less than or equal to 23 nucleotides in length.

6. The dsRNA of any one of claims 1-5, wherein the dsRNA comprises one or more modified nucleotides;
wherein at least one of the one or more modified nucleotides is optionally a 2′-O-methyl nucleotide, 5′-phosphorothioate nucleotide, or a terminal nucleotide linked to a cholesterol derivative or lipophilic moiety;
wherein at least one of the one or more modified nucleotides is optionally a 2′-fluoro, 2′-deoxy, 2′-O-methoxyethyl, constrained ethyl (cEt), deoxy, inverted deoxy, inverted dideoxy, locked nucleic acid, abasic, 2′-amino, 2′-alkyl, morpholino, phosphoramidate, or a non-natural base-containing nucleotide;
wherein the dsRNA optionally comprises one or more 2′-O-methyl nucleotides and one or more 2′-fluoro nucleotides;
wherein the dsRNA optionally comprises two or more 2′-O-methyl nucleotides and two or more 2′-fluoro nucleotides in the pattern
OMe-F-OMe-F or F-OMe-F-OMe,
wherein OMe represents a 2′-O-methyl nucleotide, and wherein F represents a 2′-fluoro nucleotide; and
wherein the dsRNA optionally comprises up to 10 contiguous nucleotides that are each a 2′-O-methyl nucleotide or up to 10 contiguous nucleotides that are each a 2′-fluoro nucleotide.

7. The dsRNA of any one of claims 1-6, wherein:
(a) the dsRNA comprises one or more phosphorothioate groups, or
(b) the dsRNA does not comprise a phosphorothioate group.

8. The dsRNA of any one of claims 1-7, wherein:
(a) the dsRNA comprises one or more phosphotriester groups, or
(b) the dsRNA does not comprise a phosphotriester group.

9. The dsRNA of any one of claims 1-8, wherein the dsRNA is attached to one or more GalNAc derivatives via a linker; wherein optionally the dsRNA is attached to three GalNAc derivatives via a trivalent branched linker; and wherein optionally at least one of the one or more GalNAc derivatives is attached to the 3′ end of the sense strand, the 3′ end of the antisense strand, or the 5′ end of the sense strand of the dsRNA.

10. The dsRNA of any one of claims 1-9, wherein one or both of the sense strand and the antisense strand further comprises:
(a) a 5′ overhang comprising one or more nucleotides, wherein the 5′ overhang optionally comprises one or more thymines; and/or
(b) a 3′ overhang comprising one or more nucleotides, wherein the 3′ overhang optionally comprises two nucleotides, and wherein the 3′ overhang optionally comprises one or more thymines.

11. The dsRNA of claim 1, wherein one or both of strands of the dsRNA comprise one or more compounds having the structure of formula (I): 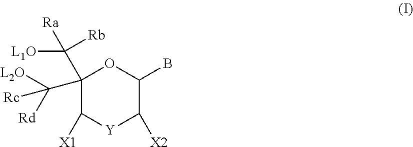
wherein:
B is a heterocyclic nucleobase;
one of L1 and L2 is an internucleoside linking group linking the compound of formula (I) to a polynucleotide and the other of L1 and L2 is H, a protecting group, a phosphorus moiety or an internucleoside linking group linking the compound of formula (I) to a polynucleotide,
Y is O, NH, NR1 or N—C(═O)—R1, wherein R1 is:
a (C1-C20) alkyl group, optionally substituted by one or more groups selected from an halogen atom, a (C1-C6) alkyl group, a (C3-C8) cycloalkyl group, a (C3-C14) heterocycle, a (C6-C14) aryl group, a (C5-C14) heteroaryl group, —O—Z1, —N(Z1)(Z2), —S—Z1, —CN, —C(=J)-O—Z1, —O—C(=J)-Z1, —C(=J)-N(Z1)(Z2), and —N(Z1)-C(=J)-Z2, wherein
J is O or S,
each of Z1 and Z2 is, independently, H, a (C1-C6) alkyl group, optionally substituted by one or more groups selected from a halogen atom and a (C1-C6) alkyl group,
a (C3-C8) cycloalkyl group, optionally substituted by one or more groups selected from a halogen atom and a (C1-C6) alkyl group,
a group —[C(═O)]m-R2-(O—CH2-CH2)p-R3, wherein
m is an integer meaning 0 or 1,
p is an integer ranging from 0 to 10,
R2 is a (C1-C20) alkylene group optionally substituted by a (C1-C6) alkyl group, —O—Z3, —N(Z3)(Z4), —S—Z3, —CN, —C(═K)—O—Z3, —O—C(═K)—Z3, —C(═K)—N(Z3)(Z4), or —N(Z3)-C(═K)—Z4, wherein
K is O or S,
each of Z3 and Z4 is, independently, H, a (C1-C6) alkyl group, optionally substituted by one or more groups selected from a halogen atom and a (C1-C6) alkyl group, and
R3 is selected from the group consisting of a hydrogen atom, a (C1-C6) alkyl group, a (C1-C6) alkoxy group, a (C3-C8) cycloalkyl group, a (C3-C14) heterocycle, a (C6-C14) aryl group or a (C5-C14) heteroaryl group, or R3 is a cell targeting moiety,
X1 and X2 are each, independently, a hydrogen atom, a (C1-C6) alkyl group, and
each of Ra, Rb, Rc and Rd is, independently, H or a (C1-C6) alkyl group,
or is a pharmaceutically acceptable salt thereof.

12. The dsRNA of claim 11, comprising one or more compounds of formula (I) wherein Y is:
a) NR1, R1 is a non-substituted (C1-C20) alkyl group;
b) NR1, R1 is a non-substituted (C1-C16) alkyl group, which includes an alkyl group selected from a group comprising methyl, isopropyl, butyl, octyl, and hexadecyl;
c) NR1, R1 is a (C3-C8) cycloalkyl group, optionally substituted by one or more groups selected from a halogen atom and a (C1-C6) alkyl group;
d) NR1, R1 is a cyclohexyl group;
e) NR1, R1 is a (C1-C20) alkyl group substituted by a (C6-C14) aryl group;
f) NR1, R1 is a methyl group substituted by a phenyl group;
g) N—C(═O)—R1, R1 is an optionally substituted (C1-C20) alkyl group; or
h) N—C(═O)—R1, R1 is methyl or pentadecyl.

13. The dsRNA of claim 11 or 12, comprising one or more compounds of formula (I) wherein B is selected from a group consisting of a pyrimidine, a substituted pyrimidine, a purine and a substituted purine, or a pharmaceutically acceptable salt thereof.

14. The dsRNA of any one of claims 11 to 13, wherein R3 is of formula (II) 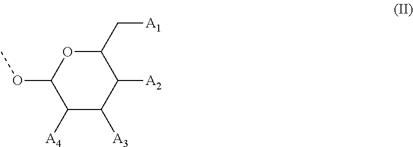
wherein A1, A2 and A3 are OH,
A4 is OH or NHC(═O)—R5, wherein R5 is a (C1-C6) alkyl group, optionally substituted by an halogen atom, or a pharmaceutically acceptable salt thereof.

15. The dsRNA of any one of claims 11 to 14, wherein R3 is N-acetyl-galactosamine, or a pharmaceutically acceptable salt thereof.

16. The dsRNA of any one of claims 11 to 15, comprising one or more nucleotides from Table A.

17. The dsRNA of any one of claims 11 to 16, comprising from 2 to 10 compounds of formula (I), or a pharmaceutically acceptable salt thereof.

18. The dsRNA of claim 17, wherein the 2 to 10 compounds of formula (I) are on the sense strand.

19. The dsRNA of any one of claims 11 to 18, wherein the sense strand comprises two to five compounds of formula (I) at the 5′ end, and/or comprises one to three compounds of formula (I) at the 3′ end.

20. The dsRNA of any one of claims 11 to 19, wherein
a) the two to five compounds of formula (I) at the 5′ end of the sense strand comprise lgT3, optionally comprising three consecutive lgT3 nucleotides; and/or
b) the one to three compounds of formula (I) at the 3′ end of the sense strand comprise lT4; optionally comprising two consecutive lT4.

21. The dsRNA of any one of claims 1 to 20, comprising one or more internucleoside linking groups independently selected from the group consisting of phosphodiester, phosphotriester, phosphorothioate, phosphorodithioate, alkyl-phosphonate and phosphoramidate backbone linking groups, or a pharmaceutically acceptable salt thereof.

22. The dsRNA of any one of claims 1 to 21, selected from the dsRNAs in Tables 2-4.

23. The dsRNA of any one of claims 1 to 22, wherein:
a) the sense strand comprises a nucleotide sequence selected from the group consisting of SEQ ID NOs: 578, 585, 587, 620, 621, 622, and 627; and/or
b) the antisense strand comprises a nucleotide sequence selected from the group consisting of SEQ ID NOs: 589, 591, 631, 632, 634, 635 and 639.

24. The dsRNA of claim 23, wherein the sense strand and antisense strand of the dsRNA respectively comprise the nucleotide sequences of:
a) SEQ ID NOs: 578 and 589; [C027.001]
b) SEQ ID NOs: 620 and 631; [C027.003]
c) SEQ ID NOs: 585 and 591; [C027.001#40]
d) SEQ ID NOs: 587 and 591; [C027.001#58]
e) SEQ ID NOs: 621 and 634; [C027.003#03]
f) SEQ ID NOs: 622 and 632; [C027.003#06]
g) SEQ ID NOs: 622 and 635; [C027.003#08] and
h) SEQ ID NOs: 627 and 639; [C027.003#47].

25. A vector encoding the dsRNA of any one of claims 1-24.

26. An isolated host cell comprising the dsRNA of any one of claims 1-24 or the vector of claim 25.

27. A composition comprising the dsRNA of any one of claims 1-24, wherein optionally the composition further comprises a pharmaceutically acceptable carrier, wherein optionally the composition further comprises a delivery vehicle, and wherein optionally the delivery vehicle is selected from the group consisting of a liposome, lipoplex, complex, and nanoparticle.

28. The dsRNA of any one of claims 1-24 or the composition of claim 27 for use in a method of inhibiting expression of a PCSK9 gene in a subject, wherein optionally the expression of the PCSK9 gene in the liver of the subject is inhibited by the dsRNA, and wherein optionally the subject is a human.

29. The dsRNA of any one of claims 1-24 or the composition of claim 27 for use in a method of treating or preventing a PCSK9-mediated disease in a subject in need thereof, wherein optionally the PCSK9-mediated disorder is hypercholesterolemia, wherein optionally the expression of the PCSK9 gene in the liver of the subject is inhibited by the dsRNA, and wherein optionally the subject is a human.
